(19) United States
(12) Patent Application Publication
(10) Pub. No.: US 2023/0384322 A1
(43) Pub. Date: Nov. 30, 2023

(54) COMPLEMENTOME ASSAY

(71) Applicant: Complement Therapeutics Limited, London (GB)

(72) Inventors: Paul BISHOP, Greater Manchester (GB); Simon CLARK, Baden-Wurttemberg (DE); Richard UNWIN, Greater Manchester (GB)

(21) Appl. No.: 18/184,427

(22) Filed: Mar. 15, 2023

Related U.S. Application Data
(63) Continuation of application No. PCT/EP21/75527, filed on Sep. 16, 2021.

(30) Foreign Application Priority Data
Sep. 16, 2020 (GB) 2014570.2
May 7, 2021 (GB) 2106570.1

(51) Int. Cl. G01N 33/68

(52) U.S. Cl. CPC G01N 33/6893; G01N 33/6848; G01N 2800/16; G01N 2333/4716

(57) ABSTRACT

Methods of identifying subjects having complement-related disorders, or at risk of such disorders, are disclosed. Also disclosed are methods for selecting subjects for treatment with complement-targeted therapies, and methods of treatment of subjects with such therapies.

### CROSS-REFERENCE TO RELATED APPLICATIONS

This application is a continuation of International Application No. PCT/EP2021/075527 filed Sep. 16, 2021, which claims priority from British Patent Application No. GB 2014570.2 filed Sep. 16, 2020 and British Patent Application No. GB 2106570.1 filed May 7, 2021, the contents and elements of which are herein incorporated by reference for all purposes.

### REFERENCE TO A SEQUENCE LISTING

This application contains a sequence listing named “405217-005US_SL.xml” in XML file format, created on Mar. 15, 2023, being 221 kb in size, which is herein incorporated by reference as though fully disclosed.

### FIELD OF THE INVENTION

The present invention relates to the fields of molecular biology and medicine. More specifically, the present invention relates to methods for detecting complement proteins and the use of such methods for diagnostic and therapeutic uses.

### BACKGROUND

The complement system contributes to innate host immune defence by assisting in the rapid recognition and elimination of microbial intruders. However, dysregulation of complement can contribute to inflammatory, immune-related, and age-related conditions. As a result inappropriate regulation of the complement system has been implicated in a wide variety of diseases in humans e.g. diseases of the eye and kidney, as well as neurological diseases and cancer (Morgan, B. P, Semin Immunopathol, 2018. 40(1): p. 113-124; Halbgebauer, R., et al., Semin Immunol, 2018. 37: p. 12-20; Ma, Y., et al., Aging Dis, 2019. 10(2): p. 429-462; and Kleczko, E. K., et al., Front Immunol, 2019. 10: p. 954.

Complement pathway activation and control is regulated by a complex interplay between pathway activators and inhibitors. These activators and inhibitors are commonly enzymes which cleave and inactivate complement molecules on biological surfaces and/or in solution to maintain steady regulation of complement activating species. The complement pathways are in a constant state of flux and balance, and disturbances to this balance can lead to inappropriate activation and the consequences above.

One activating molecule is complement component 3 (C3), a member of the alternative complement pathway and amplification loop. C3 comprises a β chain and an α′ chain which associate through interchain disulphide bonds. During complement activation, C3 is cleaved to generate two functional fragments, C3a and C3b. C3a is a potent anaphylatoxin. Deposition of C3b on biological surfaces, e.g. extracellular matrix and cell surfaces, is the central activating mechanism of the alternative pathway. C3b is a potent opsonin, targeting pathogens, antibody-antigen immune complexes and apoptotic cells for phagocytosis by phagocytes and NK cells. Surface-linked C3b also reacts with other complement proteins to form active convertase enzymes that are able to produce further (surface-attachable) C3b molecules, serving to activate and amplify complement responses (Clark, S. J., et al., J Immunol, 2014. 193(10): p. 4962-70). C3b associates with Factor B to form the C3bBb-type C3 convertase and with C3bBb to form the C3bBb3b-type C5 convertase. Proteolytic cleavage of C3 also produces C3a and C3b through the classical complement pathway and the lectin pathway.

Insufficient control of C3 convertases results in massive production of C3b and C3a molecules and a shift of the complement cascade to its terminal lytic pathway. This produces the potent anaphylatoxin, C5a, and the cell lytic protein complex termed the membrane attack complex; both providing strong inflammatory signals (Clark, S. J., et al., supra). This ultimately leads to cell/tissue destruction and a local inflammatory response.

C3b activation of complement is regulated by complement protein factor I (FI). FI prevents complement activation by cleaving C3b to a proteolytically-inactive form, designated iC3b, which is unable to participate in convertase assembly, and further to downstream products iC3dg and C3d. FI requires the presence of a cofactor, examples of which include the blood-borne Factor H (FH) protein and the membrane-bound surface co-factor ‘complement factor 1’ (CR1; CD35). FH and CR1 also help to exert decay-accelerating activity, which can assist in the deconstruction of already formed C3 convertases.

FH is encoded by the CFH gene on human chromosome 1q32 within the RCA (regulators of complement) gene cluster. There is a naturally-occurring truncated form of FH called FH-like protein 1 (FHL-1) which arises from alternative splicing of the CFH gene and has cofactor activity like FH. FH comprises 20 CCP domains. FHL-1 is identical to FH for the first seven CCP domains before terminating with a unique 4-aa C terminus.

Proteins encoded by the CFHR1-5 genes at the RCA locus also exert complement regulatory functions. The CFHR1-5 genes encode a group of five secreted plasma proteins (FHR1 to FHR5) synthesised primarily by hepatocytes. The FHR proteins retain some sequence homology with C3b binding domains of FH and are thought to enhance complement activation (Skerka et al., Mol Immunol. 2013, 56:170-180).

One complement-related disorder is macular degeneration, e.g. age-related macular degeneration (AMD). Macular degeneration is believed to be driven in part by complement-mediated attack on ocular tissues. A major driver of AMD risk is genetic variation at the RCA locus resulting in dysregulation of the complement cascade. AMD is the leading cause of blindness in the developed world: currently responsible for 8.7% of all global blind registrations. It is estimated that 196 million people will be affected by 2020, increasing to 288 million by 2040 (Wong et al. Lancet Glob Heal (2014) 2:e106-16). AMD manifests as the progressive destruction of the macula, the central part of the retina at the back of the eye, leading to loss of central visual acuity. Early stages of the disease see morphological changes in the macula such as the loss of blood vessels in the choriocapillaris (Whitmore et al., Prog Retin Eye Res (2015) 45:1-29); a layer of capillaries found in the choroid (a highly vascularized layer that supplies oxygen and nutrition to the outer retina). The choriocapillaris is separated from the metabolically active retinal pigment epithelium (RPE) by Bruch's membrane (BrM); a thin (2-4 μm), acellular, five-layered sheet of extracellular matrix. The BrM serves two major functions: the substratum of the RPE and a blood vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp 466-481, which is hereby incorporated by reference in its entirety.

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. The key characteristics of AMD are indicative of over-active complement, including cell/tissue destruction and a local inflammatory response. Hallmark lesions of early AMD, termed drusen, develop within BrM adjacent to the RPE layer (Bird et al, Surv Ophthalmol 1995, 39(5):367-374). Drusen are formed from the accumulation of lipids, proteins and cellular debris, and include a swathe of complement activation products (Anderson et al., Prog Retin Eye Res 2009, 29:95-112; Whitcup et al., Int J Inflam 2013, 1-10). The presence of drusen within BrM disrupts the flow of nutrients from the choroid across this extracellular matrix to the RPE cells, which leads to cell dysfunction and eventual death, leading to the loss of visual acuity.

‘Dry’ AMD, also known as geographic atrophy, represents around 50% of late-stage AMD cases. In the remaining percentage of late-stage cases, choroidal neovascularisation (CNV) develops, in which the increased synthesis of vascular endothelial growth factor (VEGF) by RPE cells promotes new blood vessel growth from the choroid/choriocapillaris that breaks through BrM into the retina. These new blood vessels leak and eventually form scar tissue; this is referred to as ‘wet’ (neovascular or exudative) AMD. ‘Wet’ AMD is the most virulent form of late-stage AMD and has different disease characteristics to ‘dry’ AMD. There are treatments for wet AMD, where for example the injection of anti-VEGF agents into the vitreous of the eye can slow or reverse the growth of these blood vessels, although it cannot prevent their formation in the first place. Geographic atrophy (‘dry’ AMD) remains untreatable.

FHL-1 predominates at BrM, suggesting an important role for this variant in protection of retinal tissue from complement-mediated attack (Clark, S. J., et al., supra). FH is found in the blood at a higher concentration than FHL-1. Both FH and FHL-1 protect against complement over-activation in the ECM of the choroid (the capillary network underlying BrM). The role of the five FHR proteins are less well understood, although there is some evidence that they may counter the inhibitory effects of FH and FHL-1 (Clark, S. J. and P. N. Bishop, J Clin Med, 2015. 4(1): p. 18-31).

WO2019/215330 describes that FHR4 is a positive regulator of complement activation and prevents FH-mediated C3b breakdown, leading to the formation of C3 convertase and the progression of the complement activation loop. High levels of circulating FHR4, expressed from the liver, indicate an increased risk of developing complement-related disorders.

Defining the exact molecular changes and activation state underpinning the dysregulation of complement processes in human disease tissue remains problematic, largely because it requires measurements at the protein level and an understanding of the relative quantities of the different regulators. It is critical to be able to accurately measure absolute levels of FH and related RCA locus proteins in plasma, as well as levels of FI and C3b itself, for effective diagnosis and treatment of complement-related disorders. Whilst assays have been developed for FH, distinct measurement of FHL-1 and FHR1-5 is difficult due to high sequence homology between all these proteins. This sequence similarity has meant that, with the exception of the full length FH protein, it has proven difficult to generate antibodies which are specific to only one of these family members in order to obtain useful immunoassays.

In another example, a recent study quantified levels of FH and FHR1-5 using mass spectrometry but the assay was unable to detect biologically important isoform FHL-1 that is found at significant levels in the blood and at key sites of AMD pathogenesis (Zhang, P., et al., Proteomics. 2017; 17(6):10).

The present invention seeks to address these issues.

### SUMMARY

The present invention relates to detecting complement proteins and using the results of said detection to identify the risk of onset of complement-related disorders, inform treatment and treat said disorders. The present application describes a method which is capable of distinguishing between Complement Factor H, FHL-1 and the five Complement Factor H related (FHR) proteins, despite their sequence similarity. The method can also distinguish between breakdown products derived from C3, and other complement-related proteins with high sequence similarity.

In one aspect, the invention relates to a method of identifying a subject having a complement-related disorder or at risk of developing a complement-related disorder, the method comprising:
(a) determining the level of a complement protein selected from FHR1, FHR2, FHR3, FHR-4 and/or FHR5 in a blood sample obtained from the subject;
(b) determining that the subject has or is likely to develop a complement-related disorder if the level of the complement protein determined in (a) is altered, e.g. elevated, as compared to the level of that complement protein in blood in a control subject that does not have a complement-related disorder.

Step (a) may comprise determining the level of two or three of the complement proteins selected from FHR1, FHR2, FHR3, FHR4 and/or FHR5. Step (a) may comprise determining the level of any one or more of the complement proteins selected from FHR1, FHR2, FHR3, and/or FHR5. Step (a) may comprise determining the level of two or three of the complement proteins selected from FHR1, FHR2 and/or FHR3. Step (a) may comprise, or further comprise, determining the level of FHR4 and/or FHR5. The method may further comprise determining the level of FH and/or FHL-1, i.e. with or without determining FHR4 and/or FHR5. The method may comprise determining the level of FHL-1 and determining that the subject has or is likely to develop a complement-related disorder if the level of FHL-1 is altered, e.g. elevated, as compared to the level of that complement protein in blood in a control subject that does not have a complement-related disorder. The method may comprise determining all seven proteins above, or any combination of these seven as provided herein.

In some aspects, the present invention provides a method of identifying a subject having a macular degeneration or at risk of developing a macular degeneration, the method comprising:
(a) determining the level of one or more of FHR1, FHR2, FHR5, and/or FHR3 in a blood-derived sample obtained from the subject, optionally in combination with determining the level of FHR4; and
(b) determining that the subject has or is likely to develop a macular degeneration if the level of the protein determined in (a) is elevated as compared to the level of that protein in blood in a control subject that does not have a macular degeneration.

In some embodiments, the macular degeneration is one or more of Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, and/or autoimmune uveitis.

In some embodiments, step (a) comprises determining the level of two, three, or four of FHR1, FHR2, FHR5 and/or FHR3. In some embodiments, step (a) further comprises determining the level of FHR4. In some embodiments, the method further comprises determining the level of FHL-1 and/or FH, optionally determining that the subject has or is likely to develop a macular degeneration if the level of FHL-1 is elevated as compared to the level of FHL-1 in blood in a control subject that does not have a macular degeneration.

In some embodiments, the method comprises determining the level of:
(a) FHR1 and FHR2;
(b) FHR2 and FHR3;
(c) FHR1 and FHR3;
(d) FHR1, FHR2 and FHR3;
(e) FHR1, FHR2 and FHR5;
(f) FHR1, FHR2, FHR3 and FHR4;
(g) FHR1, FHR2, FHR3 and FHR5;
(h) FHR1, FHR2, FHR3, FHR4 and FHR5; or
(i) any of (a) to (h) above, or any other combination described herein, in combination with FHL-1 and/or FH.

Other combinations of proteins, as described herein, may be detected in the methods of the present disclosure depending on the disorder of interest.

Also provided is a method of identifying a subject having a complement-related disorder or at risk of developing a complement-related disorder, the method comprising:
(a) determining the level of one or more of FHR1, FHR2, FHR3, FHR4, FHR5, FH and/or FHL-1 in a blood-derived sample obtained from the subject;
(b) determining that the subject has or is likely to develop a complement-related disorder if the level of the protein determined in (a) is elevated as compared to the level of that protein in blood in a control subject that does not have a complement-related disorder.

The complement-related disorder may be selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythernatosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, Alzheimer's disease, dementia, Lewy body disease, Amyotrophic lateral sclerosis (ALS), Huntington's disease, prion diseases, cancer, lung cancer, glioblastoma multiforme (GBM), an infectious disease, SARS-CoV-2 infection and/or COVID-19.

In some embodiments, the complement-related disorder is glioblastoma multiforme (GBM). In some embodiments, step (a) comprises determining the level of FH and/or FHL-1.

In some embodiments, the complement-related disorder is an infectious disease. In some embodiments, the complement-related disorder is caused or exacerbated by SARS-CoV-2 infection, e.g. COVID-19. In some embodiments, step (a) comprises determining the level of FHL-1, FHR1, FHR2, FHR3, FHR4 and/or FHR5.

In some embodiments, the level of the one or more complement protein(s) is determined by mass spectrometry, e.g. as described herein.

In some embodiments, the step of determining, e.g. step (a), comprises:
(i) digesting at least one complement protein in a sample obtained from the subject with endoproteinase GluC to obtain one or more peptides;
(ii) performing mass spectrometry to determine the presence and/or level of the one or more peptides; and
(iii) using the results of (ii) to determine whether or not the level of the complement protein is elevated.

The following aspects are also provided and are disclosed in combination with the embodiments described herein.

A method for selecting a subject for treatment with a complement-targeted therapy, the method comprising:
(a) determining in a blood sample obtained from the subject the level of one or more of FHR1, FHR2, FHR3, FHR4 and/or FHR5, and optionally FHL-1; or the level of FH and/or FHL-1; and
(b) selecting the subject for treatment with the complement-targeted therapy if the level of the protein(s) determined in (a) is elevated as compared to the level of that protein(s) in blood in a control subject that does not have a complement-related disorder.

A method for selecting a therapeutic agent for a subject, the method comprising:
(a) determining in a blood sample obtained from the subject the level of one or more of FHR1, FHR2, FHR3, FHR4 and/or FHR5, and optionally FHL-1; or the level of FH and/or FHL-1;
(b) determining whether the level of the protein(s) is elevated as compared to the level of that protein(s) in blood in a control subject that does not have a complement-related disorder; and
(c) selecting a therapeutic agent that targets one or more protein(s) that are elevated in the sample from the subject.

A method of treating a complement-related disorder in a subject comprising:
(a) determining in a blood sample obtained from the subject the level of one or more of FHR1, FHR2, FHR3, FHR4 and/or FHR5, and optionally FHL-1; or the level of FH and/or FHL-1;
(b) determining whether the level of the protein(s) is elevated as compared to the level of that protein(s) in blood in a control subject that does not have a complement-related disorder; and
(c) treating the subject with a complement-targeted therapy.

A complement-targeted therapeutic agent for use in a method of treating a complement-related disorder in a subject, wherein the method comprises:
(a) determining in a blood sample obtained from the subject the level of one or more of FHR1, FHR2, FHR3, FHR4 and/or FHR5, and optionally FHL-1; or the level of FH and/or FHL-1;
(b) determining whether the level of the protein(s) in (a) is elevated as compared to the level of that protein(s) in blood in a control subject that does not have a complement-related disorder; and
(c) treating the subject with the complement-targeted therapeutic agent.

Use of a complement-targeted therapeutic agent in the manufacture of a medicament for the treatment of a complement-related disorder in a subject, wherein the method of treatment comprises:
(a) determining in a blood sample obtained from the subject the level of one or more of FHR1, FHR2, FHR3, FHR4 and/or FHR5, and optionally FHL-1; or the level of FH and/or FHL-1;
(b) determining whether the level of the protein(s) in (a) is elevated as compared to the level of that protein(s) in blood in a control subject that does not have a complement-related disorder; and
(c) treating the subject with the complement-targeted therapeutic agent.

In some embodiments, the complement-related disorder is selected from macular degeneration, Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, autoimmune uveitis, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, Alzheimer's disease, dementia, Lewy body disease, Amyotrophic lateral sclerosis (ALS), Huntington's disease, prion diseases, cancer, lung cancer, glioblastoma multiforme (GBM) an infectious disease, SARS-CoV-2 infection and/or COVID-19.

In some embodiments, the therapy or therapeutic agent in the methods above is an siRNA, miRNA, shRNA, antisense oligonucleotide, gapmer, peptide, polypeptide, antibody, aptamer, SOMAmer, or small molecule. In some embodiments, the therapy or therapeutic agent may reduce the level of one or more of FHR1, FHR2, FHR3, FHR4, FHR5 and/or FHL-1 e.g. as compared to the level of the protein(s) before administration of the therapy or therapeutic agent.

In some embodiments, the level of the one or more protein(s)/peptide(s) is determined by mass spectrometry. In some embodiments the step of detecting the one or more peptides, and/or determining the level of the one or more peptides, consists of measuring the one or more peptides by mass spectrometry. In some embodiments the step of detecting the one or more peptides, and/or determining the level of the one or more peptides, involves measuring the level of the one or more peptides by mass spectrometry alone.

In some embodiments, a determining step as described herein comprises:
(i) digesting at least one complement protein in a sample obtained from the subject with endoproteinase GluC to obtain one or more peptides;
(ii) performing mass spectrometry to determine the presence and/or level of the one or more peptides; and
(iii) using the results of (ii) to determine whether or not the level of the complement protein is elevated.

Also provided is a method of determining whether a subject has, or is at risk of developing, a complement-related disorder, the method comprising:
(a) digesting at least one complement protein selected from one or more of FHR1, FHR2, FHR3, FHR4 and/or FHR5, and optionally FHL-1, in a sample obtained from the subject with endoproteinase GluC to obtain one or more peptides;
(b) determining the level of the one or more peptides by mass spectrometry, or performing mass spectrometry to determine the presence and/or level of the one or more peptides;
(c) using the results of (b) to determine the level of the one or more complement proteins; and (d) determining that the subject has, or is at risk of developing, a complement-related disorder if the level of one or more complement proteins determined in (c) is elevated as compared to the level of that complement protein in blood in a control subject that does not have a complement-related disorder.

The methods described herein may further comprise determining the level of FH.

Also described herein is a method for detecting at least one complement protein in a sample, the method comprising digesting the protein(s) with endoproteinase GluC to obtain one or more peptides; and detecting the one or more peptides by mass spectrometry, e.g. performing mass spectrometry to determine the presence and/or level of the one or more peptides. The present disclosure also provides a method for determining the level of at least one complement protein in a sample, the method comprising digesting the protein(s) with endoproteinase GluC to obtain one or more peptides; and determining the level of the one or more peptides by mass spectrometry, e.g. performing mass spectrometry to determine the presence and/or level of the one or more peptides. In another aspect the invention provides a method for preparing at least one complement protein for analysis, the method comprising digesting the protein(s) with endoproteinase GluC to obtain one or more peptides.

In some embodiments the complement protein(s) is one or more of FHR1, FHR2, FHR3, FHR4, FHR5, FHL-1 and/or FH. In some embodiments the complement protein(s) is FHR1, FHR2, FHR-5 and/or FHR3.

The methods disclosed herein may further comprise the detection of one or more further complement protein(s) selected from C3, C3b, C3a, iC3b, C3f, C3c, C3dg, and/or C3d. The further complement protein(s) may be one or more of C3, C3a, C3f, C3c, and/or C3d. In some cases the further complement protein(s) is C3b and/or iC3b. In some cases the further complement protein is FI. All combinations of the above proteins are envisaged.

The methods described herein may involve detecting/determining the level of two or more complement proteins. In some embodiments, the method comprises simultaneous detection/determination of the levels of the two or more complement proteins.

In some cases the sample has been obtained from a subject. In some embodiments the methods described herein comprise a step of obtaining the sample from a subject. In some cases the sample comprises, or is derived from, blood, lymph, plasma, serum, tissue or cells. Preferably, the sample is a plasma sample.

The peptides may be any suitable peptide disclosed herein. The one or more peptides may be selected from the group consisting of:

(a)
(SEQ ID NO: 20)
VTYKCFE;
(b)
(SEQ ID NO: 21)
NGWSPTPRCIRVSFTL;
(c)
(SEQ ID NO: 22)
ATFCDFPKINHGILYDEE;
(d)
(SEQ ID NO: 23)
RGWSTPPKCRSTISAE
or
(SEQ ID NO: 24)
AMFCDFPKINHGILYDEE;
(e)
(SEQ ID NO: 25)
VACHPGYGLPKAQTTVTCTE;
(f)
(SEQ ID NO: 26)
YQCQSYYE;
(g)
(SEQ ID NO: 27)
RGWSTPPICSFTKGE;
(h)
(SEQ ID NO: 28)
GTAFVIFGIQDGE;
(i)
(SEQ ID NO: 29)
LRRQHARASHLGLARSNLDE;
(j)
(SEQ ID NO: 30)
LRRQHARASHLGLAR
and/or
(SEQ ID NO: 156)
LRRQHARASHLGLA;
(k)
(SEQ ID NO: 31)
LNLDVSLQLPSRSSKITHRIHWE;
(l)
(SEQ ID NO: 32)
LNLDVSLQLPSR;
(m)
(SEQ ID NO: 33)
RLGRE;
(n)
(SEQ ID NO: 34)
SSKITHRIHWE;
(o)
(SEQ ID NO: 35)
SASLLR
and/or
(SEQ ID NO: 157)
SASLL;
(p)
(SEQ ID NO: 36)
RLGR;
(q)
(SEQ ID NO: 37)
HLIVTPSGCGE;
and/or
(r)
any one or more of SEQ ID NO: 38 to 60.

The one or more peptides may be any suitable peptide, such as those disclosed herein e.g. any one of SEQ ID NO:20 to 60.

In certain methods described herein, the step of detecting, by mass spectrometry, involves the use of endoproteinase GluC for preparing at least one complement protein for detection by mass spectrometry, optionally for preparing at least two complement proteins for detection by mass spectrometry, e.g. any combination of proteins described herein.

Also provided is a method of determining the presence and/or level of a complement protein in a subject, the method comprising performing a method for detecting at least one complement protein in a sample, e.g. as described herein. In some embodiments the method is performed on a sample obtained from the subject.

In some embodiments the methods described herein comprise a step of treating a subject who has been determined to be at risk of developing, or to have, a complement-related disorder. Treating a subject may comprise administering a therapeutically effective amount of a complement-targeted therapy/therapeutic agent to the subject, e.g. as described herein.

Also provided is a method of selecting a patient for treatment of a complement-related disorder with a complement-targeted therapeutic agent, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

Also provided is a method of treating a subject who is suspected to have a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and based on the results of (b), treating said disorder e.g. as described herein.

Also provided is a complement-targeted therapeutic for use in a method of treating a complement-related disorder in a subject, the method comprising:
a) digesting at least one complement protein in a sample obtained from the subject with endoproteinase GluC to obtain one or more peptides;
b) determining the presence and/or level of the one or more peptides by mass spectrometry; and
c) based on the results of (b), administering an effective amount of the complement-targeted therapeutic.

In some embodiments the complement-related disorder referred to herein is selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, stroke, Parkinson's disease, and/or Alzheimer's disease.

Also provided is a kit for use in a method of detecting and/or determining the level of one or more complement protein(s) in a sample, the kit comprising endoproteinase GluC.

### DESCRIPTION

The invention arises from the measured observation that circulating levels of all five Factor H-related (FHR) proteins in human blood are elevated in individuals suffering from macular degeneration. Circulating levels of these FHR proteins derive exclusively from the liver as their only known source of expression in the human body. FHR4 levels, and likely also FHR1, 2, 3 and 5 levels, are to a large extent genetically driven. The expected ability of FHR proteins to out-compete the negative regulators of complement activation (i.e. FH and FHL-1) means their increased concentration can pre-dispose a patient to be more complement-active. FHR4 is known to accumulate in the human eye where it causes complement over activation associated with AMD, see e.g. WO2019/215330. The other FHR proteins are expected to do the same. The detection of overexpression of one or more FHR proteins, e.g. as described herein, is therefore predictive of an individual's likelihood of developing AMD. This is in contrast to previous reports, in which lower plasma FHR1 was detected in AMD patients (see Ansari et al., Hum Mol Genet, 2013, 22(23): 4857-4869), although it is likely this measurement was affected by historical difficulties in measuring absolute levels of FHR1. The methods of the present invention overcome these difficulties.

By targeting expression levels of the FHR proteins in the liver, or by intervening directly in blood, genetically-driven excess FHR proteins can be removed from the circulation, thus preventing their accumulation in tissues and the subsequent driving of damaging complement activation. In this way, complement-related disorders such as those in the eye, kidney or CNS can be treated or prevented.

Methods disclosed herein relate to the detection and quantification of complement related proteins, particularly one or more FHR proteins and optionally FHL-1 and/or FH. Such methods are useful to identity and stratify patients with disorders related to over-activity of the complement system, including macular degeneration. Such methods may be used to stratify patients based on their risk of developing complement-related disorders. In some cases, the methods of the present invention are used to identify appropriate treatments, such as treatments targeted to the specific complement proteins that are overexpressed in the patient.

Detection, differentiation and quantitation of highly similar proteins can be achieved using mass spectrometry (MS). In order to achieve good sensitivity by MS, proteins e.g. in a sample are routinely digested into peptides using a specific protease. The industry standard protease for this purpose is trypsin. Other enzymes that are commonly used to digest proteins for MS analysis include elastase, chymotrypsin or LysN.

Trypsin cleaves C-terminal to all K and R residues, provided they are not followed by a proline residue, and yields peptides which retain a basic group at their C-terminus which subsequently helps ionisation and transmission of peptides into the gas phase in a mass spectrometer. Peptides digested by trypsin tend to be ionised more efficiently during MS and thus produce a larger signal than peptides digested by non-trypsin enzymes. Using MS, individual peptides in the sample digest can be detected with a signal proportional to its abundance. The concentration of the parent protein can be derived from the relative abundance (signal) of endogenous peptide compared to an exogenous ‘standard’ peptide e.g. containing a stable isotope.

Trypsin digestion of complement proteins FH and FHL-1 does not produce peptides that can be detected individually using MS alone. The only FHL-1 specific tryptic peptide is a 4-amino acid C-terminal sequence which is too small to be detected reliably by MS techniques. The FHR proteins also share substantial sequence identity, meaning that it is hard to distinguish between them and measure them specifically using e.g. antibody-based assays.

The inventors have developed a unique targeted mass spectrometry assay using a non-standard proteolytic enzyme, GluC (V8 protease), to produce distinct proteotypic peptides for all the FHR proteins, as well as proteotypic peptides that can be used to distinguish between FHL-1 and FH, which can be used for the simultaneous detection and accurate measurement in plasma of all seven key regulatory proteins encoded from the CFH gene cluster using a single MS assay: FH, FHL-1, and FHR1, FHR2, FHR3, FHR4 and FHR5.

FHL-1 is a distinct biological entity from FH. The proteins have a similar action but the size of FHL-1 means that its distribution in the body is likely to be distinct from FH. This is apparent in the eye where FHL-1 can cross to the retinal side of Bruch's membrane, e.g. where drusen form, but the larger FH protein cannot, see e.g. Clark et al., J Immunol 2014, 193(10) 4962-4970 and Clark et al., Frontiers in Immunology 2017 8:1778, which are hereby incorporated by reference in their entirety. In this respect, there is evidence that FHL-1 is the prime driver of complement C3b turnover in the eye, meaning that levels of FHL-1 are likely to better inform disease risk than levels of FH.

GluC is also able to produce proteotypic peptides for C3b and FI, enabling direct measurement of C3b itself as well as levels of its proteolytic enzyme and required fluid-phase cofactors. Thus, the methods described herein mean that all these complement proteins can be measured using a single assay.

Furthermore, breakdown of C3b occurs via trypsin-like cleavages at basic residues (K and R) so trypsin digestion of C3b breakdown products is unable to produce useful peptides for analysis. In contrast, C3 turnover can be measured using the MS approach of the present invention because GluC digestion also produces proteotypic neopeptides from many C3 inactivation and breakdown products generated during inactivating cleavages. The inventors demonstrate herein that a series of products produced as a result of C3/C3b cleavage can be detected and quantified using the same single GluC/MS assay. This allows the concentrations of all known C3 fragments e.g. iC3b, C3c, C3dg and C3d to be determined accurately. Thus, the methods described herein can not only measure absolute levels of regulatory complement proteins, but can also track protein products resulting from C3 inactivation and thus assess complement activation and the progression of the amplification loop.

This is advantageous because the measurement of C3 breakdown products is analytically challenging. The pattern by which C3 is broken down is complex: first into C3a and C3b, followed by cleavage of C3b into iC3b (which cannot drive formation of the membrane attack complex (MAC) but can still act as an opsonin), and then subsequent inactivating cleavage of iC3b into C3c via the release of a C3dg fragment. To approach detection of these products with antibodies is problematic. While each sequential cleavage step in this cascade generates a new proteoform (a distinct form of a protein encoded from the same gene, including cleaved forms and splice variants), they share sequence homology and likely only undergo minor structural changes. Directing antibodies at each form is likely to be unsuccessful and while there are methods which can measure single components following some form of separation, e.g. C3dg following polyethylene glycol-based enrichment, simultaneous measurement of all fragments in the same sample is not currently possible.

Thus, the present invention relates to a single methodology for concurrent determination of the presence, absolute levels and relative molar ratios of up to seven individual complement-related proteins from the CFH family plus C3b-inactivating enzyme FI, central complement component C3, and seven proteins derived from C3 breakdown, which may be referred to herein as the “complementome”. The ability to detect absolute levels of so many complement-related proteins in one assay is critical for the successful detection, diagnosis and treatment of complement-related diseases.

Complement Proteins

Complement is a central part of the innate immunity that serves as a first line of defence against foreign and altered host cells. Complement is activated upon infection with microorganisms to induce inflammation and promote elimination of the pathogens. The complement system is composed of plasma proteins produced mainly by the liver or membrane proteins expressed on cell surface. Complement operates in plasma, in tissues, or within cells. For a review of the complement system, see e.g. Merle N S et al., Front Immunol. 2015 Jun. 2; 6:262, which is hereby incorporated by reference in its entirety.

The complement system can be activated via three distinct pathways: the classical pathway (CP), alternative pathway (AP) and lectin binding pathway (LP). In a healthy individual, the AP is permanently active at low levels to survey for presence of pathogens but host cells are protected against complement attack and are resistant to persistent low-level activation. C3b molecules bound to host cells are inactivated rapidly by a group of membrane-bound or plasma complement regulators.

In response to the recognition of molecular components of microorganisms, complement proteins become sequentially activated in an enzyme cascade: the activation of one protein enzymatically cleaves and activates the next protein in the cascade.

The three pathways converge into the generation of a C3 convertase, which cleaves the central complement component C3 into activation products C3b, a large fragment that acts as an opsonin (binds to foreign microorganisms to increase their susceptibility to phagocytosis), and C3a, an anaphylatoxin that promotes inflammation. Along with factor B (FB), C3b forms the C3 convertase (C3bBb) which cleaves further C3 molecules, generates more C3b and C3a, and amplifies C3b deposition on cell surfaces. This is the complement amplification loop. C3b deposition and activation of complement may occur on acellular structures (i.e. on extracellular matrix), such as Bruch's membrane (BrM) and the intercapillary septa of the choriocapillaris in the eye.

Activated C3 can trigger the lytic pathway, which can damage the plasma membranes of cells and some bacteria. C5a, another anaphylatoxin produced by this process, attracts macrophages and neutrophils and also activates mast cells.

Once activated, the complement system needs tight control, as newly generated complement activation products, e.g. C3b, can induce severe inflammation and cell damage to the host. A number of soluble as well as membrane bound complement regulators ensure regulation of complement activation at the surface of host cells and control different activation phases and sites of action (Skerka et al., Mol Immunol 2013, 56:170-180). Complement regulators are described further herein.

“Complement protein” may be used interchangeably herein with “complement regulator”, “a regulator of complement”, or “protein of the complement system” and refers to a protein component of the complement system or complement cascade, e.g. as described in Merle et al., Front. Immunol., 2015, 6:262 and Merle et al., Front. Immunol., 2015, 6:257, which are hereby incorporated by reference in their entirety. A “complement protein” referred to herein may be involved in any of the three complement pathways and/or in the amplification loop.

In some embodiments a “complement protein” referred to herein is involved in the alternative pathway and/or the complement activation loop. In some embodiments, a “complement protein” referred to herein is involved in the breakdown, turnover and/or inactivation of C3 or C3b, or is a product of said breakdown, turnover and/or inactivation.

In some embodiments herein, a “complement protein” as used herein may refer to one or more of FH, FHL-1, FHR1, FHR2, FHR3, FHR4, FHR5, FI, C3, C3b, C3a, iC3b, C3f, C3c, C3dg, and/or C3d.

Complement Factor H (FH) Family Proteins

Factor H (FH) regulates the alternative complement pathway and the amplification loop. It inhibits C3 convertase formation by competing with FB binding to C3b and also acts as a cofactor for C3b inactivation to iC3b by Factor I (FI), thus preventing inappropriate complement activation and inflammation. FH also exerts decay-accelerating activity, which can assist in the deconstruction of already formed C3 convertases, see e.g. Clark et al., J Immunol 2014, 193(10) 4962-4970, which is hereby incorporated by reference in its entirety.

The sequence of human FH (Uniprot P08603-1) is provided herein as SEQ ID NO:1. For a review of FH structure and function see e.g. Merle N S et al., Front Immunol. 2015 Jun. 2; 6:262, which is hereby incorporated by reference in its entirety.

Human FH comprises 20 CCP domains. The CFH gene also produces a truncated form of FH, called FHL-1, comprising only the first seven CCP domains before terminating with a unique 4-amino acid C terminus (Clark et al, 2014 supra). The sequence of human FHL-1 (Uniprot: P08603-2) is provided herein as SEQ ID NO:2.

In the eye, full-length FH protein is found on the choroidal side of Bruch's membrane (BrM), with particular accumulation in the choriocapillaris (capillary layer in the choroid). Small amounts have also been found in patches on the RPE side of the BrM, but no FH was observed in the BrM itself. FHL-1 on the other hand has been observed throughout BrM and other ECM structures e.g. drusen (Clark et al, 2014 supra). It is likely that FHL-1 confers greater complement protection to BrM than does FH, whereas FH provides the main protection for the ECM of the choroid. It is thought that FHL-1 is therefore a major regulator of complement in the BrM (a key site in AMD pathogenesis). The methods described herein allow for the individual detection and quantitation of FH and FHL-1.

FH, FHL-1 and FHR1-FHR5, are described in e.g. Clark et al., J Clin Med, 2015. 4(1): 18-31, which is hereby incorporated by reference in its entirety.

FHR1, FHR2, FHR3, FHR4 and FHR5, encoded by the CFHR genes, are also described in e.g. Skerka et al., Mol Immunol 2013, 56:170-180, which is hereby incorporated by reference in its entirety. These proteins are highly related and share a high degree of sequence identity. The N termini share 36-94% sequence identity, whilst the C-terminal domains are very similar to the FH C-terminus (36-100%). The high amino acid identity among family members is demonstrated by the fact that antibodies raised against FH can detect multiple FHR proteins in plasma and that antibodies generated against FHR proteins cross-react with the other FHRs. This cross-reactivity presents a challenge for purification of FHR proteins from plasma, as well as determining their concentration.

FHR proteins are divided into two groups depending on their conserved domains. FHR1 (SEQ ID NO:3), FHR2 (SEQ ID NO:3, 4), and FHR5 (SEQ ID NO:10) form Group I and are characterised by their conserved N-termini. They exist in plasma as homo- and heterodimers, mediated by the conserved N-terminal domains. Group II contains FHR3 (SEQ ID NO:6, 7) and FHR4 (SEQ ID NO:8, 9) which lack the N-terminal dimerisation domains, but which show a high degree of sequence similarity to portions of FH. All five FHR proteins comprise C-termini sequences that act to recognise and bind C3b, and which are very similar to the C-terminus of FH.

FHR1 is known to compete with FH and FHL-1 for binding to C3b. It is also reported to bind to C3b components of the C5 convertase and interfere with the assembly of the MAC (see e.g. Heinen S et al., Blood (2009) 114 (12): 2439-2447 and Hannan J P et al., PLoS One. 2016; 11(11):e0166200, which are hereby incorporated by reference in their entirety). As used herein, the term “FHR1” includes at least one of FHR1 (SEQ ID NO:3; FHRA) and a second FHR1 isoform (FHRB) with 3 point mutations, and preferably includes both FHR1 isoforms. “FHR1” refers to FHR1 from any species and includes isoforms, fragments, variants or homologues of FHR1 from any species. In preferred embodiments, “FHR1” refers to human FHR1.

FHR2 may inhibit C3 convertase activity, acting to inhibit the amplification loop, but may also activate the amplification loop. There are two FHR2 isoforms (SEQ ID NO:4 and 5). The protein has two glycosylated forms, a single glycosylated form (24 kDa) and a double glycosylated form (28 kDa). As used herein, the term “FHR2” includes at least one of the two isoforms or at least one of the glycosylated forms, and preferably includes both isoforms and any glycosylated forms. “FHR2” refers to FHR2 from any species and includes isoforms, fragments, variants or homologues of FHR2 from any species. In preferred embodiments, “FHR2” refers to human FHR2.

FHR3 binds to C3b and C3d and may have low cofactor activity for FI-mediated cleavage of C3b. FHR3 may also upregulate complement. There are two FHR3 isoforms (SEQ ID NO:6 and 7). FHR3 is detected in plasma in multiple variants (ranging from 35 to 56 kDa), reflecting the existence of four different glycosylated variants of FHR3. As used herein, the term “FHR3” includes at least one of the two isoforms or at least one of the glycosylated variants of FHR3, and preferably includes both isoforms and any glycosylated forms. “FHR3” refers to FHR3 from any species and includes isoforms, fragments, variants or homologues of FHR3 from any species. In preferred embodiments, “FHR3” refers to human FHR3.

The human CFHR4 gene encodes two proteins: FHR4A (SEQ ID NO:8) and FHR4B (SEQ ID NO:9), an alternative splice variant. WO 2019/215330 A1, hereby incorporated by reference in its entirety, describes that FHR4 is a positive regulator of complement activation and prevents FH-mediated C3b breakdown. High levels of FHR4 in tissues are likely to promote local inflammatory responses and cell lysis, leading to disorders associated with complement activation, and circulating FHR4 levels can be used as an indicator of risk of developing complement-related disorders, see e.g. Cipriani et al., Nat Commun 11, 778 (2020), hereby incorporated by reference in its entirety. As used herein, the term “FHR4” includes at least one of FHR4A isoform 1, FHR4A isoform 2 (G20 point deletion from isoform 1) or FHR4B, and preferably includes FHR4A isoforms 1 and 2 as well as FHR4B. “FHR4” refers to FHR4 from any species and includes isoforms, fragments, variants or homologues of FHR4 from any species. In preferred embodiments, “FHR4” refers to human FHR4.

FHR5 also recognises and binds to C3b on self surfaces. FHR5 appears as a glycosylated protein of 62 kDa. As used herein, the term “FHR5” includes any glycosylated variants of FHR5, and preferably includes all isoforms and any glycosylated forms. As used herein, “FHR5” refers to FHR5 from any species and includes isoforms, fragments, variants or homologues of FHR5 from any species. In preferred embodiments, “FHR5” refers to human FHR5.

Given the different roles of the different members of the CFH family in activation and amplification of complement and pathogenesis of complement-related disorders, it is important to be able to distinguish between the presence and levels of proteins encoded by all seven CFH family members. CFH family members, particularly FHR1-5, can also be used as biomarkers for diagnosing or predicting disorders in which dysregulation of complement is pathologically implicated.

C3, C3b and Breakdown Products

C3 is the central complement component. The pathways by which C3 is processed into various downstream products can lead to activation of complement, e.g. including inflammation and immune responses, or to the inactivation and regulation of complement. It is therefore important in terms of complement pathogenesis and treatment of complement-related disorders to be able to detect and measure the levels, including relative levels, of C3, C3b and their downstream components/processing products.

Processing of C3 is described, for example, in Foley et al. J Thromb Haemostasis (2015) 13: 610-618, which is hereby incorporated by reference in its entirety. Human C3 (UniProt: P01024; SEQ ID NO:12) comprises a 1,663 amino acid sequence (including an N-terminal, 22 amino acid signal peptide). Amino acids 23 to 667 encode C3 β chain (SEQ ID NO:13), and amino acids 749 to 1,663 encode C3b α′ chain (SEQ ID NO:14). C3 β chain and C3 α′ chain associate through interchain disulphide bonds (formed between cysteine 559 of C3 β chain, and cysteine 816 of the C3 α′ chain) to form C3b. C3a is a 77 amino acid fragment corresponding to amino acid positions 672 to 748 of C3 (SEQ ID NO:15), generated by proteolytic cleavage of C3 to form C3b.

Processing of C3b to the inactive form iC3b, which cannot itself promote further complement amplification, involves proteolytic cleavage of the C3b α′ chain at amino acid positions 1303 and 1320 to form an α′ chain fragment 1 (corresponding to amino acid positions 749-1663 of C3; SEQ ID NO:16), and an α′ chain fragment 2 (corresponding to amino acid positions 1321 to 1,663 of C3; SEQ ID NO:17). Thus, iC3b comprises the C3 β chain, C3 α′ chain fragment 1 and C3 α′ chain fragment 2 (associated via disulphide bonds). Cleavage of the α′ chain also liberates C3f, which corresponds to amino acid positions 1304 to 1320 of C3 (SEQ ID NO:18).

iC3b is processed further to C3c comprising the C3 β chain, C3 α′ chain fragment 2 and C3c α′ chain fragment 1 (corresponding to amino acid positions 749-954 of C3; SEQ ID NO:19). This cleavage event produces fragment C3dg (corresponding to amino acid positions 955-1303 of C3; SEQ ID NO:142), which is itself broken down into fragments C3g (corresponding to amino acid positions 955-1001 of C3; SEQ ID NO:143) and C3d (corresponding to amino acid positions 1002-1303 of C3; SEQ ID NO:144).

Processing of C3b to iC3b is performed by Complement Factor I (FI; encoded in humans by the gene CFI). Human Complement Factor I (UniProt: P05156; SEQ ID NO:11) has a 583 amino acid sequence (including an N-terminal, 18 amino acid signal peptide). Amino acids 340 to 574 of the light chain encode the proteolytic domain of FI, which is a serine protease containing the catalytic triad responsible for cleaving C3b to produce iC3b (Ekdahl et al., J Immunol (1990) 144 (11): 4269-74).

Proteolytic cleavage of C3b by FI to yield iC3b is facilitated by co-factors, including FH, CR1 and possibly some of the FHR proteins. Co-factors for FI typically bind to C3b and/or FI, and potentiate processing of C3b to iC3b by FI.

As used herein, any reference to a complement protein, e.g. C3, C3b, C3a, FH, FI etc, refers to said protein from any species and include isoforms, fragments, variants or homologues of said protein from any species. In some embodiments, the protein is a mammalian protein (e.g. cynomolgous, human and/or rodent (e.g. rat and/or murine) protein). Isoforms, fragments, variants or homologues of the complement proteins described herein may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the immature or mature protein from a given species, e.g. human protein sequences provided herein. Isoforms, fragments, variants or homologues of complement proteins described herein may optionally be functional isoforms, fragments, variants or homologues, e.g. having a functional property/activity of the reference protein, as determined by analysis by a suitable assay for the functional property/activity.

Methods for Assessing Complement-Related Disorders

The present invention provides methods for assessing the risk of onset, risk of progression, or risk of development of a complement-related disorder. The complement related disorder may be any disorder in which the complement system, or activation/over-activation/dysregulation thereof, is pathologically implicated. The complement related disorder may be any disorder described herein. The methods described herein may be useful in monitoring the success of treatment, including past or ongoing treatment, for complement-related disorders.

In some aspects, provided is a method of identifying a subject having a complement-related disorder or at risk of developing a complement-related disorder, the method comprising:
(a) determining the level of a complement protein selected from one or more of FHR1, FHR2, FHR3, FHR4 and/or FHR5 in a blood sample obtained from the subject;
(b) determining that the subject has or is likely to develop a complement-related disorder if the level of the complement protein determined in (a) is elevated as compared to the level of that complement protein in blood in a control subject that does not have a complement-related disorder.

In some aspects, there is provided a method of determining whether a subject has, or is at risk of developing, a complement-related disorder, the method comprising:
(a) determining the level of a complement protein selected from one or more of FHR1, FHR2, FHR3, FHR4 and/or FHR5 in a blood sample obtained from the subject;
(b) determining that the subject has or is likely to develop a complement-related disorder if the level of the complement protein determined in (a) is elevated as compared to the level of that complement protein in blood in a control subject that does not have a complement-related disorder.

In some embodiments step (a) comprises determining the level of two of the complement proteins selected from FHR1, FHR2 and/or FHR3. In some embodiments step (a) comprises determining the level of three of the complement proteins selected from FHR1, FHR2 and FHR3.

In some embodiments step (a) comprises, or further comprises, determining the level of FHR4 and/or FHR5. The methods described herein may comprise determining that the subject has or is likely to develop a complement-related disorder if the level of FHR4 and/or FHR5 is elevated as compared to the level of that complement protein in blood in a control subject that does not have a complement-related disorder.

In some embodiments step (a) comprises, or further comprises, determining the level of FH and/or FHL-1. The method may comprise determining the level of FHL-1, alone or in combination with other complement protein(s), and determining that the subject has or is likely to develop a complement-related disorder if the level of FHL-1 is altered, e.g. elevated, as compared to the level of FHL-1 in blood in a control subject that does not have a complement-related disorder.

Determining the level of two or more complement proteins may be performed simultaneously, concurrently or sequentially. The complement proteins may be detected in the same assay, or in one or more separate assays. Determining the level of a second or subsequent complement protein may be performed concurrently with, prior to or after determining the level of a first complement protein. In some embodiments, steps (a) and (b) may be repeated one or more times on the same subject at appropriate time intervals in order to assess the progression of a complement-related disorder.

In any aspect or embodiment described herein the method may comprise determining the level of any one of:
a) FHR1;
b) FHR2;
c) FHR3;
d) FHR4;
e) FHR5;
f) FHR1 and FHR2;
g) FHR1 and FHR3;
h) FHR1 and FHR4;
i) FHR1 and FHR5;
j) FHR2 and FHR3;
k) FHR2 and FHR4;
I) FHR2 and FHR5;
m) FHR3 and FHR4;
n) FHR3 and FHR5;
o) FHR4 and FHR5;
p) FHR1, FHR2 and FHR3;
q) FHR1, FHR2 and FHR4;
r) FHR1, FHR2 and FHR5;
s) FHR1, FHR3 and FHR4;
t) FHR1, FHR3 and FHR5;
u) FHR1, FHR4 and FHR5;
v) FHR2, FHR3 and FHR4;
w) FHR2, FHR3 and FHR5;
x) FHR2, FHR4 and FHR5;
y) FHR3, FHR4 and FHR5;
z) FHR1, FHR2, FHR3 and FHR4;
aa) FHR1, FHR2, FHR3 and FHR5;
bb) FHR1, FHR2, FHR4 and FHR5;
cc) FHR2, FHR3, FHR4 and FHR5;
dd) FHR1, FHR3, FHR4 and FHR5; or
ee) FHR1, FHR2, FHR3, FHR4 and FHR5;

or any of (a) to (ee) in combination with determining the level of FH and/or FHL-1, e.g. FHR1, FHR2, FHR3, plus FH and/or FHL-1; or FHR1, FHR2, FHR3, FHR4, FHR5, plus FH and/or FHL-1.

In some embodiments, reference to ‘FHR1’ herein may refer to the detection of either one or both of FHR1a and/or FHR1b.

In some embodiments the complement protein(s) to be detected/determined is not FHR3. In some embodiments the complement protein(s) to be detected/determined is not FHR4. In some embodiments the complement protein(s) to be detected/determined is not FH. In some embodiments the complement protein(s) to be detected/determined is not FHL-1.

The selection or combination of complement protein(s) detected may depend on the complement-related disorder of interest and the complement protein(s) that are useful biomarkers for an individual disorder. For example, detecting one or more of FHR1, FHR2, FHR3, FHR4, FHR5 and/or FHL-1 is predictive of AMD risk, whereas other particular complement proteins and combinations thereof are predictive for other complement-related disorders, see e.g. the disorders and references described herein. The present disclosure allows the precise detection and distinction of any one or more of the complement proteins described herein, thus allowing the absolute levels of said proteins to inform the likelihood of disorder onset and/or progression according to the variations in protein levels in each disorder.

In some cases, any method described herein may comprise determining the level of any one or more complement proteins selected from FHR1, FHR2, FHR3, FHR4, FHR5, FH and/or FHL-1, e.g. in a blood sample obtained from a subject, and then determining that the subject has or is likely to develop a complement-related disorder if the level of the complement protein(s) is altered as compared to the level of that complement protein(s) in blood in a control subject that does not have a complement-related disorder. The term “altered” as used herein refers to the level of the complement protein(s) increasing or decreasing, e.g. the level of one or more complement proteins may be higher or lower as compared to the level of those complement proteins in blood in a control subject that does not have a complement-related disorder. In some cases, the level of the complement protein may be decreased as compared to the level of that complement protein in blood in a control subject that does not have a complement-related disorder. In some cases, where the level of two or more complement proteins is determined, the level of one or more complement proteins may be elevated whilst the level of one or more different complement proteins may be decreased as compared to the levels of those complement proteins in blood in a control subject that does not have a complement-related disorder.

In some embodiments the level of a complement protein is determined using any suitable technique known in the art and available to a skilled person. In some embodiments the level of a complement protein is determined by mass spectrometry and/or digesting the protein with endoproteinase GluC, e.g. as described herein. Determining the level of a complement protein(s) may involve detecting any combination of peptides produced by digestion with GluC, as described herein. The level of a complement protein may be determined using, for example, an enzyme-linked immunosorbent assay (ELISA/EIA) e.g. as described in van Beek et al., Front Immunol. 2017; 8: 1328; van Beek et al. Front Immunol. 2018; 9: 1727; and Pouw et al., PLoS One. 2016 Mar. 23; 11(3):e0152164; which are hereby incorporated by reference in their entirety. The level of a complement protein may be determined using, for example, Western blotting or dot blotting with appropriate antibodies, HPLC, protein immunoprecipitation or immunoelectrophoresis.

Any method described herein may comprise an initial step of obtaining a sample and/or at least one protein, e.g. complement protein, from the subject. Suitable sources of samples are described herein. The methods described herein may comprise determining the level of circulating FHR1, FHR2, and/or FHR3, circulating FHR4 and/or FHR5, and optionally circulating FH and/or FHL-1. Circulating proteins may be present in e.g. blood or lymph.

Any method described herein may comprise determining the level of one or more of C3, C3b, C3a, iC3b, C3f, C3c, C3dg, and/or C3d, e.g. as described herein.

In some aspects, there is provided a method of determining whether a subject has, or is at risk of developing, a complement-related disorder, the method comprising:
(a) digesting at least one complement protein selected from FHR1, FHR2, FHR3 FHR4 and/or FHR5, in a sample obtained from the subject with endoproteinase GluC to obtain one or more peptides;
(b) determining the level of the one or more peptides by mass spectrometry;
(c) using the results of (b) to determine the level of one or more complement proteins; and
(d) determining that the subject has, or is at risk of developing, a complement-related disorder if the level of one or more complement proteins determined in (c) is elevated as compared to the level of that complement protein(s) in blood in a control subject that does not have a complement-related disorder.

In some embodiments the method further comprises digesting one or both of FH and/or FHL-1 with endoproteinase GluC to obtain one or more peptides, determining the level of the one or more peptides by mass spectrometry and/or using the results of the mass spectrometry to determine the level of FH and/or FHL-1. Exemplary combinations of complement proteins for use in the methods of the present invention are described above.

Also described herein is the use of a complement protein selected from one or more of FHR1, FHR2, FHR3, FHR4 and/or FHR5, and optionally FH and/or FHL-1, for identifying a subject having a complement-related disorder or at risk of developing a complement-related disorder, or for determining whether a subject has or is at risk of developing a complement-related disorder, the use comprising:
(a) determining the level of a complement protein selected from one or more of FHR1, FHR2, FHR3, FHR4 and/or FHR5, and optionally FH and/or FHL-1, in a blood sample obtained from the subject; and
(b) determining that the subject has or is likely to develop a complement-related disorder if the level of FHR1, FHR2, FHR3, FHR4 and/or FHR5, and optionally FHL-1, is elevated as compared to the level of that complement protein in blood in a control subject that does not have a complement-related disorder.

Also provided is the use of a complement protein selected from one or more of FHR1, FHR2, FHR3, FHR4 and/or FHR5, and optionally FH and/or FHL-1, as a biomarker e.g. for identifying a subject having a complement-related disorder or at risk of developing a complement-related disorder, or for determining whether a subject has or is at risk of developing a complement-related disorder, the use comprising the steps described hereinabove.

In one aspect, provided is the use of endoproteinase GluC in a method for determining the presence and/or level of a complement protein, e.g. in a sample or a subject, e.g. according to the methods described herein. Also provided is the use of endoproteinase GluC in a method of identifying a subject having a complement-related disorder or at risk of developing a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject has or is likely to develop a complement-related disorder.

Also provided is the use of GluC in a method of selecting a subject for treatment of a complement-related disorder with a complement-targeted therapeutic, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

Also provided is the use of GluC in the diagnosis of a complement-related disorder, comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

In some embodiments the methods described herein are performed in vitro or ex vivo. For example, a sample may be obtained from a subject of interest, or a control subject, and the determining steps are performed in vitro or ex vivo.

In methods described herein the level of the complement protein(s) is compared to the level of a reference value or level, sometimes called a control. In some cases the level of the complement protein(s) is compared to the level of the same complement protein in a control subject that does not have a complement-related disorder. A reference value may be obtained from a control sample, which itself may be obtained from a control subject. Data or values obtained from the individual to be tested, e.g. from a sample, can be compared to data or values obtained from the control sample. In some cases, the control is a spouse, partner, or friend of the subject.

The level of the complement protein(s) that are determined may be elevated (i.e. higher, increased, greater) compared to the reference value or level. That is, there may be more of the complement protein(s) in the sample tested compared to the reference value. There may be a higher amount or concentration of the complement protein(s) in the tested sample compared to the reference value or in a control sample.

The level of the complement protein(s) that are determined may be reduced (i.e. lower, decreased) compared to the reference value or level. That is, there may be less of the complement protein(s) in a tested sample tested compared to the reference value. There may be a lower amount or concentration of the complement protein(s) in a tested sample compared to the reference value or in a control sample.

As used herein the term “reference value” refers to a known measurement value used for comparison during analysis. In some cases, the reference value is one or a set of test values obtained from an individual or group in a defined state of health. The reference value may be one or a set of test values obtained from a control. In some cases, the reference value is/has been obtained from determining the level of complement proteins in subjects known not to have a complement-related disorder. In some cases, the reference value is set by determining the level or amount of a complement protein previously from the individual to be tested e.g. at an earlier stage of disease progression, or prior to onset of the disease. The reference value may be taken from a sample obtained from the same subject, or a different subject or subject(s). The sample may be derived from the same tissue/cells/bodily fluid as the sample used by the present invention. The reference value may be a standard value, standard curve or standard data set. Values/levels which deviate significantly from reference values may be described as atypical values/levels.

In some cases the control may be a reference sample or reference dataset, or one or more values from said sample or dataset. The reference value may be derived from a reference sample or reference dataset. The reference value may be derived from one or more samples that have previously been obtained from one or more subjects that are known not to have a complement-related disorder and/or known or expected not to be at risk of developing a complement-related disorder. The reference value may be derived from one or more samples that have previously been obtained from one or more subjects that are known to have a complement-related disorder. The reference value may be derived from one or more samples that have previously been obtained from one or more subjects that are known to be at risk of developing a complement-related disorder. The reference value may be consensus level or an average, or mean, value calculated from a reference dataset, e.g. a mean protein level. The reference dataset/value may be obtained from a large-scale study of subjects known to have a complement-related disorder, such as AMD, e.g. as described herein.

The reference value may be derived from one or more samples that have previously been obtained from one or more subjects that are in the same family as the subject of interest, or from one or more subjects that are not in the same family as the subject of interest.

The reference value may be derived from one or more samples that have previously been obtained and/or analysed from the individual/subject/patient to be tested, e.g. a sample was obtained from the individual when they were at an earlier stage of a complement-related disorder, or a sample was obtained from the individual before the onset of a complement-related disorder.

The reference value may be obtained by performing analysis of the sample taken from a control subject in parallel with a sample from the individual to be tested. Alternatively, the control value may be obtained from a database or other previously obtained value. The reference value may be determined concurrently with the methods disclosed herein, or may have been determined previously.

Control subjects from which samples are/have been obtained may have undergone treatment for a complement-related disorder and/or received a complement-related therapy/therapeutic agent.

Controls may be positive controls in which the target molecule is known to be present, or expressed at high level, or negative controls in which the target molecule is known to be absent or expressed at low level.

Samples from one or more control subjects may comprise any one, two, three, four, five, six of seven of FHR1, FHR2, FHR3, FHR4, FHR5, FH and/or FHL-1. In some cases each complement protein is in a separate control sample. In some cases a control sample contains multiple complement proteins. In some cases the methods described herein comprise comparing the level of one of more complement proteins determined as described herein to different, e.g. one or more, samples, each sample containing one or more complement proteins. In some cases the methods described herein comprise comparing the level of one or more complement proteins determined as described herein to a single sample, wherein the sample contains one or more complement proteins.

In some cases control samples are obtained from the same tissue(s) as the sample obtained from the individual to be tested. In some cases control samples are obtained from different tissue(s) as the sample obtained from the individual to be tested. Control samples may be obtained from control subjects at certain time(s) of day, or on certain days. Sample(s) obtained from the individual to be tested are preferably obtained at the same time(s) of day and/or day(s) as the control samples.

In some cases, an increase/decrease of a complement protein, e.g. as described herein, as compared to a reference value indicates an increased risk of developing a complement-related disorder. In some cases, an increase/decrease of a complement protein, e.g. as described herein, indicates an increased risk of developing the disorder when compared to a reference value taken from the same subject at an earlier stage of the disorder, e.g. in a sample from the same subject.

In some embodiments, a method described herein may comprise determining the level of two or more complement proteins and comparing their values e.g. concentrations. The values may be compared to each other, as well as to reference values, e.g. increased levels of C3 and C3b compared to stationary or decreased levels of iC3b and further C3b breakdown products may be indicative of a higher risk of development of a complement-related disorder and/or the need to treat a subject for a complement-related disorder. Decreased levels of C3 and C3b compared to stationary or increased levels of iC3b and further C3b breakdown products may be indicative of a lower risk of development of a complement-related disorder and/or that treatment for a complement-related disorder is effective.

In some cases, a method described herein may comprise comparing the levels of any one or more of FHR1, FHR2, FHR3, FHR4 and/or FHR5, to the level of FH and/or FHL-1 in the subject tested. For example, elevated levels of FHR1, FHR2, FHR3, FHR4 and/or FHR5, and optionally FHL-1, compared to stationary levels of FH (i.e. a statistically non-significant change) in a subject may be indicative of a higher risk of the subject developing a complement-related disorder and/or the need to treat the subject for a complement-related disorder.

In some embodiments, a method provided herein comprises a step of correlating the presence of an atypical or altered amount/level of a complement protein with an increased risk of the subject developing or having a complement-related disorder.

Examples of reference values for complement proteins in human subjects known not to have a complement-related disorder include:
a) FH: ˜150 to 500 μg/ml in human blood (Clark et al., J Immunol 2014. 193(10):4962-70 and unpublished data);
   Mean: 833 nM in human plasma, SD: 149 (derived from the mass spectrometry methods described herein);
b) FHL-1: ˜0.5 to 50 μg/ml in human blood (Clark et al., J Immunol 2014. 193(10):4962-70 and unpublished data);
   Mean: 10.9 nM in human plasma, SD. 2.4 (derived from the mass spectrometry methods described herein);
c) FHR1: ˜70 to 100 μg/ml in human plasma (Heinen. S et al., Blood 114, 2439-2447);
   Mean: 33.9 nM in human plasma, SD: 16.5 (derived from the mass spectrometry methods described herein);
d) FHR2: ˜15-50 μg/ml in human plasma, or about 1/10 of FH concentration (Skerka et al., Mol Immunol 2013, 56:170-180);
   Mean: 51.2 nM in human plasma, SD: 17.0 (derived from the mass spectrometry methods described herein);
e) FHR3: ˜70 to 100 μg/ml in human plasma (Fritsche, L. G. et al., Hum. Mol. Genet. 2010.19, 4694-4704);
   Mean: 20.6 nM in human plasma. SD: 13.8 (derived from the mass spectrometry methods described herein):
f) FHR4: ≤5 μg/ml in human blood (WO 2019/215330);
   Mean: 46.1 nM in human plasma, SD: 24.4 (derived from the mass spectrometry methods described herein);
g) FHR5: ˜1.5 μg/ml in human plasma (van Beek, A E et al., Front Immunol. 2017 Oct. 18; 8:1328);
   Mean: 29.2 nM in human plasma, SD: 8.7 (derived from the mass spectrometry methods described herein),
h) FI: ˜35 μg/ml in human plasma;
i) C3: ˜0.5-16 mg/ml in human plasma (Engström, G. et al., J Hum Hypertens. 2007 April; 21(4):276-82; Lee S H et al., Am J Respir Crit Care Med. 2006 Feb. 15; 173(4):370-8);
j) C3a: 46-157 ng/ml (Lee S H et al., Am J Respir Crit Care Med. 2006 Feb. 15; 173(4):370-8);
k) iC3b: ˜0.7-5 μg/ml (Kim A H J et al., Arthritis Rheumatol. 2019 March; 71(3):420-430).

In some cases, mean reference values for circulating FH, FHL-1 and FHR1-5 in human subjects known not to have a complement-related disorder, e.g. AMD, include the following (95% CI in parentheses):
a) FH, nM: 737.3 (718.2-756.5)
b) FHL-1, nM: 10.4 (10.1-10.8)
c) FHR-1, nM: 31.2 (29.4-32.9)
d) FHR-2, nM: 45.3 (43.1-47.6)
e) FHR-3, nM: 24.1 (21.7-26.5)
f) FHR-4, nM: 46.1 (42.7-49.6)
g) FHR-5, nM: 25.5 (24.5-26.5).

An ‘elevated’ level of a complement protein, e.g. in a sample, may be elevated/increased/higher when compared to a reference value for that protein, e.g. as above. A ‘reduced’ level of a complement protein, e.g. in a sample, may be reduced/decreased/lower when compared to a reference value for that protein, e.g. as above.

The relative concentrations of one complement protein to another can be determined using their reference values. For example, the ratio of the level of one complement protein to the level of another, or others, can be inferred from the concentrations provided above, e.g. FH:FHL-1, C3:iC3b, C3:C3b etc. The relative concentrations and/or ratios of the level of one complement protein to another, or others, may be altered in complement-related disorders. In some embodiments the methods provided herein involve detecting two or more complement proteins and determining how the levels of the complement proteins change with respect to one another as compared to a reference value(s). For example, the level of a first complement protein may increase as compared to the level of a second complement protein, or vice versa, e.g. FHL-1 vs FH, FHR1 to FHR5 vs FH and/or FHL-1, C3 vs iC3b, C3 vs C3b.

Predictive and Therapeutic Applications

In some aspects the invention provides methods for selecting treatment for and/or treating subjects/patients that have a complement-related disorder or have been identified as having a complement-related disorder, e.g. by determining the level of a complement protein as described herein.

The methods described herein may be diagnostic, prognostic and/or predictive of the risk of onset or progression of a complement-related disorder. Diagnostic methods can be used to determine the diagnosis or severity of a disease, prognostic methods help to predict the likely course of disease in a defined clinical population under standard treatment conditions, and predictive methods predict the likely response to a treatment in terms of efficacy and/or safety, thus supporting clinical decision-making.

The terms “disorder”, “disease” and “condition” may be used interchangeably herein and refer to a pathological issue of a body part, organ or system which may be characterised by an identifiable group of signs or symptoms. The term “complement-related disorder” refers to disorders, diseases or conditions that comprise or arise from deficiencies or abnormalities in the complement system. In some embodiments, the complement-related disorder is a disorder driven by complement activation or complement over-activation. The terms “develop”, “developing”, and “development”, e.g. of a disorder, as used herein refer both to the onset of a disease as well as the progression, exacerbation or worsening of a disease state. The term “biomarker(s)” as used herein refers to one or more measurable indicators of a biological state or condition.

In any embodiment described herein the disorder is one in which the complement system, or activation/over-activation/dysregulation thereof, is pathologically implicated. The complement related disorder may be any disorder described herein. “Pathologically implicated” as used herein may refer to a protein level which is raised or lowered in the disorder compared with a reference value, and/or where the protein contributes towards the pathology of the disorder. The selection or combination of complement protein(s) detected/determined may depend on the complement-related disorder of interest and the complement protein(s) that are useful biomarkers for said disorder.

Subjects with elevated levels of FHR1, FHR2, FHR3, FHR4 and/or FHR5, and optionally FHL-1, and/or increased expression of a gene(s) encoding one or more of said proteins, may derive therapeutic or prophylactic benefit from said levels being reduced. This may be achieved with a complement-targeted therapy or complement-targeted therapeutic agent.

In some aspects, provided is a method for selecting a subject for treatment with a therapeutic agent, e.g. a complement-targeted therapy or therapeutic agent, the method comprising:
(a) determining the level of a complement protein selected from one or more of FHR1, FHR2, FHR3, FHR4 and/or FHR5, and optionally FH and/or FHL-1, in a blood sample obtained from the subject;
(b) selecting the subject for treatment with the therapeutic agent if the level of one or more of FHR1, FHR2, FHR3, FHR4 and/or FHR5, and optionally FHL-1, determined in (a) is elevated as compared to the level of that complement protein(s) in blood in a control subject that does not have a complement-related disorder.

In some aspects, provided is a method for selecting a therapeutic agent, e.g. a complement-targeted therapy or therapeutic agent, for a subject, the method comprising:
(a) determining the level of a complement protein selected from one or more of FHR1, FHR2, FHR3, FHR4 and/or FHR5, and optionally FH and/or FHL-1, in a blood sample obtained from the subject;
(b) determining whether the level of one or more of FHR1, FHR2, FHR3, FHR4 and/or FHR5, and optionally FHL-1, is elevated as compared to the level of that complement protein(s) in blood in a control subject that does not have a complement-related disorder; and
(c) selecting a therapeutic agent that targets one or more complement protein(s) that are elevated in the sample from the subject.

The subject may be suspected of having, or may have been determined to have, a complement-related disorder, e.g. using the methods described herein. The level(s) of FHR1, FHR2, FHR3, FHR4 and/or FHR5, may be compared to the level of FH and/or FHL-1.

Also described herein is the use of a complement protein for selecting a subject for treatment with a complement-targeted therapy or for selecting a therapeutic agent for a subject, the use comprising the steps disclosed hereinabove.

Also provided is a method of treatment comprising:
(a) determining the level of a complement protein selected from one or more of FHR1, FHR2, FHR3, FHR4 and/or FHR5, and optionally FH and/or FHL-1, in a blood sample obtained from the subject;
(b) determining whether the level of FHR1, FHR2, FHR3, FHR4 and/or FHR5, and optionally FHL-1, is elevated as compared to the level of that complement protein(s) in blood in a control subject that does not have a complement-related disorder; and
(c) treating the subject with a complement-targeted therapy or therapeutic agent.

Also provided is a complement-targeted therapy or therapeutic agent for use in a method of treatment, wherein the method comprises:
(a) determining the level of a complement protein selected from one or more of FHR1, FHR2, FHR3, FHR4 and/or FHR5, and optionally FH and/or FHL-1, in a blood sample obtained from the subject;
(b) determining whether the level of FHR1, FHR2 and/or FHR3, and optionally FHR4 and/or FHR5, is elevated as compared to the level of that complement protein in blood in a control subject that does not have a complement-related disorder; and
(c) treating the subject with the complement-targeted therapy or therapeutic agent.

Also provided is the use of a complement-targeted therapy or therapeutic agent in the manufacture of a medicament for the treatment of a complement-related disorder, e.g. Macular Degeneration, wherein the method of treatment comprises:
(a) determining the level of a complement protein selected from one or more of FHR1, FHR2, FHR3, FHR4 and/or FHR5, and optionally FH and/or FHL-1, in a blood sample obtained from the subject;
(b) determining whether the level of one or more of FHR1, FHR2, FHR3, FHR4 and/or FHR5, and optionally FHL-1, is elevated as compared to the level of that complement protein in blood in a control subject that does not have a complement-related disorder; and
(c) treating the subject with the complement-targeted therapy or therapeutic agent.

Any method described herein may comprise one or more of the steps of:
(a) determining the level of a complement protein selected from one or more of FHR1, FHR2, FHR3, FHR4 and/or FHR5, and optionally FH and/or FHL-1, in a blood sample obtained from a subject;
(b) determining whether the level of the complement protein (e.g. one or more of FHR1, FHR2, FHR3, FHR4 and/or FHR5, and optionally FHL-1) is elevated as compared to the level of that complement protein in blood in a control subject that does not have a complement-related disorder; and
(c) treating the subject by targeting the complement protein(s) that were found to be elevated in step (b).

As described herein, the methods may comprise determining whether the level of a complement protein is altered, e.g. increased or decreased, as compared to the level of that complement protein in blood in a control subject that does not have a complement-related disorder. In some cases, the methods may comprise determining the relative concentrations of complement proteins compared to each other, e.g. the level of a complement protein may be elevated as compared to the level of a different complement protein, which may be unaltered or decreased, in the same subject or as compared to a control subject.

“Targeting the complement protein(s)” refers to using any suitable agent, such as those described herein, to reduce the level(s) of the complement protein(s) that were found to be elevated in the subject, for example by reducing gene expression, preventing mRNA transcription, sequestering the protein(s), or preventing the normal activity of the protein(s).

Exemplary complement-targeted therapeutic agents and therapies are described hereinbelow. Also described are diseases and disorders which may be identified and treated as described herein.

“Treatment” may refer to treating or preventing a complement-related disorder, such as those described herein.

In some embodiments the level of a complement protein is determined using any suitable technique known in the art and available to a skilled person. In some embodiments the level of a complement protein is determined by mass spectrometry and/or digesting the protein with endoproteinase GluC, e.g. as described herein.

In some embodiments the methods described herein are performed in vitro or ex vivo. For example, a sample may be obtained from a subject of interest, or a control subject, and the steps that involve determining the level of a complement protein, determining whether a subject has or is at risk of developing a complement-related disorder, and digesting at least one complement protein are performed in vitro or ex vivo. Steps of the methods that involve treating a subject may be performed in vivo.

The methods described herein may be useful in monitoring the success of treatment, including past or ongoing treatment, for complement-related disorders. In some embodiment the methods described herein may comprise treating a subject who has/has been determined to have a complement-related disorder, and then re-determining the level of one or more of FHR1, FHR2, FHR3, FHR4 and/or FHR5, and optionally FH and/or FHL-1, after treatment. Such methods are useful for determining the efficacy of treatment and the progression of the disorder.

A complement-related disorder according to the present disclosure is one in which the complement system, or activation/over-activation/dysregulation thereof, is pathologically implicated.

The complement-related disorder may comprise disruption of the classical, alternative and/or lectin complement pathways. In some cases, the disorder may be associated with deficiencies in, abnormalities in, or absence of regulatory components of the complement system. In some embodiments, the disorder may be a disorder associated with the alternative complement pathway, disruption of the alternative complement pathway and/or associated with deficiencies in, abnormalities in, or absence of regulatory components of the alternative complement pathway. In some cases the disorder is associated with the complement amplification loop. In some cases the disorder is associated with inappropriate activation, over-activation, or dysregulation of the complement system, in whole or in part, e.g. C3 convertase assembly, C3b production, C3b deposition, and/or the amplification loop.

In some cases, the present invention provides methods for determining whether a complement-related disorder is associated with over-activation of the complement system. In some cases, the methods are capable of determining whether elevated levels of FHR1, FHR2, FHR3, FHR4 and/or FHR5 are contributing to complement over-activation and/or complement-related disorders, e.g. as compared to a control subject that does not have a complement-related disorder.

In some cases, the disorder is associated with any one or more of C3, C3b, iC3b, FI, FH, FHL-1, or FHR1-FHR5. In some cases, the disorder is associated with deficiencies or abnormalities in the activity of any one or more of C3, C3b, iC3b, FI, FH, FHL-1, or FHR1-FHR5. In some cases one or more of these proteins are pathologically implicated in the disorder, e.g. have raised or lower levels compared with a reference value.

In some cases, the disorder is associated with one or more of CR1, CD46, CD55, C4BP, Factor B (FB), Factor D (FD), SPICE, VCP (or VICE) and/or MOPICE. In some cases, the disorder is associated with deficiencies or abnormalities in the activity of one or more of CR1, CD46, CD55, C4BP, Factor B, Factor D, SPICE, VCP (or VICE) and/or MOPICE, or where one or more of these proteins are pathologically implicated.

In some embodiments, the disorder may be a disorder associated with C3 or a C3-containing complex, an activity/response associated with C3 or a C3-containing complex, or a product of an activity/response associated with C3 or a C3-containing complex. That is, in some embodiments, the disorder is a disorder in which C3, a C3-containing complex, an activity/response associated with C3 or a C3-containing complex, or the product of said activity/response is pathologically implicated. In some embodiments, the disorder may be associated with an increased level of C3 or a C3-containing complex, an increased level of an activity/response associated with C3 or a C3-containing complex, or an increased level of a product of an activity/response associated with C3 or a C3-containing complex as compared to the control state. In some embodiments, the disorder may be associated with a decreased level of C3 or a C3-containing complex, a decreased level of an activity/response associated with C3 or a C3-containing complex, or a decreased level of a product of an activity/response associated with C3 or a C3-containing complex as compared to the control state.

In some embodiments, the disorder may be a disorder associated with C3b or a C3b-containing complex, an activity/response associated with C3b or a C3b-containing complex, or a product of an activity/response associated with C3b or a C3b-containing complex. That is, in some embodiments, the disorder is a disorder in which C3b, a C3b-containing complex, an activity/response associated with C3b or a C3b-containing complex, or the product of said activity/response is pathologically implicated. In some embodiments, the disorder may be associated with an increased level of C3b or a C3b-containing complex, an increased level of an activity/response associated with C3b or a C3b-containing complex, or increased level of a product of an activity/response associated with C3b or a C3b-containing complex as compared to the control state. In some embodiments, the disorder may be associated with a decreased level of C3b or a C3b-containing complex, a decreased level of an activity/response associated with C3b or a C3b-containing complex, or a decreased level of a product of an activity/response associated with C3b or a C3b-containing complex as compared to the control state.

In some embodiments, the disorder may be a disorder associated with any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46 or a product of an activity/response associated with any one or more of FH, FHL-1, FI, FHR1-FHR5, FB, FD, CR1 and/or CD46. In some embodiments, the disorder is a disorder in which any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46, or the product of said activity/response is pathologically implicated. In some embodiments, the disorder may be associated with a decreased level of any one or more of FH, FHL-1, FI, FHR1-FHR5, FB, FD, CR1 and/or CD46, a decreased level of an activity/response associated with any one or more of FH, FHL-1, FI, FHR1-FHR5, FB, FD, CR1 and/or CD46, or a decreased level of a product of an activity/response associated with any one or more of FH, FHL-1, FI, FHR1-FHR5, FB, FD, CR1 and/or CD46 as compared to a control state.

In some embodiments, the disorder may be associated with an increased level of any one or more of FHR1, FHR2, FHR3, FHR4 and/or FHR5, an increased level of an activity/response associated with any one or more of FHR1, FHR2, FHR3, FHR4 and/or FHR5, or an increased level of a product of an activity/response associated with any one or more of FHR1, FHR2, FHR3, FHR4 and/or FHR5 as compared to a control state, see e.g. Zhu et al., Kidney Int. 2018 July; 94(1):150-158; Pouw et al., Front Immunol. 2018 Apr. 24; 9:848; both hereby incorporated by reference in their entirety. The methods may comprise determining the systemic level of any combination of FHR1 to FHR5.

In some embodiments, the disorder may be associated with an increased level of any one or more of FHR1, FHR2 and/or FHR3, an increased level of an activity/response associated with any one or more of FHR1, FHR2 and/or FHR3, or an increased level of a product of an activity/response associated with any one or more of FHR1, FHR2 and/or FHR3. In some embodiments the disorder may be associated with an increased level of FHR4, an increased level of an activity/response associated with FHR4, or an increased level of a product of an activity/response associated with FHR4 as compared to a control state, see e.g. WO 2019/215330 and Cipriani et al., Nat Commun 11, 778 (2020), both hereby incorporated by reference in their entirety. In some embodiments the disorder may be associated with an increased level of FHL-1.

In some embodiments the disorder is associated with increased levels of any one or more of C3, C3b, C3 convertase and/or C3bBb as compared to a control state. In some embodiments the disorder is associated with decreased levels of any one or more of C3, C3b, C3 convertase and/or C3bBb as compared to a control state. In some embodiments, the disorder is associated with increased levels of iC3b as compared to a control state. In some embodiments, the disorder is associated with decreased levels of iC3b as compared to a control state. In some embodiments the disorder is associated with increased levels of any one or more of C3a, C3f, C3c, C3dg, C3d, and/or C3g as compared to a control state. In some embodiments the disorder is associated with decreased levels of any one or more of C3a, C3f, C3c, C3dg, C3d, and/or C3g as compared to a control state.

In some cases, the methods described herein find use in diagnosing, treating or preventing, or selecting a subject for treatment or prevention of, a disorder which would benefit from one or more of: a reduction in the level or activity of one or more of C3bBb-type C3 convertase, C3bBb3b-type C5 convertase and/or C4b2a3b-type C5 convertase; a reduction in the level of one or more of C3, C3b, C3a, iC3b, FHR1, FHR2, FHR3, FHR4, FHR5, C5b and/or C5a; or an increase in the level of one or more of iC3b, C3f, C3c, C3dg, C3d, C3g, FH, FHL-1, FI, FH, FHL-1, FHR1, FHR2, FHR3, FHR4 and/or FHR5 as compared to reference value(s).

In some cases, the methods described herein find use in treating or preventing, or selecting a subject for treatment or prevention of, a disorder which would benefit from a reduction in the level or activity of one or more of FHR1, FHR2, FHR3, FHR4 and/or FHR5, and optionally FHL-1.

The disorder may be an ocular disorder. In some embodiments, a disease or condition to be assessed, diagnosed, treated or prevented as described herein is a complement-related ocular disease. In some embodiments, the disorder is macular degeneration. In some embodiments, the disorder may be selected from, i.e. is one or more of, age-related macular degeneration (AMD), choroidal neovascularisation (CNV), macular dystrophy, and diabetic maculopathy. As used herein, the term “AMD” includes early AMD, intermediate AMD, late/advanced AMD, geographic atrophy (‘dry’ (i.e. non-exudative) AMD), and ‘wet’ (i.e. exudative or neovascular) AMD, each of which may be a disorder in its own right that is detected, treated and/or prevented as described herein. In some embodiments the disease or condition to be treated or prevented is a combination of the diseases/conditions above, e.g. ‘dry’ and ‘wet’ AMD. In some embodiments the disease or condition to be treated or prevented is not ‘wet’ AMD or choroidal neovascularisation. AMD is commonly-defined as causing vision loss in subjects age 50 and older. In some embodiments a subject to be treated is age 50 or older, i.e. is at least 50 years old.

As used herein “early AMD” refers to a stage of AMD characterised by the presence of medium-sized drusen, commonly having a diameter of up to ˜200 μm, within Bruch's membrane adjacent to the RPE layer. Subjects with early AMD typically do not present with significant vision loss. As used herein “intermediate AMD” refers to a stage of AMD characterised by large drusen and/or pigment changes in the retina. Intermediate AMD may be accompanied by some vision loss. As used herein “late AMD” refers to a stage of AMD characterised by the presence of drusen and vision loss, e.g. severe central vision loss, due to damage to the macula. In all stages of AMD, ‘reticular pseudodrusen’ (RPD) or ‘reticular drusen’ (also referred to as subretinal drusenoid deposits (SDD)) may be present, referring to the accumulation of extracellular material in the subretinal space between the neurosensory retina and RPE. “Late AMD” encompasses ‘dry’ and ‘wet’ AMD. In ‘dry’ AMD (also known as geographic atrophy), there is a gradual breakdown of the light-sensitive cells in the macula that convey visual information to the brain and of the supporting tissue beneath the macula. In ‘wet’ AMD (also known as choroidal neovascularization, neovascular and exudative AMD), abnormal blood vessels grow underneath and into the retina. These vessels can leak fluid and blood which can lead to swelling and damage of the macula and subsequent scar formation. The damage may be rapid and severe.

In some embodiments the disorder is early-onset macular degeneration (EOMD). As used herein “EOMD” refers to a phenotypically severe sub-type of macular degeneration that demonstrates a much earlier age of onset than classical AMD and results in many more years of substantial visual loss. Sufferers may show an early-onset drusen phenotype comprising uniform small, slightly raised, yellow subretinal nodules randomly scattered in the macular, also known as ‘basal laminar drusen’ or ‘cuticular drusen’. EOMD may also be referred to as “middle-onset macular degeneration”. The EOMD subset is described in e.g. Boon C J et al. Am J Hum Genet 2008; 82(2):516-23; van de Ven J P, et al. Arch Ophthalmol 2012; 130(8):1038-47; and Taylor, R. L. et al., Ophthalmol. 2019, 126, 1410-1421, all of which are hereby incorporated by reference in their entirety. As with other types of macular degeneration, EOMD is related to complement dysregulation and disrupted Factor H activity. In some embodiments a subject to be treated is age 49 or younger. In some embodiments a subject to be treated is between ages 15 and 49, i.e. is between 15 and 49 years old. In some embodiments the disease or condition to be treated is a macular dystrophy. A macular dystrophy can be a genetic condition, usually caused by a mutation in a single gene, that results in degeneration of the macula.

The methods described herein may be used for determining whether a subject is at risk of onset of macular degeneration, e.g. EOMD and/or AMD, and/or is at risk of EOMD and/or AMD progression. In some cases, the disorder is selected from EOMD, AMD, geographic atrophy (‘dry’ (i.e. non-exudative) AMD), early AMD, intermediate AMD, late/advanced AMD, ‘wet’ (neovascular or exudative) AMD, choroidal neovascularisation (CNV) and retinal dystrophy. In some cases, the subject has or is suspected to have a complement-related disorder. In some cases the disorder is AMD. In some cases the disorder is EOMD.

Thus the present invention provides a method for determining whether a subject is at risk of developing macular degeneration, e.g. EOMD and/or AMD, the method comprising:
(a) digesting one or more complement proteins in a sample obtained from the subject with endoproteinase GluC to obtain one or more peptides, e.g. wherein the one or more complement proteins is selected from one or more of FHR1, FHR2, FHR3, FHR4 and/or FHR5, and optionally FH and/or FHL-1;
(b) determining the level of the one or more peptides by mass spectrometry; and
(c) using the results of (b) to determine the level of the one or more complement proteins; and
(d) determining that the subject has, or is at risk of developing, macular degeneration if the level of the FHR1, FHR2, FHR3, FHR4 and/or FHR5, and optionally FHL-1, is elevated as compared to the level of that complement protein(s) in blood in a control subject that does not have a complement-related disorder.

Also provided herein is a method for assessing the propensity or predisposition of a subject to develop a complement-related disorder, comprising steps (a) to (d) above.

In some embodiments the disorder is one associated with the kidney, e.g. nephropathy/a nephropathic disorder. In some cases, the disorder is a neurological and/or neurodegenerative disorder. In some cases, the disorder is associated with autoimmunity, e.g. an autoimmune disease. In some cases, the disorder is associated with inflammation, e.g. an inflammatory disease. In some cases the disorder is characterised by the deposition of C3, e.g. the glomerular pathologies (see e.g. Skerka et al 2013, supra).

In some embodiments the disorder may be selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, and Alzheimer's disease.

In some cases, the disorder is cancer. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine. In some cases the cancer is lung cancer. In some embodiments the complement-related disorder is an indoleamine 2,3-dioxygenase 1 (IDO)-expressing cancer.

In some cases the cancer is glioblastoma e.g. glioblastoma multiforme (GBM). Glioblastoma (GBM) is the most common malignant primary central nervous system (CNS) cancer in adults. GBM is among the malignancies that are uniquely unresponsive to cancer immunotherapy.

As described herein, indoleamine 2,3-dioxygenase 1 (IDO) activity in tumour cells increased expression of FH and FHL-1, which were found to be associated with expression of immunosuppressive genes, suppression of anti-tumour immune responses, poorer survival outcomes for glioma patients and a faster rate to GBM recurrence. See also Zhai et al., Clin Cancer Res, 2021 DOI: 10.1158/1078-0432.CCR-21-1392. Thus, in some embodiments the complement-related disorder is IDO-expressing GBM. In some embodiments the complement-related disorder is isocitrate dehydrogenase (IDH)-expressing GBM. In some embodiments the GBM expresses both IDO and IDH. In some embodiments the cancer, e.g. GBM, comprises tumour cells with increased expression of FH and/or FHL-1.

Thus, the present disclosure provides a method for determining whether a subject has, is at risk of developing, or is at risk of progression of, glioblastoma e.g. glioblastoma multiforme (GBM), the method comprising:
(a) digesting one or more complement proteins in a sample obtained from the subject with endoproteinase GluC to obtain one or more peptides, e.g. wherein the one or more complement proteins is selected from FH and/or FHL-1;
(b) determining the level of the one or more peptides by mass spectrometry; and
(c) using the results of (b) to determine the level of the one or more complement proteins; and
(d) determining that the subject has, or is at risk of developing, glioblastoma if the level of FH and/or FHL-1, is elevated as compared to the level of that complement protein(s) in blood in a control subject that does not have a complement-related disorder e.g. glioblastoma.

In some cases the disorder is neurodegeneration or neurodegenerative disease. The disorder may comprise progressive atrophy and loss of function of neurons. The disorder may be selected from Parkinson's disease, Alzheimer's disease, dementia, stroke, Lewy body disease, Amyotrophic lateral sclerosis (ALS), multiple sclerosis (MS), Huntington's disease and prion diseases.

The role of complement in various diseases is described in e.g. Morgan, B. P., Complement in the pathogenesis of Aizheimer's disease. Semin Immunopathol, 2018. 40(1): p. 113-124; Halbgebauer, R., et al., Janus face of complement-driven neutrophil activation during sepsis. Sernin Immunol, 2018. 37: p. 12-20; Ma, Y., et al., Significance of Complement System in lschemic Stroke: A Comprehensive Review. Aging Dis, 2019. 10(2): p. 429-462; Bonifati and Kishore, Role of complement in neurodegeneration and neuroinflammation. Mol Immunol. 2007 February; 44(5):999-1010; Kleczko, E. K., et al., Targeting the Complement Pathway as a Therapeutic Strategy in Lung Cancer. Front Immunol, 2019. 10: p. 954; and Schafer N. et al., Complement Regulator FHR-3 Is Elevated either Locally or Systemically in a Selection of Autoimmune Diseases, Front Immunol. 2016; 7: 542, which are all hereby incorporated by reference in their entirety. For example, FHL-1 is expressed more in certain tumour cell lines than FH (Junnikkala et al (2000) J. Immunol. 164: 6075-81) and glioblastoma tumours have been shown to express FHR proteins (DeCordova et al. (2019) immunobiology 224: 625-631), both references hereby incorporated in their entirety. Being able to measure and differentiate between FH family proteins is advantageous.

Thus, in some embodiments the complement-related disorder is selected from macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, glioblastoma e.g. glioblastoma multiforme (GBM), stroke, Parkinson's disease, and/or Alzheimer's disease.

In some embodiments, the complement-related disorder is an infectious disease. Complement is a major component of the innate immune system involved in defending against foreign pathogens, including bacteria, viruses, fungi and parasites. Activation of complement leads to robust and efficient proteolytic cascades, which result in opsonization and lysis of the pathogen as well as in the generation of the classical inflammatory response through the production of potent proinflammatory molecules. The role of complement in innate and adaptive immune responses is reviewed in e.g. Dunkelberger, J., Song, W C. Cell Res 2010; 20, 34-50, and Rus H et al., Immunol Res. 2005; 33(2):103-12, which are hereby incorporated by reference in their entirety.

In some embodiments the complement-related disorder is infection by severe acute respiratory syndrome-related coronavirus (SARSr-CoV). In some embodiments the complement-related disorder is infection with SARS-CoV-2. In some embodiments the complement-related disorder is a disease/condition caused or exacerbated by SARS-CoV-2 infection, e.g. COVID-19 or another disease/condition for which infection with SARS-CoV-2 is a contributing factor.

The virology of SARSr-CoV and epidemiology of disease associated with SARSr-CoV infection is reviewed, for example, in Cheng et al., Clin Microbiol Rev (2007) 20(4): 660-694 and de Wit et al., Nat Rev Microbiol (2016) 14: 523-534, both of which are hereby incorporated by reference in their entirety. SARSr-CoV is a species of coronavirus of the genus Betacoronavirus and subgenus Sarbecoronavirus that infects humans, bats and certain other mammals. It is an enveloped positive-sense single-stranded RNA virus. Two strains of SARSr-CoV have caused serious outbreaks of severe respiratory diseases in humans: SARS-CoV, which caused an outbreak of severe acute respiratory syndrome (SARS) between 2002 and 2003, and SARS-CoV-2, which has caused the coronavirus disease 2019 (COVID-19) pandemic.

As used herein, “SARS-CoV-2” refers to the SARSr-CoV having the nucleotide sequence of GenBank: MN996527.1 (“Severe acute respiratory syndrome coronavirus 2 isolate WIV02, complete genome”), reported in Zhou et al., Nature (2020) 579: 270-273, and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: MN996527.1. Variants of SARS-CoV-2 of particular interest include: (i) the variant designated VUI-202012/01, which belongs to the B.1.1.7 lineage, having the canonical nucleotide sequence of GISAID accession EPI_ISL_601443; (ii) the variant designated 501Y.V2/B.1.351, having the canonical nucleotide sequence of GISAID accession EPI_ISL_768642; (iii) the variant known as B.1.1.248/P.1, having the canonical nucleotide sequence of GISAID accession EPI_ISL_792680; (iv) the variant known as B.1.617.1; and (v) the variant known as B.1.617.2. In some embodiments, the complement-related disorder is a disease/condition caused by infection of SARS-CoV-2 variants B.1.1.7, B.1.1.248/P.1, B.1.617.1 and/or B.1.617.2.

The clinical features of COVID-19 are described in Lechien et al., Journal of Internal Medicine (2020) 288(3): 335-344, International Severe Acute Respiratory and Emerging Infections Consortium (ISARIC). COVID-19 Report: 19 May 2020: ISARIC; 2020 and Docherty et al., BMJ (2020) 369:m1985, which are hereby incorporated by reference in their entirety. Common symptoms include cough, fever, headache, dyspnoea, anosmia, pharyngitis, nasal obstruction, rhinorrhoea, asthenia, myalgia, joint pain, gustatory dysfunction, abdominal pain, vomiting, and diarrhoea. The majority patients present with mild/moderate disease, however hospitalisation is sometimes required in particularly in elderly patients and/or patients having comorbidities such as diabetes and cardiovascular disease. A major complication in COVID-19 is progression to acute respiratory distress syndrome (ARDS), which presents as dyspnoea and acute respiratory failure, with patients requiring mechanical ventilation. In some embodiments the complement-related disorder is ARDS or acute respiratory failure.

Complement activation has been implicated in the pathogenesis of severe SARS-CoV-2 infection. Circulating markers of complement activation are elevated in patients with COVID-19 compared to those with influenza and to patients with non-COVID-19 respiratory failure. Patients hospitalized with COVID-19 reportedly have significantly higher median plasma sC5b-9, C5a, and Factor B levels compared to those with influenza, pneumonia or sepsis, and certain markers of complement activation have been associated with worse outcomes in COVID-19 patients, see e.g. Ma L et al., Sci Immunol. 2021 May 13; 6(59): eabh2259. Patients requiring ICU treatment, or who died from COVID-19 infection, were found to have significantly higher Factor D levels.

The levels of SARS-CoV-2 antigens and SARS-CoV-2 RNA in the blood reportedly correlates with the level of IL-6, inflammation, respiratory failure and death, see e.g. Brasen C L et al., Clin Chem Lab Med. 2021 Aug. 27. doi: 10.1515/cclm-2021-0694.

As described herein, elevated levels of FHL1, FHR1, FHR2, FHR3, FHR4 and FHR5 were observed to correlate with increasing COVID-19 severity, with the highest levels being observed in subjects with clinically severe COVID-19 requiring assisted ventilation. Thus, detection of one or more of FHL1, FHR1, FHR2, FHR3, FHR4 and/or FHR5 may predict the likelihood of a subject developing severe COVID-19. Appropriate treatment and monitoring can thus be deployed.

The methods described herein are useful for predicting the risk of development of conditions associated with SARS-CoV-2 infection, as well as predicting the severity of such infection, e.g. the likelihood of developing severe or critical COVID-19 and associated complications such as ARDS.

The present disclosure provides a method for determining/identifying whether a subject has, or is at risk of developing, a complement-related disorder associated with SARS-CoV-2 infection, e.g. COVID-19 or ARDS, the method comprising:
(a) digesting one or more complement proteins in a sample obtained from the subject with endoproteinase GluC to obtain one or more peptides, e.g. wherein the one or more complement proteins is selected from FHL-1, FHR1, FHR2, FHR3, FHR4 and/or FHR5;
(b) determining the level of the one or more peptides by mass spectrometry; and
(c) using the results of (b) to determine the level of the one or more complement proteins; and
(d) determining that the subject has or is at risk of developing a complement-related disorder associated with SARS-CoV-2 infection if the level of one or more of FHL-1, FHR1, FHR2, FHR3, FHR4 and/or FHR5, is elevated as compared to the level of that complement protein(s) in blood in a control subject that does not have COVID-19 or infection with SARS-CoV-2.

Methods provided herein may be useful for determining the risk of a subject developing a serious complement-related disorder, e.g. the methods are useful for distinguishing between subjects who may develop a mild complement-related disorder and subjects who are at risk of serious disease, and/or identifying subjects who are likely to develop serious disease.

In some embodiments the methods described herein can be used to identify subjects that are at risk of developing a severe disorder associated with SARS-CoV-2 infection, e.g. severe COVID-19 or critical COVID-19. Cases of COVID-19 can generally be categorised into five groups: asymptomatic, mild, moderate, severe and critical. Severe COVID-19 includes pneumonia and patients may require supplemental oxygen. Critical COVID-19 includes severe pneumonia and ARDS, and in some cases sepsis. Patients with critical COVID-19 require assisted ventilation.

In some aspects, the present invention provides methods of predicting, based on the analysis described herein of a sample from a subject, whether a subject is at risk of developing a complement-related disorder, has a complement-related disorder, is in need of treatment for a complement-related disorder, will respond to treatment for a complement-related disorder, and/or is responding/has responded to treatment for a complement-related disorder. The methods may be used for determining whether a subject is at risk of onset of the disorder, and/or is at risk of progression, exacerbation or worsening of the disorder.

Methods described herein may also be useful for assessing whether treatment for a complement-related disorder is/has been effective or successful.

In some aspects, the methods described herein may be useful for determining whether a subject is likely to respond or not respond to a therapeutic treatment, or whether a subject is responding to a therapeutic treatment. The methods should enable patients to receive the most effective therapy for their particular pathological requirements.

In some cases, the subject has or is suspected to have a complement-related disorder. In some cases the disorder is AMD. In some cases the disorder is EOMD. In some cases the disorder is glioblastoma e.g. glioblastoma multiforme (GBM). In some cases the disorder is a complement-related disorder associated with SARS-CoV-2 infection, e.g. COVID-19 or ARDS.

In some aspects, the present invention provides a method for treating or preventing a complement-related disorder in a subject, the method comprising administering an effective amount of a complement-targeted therapy/therapeutic agent, wherein the subject to be treated has been determined to have atypical presence or levels of one or more complement proteins, e.g. detected/determined as described herein, as compared to a control subject and/or reference value(s). In some aspects the subject has been determined to be at risk of developing a complement-related disorder, and/or identified as having a complement-related disorder.

In other aspects, the present invention provides a complement-targeted therapy/therapeutic agent for use in a method of treating or preventing a complement-related disorder in a subject, the method comprising administering an effective amount of the therapy/therapeutic agent, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In some aspects, provided is the use of a complement-targeted therapy/therapeutic agent in the manufacture of a medicament for treating or preventing a complement-related disorder in a subject, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

Also provided is a method of treating or preventing a complement-related disorder in a subject, or a complement-targeted therapy/therapeutic agent for use in a method of treating or preventing a complement-related disorder in a subject, the method comprising administering an effective amount of a complement-targeted therapy/therapeutic agent wherein the subject is selected for treatment if the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In various aspects provided herein, the subject to be treated has atypical presence or levels of at least one complement protein, preferably one or more of FH, FHL-1, FHR1, FHR2, FHR3, FHR4, FHR5, FI, C3, C3b, C3a, iC3b, C3f, C3c, C3dg, and/or C3d. In some embodiments, the subject to be treated has atypical presence of levels of one or more of FHR1, FHR2, and/or FHR3, and optionally FHR4 and/or FHR5, and/or FHL-1. The subject may benefit from treatment to reduce the level of any complement proteins that are increased as compared to a reference value(s) and/or from treatment to increase the level of any complement proteins that are decreased as compared to a reference value(s).

As used herein, ‘treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

Methods provided herein for assessing the risk of development, i.e. the onset or risk of progression of, or for identifying subjects having/at risk of, a complement-related disorder may be performed in conjunction with additional diagnostic methods and/or tests for such disorders that will be known to one skilled in the art. In some cases, methods for assessing the risk of development of a complement-related disorder comprise further techniques selected from: CH50 or AH50 measurement via haemolytic assay, measurement of neoantigen formation during MAC complex (C5b, C6, C7, C8, C9) generation, C3 deficiency screening, mannose-binding lectin assays, immunochemical assays to quantify individual complement components, flow cytometry to assess cell-bound regulatory proteins e.g. CD55, CD59 and CD35, and/or renal function tests, see e.g. Shih A R and Murali M R, Am. J. Hematol. 2015, 90(12):1180-1186, Ogedegbe H O, Laboratory Medicine, 2007, 38(5):295-304, and Gowda S et al., N Am J Med Sci. 2010, 2(4): 170-173, which are herein incorporated by reference in their entirety.

In some cases, methods provided herein for assessing the risk of development of AMD and/or EOMD comprise further assessment techniques selected from: dark adaptation testing, contrast sensitivity testing e.g. Pelli Robson, visual acuity testing using e.g. a Snellen chart and/or Amsler grid, Farnsworth-Munsell 100 hue test and Maximum Color Contrast Sensitivity test (MCCS) for assessing colour acuity and colour contrast sensitivity, preferential hyperacuity perimetry (PHP), fundus photography of the back of the eye, fundus examination, fundus autofluorescence, optical coherence tomography, angiography e.g. fluorescence angiography, fundus fluorescein angiography, indocyanine green angiography, optical coherence tomography angiography, adaptive optics retinal imaging, deep learning analysis of fundus images, electroretinogram methods, and/or methods to measure histological changes such as atrophy, retinal pigment changes, exudative changes e.g. hemorrhages in the eye, hard exudates, subretinal/sub-RPE/intraretinal fluid, and/or the presence of drusen.

Methods described herein may take into account lifestyle factors known to contribute to risk of developing complement-related disorders. For example, lifestyle factors that may cause or contribute to AMD include smoking, being overweight, high blood pressure and having a family history of AMD.

The methods provided herein may comprise determining in a subject the presence or absence of a genetic profile characterised by polymorphisms in the subject's genome associated with complement dysregulation. The polymorphisms may be found within or near genes such as CCL28, FBN2, ADAM12, PTPRC, IGLC1, HS3ST4, PRELP, PPID, SPOCK, APOB, SLC2A2, COL4A1, MYOC, ADAM19, FGFR2, C8A, FCN1, IFNAR2, C1 NH, C7 and ITGA4. A genetic profile associated with complement dysregulation may comprise one or more, often multiple, single nucleotide polymorphisms, e.g. as set out in Tables I and II of US 2010/0303832, which is hereby incorporated by reference in its entirety.

Genetic factors are thought to play a role in the development of AMD and EOMD. Thus, any of the assessment or therapeutic methods described herein may be performed in conjunction with methods to assess AMD-associated and/or EOMD-associated and/or macular dystrophy-associated genetic variants. In some cases a complement-related disorder described herein may comprise a genetic element and/or a genetic risk factor.

In some aspects of the present disclosure, there is provided a method of identifying a subject having a complement-related disorder or at risk of developing a complement-related disorder, the method comprising determining in a subject the presence or absence of one or more genetic factors associated with AMD and/or EOMD, e.g. one or more AMD- or EOMD-associated genetic variants.

In some cases, any method provided herein may comprise determining in a subject the presence or absence of one or more genetic factors associated with AMD and/or EOMD, e.g. one or more AMD- or EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors, e.g. by obtaining a sample from the subject, or in a sample obtained from the subject.

In some embodiments, the one or more genetic factors may be located on chromosome 1 at or near the RCA locus, e.g. in the CFH/CFHR genes/the CFH locus. In some embodiments, the presence of one or more CFH locus AMD-risk variants increase disease risk via increase of FHR protein levels.

The one or more genetic factors may be located in one or more of: CFH e.g. selected from Y402H (i.e. rs1061170^{C}), rs1410996c, I62V (rs800292), A473A (rs2274700), R53^{C}, D90G, D936E (rs1065489), R1210C, IVS1 (rs529825), IVS2 insTT, IVS6 (rs3766404), A307A (rs1061147), IVS10 (rs203674), rs3753396, R1210C, rs148553336, rs191281603, rs35292876, and rs800292; CFHR4 e.g. selected from rs6685931, and rs1409153; CFI e.g. selected from G119R, and rs141853578; CFB e.g. rs4151667, C2 e.g. rs9332739, C9 e.g. P167S; and/or C3 e.g. K155Q. In some embodiments, a genetic factor is Y402H (i.e. rs1061170^{C}). In some embodiments, a genetic factor is rs3753396. In some embodiments, a genetic factor is rs6685931 and/or rs1409153. In some embodiments, a genetic factor is at intronic KCNT2 rs61820755. In some embodiments, a genetic factor is not rs6685931.

In some embodiments a genetic factor is rs61820755, and may be associated with FHL-1.

In any embodiment herein, the genetic risk factors may be present in combination with elevated levels of one or more FHR proteins. The one or more genetic factors at the CFH locus may be selected from intergenic CFHR1/CFHR4 rs149369377 and/or rs61820755 for FHR-1, CFHR2 rs4085749 for FHR-2, intronic CFH rs70620 for FHR-3, rs12047098 for FHR-4, intronic KCNT2 rs72732232 for FHR-5. The presence of any one or more of these genetic factors indicates that the subject has or is likely to develop a complement-related disorder.

The one or more genetic risk factors may be selected from rs10922109, rs570618, rs121913059 (R1210C), rs148553336, rs187328863, rs61818925, rs35292876, and rs191281603.

The one or more genetic factors may be selected from one or more of rs13721756 on chromosome 10, rs111260777 on chromosome 11, rs117468955 on chromosome 12, rs200404865 on chromosome 13, rs4790395 on chromosome 17 and rs117115124 on chromosome 19. These factors may be present separately, or in addition to, genetic factors at the CFH locus. These factors may be present in combination with elevated FHR-3 levels.

Any and all combinations of genetic factors are envisaged, e.g. those described herein or additional factors, including their detection/assessment as below).

The methods described herein may involve detecting combinations of risk factors to assess the risk of a subject developing a complement related disorder, e.g. if one or both of the risk factors are present in a subject. For example:
rs10922109 and intergenic CFHR1/CFHR4 rs149369377
rs10922109 and CFHR2 rs4085749
rs10922109 and intronic CFH rs70620
rs10922109 and intergenic CFHR1-CFHR4 rs12047098
rs570618 and intergenic CFHR1/CFHR4 rs149369377
rs570618 and CFHR2 rs4085749
rs148553336 and intronic KCNT2 rs72732232
rs61818925 and CFHR2 rs4085749; and/or
rs61818925 and intergenic CFHR1-CFHR4 rs12047098.

Assessment of the presence of any genetic risk factor provided herein may be combined with the detection of any one or more of FHR1, FHR2, FHR3, FHR4 and/or FHR5, and optionally FHL-1 as described herein. For example, the presence of genetic factor rs10922109 may be assessed in combination with the detection of any one or more of FHR-1, FHR-2, FHR-3, and/or FHR-4; rs570618 may be assessed in combination with the detection of FHR-1 and/or FHR-2; rs61818925 may be assessed in combination with the detection of FHR-2 and/or FHR-4; and rs148553336 may be assessed in combination with the detection of FHR-5.

In some embodiments, the subject may comprise high risk CFH polymorphism T1277C. In some embodiments, a method according to the present disclosure does not comprise detecting the T1277C polymorphism. In some embodiments, the subject does not comprise high risk CFH polymorphism T1277C.

In any embodiment or method herein, the method may comprise a step of determining that the subject has or is likely to develop a complement-related disorder if one or more genetic factors, e.g. those described herein, are present.

Thus provided herein is a method of identifying a subject having a complement-related disorder or at risk of developing a complement-related disorder, the method comprising assessing the subject for one or more genetic risk factors, e.g. any of those described herein or others, and determining that the subject has or is likely to develop a complement-related disorder if the one or more genetic risk factors are present in the subject.

Provided herein is a method of determining whether a subject has, or is at risk of developing, a complement-related disorder, the method comprising assessing the subject for one or more genetic risk factors, e.g. any of those described herein or others, and determining that the subject has or is likely to develop a complement-related disorder if the one or more genetic risk factors are present in the subject.

Also provided is a method for selecting treatment for and/or treating subjects/patients that have a complement-related disorder or have been identified as having a complement-related disorder, e.g. using the steps above.

Also provided is a method for selecting a subject for treatment with a therapeutic agent, e.g. a complement-targeted therapy or therapeutic agent; a method for selecting a therapeutic agent, e.g. a complement-targeted therapy or therapeutic agent, for a subject; methods of treatment; a complement-targeted therapy or therapeutic agent for use in a method of treatment; and the use of a complement-targeted therapy or therapeutic agent in the manufacture of a medicament for the treatment of a complement-related disorder, wherein the method uses the steps above to assess genetic risk factors (either alone or in combination with determining the level of a complement protein e.g. an FHR protein as described herein and/or determining whether the level of one or more of FHR1, FHR2, FHR3, FHR4 and/or FHR5, and optionally FHL-1, is elevated as compared to the level of that complement protein in blood in a control subject that does not have a complement-related disorder, as described herein).

Any such method comprising detecting and assessing genetic risk factors may comprise a treatment step as described herein, e.g. treating a subject that has been determined to have or be likely to develop a complement-related disorder.

Other suitable genetic risk factors and genetic variants will be known in the art and may be as described in e.g. Edwards A O et al., Science 2005, 308(5720):421-4; Hageman G S et al., Proc Nat/Acad Sci USA. 2005, 102(20):7227-7232; Haines J L et al., Science 2005, 308(5720):419-21, Klein R J et al., Science 2005, 308(5720):385-389; Fritsche et al., Nat Genet. 2016, 48(2):134-43; US 2010/0303832; Clark S et al., J Clin Med. 2015, 4(1):18-31, Cipriani, V. et al., Nat Commun. 2020, 11, 778; or Hageman G S et al, Hum Genomics. 2011, 5, 420 (2011), each hereby incorporated by reference in its entirety.

In some cases, the methods provided herein further comprise determining in a subject the presence or absence of one or more genetic factors associated with EOMD, e.g. one or more EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors. In some embodiments the subject may possess one or more risk factors for early-onset macular degeneration (EOMD).

EOMD is thought to be caused by monogenic inheritance of rare variants of the CFH gene (see e.g. Boon C J et al. Am J Hum Genet 2008; 82(2):516-23; van de Ven J P, et al. Arch Ophthalmol 2012; 130(8):1038-47; Yu Y et al. Hum Mol Genet 2014; 23(19):5283-93; Duvvari M R, et al. Mol Vis 2015; 21:285-92; Hughes A E, et al. Acta Ophthalmol 2016; 94(3):e247-8; Wagner et al. Sci Rep 2016; 6:31531; Taylor R L et al, Ophthalmology. 2019 Mar. 21. pii: S0161-6420(18):33171-3). In some embodiments, the subject may possess one or more of EOMD-associated genetic variants. EOMD-associated genetic variants are described in e.g. Servais A et al. Kidney Int, 2012; 82(4):454-64 and Dragon-Durey M A, et al. J Am Soc Nephrol 2004; 15(3):787-95; which are hereby incorporated by reference in their entirety. In some embodiments, the subject may possess one or more of the following EOMD-associated genetic variants: CFH c.1243del, p.(Ala415Profs*39) het; CFH c.350+1G>T het; CFH c.619+1G>A het; CFH c.380G>A, p.(Arg127His); CFH c.694C>T, p.(Arg232Ter); or CFH c.1291T>A, p.(Cys431Ser).

In some cases, the methods provided herein comprise screening for deletions within the RCA locus (a region of DNA sequence located on chromosome one that extends from the CFH gene through to the CD46 (MCP) gene) that are associated with AMD and/or EOMD risk or protection.

Methods for determining the presence or absence of genetic factors include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, PCR, DNA sequencing e.g. Sanger sequencing or Next-Generation sequencing, allele specific oligonucleotide (ASO) probes, and oligonucleotide microarrays or beads. Other suitable methods are described in e.g. Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

In some embodiments, the subject is selected for therapeutic or prophylactic treatment with a complement-targeted therapy/therapeutic agent based on their being determined to possess one or more genetic factors for AMD and/or EOMD, e.g. one or more AMD-associated and/or EOMD-associated genetic variants, or for a macular dystrophy. In some embodiments, the subject has been determined to have one or more such genetic factors. In some embodiments, the methods provided herein comprise determining whether a subject possesses one or more such genetic factors. Such methods and genetic factors are described herein. Thus, provided herein is a method of diagnosing, treating or preventing a complement-related disorder in a subject, wherein the subject has/has been determined/is determined to possess one or more genetic factors for AMD and/or EOMD, and wherein the subject has/has been determined/is determined to have atypical presence or levels of one or more complement proteins, e.g. detected/determined as described herein, as compared to a reference value(s); optionally wherein the method comprises administering a complement-targeted therapy/therapeutic agent.

The term “subject” refers to a subject, patient or individual and may be any animal or human. The subject is preferably mammalian, more preferably human. The subject may be a non-human mammal, but is more preferably human. The subject may be male or female. The subject may be a patient. Therapeutic uses may be in human or animals (veterinary use). The subject to be treated with a therapeutic substance described herein may be a subject in need thereof.

The subject may be identified, or may have been identified, as having a complement-related disorder or being at risk of developing a complement-related disorder, e.g. by a method described herein.

A subject described herein may belong to a patient subpopulation i.e. the subject may be part of an identifiable, specific portion or subdivision of a population. The population and/or subpopulation may have or be suspected to have a complement-related disorder. The subpopulation may display atypical presence or levels of one or more complement proteins, e.g. detected/determined as described herein, as compared to the population as a whole. The population and/or subpopulation may have or be suspected to have AMD, EOMD or a macular dystrophy.

In some aspects provided herein, the subject is characterised as having an atypical presence or level of one or more complement proteins, e.g. detected/determined/measured as described herein.

A subject may have, have been determined to have, or be characterised as having elevated levels of a complement protein selected from one or more of FHR1, FHR2, FHR3, FHR4 and/or FHR5, and optionally FHL-1.

Provided is a method of treating or preventing a complement-related disorder in a subject, wherein the subject is characterised as having an atypical presence or levels of one or more complement proteins, e.g. detected/determined as described herein.

Also provided is a complement-targeted therapeutic agent for use in a method of treating or preventing a complement-related disease in a subject, wherein the subject is characterised as having an atypical presence or levels of one or more complement proteins, e.g. detected/determined as described herein.

Methods according to the present invention may be performed outside the human or animal body. Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism. In some embodiments, the determining, detecting, measuring, quantifying, predicting and/or diagnosing steps of the methods provided herein are performed in vitro.

Complement-Targeted Therapies and Therapeutic Agents

The methods of the present invention include treating a subject who is at risk of developing, predicted to be at risk of developing, who has been determined or identified to be at risk of developing, or who has, has been identified as having, has been determined to have, or has been diagnosed as having a complement-related disorder, e.g. as described herein. Such treatment may comprise administering a therapy or agent that targets the complement system and/or specific complement components. In some embodiments, an agent that “targets” a complement component acts to inhibit, degrade, silence, knock down, reduce or otherwise decrease expression and/or activity of said component.

Any method described herein for determining/identifying whether a subject has, or is at risk of developing, a complement-related disorder may additionally comprise a treatment step to treat said disorder. For example, a method provided herein for identifying whether a subject has or is at risk of developing a complement-related disorder may comprise a treatment step to treat or prevent said disorder, wherein the subject has been determined to have atypical presence or levels of one or more complement proteins, e.g. detected/determined as described herein, as compared to a reference value(s). In some cases the treatment is only administered if the subject has been determined to have, or be likely to have/at risk of having/at risk of developing, a complement-related disorder by a method described herein.

The terms ‘complement-targeted therapy’ and ‘complement-targeted therapeutic agent’ may be used interchangeably herein. In some instances, a complement-targeted therapeutic agent may also be a complement-targeted therapy, and vice versa. ‘Complement-targeted therapeutic agents’ may be referred to herein as ‘therapeutic agents’ or ‘agents’.

A treatment step may comprise administering to a subject a therapeutically or prophylactically effective amount of one or more complement-targeted therapies/therapeutic agents (also known as anti-complement therapy), for example, one or more C1 inhibitors, C5 inhibitors, C5a inhibitors, C5aR antagonists, C3 inhibitors, C3a inhibitors, C3b inhibitors, C3aR antagonists, classical pathway inhibitors, alternative pathway inhibitors, FH-supplementation therapy and/or MBL pathway inhibitors. Specific complement-targeted therapeutics include without limitation one or more of human C1 esterase inhibitor (C1-INH), eculizumab (Soliris®, Alexion; a humanized monoclonal IgG2/4-antibody targeting C5), APL-2 (Apellis), mubodina (Adienne Pharma and Biotech), ergidina (Adienne Pharma and Biotech), POT-4 (a cyclic peptide inhibitor of C3; Alcon), rituximab (Biogen Idec, Genentech/Roche), ofatumumab (Genmab, GSK), compstatin analogues, soluble and targeted forms of CD59, PMX53 and PMX205, (Cephalon/Teva), JPE-1375 (Jerini), CCX168 (ChemoCentryx), NGD-2000-1 (former Neurogen), Cinryze (Shire), Berinert (CSL Behring), Cetor (Sanquin), Ruconest/Conestat alfa (Pharming), TNT009 (True North), OMS721 (Omeros), CLG561 (Novartis), AMY-101 (Amyndas), APL-1 (Apellis), APL-2 (Apellis), Mirococept (MRC), Lampalizumab (FCD4514S, Genentech/Roche), ACH-4471 (Achillion), ALXN1210 (Alexion), Tesidolumab/LFG316 (Novartis/Morphosys), Coversin (Akari), RA101495 (Ra Pharma), Zimura (ARC1905, Ophthotech), ALN-CC5 (Alnylam), IFX-1 (InflaRx), ALXN1007 (Alexion), Avacopan/CCX168 (Chemocentryx) and/or one or more therapeutic agents as described in e.g. Ricklin and Lambris, Adv Exp Med Biol. 2013, 734: 1-22; Ricklin and Lambris, Semin Immunol. 2016, 28(3):208-22; Melis J P M et al., Mol Immunol. 2015 67(2):117-130; Thurman J M, Nephrol Dial Transplant, 2017 32: i57-i64; Cashman S M et al., PLoS One. 2011, 6(4):e19078; Bora N S et al., J Biol Chem. 2010, 285(44):33826-33; and Clark et al., J Clin Med 2015, 4(1):18-31 which are herein incorporated by reference in their entirety.

In some cases a complement-targeted therapeutic for use in the methods provided herein comprises a peptide or a polypeptide, e.g. that targets one or more complement proteins.

In some cases a treatment step comprises administering to a subject a therapeutically or prophylactically effective amount of one or more complement-targeted therapeutics described in WO 2018/224663 and/or WO 2019/138137, both hereby incorporated by reference in their entirety.

In some cases a complement-targeted therapeutic for use in the methods provided herein comprises a polypeptide which is capable of binding C3b, e.g. comprising an amino acid sequence having at least 85% identity to SEQ ID NO:145, 146, 147 or 148 and wherein the polypeptide has a total length of 450 amino acids or fewer, as described in WO 2019/138137. SEQ ID NO:145 to 148 described herein correspond to SEQ ID NO:4, 2, 3 and 13, respectively, described in WO 2019/138137. In some cases the complement-targeted therapeutic may have one or more of the following properties: binds to C3b, binds to C3b in the region of C3b bound by Complement Receptor 1, acts as a cofactor for FI, enables FI-mediated inactivation of C3b, reduces the amount of C3b via FI, increases the amount of C3b breakdown products e.g. iC3b, C3dg, C3d, C3f, e.g. via FI, and/or diffuses through BrM.

In some cases a complement-targeted therapeutic agent for use in the methods provided herein comprises a polypeptide comprising a C3b binding region and a C3b inactivating region, e.g. as described in WO 2018/224663, e.g. wherein the C3b inactivating region comprises, or consists of, an amino acid sequence having at least 65% sequence identity to the amino acid sequence of SEQ ID NO:149 and/or wherein the C3b binding region comprises, or consists of, an amino acid sequence having at least 65% sequence identity to the amino acid sequence of SEQ ID NO:150, 151 or 152. SEQ ID NO:149 to 152 described herein correspond to SEQ ID NO:9, 11, 13 and 14, respectively, described in WO 2018/224663. The polypeptide may comprise a linker between the C3b binding region and the C3b inactivating region. In some cases the polypeptide comprises a sequence comprising or consisting of an amino acid sequence having at least 65% sequence identity to the amino acid sequence of SEQ ID NO:32, 33 or 34 disclosed in WO 2018/224663. In some cases the complement-targeted therapeutic may have one or more of the following properties: binds to C3b, binds to C3b in the region of C3b bound by a cofactor for FI, acts as a cofactor for FI, enables FI-mediated inactivation of C3b, reduces the amount of C3b via FI, increases the amount of C3b breakdown products e.g. iC3b, C3dg, C3d, C3f, e.g. via FI, and/or diffuses through BrM.

In some cases a complement-targeted therapeutic agent for use in the methods provided herein is capable of increasing the level of FH and/or FHL-1 in the subject, e.g. a nucleic acid encoding FH or FHL-1, or a polypeptide corresponding to or derived from FH or FHL-1.

In some cases, subjects with elevated levels of FH and/or FHL-1, and/or increased expression of a gene(s) encoding FH and/or FHL-1, e.g. subjects suffering from glioblastoma, may derive therapeutic or prophylactic benefit from said levels being reduced. This may be achieved by administering any suitable agent that decreases the level of FH and/or FHL-1 and/or decreases the level of expression of the CFH gene.

Subjects with elevated levels of FHR1, FHR2, FHR3, FHR4 and/or FHR5, (and optionally FHL-1) and/or increased expression of a gene(s) encoding one or more FHR proteins/FHL-1, may derive therapeutic or prophylactic benefit from said levels being reduced. This may be achieved by administering any suitable agent that decreases the level of one or more of the FHR proteins/FHL-1 and/or decreases the level of expression of one or more CFHR/CFH genes.

The level of expression of a CFHR gene may be measured using techniques described herein and/or well known in the art, as reviewed in, for example, Roth C M, Curr. Issues Mol. Biol. 2002 4:93-100 and Kukurba K R and Montgomery S B, Cold Spring Harb Protoc. 2015, (11):951-969, which are hereby incorporated by reference in their entirety. For example, gene expression can be measured using quantitative PCR, real-time PCT, sequencing techniques e.g. RNA-seq, next-generation sequencing, microarrays, Northern blot, and ribonuclease protection assay (RPA). One skilled in the art will be able to appreciate a suitable technique(s) for measuring expression of CFHR1-5, as required. In some cases, the total RNA or cDNA may be extracted and isolated first from a cell sample.

Methods described herein may also comprise administering one or more agents that decrease the level of and/or decrease the expression of one or more complement proteins that are/have been determined to be elevated. Agents may be referred to a “complement-targeted therapeutic agents”.

In some embodiments, the agent is capable of decreasing the level of FHR1, FHR2, FHR3, FHR4 and/or FHR5, and/or capable of decreasing the level of expression of a gene encoding FHR1, FHR2, FHR3, FHR4 and/or FHR5. In some cases, the agent inhibits, degrades, silences, knocks down, reduces or otherwise decreases expression and/or activity of FHR mRNA or protein.

In some embodiments, the agent is capable of decreasing the level of FHL-1, and/or capable of decreasing the level of expression of a gene encoding FHL-1. In some cases, the agent inhibits, degrades, silences, knocks down, reduces or otherwise decreases expression and/or activity of FHL-1 mRNA or protein.

In some cases the complement-targeting therapeutic agent targets the same complement protein that is detected using a method described herein. In some cases the complement protein detected is FHR1 and the agent decreases the level of FHR1 and/or decreases the level of expression of a gene encoding FHR1. In some cases the complement protein detected is FHR2 and the agent decreases the level of FHR2 and/or decreases the level of expression of a gene encoding FHR2. In some cases the complement protein detected is FHR3 and the agent decreases the level of FHR3 and/or decreases the level of expression of a gene encoding FHR3. In some cases the complement protein detected is FHR4 and the agent decreases the level of FHR4 and/or decreases the level of expression of a gene encoding FHR4. In some cases the complement protein detected is FHR5 and the agent decreases the level of FHR5 and/or decreases the level of expression of a gene encoding FHR5. In some cases the complement protein detected is FHL-1 and the agent decreases the level of FHL-1 and/or decreases the level of expression of a gene encoding FHL-1. The present disclosure is intended to encompass targeting any one or combination of FHR1, FHR2, FHR3, FHR4 and/or FHR5, and optionally FHL-1, or genes encoding said proteins, using any suitable targeting agent(s).

In some cases the complement-targeting therapeutic agent targets a different complement protein to the protein detected using a method described herein. For example, a method may identify a subject as having a complement-related disorder by determining the level of one or more of FHR1 to FHR5, and then said subject may be treated with an agent that targets a different complement protein, such as FH, FHL-1, C3, C3b etc, such as the complement-targeted therapeutics described in WO 2018/224663 and/or WO 2019/138137. As another example, a method may identify a subject as having a complement-related disorder by determining an elevated level of FHR1, and said subject may be treated with an agent that decreases the level of one or more of FHR2, FHR3, FHR4 and/or FHR5 (or an equivalent situation with other combinations of FHR proteins).

A complement-targeting therapeutic agent may possess one or more of the following properties: acts to inhibit expression of one or more of the CFHR genes; interferes with transcription of the CFHR genes; interferes with translation of mRNA encoding FHR1, FHR2, FHR3, FHR4 and/or FHR5 protein; degrades mRNA encoding FHR1, FHR2, FHR3, FHR4 and/or FHR5; binds to FHR1, FHR2, FHR3, FHR4 and/or FHR5 protein; sequesters FHR1, FHR2, FHR3, FHR4 and/or FHR5 protein; sequesters FHR1, FHR2, FHR3, FHR4 and/or FHR5 protein in the blood; competes for binding of FHR1, FHR2, FHR3, FHR4 and/or FHR5 protein; blocks activity of FHR1, FHR2, FHR3, FHR4 and/or FHR5 protein; reduces the concentration of FHR1, FHR2, FHR3, FHR4 and/or FHR5 in the blood; reduces the ability of FHR1, FHR2, FHR3, FHR4 and/or FHR5 protein to leave the blood e.g. reduces their ability to enter tissue; reduces the ability of FHR1, FHR2, FHR3, FHR4 and/or FHR5 protein to reach the eye; reduces the amount of FHR1, FHR2, FHR3, FHR4 and/or FHR5 protein that enters the eye; reduces the amount of FHR1, FHR2, FHR3, FHR4 and/or FHR5 protein in the eye; reduces the ability of FHR1, FHR2, FHR3, FHR4 and/or FHR5 protein to enter or cross BrM; inhibits FHR1-, FHR2-, FHR3-, FHR4- and/or FHR5-mediated signalling; modulates a reaction involving C3b; modulates a reaction involving FHR1, FHR2, FHR3, FHR4 and/or FHR5 and C3b; reduces the ability of FHR1, FHR2, FHR3, FHR4 and/or FHR5 protein to bind to C3b; competes with FHR1, FHR2, FHR3, FHR4 and/or FHR5 protein for C3b binding; encourages dissociation of FHR1, FHR2, FHR3, FHR4 and/or FHR5 from C3b; binds to C3b e.g. in the region of C3b bound by a cofactor for Factor I; acts as a co-factor to enable Complement Factor I-mediated inactivation of C3b; reduces the amount of C3b and/or increases the amount C3b breakdown products, reduces C3 convertase activation; reduces production of C3bBb; increases C3 deactivation; reduces C3 activation; cleaves or helps to cleave C3b e.g. via FI; increases production of iC3b; decreases complement activation; and/or inactivates a complement pathway e.g. alternative complement pathway.

Herein, “inhibits”, “inhibition”, “reduces” or “reduction” refers to a reduction, decrease or lessening relative to a control condition. Herein, “decreases the level of FHR1, FHR2, FHR3, FHR4 and/or FHR5” refers to reduction or lessening relative to a control condition. The level of FHR1, FHR2, FHR3, FHR4 and/or FHR5 may be measured by determining the level, amount or concentration of FHR1, FHR2, FHR3, FHR4 and/or FHR5 in the blood of a subject relative to a reference level. A decrease in the level of FHR1, FHR2, FHR3, FHR4 and/or FHR5 and/or a decrease in the level of expression of a gene encoding FHR1, FHR2, FHR3, FHR4 and/or FHR5 may be measured by determining the level of the protein/the level of expression of a gene encoding the protein in the subject after treatment with the agent and comparing it to the level in the subject before treatment or comparing it with the level of the protein/the level of expression of a gene encoding the protein in a control subject that does not have a complement-related disorder, e.g. the level may be less elevated after treatment as compared to the control subject when compared to the elevation before treatment. The decrease in the level of FHR1, FHR2, FHR3, FHR4 and/or FHR5 may also refer to the sequestration or binding of FHR1, FHR2, FHR3, FHR4 and/or FHR5 by agents in such a way that circulating levels/amount/concentration of FHR1, FHR2, FHR3, FHR4 and/or FHR5 are decreased. Thus, in some cases, an agent may be described as an agent that decreases the level of circulating FHR1, FHR2, FHR3, FHR4 and/or FHR5.

The complement-targeted therapeutic agent may be nucleic acid-based or comprise nucleic acid elements. The agent may promote silencing of gene expression via RNA-mediated interference (RNAi) or antisense degradation mechanisms, e.g. via RNase H.

In some cases the agent is, or comprises, an antisense nucleic acid. An “antisense nucleic acid” as referred to herein is a nucleic acid (e.g. DNA or RNA molecule) that is complementary to at least a portion of a specific target nucleic acid (e.g. an mRNA translatable into a protein, such as an FHR protein) and is capable of reducing transcription of the target nucleic acid (e.g. mRNA from DNA), reducing the translation of the target nucleic acid (e.g. mRNA) or altering transcript splicing (e.g. by a single stranded morpholino oligo). Antisense nucleic acids may be single stranded, e.g. gapmers, or may be double stranded e.g. siRNA. Antisense nucleic acids are capable of hybridizing to (e.g. selectively hybridizing to) a target nucleic acid (e.g. target mRNA) via Watson-Crick base pairing. In some cases the antisense nucleic acids specifically bind to the target nucleic acid. In some cases, the antisense nucleic acid hybridizes to the target nucleic acid sequence (e.g. mRNA) under stringent hybridization conditions. In some cases, the antisense nucleic acid hybridizes to the target nucleic acid (e.g. mRNA) under moderately stringent hybridization conditions.

The nucleotide sequence of an antisense nucleic acid is sufficiently complementary to the target nucleic acid of interest such that it binds or hybridises to the target nucleic acid. Thus, if a skilled person knows the sequence of the target nucleic acid, it is easy and routine to design a suitable antisense nucleic acid that will hybridize to the target to achieve the desired effect.

The target RNA may be an mRNA that encodes for all or part of a complement protein, such as FHR1, FHR2, FHR3, FHR4 or FHR5, or FHL-1, mRNA. A nucleic acid that acts as a complement-targeted therapeutic agent may target one of the five FHR mRNAs/proteins, i.e. specifically target FHR1, FHR2, FHR3, FHR4 or FHR5, or it may target more than one of FHR1, FHR2, FHR3, FHR4 or FHR5 as a result of the sequence similarity between the FHR proteins.

In some cases the agent is capable of promoting RNA interference (RNAi). RNAi uses small double-stranded RNA molecules to cause degradation of target mRNA. Non-limiting examples of antisense nucleic acids for use as agents according to the present invention include siRNAs (including their derivatives or pre-cursors, such as nucleotide analogs), short hairpin RNAs (shRNA), micro RNAs (miRNA, including their long primary transcripts (pri-miRNAs) and partially processed 60-70 base pair hairpin transcripts (pre-miRNAs)), saRNAs (small activating RNAs) and small nucleolar RNAs (snoRNA) or certain of their derivatives or pre-cursors. Antisense nucleic acid molecules may stimulate RNA interference (RNAi). siRNA nucleic acids are ˜21-25 nucleotides in length and comprise a guide strand which hybridizes with the target mRNA, plus a complementary passenger strand (e.g., each complementary sequence of the double stranded siRNA is 21-25 nucleotides in length, and the double stranded siRNA is about 21-25 base pairs in length). They promote degradation of the target mRNA via RISC. Structure and function of siRNAs are well known in the art and are described in e.g. Kim and Rossi, Biotechniques. 2008 April; 44(5): 613-616. Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

microRNAs (miRNAs) also regulate gene expression via RISC. They are initially expressed as long primary transcripts (pri-miRNAs), which are processed within the nucleus into 60-70 nucleotide hairpins (pre-miRNAs), which are further processed in the cytoplasm into small double stranded nucleic acids that interact with RISC and target mRNA. miRNAs comprise “seed sequences” that are essential for binding to target mRNA. “Seed sequences” usually comprise six nucleotides and are situated at positions 2-7 at the miRNA 5′ end.

In some embodiments the agent comprises a double stranded nucleic acid molecule in which one strand is wholly or partially complementary to, or hybridizes with, an mRNA sequence encoding all or part of FHR1, FHR2, FHR3, FHR4 or FHR5, or FHL-1. In some embodiments the agent comprises a siRNA molecule comprising a guide strand complementary to, or that hybridizes with, a portion of an mRNA sequence that encodes all or part of FHR1, FHR2, FHR3, FHR4 or FHR5, or FHL-1. In some embodiments the agent comprises a miRNA molecule (pri-, pre- or mature miRNA) comprising a seed sequence capable of hybridizing to a portion of an mRNA sequence that encodes all or part of FHR1, FHR2, FHR3, FHR4 and/or FHR5, or FHL-1.

In some cases the agent is a single stranded antisense oligonucleotide (ASO). ASOs modify expression of a target RNA, either by altering splicing or by recruiting RNase H to degrade the target RNA. RNase H recognises DNA:RNA hybrids formed when the ASO binds to the target RNA. ASOs tend to be 18-30 base pairs in length. Many ASOs are designed as chimeras, comprising a mix of bases with different chemistries, or as gapmers, comprising a central DNA portion surrounded by ‘wings’ of modified bases. ASOs are described in e.g. Scoles et al., Neurol Genet. 2019 April; 5(2): e323.

Antisense nucleic acids may comprise naturally occurring nucleotides or modifications such as e.g. phosphorothioate linkages, phosphorodiamidate linkages, methoxyethyl nucleotide modifications e.g. 2-MOE, ‘locked’ nucleic acids e.g. LNAs, peptide nucleic acids (PNAs), and/or 5′-methylcytosine modifications.

In some embodiments the agent comprises an antisense oligonucleotide that is capable of hybridizing to a portion of an mRNA sequence that encodes all or part of FHR1, FHR2, FHR3, FHR4 and/or FHR5, or FHL-1.

Antisense nucleic acids described herein may comprise or consist of nucleotide sequences having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% complementarity to their target nucleic acid. Complementarity may be calculated over the whole length of the antisense nucleic acids and/or over all or part of the target nucleic acid to which the antisense nucleic acid binds.

A nucleic acid for use as a complement-targeted therapeutic agent may be an siRNA as described in WO 2019/215330 A1, which is hereby incorporated by reference in its entirety.

A nucleic acid for use as a complement-targeted therapeutic agent may be an antisense molecule, e.g. siRNA, comprising SEQ ID NO: 174 and/or SEQ ID NO: 175.

The nucleic acid molecule may be an aptamer. The term “aptamer” as used herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W, Nature 1990, 346:818-822; Tuerk C, Gold L. Science 1990, 249:505-510) or by developing SOMAmers (slow off-rate modified aptamers) (Gold L et al. (2010) Aptamer-based multiplexed proteomic technology for biomarker discovery. PLoS ONE 5(12):e15004). SOMAmers are short, single stranded deoxyoligonucleotides with protein-like properties thanks to functional groups that mimic amino acid side chains. Applying the SELEX and the SOMAmer technology includes for instance adding functional groups that mimic amino acid side chains to expand the aptamer's chemical diversity. As a result high affinity aptamers for a target may be enriched and identified.

Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12): 2223).

Aptamers may be peptides selected or engineered to bind specific target molecules. Peptide aptamers and methods for their generation and identification are reviewed in Reverdatto et al., Curr Top Med Chem. (2015) 15(12):1082-101, which is hereby incorporated by reference in its entirety. Peptide aptamers may optionally have a minimum length of one of 2, 3, 4, 5, 6, 7, 8, 9 or 10 amino acids. Peptide aptamers may optionally have a maximum length of one of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. Suitable peptide aptamers may optionally have a length of one of 2-30, 2-25, 2-20, 5-30, 5-25 or 5-20 amino acids.

Aptamers may have K_{D}'s in the nM or pM range, e.g. less than one of 500 nM, 100 nM, 50 nM, 10 nM, 1 nM, 500 pM, 100 pM.

An aptamer or SOMAmer suitable for use as described herein may bind to FHR1, FHR2, FHR3, FHR4 and/or FHR5. An aptamer or SOMAmer suitable for use as described herein may display specific binding for FHR1, FHR2, FHR3, FHR4 or FHR5. The aptamer may inhibit the function of FHR1, FHR2, FHR3, FHR4 or FHR5, for example blocking their binding to other complement proteins such as C3b.

In some embodiments, the agent is an antibody or antigen-binding molecule (both referred to herein as “antigen-binding molecule”) e.g. an anti-FHR1 antibody. In some cases, the antigen-binding molecule is specific for FHR1, FHR2, FHR3, FHR4 and/or FHR5. In some cases, the antigen-binding molecule displays specific binding to FHR1, FHR2, FHR3, FHR4 and/or FHR5. In some cases, the antigen-binding molecule displays specific binding to FHR1, FHR2, FHR3, FHR4 and/or FHR5, and optionally to FH and FHL-1. An antibody that acts as a complement-targeted therapeutic agent may target one of the five FHR mRNAs/proteins, i.e. specifically target FHR1, FHR2, FHR3, FHR4 or FHR5, or it may target more than one of FHR1, FHR2, FHR3, FHR4 or FHR5 e.g. as a result of the sequence similarity between the FHR proteins.

In some cases, the antigen-binding molecule is specific for C3b. In some cases, the antigen-binding molecule displays specific binding to C3b. In some cases, the antigen-binding molecule displays specific binding to FH and/or FHL-1. As used herein, “specific binding” refers to binding which is selective for the antigen, and which can be discriminated from non-specific binding to non-target antigen. An antigen-binding molecule that specifically binds to a target molecule preferably binds the target with greater affinity, and/or with greater duration than it binds to other, non-target molecules. In some cases, the antigen-binding molecule displays specific binding for FHR1, FHR2, FHR3, FHR4 or FHR5, and optionally FH and/or FHL-1, over other complement proteins. An antigen-binding molecule may bind to human FHR1, FHR2, FHR3, FHR4 and/or FHR5, and optionally to FH and FHL-1, with a KD of 1 μM or less, preferably one of ≤1 μM, ≤100 nM, ≤510 nM, ≤51 nM or ≤100 pM.

Anti-FHR antigen-binding molecules may be antagonist antigen-binding molecules that inhibit or reduce a biological activity of FHR1, FHR2, FHR3, FHR4 and/or FHR5. Anti-FHR antigen-binding molecules may be neutralising antigen-binding molecules that neutralise the biological effect of FHR1, FHR2, FHR3, FHR4 and/or FHR5, e.g. an ability to stimulate production of C3 convertase via C3b.

The antigen-binding molecule may bind to a particular region of interest of FHR1, FHR2, FHR3, FHR4 and/or FHR5, FH/FHL-1 or C3b. The antigen-binding region of an antigen-binding molecule may bind to a linear epitope of FHR1, FHR2, FHR3, FHR4 and/or FHR5, FH/FHL-1 or C3b, consisting of a contiguous sequence of amino acids (i.e. an amino acid primary sequence). In some embodiments, the antigen-binding region molecule may bind to a conformational epitope of FHR1, FHR2, FHR3, FHR4 and/or FHR5, FH/FHL-1 or C3b, consisting of a discontinuous sequence of amino acids of the amino acid sequence.

The antigen-binding molecule may be a multispecific antigen-binding molecule. By “multispecific” it is meant that the antigen-binding molecule displays specific binding to more than one target. In some embodiments the antigen-binding molecule is a bispecific antigen-binding molecule. In some embodiments the antigen-binding molecule comprises at least two different antigen-binding domains (i.e. at least two antigen-binding domains, e.g. comprising non-identical VHs and VLs). Multispecific antigen-binding molecules may be provided in any suitable format, such as those formats described in described in Brinkmann and Kontermann MAbs (2017) 9(2): 182-212, which is hereby incorporated by reference in its entirety.

In some embodiments the antigen-binding molecule binds to FHR1, FHR2, FHR3, FHR4 and/or FHR5, and optionally FH/FHL-1, and another target (e.g. an antigen other than the FHR/FH proteins), and so is at least bispecific. The term “bispecific” means that the antigen-binding molecule is able to bind specifically to at least two distinct antigenic determinants.

The ability of a given polypeptide to bind specifically to a given molecule or another given peptide/polypeptide can be determined by analysis according to methods known in the art, such as by ELISA, Surface Plasmon Resonance (SPR; see e.g. Hearty et al., Methods Mol Biol 2012, 907:411-442), Bio-Layer Interferometry (see e.g. Lad et al., J Biomol Screen. 2015, 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay. Binding affinity may be expressed in terms of dissociation constant (KD).

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, the antigen-binding molecule decreases the concentration of FHR1, FHR2, FHR3, FHR4 and/or FHR5 in the blood. In some embodiments, the antigen-binding molecule decreases the amount of circulating FHR1, FHR2, FHR3, FHR4 and/or FHR5 e.g. in the blood. In some embodiments, the antigen-binding molecule may sequester FHR1, FHR2, FHR3, FHR4 and/or FHR5 protein. In some embodiments, the antigen-binding molecule binds to FHR1, FHR2, FHR3, FHR4 and/or FHR5 and reduces the ability of FHR1, FHR2, FHR3, FHR4 and/or FHR5 to reach the eye, enter the BrM, and/or enter the intercapillary septa of the choriocapillaris. In some embodiments, the antigen-binding molecule reduces binding of FHR1, FHR2, FHR3, FHR4 and/or FHR5 to C3b.

The ability of an antigen-binding molecule to inhibit interaction between two binding partners can also be determined by analysis of the downstream functional consequences of such interaction. For example, the ability of an antigen-binding molecule to inhibit interaction of FHR1, FHR2, FHR3, FHR4 and/or FHR5 and C3b may be determined by analysis of production of C3bBb and/or iC3b in an appropriate assay e.g. by detecting the production of protein from a reaction using ELISA, Western blotting or electrophoresis methods.

A person skilled in the art will be able to produce suitable antigen binding molecules using e.g. techniques as described herein or those known in the art, see e.g. Chiu and Gilliland, Curr Opin Struct Biol. 2016, 38:163-173, Jakobovits A, Curr Opin Biotechnol. 1995 October; 6(5):561-6, and Brüggemann M et al., Arch Immunol Ther Exp (Warsz). 2015; 63(2): 101-108. One suitable technique is phage display technology, see e.g. Hammers and Stanley, J Invest Dermatol. 2014, 134(2): e17 and Bazan J et al., Hum Vaccin Immunother. 2012, 8(12): 1817-1828. Antigen-binding polypeptide chains may also be produced by techniques such as chemical synthesis (see e.g. Chandrudu et al., Molecules (2013), 18: 4373-4388), recombinant expression such as the techniques set out in Green and Sambrook, Molecular Cloning: A Laboratory Manual (4th Edition), Cold Spring Harbor Press, 2012, and in Nat Methods. (2008); 5(2): 135-146, or cell-free-protein synthesis (CFPS; see e.g., Zemella et al. Chembiochem (2015) 16(17): 2420-2431), all of which are hereby incorporated by reference in their entirety. The antigen-binding molecule may be monoclonal, i.e. a homogenous population of antibodies specifically targeting a single epitope on an antigen. Monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Suitable polyclonal antibodies can also be prepared using methods well known in the art.

The antigen-binding portion may be a part of an antibody (for example a Fab fragment) or a synthetic antibody fragment (for example a single chain Fv fragment [ScFv]). Antigen-binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

Antibodies and antigen-binding fragments according to the present disclosure comprise the complementarity-determining regions (CDRs) of an antibody which is capable of binding to the relevant target molecule, i.e. a complement protein such as those described herein.

The skilled person is well aware of techniques for producing antibodies suitable for therapeutic use in a given species/subject. For example, procedures for producing antibodies suitable for therapeutic use in humans are described in Park and Smolen Advances in Protein Chemistry (2001) 56: 369-421 (hereby incorporated by reference in its entirety). Antibodies to a given target protein (e.g. FHR1, FHR2 etc) can be raised in model species (e.g. rodents, lagomorphs), and subsequently engineered in order to improve their suitability for therapeutic use in a given species/subject. For example, one or more amino acids of monoclonal antibodies raised by immunisation of model species can be substituted to arrive at an antibody sequence which is more similar to human germline immunoglobulin sequences (thereby reducing the potential for anti-xenogenic antibody immune responses in the human subject treated with the antibody). Modifications in the antibody variable domains may focus on the framework regions in order to preserve the antibody paratope. Antibody humanisation is a matter of routine practice in the art of antibody technology, and is reviewed e.g. in Almagro and Fransson, Frontiers in Bioscience (2008) 13:1619-1633, Safdari et al., Biotechnology and Genetic Engineering Reviews (2013) 29(2): 175-186 and Lo et al., Microbiology Spectrum (2014) 2(1), all of which are hereby incorporated by reference in their entirety. The requirement for humanisation can be circumvented by raising antibodies to a given target protein (e.g. FHR1, FHR2 etc) in transgenic model species expressing human immunoglobulin genes, such that the antibodies raised in such animals are fully-human (described e.g. in Bruggemann et al., Arch Immunol Ther Exp (Warsz) (2015) 63(2):101-108, which is hereby incorporated by reference in its entirety).

Phage display techniques may also be employed to the identification of antibodies to a given target protein (e.g. IL-11 or IL-11 Rα), and are well known to the skilled person. The use of phage display for the identification of fully human antibodies to human target proteins is reviewed e.g. in Hoogenboom, Nat. Biotechnol. (2005) 23, 1105-1116 and Chan et al., International Immunology (2014) 26(12): 649-657, which are hereby incorporated by reference in their entirety.

Suitable antibodies for use as complement-targeted therapies may include those described in e.g. Irmscher et al., Nat Commun 10, 2961 (2019); Heinen et al., Blood 2009; 114 (12): 2439-2447; Schäfer et al., Front. Immunol. 7:542; Campa et al., Cancer Immunol Res. 2015; 3(12):1325-1332; Oppermann et al., Clinical & Experimental Immunology, 2006, 144: 342-352; or humanised versions thereof. Other antibodies that may be used in the present invention include MBS8531469 (MyBioSource), ab182652 (Abcam), HPA049813 or SAB1409184 (Sigma), GTX32603 (GeneTex), EGO017/EGO018/EGO019 (Kerafast), PA5-41991 (ThermoFisher), HP9058 (HycultBiotech), SAB1401859 (Sigma), MBS600911 (MyBioSource), TAB-063MZ (Creative Biolabs), or ab49241 (Abcam), or humanised versions thereof e.g. comprising same/similar CDR sequences but with human framework regions.

An agent may be a sequestering agent, e.g. of FHR1, FHR2, FHR3, FHR4 and/or FHR5. The agent may be a protein molecule. An example is an antigen-binding molecule e.g. as described herein, which sequesters FHR1, FHR2, FHR3, FHR4 and/or FHR5 in the blood.

The agent may be a small molecule. For example, the small molecule may bind to FHR1, FHR2, FHR3, FHR4 and/or FHR5 protein and prevent/reduce the ability of FHR1, FHR2, FHR3, FHR4 and/or FHR5 to reach sites of complement activation and/or prevent/reduce an interaction between FHR1, FHR2, FHR3, FHR4 and/or FHR5 and a normal binding partner e.g. C3b. The small molecule may prevent/reduce correct folding of the FHR1, FHR2, FHR3, FHR4 and/or FHR5 protein. In some cases, the small molecule prevents/reduces binding between FHR1, FHR2, FHR3, FHR4 and/or FHR5 and a binding partner. In some cases, the small molecule binds to FHR1, FHR2, FHR3, FHR4 and/or FHR5.

An agent may be a decoy receptor. In some embodiments, a decoy receptor refers to a peptide or polypeptide capable of binding FHR1, FHR2, FHR3, FHR4 and/or FHR5. The receptor may be a receptor, including fragments and derivatives thereof, for FHR1, FHR2, FHR3, FHR4 and/or FHR5. A decoy receptor may be able to recognise and bind a specific ligand but may not be able to signal or activate a subsequent response. A decoy receptor may bind FHR1, FHR2, FHR3, FHR4 and/or FHR5 to form a complex. A decoy receptor may act as an inhibitor of FHR1, FHR2, FHR3, FHR4 and/or FHR5 by binding FHR1, FHR2, FHR3, FHR4 and/or FHR5 and preventing/reducing the ability or availability of the proteins to bind to their receptor(s). Thus the agent may be a molecule which binds FHR1, FHR2, FHR3, FHR4 and/or FHR5 to prevent activation of C3/C3b. A decoy receptor may act as an inhibitor of FHR1, FHR2, FHR3, FHR4 and/or FHR5 by binding to a binding partner of one or more of the proteins, e.g. in the region that would usually be bound by FHR1, FHR2, FHR3, FHR4 and/or FHR5, and preventing interaction between FHR1, FHR2, FHR3, FHR4 and/or FHR5 and one or more binding partners. The agent may be based on C3b, for example the receptor may be an inactive form of C3b. The agent may be based on C3c and/or C3d.

The agent may be administered to/present in the blood, or attached to a tissue e.g. in or near the eye. The receptor may be capable of inhibiting complement activation. The receptor may be capable of inhibiting interaction between FHR1, FHR2, FHR3, FHR4 and/or FHR5 and C3b. The receptor may be capable of inhibiting the activation of C3b, and/or inhibiting the formation of C3 convertase.

A decoy receptor may be soluble (not membrane bound), or may be membrane bound e.g. expressed on a cell surface. Decoy receptors may be presented and/or administered on a surface of a nanocarrier, for example, a nanoparticle, liposome, bead, polymer, metal particle, dendrimer, nanotube or micro-sized silica rods, see e.g. Wilczewska A Z et al., Pharmacol Rep. 2012, 64(5):1020-1037.

Methods for detecting whether a decoy receptor competes for FHR1, FHR2, FHR3, FHR4 and/or FHR5 binding are described herein e.g. SPR (see e.g. Hearty et al., Methods Mol Biol 2012, 907:411-442), competition ELISA assay or solid phase binding assays. Other suitable methods will be known in the art.

Agents that decrease the amount of FHR1, FHR2, FHR3, FHR4 and/or FHR5 and/or decrease expression of a gene encoding FHR1, FHR2, FHR3, FHR4 and/or FHR5 may fall into more than one of the categories above. For example, an antigen binding molecule or decoy receptor may also be a sequestering agent.

Any of the agents described herein may be optionally isolated and/or substantially purified.

Complement-targeted therapeutic agents may be formulated as pharmaceutical compositions or medicaments for clinical use and may comprise a pharmaceutically acceptable carrier, diluent, excipient or adjuvant. In accordance with the present invention methods are also provided for the production of pharmaceutically useful compositions, such methods of production may comprise one or more steps selected from: isolating an agent; and/or mixing an agent with a pharmaceutically acceptable carrier, adjuvant, excipient or diluent. The agent or composition may be formulated for topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal routes of administration which may include injection or infusion, or administration as an eye drop (i.e. ophthalmic administration). Suitable formulations may comprise the agent in a sterile or isotonic medium. Medicaments and pharmaceutical compositions may be formulated in fluid, including gel, form. Fluid formulations may be formulated for administration by injection or infusion (e.g. via catheter) to a selected organ or region of the human or animal body. A further aspect of the present invention relates to a method of formulating or producing a medicament or pharmaceutical composition for use in a method of medical treatment, the method comprising formulating a pharmaceutical composition or medicament by mixing an agent with a pharmaceutically acceptable carrier, adjuvant, excipient or diluent.

In some cases, the agent is administered to the liver, e.g. to one or more hepatocytes. In some cases, the agent is administered to the blood (i.e. intravenous/intra-arterial administration). In some cases, the agent is administered subcutaneously.

In some cases, the methods comprise targeted delivery of the agent i.e. wherein the concentration of the agent in the subject is increased in some parts of the body relative to other parts and/or wherein the agent is delivered via a controlled-release technique. In some cases, the methods comprise intravenous, intra-arterial, intramuscular or subcutaneous administration and wherein the agent is formulated in a targeted agent delivery system. Suitable targeted agent delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and agent delivery systems will be apparent to one skilled in the art. In some cases, the agent is formulated for targeted delivery to a specific organ(s) or tissue(s). In some cases, the agent is delivered to the liver. In some cases, the methods comprise intravenous, intra-arterial, intramuscular or subcutaneous administration and wherein the agent is formulated for targeted delivery to the liver.

In some cases, RNA, e.g. nanoparticle based formulations, may be formulated for pulmonary administration for subsequent delivery to non-lung tissues, see e.g. US 2015/0157565 A1, which is herein incorporated in its entirety.

The particular mode and/or site of administration may be selected in accordance with the location where reduction of FHR protein levels and/or reduction of the level of CFHR expression is required. In some cases, the methods comprise intravenous and/or intra-arterial administration. In some cases, the methods comprise administration to the eye. Should reduction of CFHR expression be required, then an agent that decreases expression of one or more CFHR genes may be administered to the liver. In some cases, the agent is delivered to one or more hepatocytes.

Methods for RNA delivery are described herein and are known in the art and can be found, for example, in Tatiparti K et al. “siRNA Delivery Strategies: A Comprehensive Review of Recent Developments.” Ed. Thomas Nann. Nanomaterials 7.4 (2017): 77, and Lehto T et al., Adv Drug Deliv Rev. 2016, 106(Pt A): 172-182, which are herein incorporated by reference in their entirety. For example, RNA may be delivered naked, or by using nanoparticles, polymers, peptides e.g. cell-penetrating peptides, or by ex vivo transfection. Nanoparticles may be organic, e.g. micelles, liposomes, proteins, solid-lipid particles, solid polymer particles, dendrimers, and polymer therapeutics. Nanoparticles may be inorganic such as nanotubes or metal particles, optionally with organic molecules added. Viruses present another nanoparticle delivery option. Nanoparticles may be optimised to improve rate of endocytosis, avoid renal clearance and filtration, improve thermal stability, improve pH stability, prevent toxic effects, and improve RNA loading efficiency. Further encapsulation methods are described in e.g. US 2015/0157675 A1.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments (e.g. other therapeutic or prophylactic intervention), either simultaneously or sequentially depending on the condition to be treated. A complement-targeted therapeutic agent and another therapeutic agent may be administered simultaneously or sequentially. In some cases, two or more complement-targeted therapeutic agents, e.g. as described herein, are administered simultaneously or sequentially.

Other therapeutic agents or techniques suitable for use with the present invention may comprise nutritional therapy, photodynamic therapy (PDT), laser photocoagulation, anti-VEGF (vascular endothelial growth factor) therapy, and/or additional therapies known in the art, see e.g. Al-Zamil W M and Yassin S A, Clin Interv Aging. 2017 Aug. 22; 12:1313-1330). Anti-VEGF therapy may comprise agents such as ranibizumab (Lucentis, made by Genentech/Novartis), Avastin (Genentech), bevacizumab (off label Avastin), and aflibercept (Eylea®/VEGF Trap-Eye from Regeneron/Bayer). Further agents or techniques suitable for use with the present invention include APL-2 (Apellis), AdPEDF (GenVec), encapsulated cell technology (ECT; Neurotech), squalamine lactate (EVIZON™, Genaera), OT-551 (antioxidant eye drops, Othera), anecortave actate (Retaane®, Alcon), bevasiranib (siRNA, Acuity Pharmaceuticals), pegaptanib sodium (Macugen®), and AAVCAGsCD59 (Clinical trial identifier: NCT03144999).

Simultaneous administration refers to administration of the complement-targeted therapeutic agent and another therapeutic agent together, for example as a pharmaceutical composition containing both agents (combined preparation), or immediately after each other and optionally via the same route of administration, e.g. to the same tissue, artery, vein or other blood vessel. Sequential administration refers to administration of one of the polypeptide, nucleic acid, vector, cell or composition or therapeutic agent followed after a given time interval by separate administration of the other agent. It is not required that the two agents are administered by the same route, although this is the case in some embodiments. The time interval may be any time interval.

Multiple doses of a complement-targeted therapeutic agent may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

An agent described herein may be formulated in a sustained release delivery system, in order to release the polypeptide, nucleic acid, vector or composition at a predetermined rate. Sustained release delivery systems may maintain a constant drug/therapeutic concentration for a specified period of time. In some embodiments, an agent described herein is formulated in a liposome, gel, implant, device, or drug-polymer conjugate e.g. hydrogel.

Methods for Detecting/Determining the Level of Complement Proteins

The present invention involves detecting the presence of, and/or determining the level of, one or more complement proteins using suitable analytical techniques, e.g. as described herein.

In some embodiments a method described herein comprises contacting the complement protein with endoproteinase GluC to obtain one or more peptides, and detecting the one or more peptides by mass spectrometry.

In some embodiments a method described herein comprises contacting, e.g. digesting, the protein with GluC to obtain one or more peptides, and determining the level of the one or more peptides by mass spectrometry. In some cases, the methods involves both detecting a complement protein and determining the level of a complement protein. The protein may be the same protein, or the methods may involve detection of a first complement protein and determining the level of a second complement protein.

In any and all methods described herein, the step of detecting/determining the level of the one or more peptides consists of detecting/determining the level of/measuring the peptide(s) by mass spectrometry. That is, the step of detecting/determining the level of/measuring the peptide(s) is performed by mass spectrometry only. Measuring the peptide(s) may include detecting the presence or absence of the one or more peptides, and/or determining the level, amount and/or concentration of each peptide in the sample.

In some embodiments, the step of determining in any method described herein comprises:
(i) digesting at least one complement protein in a sample e.g. blood sample obtained from the subject with endoproteinase GluC to obtain one or more peptides;
(ii) determining the presence and/or level of the one or more peptides by mass spectrometry, or performing mass spectrometry to determine the presence and/or level of the one or more peptides; and
(iii) using the results of (ii) to determine whether or not the level of the complement protein(s) is elevated, e.g. as compared to the level of that complement protein(s) in a blood sample in a control subject that does not have a complement-related disorder.

In some cases, the step of determining in any method described herein comprises:
(i) obtaining a blood-derived sample from a subject, wherein the sample comprises at least one complement protein that has been digested with endoproteinase GluC to obtain one or more peptides;
(ii) determining the presence and/or level of the one or more peptides by mass spectrometry, or performing mass spectrometry to determine the presence and/or level of the one or more peptides; and
(iii) using the results of (ii) to determine whether or not the level of the complement protein(s) is elevated, e.g. as compared to the level of that complement protein(s) in a blood sample in a control subject that does not have a complement-related disorder.

Thus, provided herein is a blood-derived sample from a subject, e.g. a subject that has or is suspected to have a complement-related disorder, that comprises at least one complement protein that has been digested with endoproteinase GluC. That is, the sample comprises peptides from complement proteins that have been digested with GluC.

The complement protein(s) may be selected from one or more of FHR1, FHR2, FHR3, FHR4 and/or FHR5, in any combination as described herein. The presence and/or level of FH and/or FHL-1 may also be determined.

The term “digesting” as used herein refers to placing the protein in contact with GluC under suitable conditions, e.g. temperature, pH etc, and for a suitable time such that the protein is digested, i.e. cleaved, into two or more fragments. In some cases, the digesting involves incubating the protein with GluC under suitable conditions, e.g. as described herein. The protein, e.g. a complement-related protein according to the present disclosure, may be contacted with GluC. That is, the methods provided herein may comprise a step of contacting the protein to be digested with GluC, e.g. at a concentration suitable for digesting the protein into peptides detectable by mass spectrometry.

In some aspects, provided is a method for preparing a complement protein for analysis, the method comprising contacting/digesting the protein with endoproteinase GluC to obtain one or more peptides. In some cases, the method comprises preparing a complement protein for subsequent analysis. The one or more peptides may then be subjected to an analytical technique, e.g. mass spectrometry or any other suitable analytical technique. In some cases the method comprises preparing a complement protein for analysis by mass spectrometry. The analytical technique may be used to detect the presence and/or level of the one or more peptides.

It will be appreciated that where “complement protein” is referred to herein in the singular (i.e. “a/the complement protein”), pluralities/groups/populations of different complement proteins are also contemplated. For example, any disclosure herein comprising a complement protein also comprises more than one complement protein, i.e. at least one protein, or one or more proteins. In all aspects and embodiments described herein, “a/the complement protein” may refer to “at least one complement protein”.

“Detecting” a protein as used herein refers to identifying/observing the presence, existence or level of the protein, e.g. in a sample, cell, tissue or subject.

The “level” of a complement protein used herein refers to the level, amount or concentration of said protein, e.g. in a sample, cell, tissue or subject. The term “determining the level”, e.g. of a protein, used herein refers to the measurement and/or quantification of the level, amount or concentration of a protein.

In some cases, “determining the level” includes calculating the level, amount or concentration of a protein in a sample. The sample may be from a subject. In some cases, “determining the level” includes calculating the level, amount or concentration of a protein in a subject, e.g. using a sample taken from the subject. “Determining the level” of a protein may include digesting the protein with GluC to obtain one or more peptides, detecting the one or more peptides as described herein and then calculating the level, amount or concentration of the protein/peptide, e.g. in a sample.

In some cases, “determining the level” comprises quantifying, i.e. measuring the quantity of, the level, amount or concentration of a protein e.g. in a sample or in a subject. “Determining the level” may include determining the concentration of a protein. Quantification/measuring may include comparing the level, amount or concentration of a protein with a reference value, and/or comparing the level, amount or concentration of a protein with that in a control sample e.g. taken from the subject at a different time point, or taken from a healthy subject, e.g. one known not to have a complement-related disorder.

In some embodiments the methods comprise detecting/determining the level of a complement protein in a sample. The sample may be in vitro or ex vivo. A sample may have been taken from a subject, e.g. from a subject of interest or from a control subject. A sample may be taken from any tissue or bodily fluid. In preferred arrangements the sample is taken from a bodily fluid, more preferably one that circulates through the body. The sample may be referred to as a circulating sample. Accordingly, the sample may be a blood sample or lymph sample. In a particularly preferred arrangement the sample is a blood sample or blood-derived sample. The blood-derived sample may be a selected fraction of a subject's blood, e.g. a selected cell-containing fraction or a plasma or serum fraction. A selected serum fraction may comprise the fluid portion of the blood obtained after removal of the fibrin clot and blood cells. Alternatively the sample may comprise or may be derived from a tissue sample, biopsy or isolated cells from said individual. The sample may be taken from the eye, kidney, brain or liver, e.g. comprising cells from the eye, kidney, brain or liver. The sample may comprise retinal tissue. The sample may comprise RPE cells or tissue from Bruch's membrane or the choroid. The sample may comprise drusen or other deposits of complement-related components.

In some embodiments the methods described herein comprise taking or obtaining a sample from a subject, e.g. blood, tissue etc. In some embodiments the methods described herein are performed on a sample that has been obtained/was obtained from a subject, e.g. that has been obtained previously and stored prior to use. Storage of samples, e.g. tissue and/or blood samples, are well known to a skilled person. In some cases the sample is a blood sample. The blood sample may undergo/have undergone processing to obtain a plasma sample or a serum sample. In some cases, the methods comprise obtaining a blood-derived sample from a subject. In some cases, the methods comprise obtaining a plasma or serum sample from a subject. In some embodiments the methods comprise isolating protein, e.g. total protein, from the sample. Suitable techniques to isolate protein from biological samples are well known in the field. In some embodiments the methods do not comprise isolating protein from the sample, e.g. the methods are performed on the unprocessed sample.

In some embodiments, the methods are performed in vitro. For example, the presence, level, amount and/or concentration of the complement protein(s) may be detected/determined in vitro.

In some cases the methods involve determining the presence, level, amount and/or concentration of the complement protein(s) in a subject. This may involve performing the methods described herein in vitro, and using the results to calculate the presence, level, amount and/or concentration of the protein(s) in the subject.

Also provided is a method for detecting at least one complement protein in a sample, the method comprising digesting the protein(s) in the sample with endoproteinase GluC to obtain one or more peptides; and using mass spectrometry to detect the one or more peptides in the sample. Any method described herein may comprise a step of detecting at least one complement protein, e.g. detecting the presence of the complement protein.

Also provided is a method for determining the level of at least one complement protein in a sample, the method comprising digesting the protein(s) in the sample with endoproteinase GluC to obtain one or more peptides and using mass spectrometry to determine the level of the one or more peptides in the sample.

Using mass spectrometry to detect one or more peptides in a sample, or detecting and/or determining the level of one or more peptides by mass spectrometry, e.g. by the methods described herein, may include applying a mass spectrometry technique to the sample, e.g. by putting the sample in a mass spectrometer, and instructing the mass spectrometer to analyse the sample. Various suitable mass spectrometry techniques are disclosed herein and are within the routine tasks of a skilled person.

In any aspect provided herein, the methods described herein may comprise both detecting at least one complement protein and determining the level of at least one complement protein. The complement protein may be the same protein, and/or the methods may comprise detecting a least a first complement protein and determining the level of at least a second complement protein.

In some embodiments, the methods described herein comprise detecting/determining the level of one complement protein. In some embodiments, the methods described herein comprise detecting/determining the level of at least one complement protein, one or more complement proteins, and/or groups or complement proteins e.g. as provided herein.

In some embodiments the complement protein is encoded from the RCA (regulators of complement) gene cluster, or RCA locus, on human chromosome 1. The RCA cluster is located on chromosome 1q32 and includes the CFH and CFHR1-5 genes. The gene cluster also includes the membrane bound proteins CR1 (CD35), CR2 (CD21), decay-accelerating factor (DAF; CD55), and membrane cofactor protein (MCP; CD46), as well as soluble C4b-binding protein (C4 bp).

The methods described herein are suitable for detecting/determining the level of multiple complement proteins via a single assay: i.e. using a single enzyme, GluC, to obtain analysable peptides and then using a single analytical technique, mass spectrometry, to detect and/or determine the levels of said peptides. In this way, the complementome of a sample or a subject can be determined via a single assay.

In some embodiments the methods described herein comprise detecting/determining the level of any one or more, e.g. any or all combinations, of FH, FHL-1, FHR1, FHR2, FHR3, FHR4, and/or FHR5. In some embodiments the complement protein(s) is/are selected from the group consisting of FH, FHL-1, FHR1, FHR2, FHR3, FHR4, and/or FHR5. In some cases the methods comprise detecting/determining the level of any one, two, three, four, five, six and/or seven of FH, FHL-1, FHR1, FHR2, FHR3, FHR4, and FHR5, alone or in combination. In some cases the methods described herein are able to differentiate (i.e. distinguish, discriminate, separate) between the presence of (or levels of) each of FH, FHL-1, FHR1, FHR2, FHR3, FHR4 and/or FHR5.

In some cases the complement protein is any one or more, e.g. any or all combinations, of FHR1, FHR2, FHR3, FHR4 and/or FHR5. In some cases the complement protein is FHR1. In some cases the complement protein is FHR2. In some cases the complement protein is FHR3. In some cases the complement protein is FHR4. In some cases the complement protein is FHR5. In some cases the methods described herein are able to differentiate (i.e. distinguish, discriminate, separate) between the presence of (or levels of) each of FHR1, FHR2, FHR3, FHR4 and/or FHR5. Exemplary combinations of FHR proteins that may be detected in the present invention are described herein.

In some cases, the methods described herein permit or allow the detection of FHR1 alone, i.e. without detecting FHR2-FHR5. In some cases, the methods described herein permit or allow the detection of FHR2 alone, i.e. without detecting FHR1 or FHR3-FHR5. In some cases, the methods described herein permit or allow the detection of FHR3 alone, i.e. without detecting FHR1, FHR2, FHR4, or FHR5. In some cases, the methods described herein permit or allow the detection of FHR4 alone, i.e. without detecting FHR1-FHR3 or FHR5. In some cases, the methods described herein permit or allow the detection of FHR5 alone, i.e. without detecting FHR1-FHR4.

In some cases the complement protein is to be detected/determine the level of FH and/or FHL-1. In some cases the methods described herein comprise detecting/determining the level of both FH and FHL-1. In some cases the methods described herein differentiate (i.e. distinguish, discriminate, separate) between the presence of FH and the presence of FHL-1 and/or between the level/concentration of FH and the level/concentration of FHL-1. In some cases the methods described herein permit or allow the detection of FH alone, i.e. without detecting FHL-1. In some cases the methods described herein permit or allow the detection of FHL-1 alone, i.e. without detecting FH.

In some embodiments the complement protein to be detected/the level of which is determined is involved with breakdown, turnover and/or inactivation of C3/C3b. In some embodiments, the complement protein is produced by the breakdown and/or inactivation of C3/C3b, i.e. is a product of C3b inactivation/breakdown. In some embodiments the methods described herein include determining the presence, rate and/or progression of C3b turnover. In some embodiments the methods described herein involve detecting/determining the level of a protein involved in, or produced as a result of, the complement amplification loop. In some embodiments the methods described herein involve detecting/determining the level of a protein involved in the generation or breakdown of C3 convertase. In some cases the protein is a cofactor for FI, e.g. FH, CR1, or the FHR proteins. Any method disclosed herein, e.g. a method for detecting at least one complement protein in a sample comprising digesting proteins with GluC and detecting the resulting peptides by mass spectrometry, may be described in the alternative as a method for detecting C3 turnover, a method for detecting C3 breakdown, a method for measuring C3b turnover or C3b breakdown, or a method for measuring the progress of C3b turnover or C3b breakdown.

Thus, in some aspects the present invention provides a method for detecting turnover or breakdown of C3b, comprising the steps described herein, e.g. digesting at least one complement protein with endoproteinase GluC to obtain one or more peptides and detecting the peptide(s) by mass spectrometry. In some cases the method comprises digesting and then detecting at least two, three, four or more, up to 16, of the 16 complement proteins described herein.

In some embodiments the methods described herein comprise/further comprise detecting/determining the level of FI, either alone or in combination with other complement proteins such as those described herein.

In some embodiments the methods described herein comprise detecting/determining the level of any one or more, e.g. any or all combinations, of C3, C3b, C3a, iC3b, C3f, C3c, C3dg, and/or C3d. In some embodiments the complement protein(s) is/are selected from the group consisting of C3, C3b, C3a, iC3b, C3f, C3c, C3dg, and/or C3d. In some cases the methods comprise detecting/determining the level of any one, two, three, four, five, six, seven and/or eight of C3, C3b, C3a, iC3b, C3f, C3c, C3dg, and/or C3d, in any combination. In some embodiments the methods described herein comprise detecting/determining the level of one or more of C3, C3a, C3f, C3c, and/or C3d. In some cases the methods described herein comprise determining the presence and/or level of C3b, iC3b, and/or C3dg, e.g. via the methodology in Table 3. In some cases the methods described herein comprise detecting/determining the level of C3, C3b and/or iC3b. In some cases the methods described herein are able to differentiate (i.e. distinguish, discriminate, separate) between the presence of (or levels of) two or more, or all, of C3, C3b, C3a, iC3b, C3f, C3c, C3dg, and/or C3d.

The methods described herein can detect multiple complement proteins, and distinguish between said complement proteins, using one enzyme e.g. GluC and one analytical method e.g. mass spectrometry. The methods described herein may be used to detect/determine the level of any one of the individual proteins described herein, as well as any and all combinations of FH, FHL-1, FHR1, FHR2, FHR3, FHR4, FHR5, FI, C3, C3b, C3a, iC3b, C3f, C3c, C3dg, and/or C3d, i.e. any one, two, three, four, five, six, seven, eight, nine, ten, eleven, twelve, thirteen, fourteen, fifteen and/or sixteen of these proteins in any combination. In some embodiments the complement protein(s) is/are selected from the group consisting of FH, FHL-1, FHR1, FHR2, FHR3, FHR4, FHR5, FI, C3, C3b, C3a, iC3b, C3f, C3c, C3dg, and/or C3d.

In some cases the methods described herein may be used to detect/determine the level of FHL-1 and to detect/determine the level of any one or more of FH, FHR1, FHR2, FHR3, FHR4, FHR5, FI, C3, C3b, C3a, iC3b, C3f, C3c, C3dg, and/or C3d. In some cases the method comprises distinguishing (i.e. differentiating, discriminating, separating) between the presence/level of FHL-1 and the presence/level of any one or more of FH, FHR1, FHR2, FHR3, FHR4, FHR5, FI, C3, C3b, C3a, iC3b, C3f, C3c, C3dg, and/or C3d. The terms “distinguishing”, “differentiating”, “discriminating”, and “separating” are used interchangeably herein.

In some cases the methods provided herein allow for simultaneous detection of one or more of FH, FHL-1, FHR1, FHR2, FHR3, FHR4, FHR5, FI, C3, C3b, C3a, iC3b, C3f, C3c, C3dg, and/or C3d, including any combination thereof. In some cases the methods provided herein allow for detection/determination of the level of one or more of FH, FHL-1, FHR1, FHR2, FHR3, FHR4, FHR5, FI, C3, C3b, C3a, iC3b, C3f, C3c, C3dg, and/or C3d, including any combination thereof, in a single assay. The methods provided herein allow for distinct, separable and detectable peptides to be produced from every protein listed above such that the presence and/or level of each protein can be distinguished from the others.

In some cases the methods provided herein allow for simultaneous detection of one or more of FHR1, FHR2, FHR3, FHR4, FHR5, FH, and/or FHL-1, including any combination thereof. In some cases the methods provided herein allow for detection/determination of the level of one or more of FHR1, FHR2, FHR3, FHR4, FHR5, FH, and/or FHL-1, including any combination thereof, in a single assay.

In some cases, the present invention provides a method for detecting and/or determining the level of at least two complement proteins in a sample simultaneously and/or in one assay, the method comprising:
digesting the proteins with endoproteinase GluC to obtain one or more peptides; and
detecting and/or determining the level of the one or more peptides by mass spectrometry.

In any method described herein, the complement protein may be any protein involved in one or more of the complement system pathways. For example, the complement protein may be one or more of C1, C2, C4b2a C4, C4a, C5, C5a, FB, FD, C3Bb, MASP1, MASP2, C1q, C1r, C1s, C6, C7, C8, C9, CD59, Clusterin, Properdin, and/or Compstatin. In any embodiment described herein, the complement protein to be detected (or the protein whose level is determined) is not one or more of C1, C2, C4b2a C4, C4a, C5, C5a, FB, FD, C3Bb, MASP1, MASP2, C1q, C1r, C1s, C6, C7, C8, C9, CD59, Clusterin, Properdin, and/or Compstatin.

In some cases, the present invention provides endoproteinase GluC for preparing at least one complement protein for detection by mass spectrometry. In some cases the invention provides endoproteinase GluC for preparing at least two, i.e. multiple or a plurality of, complement proteins for detection by mass spectrometry. The at least two complement proteins may be any two, three, four or more, up to 16, of FH, FHL-1, FHR1, FHR2, FHR3, FHR4, FHR5, FI, C3, C3b, C3a, iC3b, C3f, C3c, C3dg, and/or C3d, in any combination, as described herein.

Endoproteinase GluC, also known as glutamyl endopeptidase, is a serine proteinase which preferentially cleaves peptide bonds C-terminal to glutamic acid residues. It also cleaves at aspartic acid residues at a rate 100-300 times slower than at glutamic acid residues. The specificity of GluC depends on the pH and the buffer composition. At pH 4, the enzyme preferentially cleaves at the C terminus of E, whereas at pH 8 it additionally cleaves at D residues. The sequence of GluC is provided in SEQ ID NO:153 and 154.

In preferred embodiments, the methods described herein use GluC alone (i.e. only GluC) to digest the one or more complement proteins. In preferred embodiments, a step of digesting the protein(s) in the described methods consists of digesting the protein(s) with GluC. In some embodiments, where a step of digesting the protein(s) in the described methods involves digesting FHR1, the methods may comprise digesting the protein(s) with GluC and trypsin, e.g. to distinguish between FHR1a and FHR1b.

In preferred embodiments, any method described herein does not employ/use any other protease alone or in combination with GluC. For example, in some embodiments the digestion step of any method described herein does not use, or is not performed by, any one or more of the following enzymes or agents: trypsin, chymotrypsin (high specificity or low specificity), Lys-C, Lys-N, Arg-C, Asp-N, elastase, LysargiNase, pepsin, Sap9, OmpT, BNPS-skatole, any caspase, clostripain (clostridiopeptidase B), CNBr, enterokinase, factor Xa, granzymeB, neutrophil elastase, proteinase K, thermolysin, non-GluC glutamyl endopeptidase e.g. GluBI or GluSGB, proline endopeptidase, TEV protease, thrombin, formic acid, hydroxylamine, iodosobenzoic acid, and/or NTCB (or any combination thereof).

GluC is obtainable from standard reagent providers e.g. Sigma Aldrich, NEB etc, and may be used according to the accompanying instructions or according to protocols well known in the field. An example protocol is described herein. Obtaining proteins from biological samples and suitable buffers to prepare samples/proteins for GluC digestion will also be known to the skilled person. An example cell lysis buffer comprises: 8 M urea (4.8 g per 10 ml) in 50 mM NH₄HCO₃ and 20 mM methylamine, diluted to a urea concentration of <2 M, pH 8 (40 mg per 10 ml), containing 1 tablet of cOmplete™ Mini EDTA-free protease inhibitor cocktail per 10 ml of lysis buffer.

In some cases, a complement protein is contacted/incubated/digested with GluC enzyme for at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 hours. In some cases a complement protein is contacted/incubated/digested with GluC enzyme for at least 12 hours. In some cases a complement protein is contacted/incubated/digested with GluC enzyme for about 12 hours, e.g. 12 hours. In some cases a complement protein is contacted/incubated/digested with GluC enzyme for about 16 hours, e.g. 16 hours. The terms contacted, incubated and digested are used interchangeably herein.

In some cases a complement protein is contacted/incubated/digested with GluC enzyme at a temperature of at least 20° C., at least 21° C., at least 22° C., at least 23° C., at least 24° C., at least 25° C., at least 26° C., at least 27° C., at least 28° C., at least 29° C., or at least 30° C. In some cases a complement protein is contacted/incubated/digested with GluC enzyme at a temperature of at least 25° C. In some cases a complement protein is contacted/incubated/digested with GluC enzyme at a temperature of about 25° C., e.g. 25° C.

In some cases a complement protein is contacted/incubated/digested with GluC enzyme at a pH of at least 7.0, at least 7.1, at least 7.2, at least 7.3, at least 7.4, at least 7.5, at least 7.6, at least 7.7, at least 7.8, at least 7.9, at least 8.0, at least 8.1, at least 8.2, at least 8.3, at least 8.4, at least 8.5, at least 8.6, at least 8.7, at least 8.8, at least 8.9, or at least 9.0. In some cases a complement protein is contacted/incubated/digested with GluC enzyme at a pH of at least 8.0. In some cases a complement protein is contacted/incubated/digested with GluC enzyme at a pH of about 8.0, e.g. a pH of 8.0.

In some cases the GluC enzyme and complement protein are contacted/incubated at a wt/wt ratio of 1/75. The incubation step may comprise gentle shaking, e.g. at 400 rpm.

The methods described herein may comprise a contacting/incubation/digestion step comprising any combination of temperature, pH, and/or time as described above. In some cases, contacting/incubating/digesting is performed at 25° C. at pH8 for 12 hours.

In some embodiments the invention provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 12 hours; and
detecting the one or more peptides by mass spectrometry.

In some embodiments the invention provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 16 hours; and
detecting the one or more peptides by mass spectrometry.

The following peptides may be produced by GluC digestion of complement proteins, e.g. as described herein. In some embodiments, the methods described herein comprise detecting/determining the level of any one or more of these peptides, i.e. any one or more of SEQ ID NO:20 to 141, or 155, 156 or 157, in any combination. All combinations of peptides are envisaged. The mass of peptides represented by SEQ ID NOs 20-27 can be found in Table 1.

In some embodiments, the FH peptide is VTYKCFE (SEQ ID NO:20).

In some embodiments the FH peptide is any one or more of SNTGSTTGSIVCGYNGWSDLPICYE (SEQ ID NO:112; mass 2623.1206), NGWSPTPRCIRVKTCSKSSIDIE (SEQ ID NO:113; mass 2576.2839), LPKIDVHLVPDRKKDQYKVGE (SEQ ID NO:114; mass 2476.3801), YYCNPRFLMKGPNKIQCVDGE (SEQ ID NO:115; mass 2474.1545), NYNIALRWTAKQKLYSRTGE (SEQ ID NO:116; mass 2411.2709), KWSHPPSCIKTDCLSLPSFE (SEQ ID NO:117; mass 2274.0813), HGWAQLSSPPYYYGDSVE (SEQ ID NO:118; mass 2054.9010), ISHGVVAHMSDSYQYGEE (SEQ ID NO:119; mass 2007.8632), FDHNSNIRYRCRGKE (SEQ ID NO:120; mass 1893.9016), ITCKDGRWQSIPLCVE (SEQ ID NO:121; mass 1846.9069), GWIHTVCINGRWDPE (SEQ ID NO:122; mass 1781.8307), KAKYQCKLGYVTADGE (SEQ ID NO:123; mass 1772.8767), TTCYMGKWSSPPQCE (SEQ ID NO:124; mass 1716.6946), SYAHGTKLSYTCE (SEQ ID NO:125, mass 1458.6449), RVRYQCRSPYE (SEQ ID NO:126; mass 1455.7041), GFGIDGPAIAKCLGE (SEQ ID NO:127; mass 1446.7176), HGTINSSRSSQE (SEQ ID NO:128; mass 1301.5960), YQCQNLYQLE (SEQ ID NO:129; mass 1300.5758), WTTLPVCIVEE (SEQ ID NO:130; mass 1288.6373), KIPCSQPPQIE (SEQ ID NO:131; mass 1238.6329), SQYTYALKE (SEQ ID NO:132; mass 1101.5342), QVQSCGPPPE (SEQ ID NO:133; mass 1040.4597), KKDVYKAGE (SEQ ID NO:134; mass 1036.5553), GLPCKSPPE (SEQ ID NO:135; mass 926.4531), KVSVLCQE (SEQ ID NO:136; mass 904.4688), HLKNKKE (SEQ ID NO:137; mass 895.5239), GGFRISEE (SEQ ID NO:138; mass 893.4243), LLNGNVKE (SEQ ID NO: 139; mass 885.4920), YPTCAKR (SEQ ID NO:140; mass 837.4167), or STCGDIPE (SEQ ID NO:141; mass 820.3273).

In some embodiments, the FHL-1 peptide is NGWSPTPRCIRVSFTL (SEQ ID NO:21).

In some embodiments, the FHR1 peptide is ATFCDFPKINHGILYGEE (SEQ ID NO:22).

In some embodiments the FHR1 peptide is NYNIALRWTAKQKLYLRTGE (SEQ ID NO:91; mass 2437.3230).

In some embodiments the FHR2 peptide is RGWSTPPKCRSTISAE (SEQ ID NO:23).

In some embodiments the FHR2 peptide is AMFCDFPKINHGILYDEE (SEQ ID NO:24). In some embodiments the FHR2 peptide is YNFVSPSKSFWTRITCAEE (SEQ ID NO:92; mass 2264.0572).

In some embodiments the FHR3 peptide is VACHPGYGLPKAQTTVTCTE (SEQ ID NO:25).

In some embodiments the FHR3 peptide is any one or more of KGWSPTPRCIRVRTCSKSDIE (SEQ ID NO:93; mass 2418.2260), NGYNQNYGRKFVQGNSTE (SEQ ID NO:94; mass 2074.9457), QVKPCDFPDIKHGGLFHE (SEQ ID NO:95; mass 2066.0043), FMCKLGYNANTSILSFQAVCRE (SEQ ID NO:96; mass 2494.1807), orYQCQPYYE (SEQ ID NO:97; mass 1092.4222).

In some embodiments the FHR4 peptide is YQCQSYYE (SEQ ID NO:26).

In some embodiments the FHR4 peptide is any one or more of NSRAKSNGMRFKLHDTLDYE (SEQ ID NO: 98; mass 2381.1546), DGWSHFPTCYNSSE (SEQ ID NO:99; mass 1628.6202), ISYGNTTGSIVCGE (SEQ ID NO:100; mass 1399.6289), or FMCKLGYNANTSVLSFQAVCRE (SEQ ID NO:101; mass 2480.1650).

In some embodiments the FHR5 peptide is RGWSTPPICSFTKGE (SEQ ID NO:27).

In some embodiments the FHR5 peptide is any one or more of GTLCDFPKIHHGFLYDEE (SEQ ID NO:102; mass 2119.9673), YAMIGNNMITCINGIWTE (SEQ ID NO:103; mass 2042.9264), YGYVQPSVPPYQHGVSVE (SEQ ID NO:104; mass 2004.9581), GDTVQIICNTGYSLQNNE (SEQ ID NO:105; mass 1967.8895), IVCKDGRWQSLPRCVE (SEQ ID NO:106; mass 1887.9447), DYNPFSQVPTGE (SEQ ID NO:107; mass 1352.5884), QVKTCGYIPE (SEQ ID NO:108; mass 1136.5536), ANVDAQPKKE (SEQ ID NO:109; mass 1098.5669), WTTLPTCVE (SEQ ID NO:110; mass 1048.4899), or KVAVLCKE (SEQ ID NO: 111; mass 888.5102).

In some cases, the methods described herein comprise detecting/determining the level of one or more of SEQ ID NOs 21-27, in any combination.

In some cases, any method described herein may comprise detecting/determining the level of one or more of SEQ ID NOs 28-37, 156 or 157, in any combination. In some cases, the methods provided herein are used to detect C3, C3b and breakdown products using one or more or all of the peptides in Table 2 in any combination, plus optionally SEQ ID NO:156 and/or 157, for example according to the methodology in Table 3.

In some embodiments the FI peptide is any one or more of VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), VGCAGFASVTQE (SEQ ID NO:155; mass 1168.272), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), or KVTYTSQE (SEQ ID NO:56; mass 955.4731).

In some embodiments the FI peptide is any one or more of CAGTYDGSIDACKGDSGGPLVCMDANNVTYVWGVVSWGE (SEQ ID NO:38; mass 3996.7183), GTCVCKLPYQCPKNGTAVCATNRRSFPTYCQQKSLE (SEQ ID NO:39; mass 3994.8853), FPGVYTKVANYFDWISYHVGRPFISQYNV (SEQ ID NO:40; mass 3467.7211), ANVACLDLGFQQGADTQRRFKLSDLSINSTE (SEQ ID NO:41, mass 3397.6804), LPRSIPACVPWSPYLFQPNDTCIVSGWGRE (SEQ ID NO:42, mass 3388.6605), KKCLAKKYTHLSCDKVFCQPWQRCIE (SEQ ID NO:43; mass 3155.5773), LCCKACQGKGFHCKSGVCIPSQYQCNGE (SEQ ID NO:44; mass 2991.2861), VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), KVTYTSQE (SEQ ID NO:56; mass 955.4731), VDCITGE (SEQ ID NO:57; mass 736.3182), NCGKPE (SEQ ID NO:58; mass 647.2817), TSLAE (SEQ ID NO:59; mass 520.2613), or KDNE (SEQ ID NO:60; mass 505.2252).

Peptides detected by the methods described herein may optionally have at least 70%, preferably one of 80%, 85%, 90%, 91%, 92%, 93%, 94%, 95%, 96%, 97%, 98%, 99% or 100% amino acid sequence identity to the amino acid sequences of the peptides described herein, e.g. any one of SEQ ID NOs 21-141. Other suitable peptides may be readily determined by a skilled person and may be employed in the methods described herein. In preferred embodiments, the peptides used according to the methods herein permit mass spectrometry techniques to distinguish or differentiate between two or more complement proteins in a sample.

Mass Spectrometry

Methods provided herein comprising detecting and/or determining the levels of proteins may involve using mass spectrometry to detect and/or determine the levels of proteins in a sample.

In some embodiments, any method provided herein may comprise performing mass spectrometry to determine the presence and/or level of one or more peptides as described herein. That is, any step described herein that comprises determining the level of one or more proteins/peptides may comprise performing mass spectrometry to determine the presence and/or level of the one or more proteins/peptides.

In preferred embodiments, any method described herein involves using only mass spectrometry (i.e. mass spectrometry alone) to detect/determine the level of the one or more peptides. That is, in some embodiments, the methods provided herein do not employ multiple analytical techniques and the peptide(s) are detected/determined/measured using a single assay. In preferred embodiments, the methods described herein do not detect/determine the level of/measure the peptide(s) using mass spectrometry in combination with another analytical technique suitable for detecting proteins/peptides. In preferred embodiments, detection/determination of the level of the one or more peptides is not performed at any stage using a non-mass spectrometry technique, e.g. detection/determination of the level of the peptide(s) is not performed using high performance liquid chromatography (HPLC), immunological-based methods such as quantitative enzyme-linked immunosorbent assays (ELISA), Western blotting, protein immunoprecipitation, dot blotting or immunoelectrophoresis, electrophoresis or autoradiography. In some cases, liquid chromatography-mass spectrometry (LC/MS) is not used.

As used herein, “detecting and/or determining the level of” e.g. a complement protein or peptide “by mass spectrometry” is the same as “using mass spectrometry to detect and/or determine the level of” e.g. a complement protein or peptide.

Mass spectrometry is a well-known analytical technique for analysing a sample that typically comprises generating ions from the sample, optionally fragmenting the ions, separating the ions according to their mass/charge ratio (in time and/or space), and detecting the ions to provide information regarding the content of the sample.

For the purpose of detecting and/or determining the levels of proteins, at least one fragmentation step may be included.

Mass spectrometry techniques are well-known in the field and any suitable mass spectrometry technique may be employed for detecting and/or determining the levels of proteins in a sample, e.g. LC/MS, GC/MS, tandem mass spectrometry (MS/MS), quadrupole MS e.g. triple quadrupole MS (TQMS), time-of-flight MS e.g. MALDI-TOF, targeted MS e.g. selected reaction monitoring MS (SRM-MS)/multiple reaction monitoring (MRM-MS), parallel-reaction monitoring (PRM-MS), trapped-ion based methods e.g. three-dimensional quadrupole ion traps (“dynamic” traps) and ion cyclotron resonance mass spectrometers (“static” traps), quadrupole trap MS, hybrid linear trap orbitrap MS, quadrupole-Orbitrap MS, electrospray Ionization mass spectrometry (ESI-MS), or electron transfer dissociation MS (ETD).

In some embodiments, the mass spectrometry technique may be a liquid chromatography-selected reaction monitoring mass spectrometry (LC-SRM-MS)-based assay.

Fragmenting the ions may be achieved using any suitable fragmentation technique, e.g. collision-induced dissociation (CID)/collisionally activated dissociation (CAD), electron-capture dissociation (ECD), electron transfer dissociation (ETD), in-source decay (ISD), infrared multiple photon dissociation (IRMPD) etc. Again, such techniques are well known.

The mass spectrometry techniques useful in the present invention may comprise quantitative analysis. Mass spectrometry methods comprising quantitative analysis may comprise a targeted approach to detect and measure peptides of interest and their corresponding fragments. This may allow for greater specificity and sensitivity for quantification. Quantitative mass spectrometry in proteomics is reviewed in e.g. Bantscheff, M., et al. Anal Bioanal Chem 2007, 389, 1017-1031, which is hereby incorporated by reference in its entirety.

For example, input peptides may undergo fragmentation in a collision cell, thus generating product ions exclusive to the peptides. Both the intact peptide mass and one or more specific fragment ions of that peptides can be monitored over the course of an MS experiment e.g. using SRM/MRM, PRM etc.

The observed m/z ratio of a peptide and its corresponding product ion m/z ratio are referred to as a “transition”, i.e. a mass pair representing the m/z of an analyte (the parent ion) and the m/z of one of its product ions which is formed upon fragmentation of the parent ion.

It is well within the routine work of a skilled person to develop suitable transitions for quantitative mass spectrometry techniques, e.g. SRM/MRM-MS and PRM-MS. Mead et al., Mol Cell Proteomics. 2009 April; 8(4): 696-705, hereby incorporated by reference in its entirety, describes one such technique for designing transitions.

Tables 7 and 8 provide examples of transitions for the complement proteins described herein, based on fragmentation of synthetic versions of each peptide of interest. Suitable alternative transitions may also be used, the identification of which is well within the routine remit of a skilled person.

Quantitation can be achieved by ‘spiking’ the sample with known quantities of labelled synthetic peptides. The combination of retention time, peptide mass, and fragment mass practically eliminates ambiguities in peptide assignments and extends the quantification range to 4-5 orders of magnitude. In some cases, the methods provided herein comprise a step of determining optimised MS settings and/or quantitation reference values using stable isotopic standards.

Mass spectrometry techniques that may be used in the present invention may comprise targeted or semi-targeted MS workflows and/or data-dependent acquisition (DDA) or data-independent acquisition (DIA) techniques.

DDA uses knowledge obtained during the acquisition to decide which MS1 peptide precursors to subject for fragmentation (MS/MS) in the collision cell. DIA, in contrast, performs predefined MS/MS fragmentation and data collection regardless of sample content, which allows for more sensitive and accurate protein quantification compared to DDA. DIA strategies can be further segregated into targeted or untargeted acquisitions. Targeted DIA methods fragment predefined precursor ions that correspond to the peptide analytes, usually at known (measured or predicted) retention times. Targeted DIA has become widely used in academic, pharmaceutical, and biotechnology research for quantification of small molecules (metabolites), peptides, and post-translational modifications (PTMs). For example, selected-reaction monitoring (SRM), a type of targeted DIA, is currently considered the gold standard method for mass spectrometric quantification due to its high accuracy and precision. For a review of DIA techniques, see e.g. Meyer and Schilling, Expert Rev Proteomics. 2017 May; 14(5): 419-429, hereby incorporated in its entirety.

Other suitable DIA methods include e.g. Sequential Window Acquisition of All Theoretical mass spectrometry (SWATH MS; see e.g. Ludwig et al., Mol Syst Biol (2018)14:e8126), SONAR (Waters.com), or Online Parallel Accumulation-Serial Fragmentation (PASEF; see e.g. Meier et al., J Proteome Res. 2015 Dec. 4; 14(12):5378-87 and Meier et al., Mol Cell Proteomics. 2018 December; 17(12): 2534-2545). In some embodiments, the methods according to the present disclosure do not employ SWATH MS, i.e. the mass spectrometry technique is not SWATH MS.

Kits

Aspects of the disclosure include in vitro diagnostic methods and in vitro kits for performing such methods. In some embodiments, the present invention provides a kit comprising endoproteinase GluC for use in a method of detecting and/or determining the level of one or more complement protein(s) e.g. in a sample. The kit may be used for any of the methods described herein and/or for detecting/determining the level of any one or combination of proteins described herein. The kit may be suitable for, used for, or intended/sold/distributed for detecting at least one complement protein in a sample, determining the level of at least one complement protein in a sample, preparing at least one complement protein for analysis and/or detection, determining the presence and/or level of a complement protein in a subject, determining whether a subject is at risk of developing a complement-related disorder, identifying a subject having a complement-related disorder, selecting a subject for treatment of a complement-related disorder, and/or treating a subject who is suspected to have a complement-related disorder. The kit and components thereof may be suitable for use with MS techniques.

A kit provided herein comprises one, two, or more components suitable for performing the methods described herein, in whole or in part. The kit may comprise standards or controls, e.g. labelled peptide standard(s) for each protein to be detected using the kit. The kit may comprise a predetermined quantity of labelled peptide standards. The kit may comprise a predetermined quantity of GluC enzyme, optionally with the necessary buffers and reagents for enzyme digestion. The components of the kit may be provided in a single composition, or may be provided as plural compositions.

The kit may be suitable for a point-of-care in vitro diagnostic test. It may be a kit for laboratory-based testing. The kit may include instructions for use, such as an instruction booklet or leaflet. The instructions may include a protocol for performing any one or more of the methods described herein e.g. for enzyme digestion, recommended MS settings, and/or data analysis templates. The kit may comprise components for separating proteins in a sample and/or performing MS techniques e.g. liquid chromatography columns.

The kit may be adapted for use with dry samples, wet samples, frozen samples, fixed samples, urine samples, saliva samples, tissue samples, blood samples, or any other type of sample, including any of the sample types disclosed herein. The kit may comprise a device for obtaining or processing a blood, serum, plasma, cell or tissue sample.

Sequence Identity

As used herein, an amino acid sequence which corresponds to a reference amino acid sequence may comprise at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the reference sequence.

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof. The invention includes the combination of the aspects and preferred features described except where such a combination is clearly impermissible or expressly avoided.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The phase “and/or” as used herein encompasses each member of the list individually, as well as any combination of one or members of the list up to and including every member of the list.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

Sequences

| SEQ | | |
|---|---|---|
| ID | | |
| No: | Description | Sequence |
| 1 | Human Complement | MRLLAKIICLMLWAICVAEDCNELPPRRNT |
| | Factor H (FH) | EILTGSWSDQTYPEGTQAIYKCRPGYRSLG |
| | Uniprot: P08603-1 | NVIMVCRKGEWVALNPLRKCQKRPCGHPGD |
| | Entry version 229 | TPFGTFTLTGGNVFEYGVKAVYTCNEGYQL |
| | (11 Dec. 2019) | LGEINYRECDTDGWTNDIPICEVVKCLPVT |
| | Sequence version 4 | APENGKIVSSAMEPDREYHFGQAVRFVCNS |
| | (11 Sep. 2007) | GYKIEGDEEMHCSDDGFWSKEKPKCVEISC |
| | | KSPDVINGSPISQKIIYKENERFQYKCNMG |
| | | YEYSERGDAVCTESGWRPLPSCEEKSCDNP |
| | | YIPNGDYSPLRIKHRTGDEITYQCRNGFYP |
| | | ATRGNTAKCTSTGWIPAPRCTLKPCDYPDI |
| | | KHGGLYHENMRRPYFPVAVGKYYSYYCDEH |
| | | FETPSGSYWDHIHCTQDGWSPAVPCLRKCY |
| | | FPYLENGYNQNYGRKFVQGKSIDVACHPGY |
| | | ALPKAQTTVTCMENGWSPTPRCIRVKTCSK |
| | | SSIDIENGFISESQYTYALKEKAKYQCKLG |
| | | YVTADGETSGSITCGKDGWSAQPTCIKSCD |
| | | IPVFMNARTKNDFTWFKLNDTLDYECHDGY |
| | | ESNTGSTTGSIVCGYNGWSDLPICYERECE |
| | | LPKIDVHLVPDRKKDQYKVGEVLKFSCKPG |
| | | FTIVGPNSVQCYHFGLSPDLPICKEQVQSC |
| | | GPPPELLNGNVKEKTKEEYGHSEVVEYYCN |
| | | PRFLMKGPNKIQCVDGEWTTLPVCIVEEST |
| | | CGDIPELEHGWAQLSSPPYYYGDSVEFNCS |
| | | ESFTMIGHRSITCIHGVWTQLPQCVAIDKL |
| | | KKCKSSNLIILEEHLKNKKEFDHNSNIRYR |
| | | CRGKEGWIHTVCINGRWDPEVNCSMAQIQL |
| | | CPPPPQIPNSHNMTTTLNYRDGEKVSVLCQ |
| | | ENYLIQEGEEITCKDGRWQSIPLCVEKIPC |
| | | SQPPQIEHGTINSSRSSQESYAHGTKLSYT |
| | | CEGGFRISEENETTCYMGKWSSPPQCEGLP |
| | | CKSPPEISHGVVAHMSDSYQYGEEVTYKCF |
| | | EGFGIDGPAIAKCLGEKWSHPPSCIKTDCL |
| | | SLPSFENAIPMGEKKDVYKAGEQVTYTCAT |
| | | YYKMDGASNVTCINSRWTGRPTCRDTSCVN |
| | | PPTVQNAYIVSRQMSKYPSGERVRYQCRSP |
| | | YEMFGDEEVMCLNGNWTEPPQCKDSTGKCG |
| | | PPPPIDNGDITSFPLSVYAPASSVEYQCQN |
| | | LYQLEGNKRITCRNGQWSEPPKCLHPCVIS |
| | | REIMENYNIALRWTAKQKLYSRTGESVEFV |
| | | CKRGYRLSSRSHTLRTTCWDGKLEYPTCAK |
| | | R |
| 2 | Human FH-like | MRLLAKIICLMLWAICVAEDCNELPPRRNT |
| | protein 1 (FHL-1) | EILTGSWSDQTYPEGTQAIYKCRPGYRSLG |
| | Uniprot: P08603-2 | NVIMVCRKGEWVALNPLRKCQKRPCGHPGD |
| | Entry version 229 | TPFGTFTLTGGNVFEYGVKAVYTCNEGYQL |
| | (11 Dec. 2019) | LGEINYRECDTDGWTNDIPICEVVKCLPVT |
| | Sequence version 4 | APENGKIVSSAMEPDREYHFGQAVRFVCNS |
| | (11 Sep. 2007) | GYKIEGDEEMHCSDDGFWSKEKPKCVEISC |
| | | KSPDVINGSPISQKIIYKENERFQYKCNMG |
| | | YEYSERGDAVCTESGWRPLPSCEEKSCDNP |
| | | YIPNGDYSPLRIKHRTGDEITYQCRNGFYP |
| | | ATRGNTAKCTSTGWIPAPRCTLKPCDYPDI |
| | | KHGGLYHENMRRPYFPVAVGKYYSYYCDEH |
| | | FETPSGSYWDHIHCTQDGWSPAVPCLRKCY |
| | | FPYLENGYNQNYGRKFVQGKSIDVACHPGY |
| | | ALPKAQTTVTCMENGWSPTPRCIRVSFTL |
| 3 | Complement Factor | MWLLVSVILISRISSVGGEATFCDFPKINH |
| | H-related 1 (FHR1) | GILYDEEKYKPFSQVPTGEVFYYSCEYNFV |
| | Uniprot: Q03591 | SPSKSFWTRITCTEEGWSPTPKCLRLCFFP |
| | Entry version 166 | FVENGHSESSGQTHLEGDTVQIICNTGYRL |
| | (11 Dec. 2019) | QNNENNISCVERGWSTPPKCRSTDTSCVNP |
| | Sequence version 2 | PTVQNAHILSRQMSKYPSGERVRYECRSPY |
| | (16 Dec. 2008) | EMFGDEEVMCLNGNWTEPPQCKDSTGKCGP |
| | | PPPIDNGDITSFPLSVYAPASSVEYQCQNL |
| | | YQLEGNKRITCRNGQWSEPPKCLHPCVISR |
| | | EIMENYNIALRWTAKQKLYLRTGESAEFVC |
| | | KRGYRLSSRSHTLRTTCWDGKLEYPTCAKR |
| 4 | Complement Factor | MWLLVSVILISRISSVGGEAMFCDFPKINH |
| | H-related 2 (FHR2), | GILYDEEKYKPFSQVPTGEVFYYSCEYNFV |
| | isoform 1 | SPSKSFWTRITCAEEGWSPTPKCLRLCFFP |
| | Uniprot: P36980-1 | FVENGHSESSGQTHLEGDTVQIICNTGYRL |
| | Entry version 168 | QNNENNISCVERGWSTPPKCRSTISAEKCG |
| | (11 Dec. 2019) | PPPPIDNGDITSFLLSVYAPGSSVEYQCQN |
| | Sequence version 1 | LYQLEGNNQITCRNGQWSEPPKCLDPCVIS |
| | (01 Jun. 1994) | QEIMEKYNIKLKWTNQQKLYSRTGDIVEFV |
| | | CKSGYHPTKSHSFRAMCQNGKLVYPSCEEK |
| 5 | Complement Factor | MWLLVSVILISRISSVGGEAMFCDFPKINH |
| | H-related 2 (FHR2), | GILYDEEKYKPFSQVPTGEVFYYSCEYNFV |
| | isoform 2 | SPSKSFWTRITCAEEGWSPTPKCLRLCFFP |
| | Uniprot: P36980-2 | FVENGHSESSGQTHLEGDTVQIICNTGYRL |
| | Entry version 168 | QNNENNISCVERGWSTPPKCRSTSSSVEYQ |
| | (11 Dec. 2019) | CQNLYQLEGNNQITCRNGQWSEPPKCLDPC |
| | Sequence version 1 | VISQEIMEKYNIKLKWTNQQKLYSRTGDIV |
| | (01 Jun. 1994) | EFVCKSGYHPTKSHSFRAMCQNGKLVYPSC |
| | | EEK |
| 6 | Complement Factor | MLLLINVILTLWVSCANGQVKPCDFPDIKH |
| | H-related 3 (FHR3), | GGLFHENMRRPYFPVAVGKYYSYYCDEHFE |
| | isoform 1 | TPSGSYWDYIHCTQNGWSPAVPCLRKCYFP |
| | Uniprot: Q02985-1 | YLENGYNQNYGRKFVQGNSTEVACHPGYGL |
| | Entry version 161 | PKAQTTVTCTEKGWSPTPRCIRVRTCSKSD |
| | (11 Dec. 2019) | IEIENGFISESSSIYILNKEIQYKCKPGYA |
| | Sequence version 2 | TADGNSSGSITCLQNGWSAQPICINSSEKC |
| | (21 Feb. 2001) | GPPPPISNGDTTSFLLKVYVPQSRVEYQCQ |
| | | PYYELQGSNYVTCSNGEWSEPPRCIHPCII |
| | | TEENMNKNNIKLKGRSDRKYYAKTGDTIEF |
| | | MCKLGYNANTSILSFQAVCREGIVEYPRCE |
| 7 | Complement Factor | MLLLINVILTLWVSCANGQVKPCDFPDIKH |
| | H-related 3 (FHR3), | GGLFHENMRRPYFPVAVGKYYSYYCDEHFE |
| | isoform 2 | TPSGSYWDYIHCTQNGWSPAVPCLRKCYFP |
| | Uniprot: Q02985-2 | YLENGYNQNYGRKFVQGNSTEVACHPGYGL |
| | Entry version 161 | PKAQTTVTCTEKGWSPTPRCIRVNSSEKCG |
| | (11 Dec. 2019) | PPPPISNGDTTSFLLKVYVPQSRVEYQCQP |
| | Sequence version 2 | YYELQGSNYVTCSNGEWSEPPRCIHPCIIT |
| | (21 Feb. 2001) | EENMNKNNIKLKGRSDRKYYAKTGDTIEFM |
| | | CKLGYNANTSILSFQAVCREGIVEYPRCE |
| 8 | Complement Factor | MLLLINVILTLWVSCANGQEVKPCDFPEIQ |
| | H-related 4 (FHR4A) | HGGLYYKSLRRLYFPAAAGQSYSYY CDQN |
| | Uniprot: Q92496-1 | FVTPSGSYWDYIHCTQDGWSPTVPCLRTCS |
| | Entry version 157 | KSDVEIENGFISESSSIYILN EETQYNCK |
| | (11 Dec. 2019) | PGYATAEGNSSGSITCLQNGWSTQPICIKF |
| | Sequence version 3 | CDMPVFENSRAKSNGMWFKLHDTLDYECYD |
| | (22 Jan. 2014) | GYESSYGNTTDSIVCGEDGWSHLPTCYNSS |
| | | ENCGPPPPISNGDTTSFPQKVYLPWSRVEY |
| | | QCQSYYELQGSKYVTCSNGDWSEPPRCISM |
| | | KPCEFPEIQHGHLYYENTRRPYFPVATGQS |
| | | YSYYCDQNFVTPSGSYWDYIHCTQDGWLPT |
| | | VPCLRTCSKSDIEIENGFISESSSIYILNK |
| | | EIQYKCKPGYATADGNSSGSITCLQNGWSA |
| | | QPICIKFCDMPVFENSRAKSNGMRFKLHDT |
| | | LDYECYDGYEISYGNTTGSIVCGEDGWSHF |
| | | PTCYNSSEKCGPPPPISNGDTTSFLLKVYV |
| | | PQSRVEYQCQSYYELQGSNYVTCSNGEWSE |
| | | PPRCIHPCIITEENMNKNNIQLKGKSDIKY |
| | | YAKTGDTIEFMCKLGYNANTSVLSFQAVCR |
| | | EGIVEYPRCE |
| 9 | Complement Factor | MLLLINVILTLWVSCANGQEVKPCDFPEIQ |
| | H-related 4 (FHR4B) | HGGLYYKSLRRLYFPAAAGQSYSYYCDQNF |
| | Uniprot: Q92496-3 | VTPSGSYWDYIHCTQDGWSPTVPCLRTCSK |
| | Entry version 157 | SDIEIENGFISESSSIYILNKEIQYKCKPG |
| | (11 Dec. 2019) | YATADGNSSGSITCLQNGWSAQPICIKFCD |
| | Sequence version 3 | MPVFENSRAKSNGMRFKLHDTLDYECYDGY |
| | (22 Jan. 2014) | EISYGNTTGSIVCGEDGWSHFPTCYNSSEK |
| | | CGPPPPISNGDTTSFLLKVYVPQSRVEYQC |
| | | QSYYELQGSNYVTCSNGEWSEPPRCIHPCI |
| | | ITEENMNKNNIQLKGKSDIKYYAKTGDTIE |
| | | FMCKLGYNANTSVLSFQAVCREGIVEYPRC |
| | | E |
| 10 | Complement Factor | MLLLFSVILISWVSTVGGEGTLCDFPKIHH |
| | H-related 5 (FHR5) | GFLYDEEDYNPFSQVPTGEVFYYSCEYNFV |
| | Uniprot: Q9BXR6 | SPSKSFWTRITCTEEGWSPTPKCLRMCSFP |
| | | FVKNGHSESSGLIHLEGDTVQIICNTGYSL |
| | | QNNEKNISCVERGWSTPPICSFTKGECHVP |
| | | ILEANVDAQPKKESYKVGDVLKFSCRKNLI |
| | | RVGSDSVQCYQFGWSPNFPTCKGQVRSCGP |
| | | PPQLSNGEVKEIRKEEYGHNEVVEYDCNPN |
| | | FIINGPKKIQCVDGEWTTLPTCVEQVKTCG |
| | | YIPELEYGYVQPSVPPYQHGVSVEVNCRNE |
| | | YAMIGNNMITCINGIWTELPMCVATHQLKR |
| | | CKIAGVNIKTLLKLSGKEFNHNSRIRYRCS |
| | | DIFRYRHSVCINGKWNPEVDCTEKREQFCP |
| | | PPPQIPNAQNMTTTVNYQDGEKVAVLCKEN |
| | | YLLPEAKEIVCKDGRWQSLPRCVESTAYCG |
| | | PPPSINNGDTTSFPLSVYPPGSTVTYRCQS |
| | | FYKLQGSVTVTCRNKQWSEPPRCLDPCVVS |
| | | EENMNKNNIQLKWRNDGKLYAKTGDAVEFQ |
| | | CKFPHKAMISSPPFRAICQEGKFEYPICE |
| 11 | Complement Factor | MKLLHVFLLFLCFHLRFCKVTYTSQEDLVE |
| | I (FI) | KKCLAKKYTHLSCDKVFCQPWQRCIEGTCV |
| | Uniprot: P05156 | CKLPYQCPKNGTAVCATNRRSFPTYCQQKS |
| | Entry version 147 | LECLHPGTKFLNNGTCTAEGKFSVSLKHGN |
| | (11 Dec. 2019) | TDSEGIVEVKLVDQDKTMFICKSSWSMREA |
| | Sequence version 1 | NVACLDLGFQQGADTQRRFKLSDLSINSTE |
| | (01 Jun. 2001) | CLHVHCRGLETSLAECTFTKRRTMGYQDFA |
| | | DVVCYTQKADSPMDDFFQCVNGKYISQMKA |
| | | CDGINDCGDQSDELCCKACQGKGFHCKSGV |
| | | CIPSQYQCNGEVDCITGEDEVGCAGFASVT |
| | | QEETEILTADMDAERRRIKSLLPKLSCGVK |
| | | NRMHIRRKRIVGGKRAQLGDLPWQVAIKDA |
| | | SGITCGGIYIGGCWILTAAHCLRASKTHRY |
| | | QIWTTVVDWIHPDLKRIVIEYVDRIIFHEN |
| | | YNAGTYQNDIALIEMKKDGNKKDCELPRSI |
| | | PACVPWSPYLFQPNDTCIVSGWGREKDNER |
| | | VFSLQWGEVKLISNCSKFYGNRFYEKEMEC |
| | | AGTYDGSIDACKGDSGGPLVCMDANNVTYV |
| | | WGVVSWGENCGKPEFPGVYTKVANYFDWIS |
| | | YHVGRPFISQYNV |
| 12 | Complement factor | MGPTSGPSLLLLLLTHLPLALGSPMYSIIT |
| | C3 | PNILRLESEETMVLEAHDAQGDVPVTVTVH |
| | (Uniprot: P01024) | DFPGKKLVLSSEKTVLTPATNHMGNVTFTI |
| | Entry version 242 | PANREFKSEKGRNKFVTVQATFGTQVVEKV |
| | (11 Dec. 2019) | VLVSLQSGYLFIQTDKTIYTPGSTVLYRIF |
| | Sequence version 2 | TVNHKLLPVGRTVMVNIENPEGIPVKQDSL |
| | (12 Dec. 2006) | SSQNQLGVLPLSWDIPELVNMGQWKIRAYY |
| | | ENSPQQVFSTEFEVKEYVLPSFEVIVEPTE |
| | | KFYYIYNEKGLEVTITARFLYGKKVEGTAF |
| | | VIFGIQDGEQRISLPESLKRIPIEDGSGEV |
| | | VLSRKVLLDGVQNPRAEDLVGKSLYVSATV |
| | | ILHSGSDMVQAERSGIPIVTSPYQIHFTKT |
| | | PKYFKPGMPFDLMVFVTNPDGSPAYRVPVA |
| | | VQGEDTVQSLTQGDGVAKLSINTHPSQKPL |
| | | SITVRTKKQELSEAEQATRTMQALPYSTVG |
| | | NSNNYLHLSVLRTELRPGETLNVNFLLRMD |
| | | RAHEAKIRYYTYLIMNKGRLLKAGRQVREP |
| | | GQDLVVLPLSITTDFIPSFRLVAYYTLIGA |
| | | SGQREVVADSVWVDVKDSCVGSLVVKSGQS |
| | | EDRQPVPGQQMTLKIEGDHGARVVLVAVDK |
| | | GVFVLNKKNKLTQSKIWDVVEKADIGCTPG |
| | | SGKDYAGVFSDAGLTFTSSSGQQTAQRAEL |
| | | QC |
| | | PQPAARRRRSVOLTEKRMDKVGKYPKELRK |
| | | CCEDGMRENPMRFSCQRRTRFISLGEACKK |
| | | VELDCCNYITELRRQHARASHLGLARSNLD |
| | | EDIIAEENIVSRSEFPESWLWNVEDLKEPP |
| | | KNGISTKLMNIFLKDSITTWEILAVSMSDK |
| | | KGICVADPFEVTVMQDFFIDLRLPYSVVRN |
| | | EQVEIRAVLYNYRQNQELKVRVELLHNPAF |
| | | CSLATTKRRHQQTVTIPPKSSLSVPYVIVP |
| | | LKTGLQEVEVKAAVYHHFISDGVRKSLKVV |
| | | PEGIRMNKTVAVRTLDPERLGREGVQKEDI |
| | | PPADLSDQVPDTESETRILLQGTPVAQMTE |
| | | DAVDAERLKHLIVTPSGCGEQNMIGMTPTV |
| | | IAVHYLDETEQWEKFGLEKRQGALELIKKG |
| | | YTQQLAFRQPSSAFAAFVKRAPSTWLTAYV |
| | | VKVFSLAVNLIAIDSQVLCGAVKWLILEKQ |
| | | KPDGVFQEDAPVIHQEMIGGLRNNNEKDMA |
| | | LTAFVLISLQEAKDICEEQVNSLPGSITKA |
| | | GDFLEANYMNLQRSYTVAIAGYALAQMGRL |
| | | KGPLLNKFLTTAKDKNRWEDPGKQLYNVEA |
| | | TSYALLALLQLKDFDFVPPVVRWLNEQRYY |
| | | GGGYGSTQATFMVFQALAQYQKDAPDHQEL |
| | | NLDVSLQLPSRSSKITHRIHWESASLLRSE |
| | | ETKENEGFTVTAEGKGQGTLSVVTMYHAKA |
| | | KDQLTCNKFDLKVTIKPAPETEKRPQDAKN |
| | | TMILEICTRYRGDQDATMSILDISMMTGFA |
| | | PDTDDLKQLANGVDRYISKYELDKAFSDRN |
| | | TLIIYLDKVSHSEDDCLAFKVHQYFNVELI |
| | | QPGAVKVYAYYNLEESCTRFYHPEKEDGKL |
| | | NKLCRDELCRCAEENCFIQKSDDKVTLEER |
| | | LDKACEPGVDYVYKTRLVKVQLSNDFDEYI |
| | | MAIEQTIKSGSDEVQVGQQRTFISPIKCRE |
| | | ALKLEEKKHYLMWGLSSDFWGEKPNLSYII |
| | | GKDTWVEHWPEEDECQDEENQKQCQDLGAF |
| | | TESMVVFGCPN |
| 13 | Human C3 β chain | SPMYSIITPNILRLESEETMVLEAHDAQGD |
| | UniProt: P01024; | VPVTVTVHDFPGKKLVLSSEKTVLTPATNH |
| | Entry version 242 | MGNVTFTIPANREFKSEKGRNKFVTVQATF |
| | (11 Dec. 2019) | GTQVVEKVVLVSLQSGYLFIQTDKTIYTPG |
| | Sequence version 2 | STVLYRIFTVNHKLLPVGRTVMVNIENPEG |
| | (12 Dec. 2006); | IPVKQDSLSSQNQLGVLPLSWDIPELVNMG |
| | residues 23-667 | QWKIRAYYENSPQQVFSTEFEVKEYVLPSF |
| | | EVIVEPTEKFYYIYNEKGLEVTITARFLYG |
| | | KKVEGTAFVIFGIQDGEQRISLPESLKRIP |
| | | IEDGSGEVVLSRKVLLDGVQNPRAEDLVGK |
| | | SLYVSATVILHSGSDMVQAERSGIPIVTSP |
| | | YQIHFTKTPKYFKPGMPFDLMVFVTNPDGS |
| | | PAYRVPVAVQGEDTVQSLTQGDGVAKLSIN |
| | | THPSQKPLSITVRTKKQELSEAEQATRTMQ |
| | | ALPYSTVGNSNNYLHLSVLRTELRPGETLN |
| | | VNFLLRMDRAHEAKIRYYTYLIMNKGRLLK |
| | | AGRQVREPGQDLVVLPLSITTDFIPSFRLV |
| | | AYYTLIGASGQREVVADSVWVDVKDSCVGS |
| | | LVVKSGQSEDRQPVPGQQMTLKIEGDHGAR |
| | | VVLVAVDKGVFVLNKKNKLTQSKIWDVVEK |
| | | ADIGCTPGSGKDYAGVFSDAGLTFTSSSGQ |
| | | QTAQRAELQCPQPAA |
| 14 | Human C3 α′ chain | SNLDEDIIAEENIVSRSEFPESWLWNVEDL |
| | UniProt: P01024; | KEPPKNGISTKLMNIFLKDSITTWEILAVS |
| | Entry version 242 | MSDKKGICVADPFEVTVMQDFFIDLRLPYS |
| | (11 Dec. 2019) | VVRNEQVEIRAVLYNYRQNQELKVRVELLH |
| | Sequence version 2 | NPAFCSLATTKRRHQQTVTIPPKSSLSVPY |
| | (12 Dec. 2006); | VIVPLKTGLQEVEVKAAVYHHFISDGVRKS |
| | residues 749-1663 | LKVVPEGIRMNKTVAVRTLDPERLGREGVQ |
| | | KEDIPPADLSDQVPDTESETRILLQGTPVA |
| | | QMTEDAVDAERLKHLIVTPSGCGEQNMIGM |
| | | TPTVIAVHYLDETEQWEKFGLEKRQGALEL |
| | | IKKGYTQQLAFRQPSSAFAAFVKRAPSTWL |
| | | TAYVVKVFSLAVNLIAIDSQVLCGAVKWLI |
| | | LEKQKPDGVFQEDAPVIHQEMIGGLRNNNE |
| | | KDMALTAFVLISLQEAKDICEEQVNSLPGS |
| | | ITKAGDFLEANYMNLQRSYTVAIAGYALAQ |
| | | MGRLKGPLLNKFLTTAKDKNRWEDPGKQLY |
| | | NVEATSYALLALLQLKDFDFVPPVVRWLNE |
| | | QRYYGGGYGSTQATFMVFQALAQYQKDAPD |
| | | HQELNLDVSLQLPSRSSKITHRIHWESASL |
| | | LRSEETKENEGFTVTAEGKGQGTLSVVTMY |
| | | HAKAKDQLTCNKFDLKVTIKPAPETEKRPQ |
| | | DAKNTMILEICTRYRGDQDATMSILDISMM |
| | | TGFAPDTDDLKQLANGVDRYISKYELDKAF |
| | | SDRNTLIIYLDKVSHSEDDCLAFKVHQYFN |
| | | VELIQPGAVKVYAYYNLEESCTRFYHPEKE |
| | | DGKLNKLCRDELCRCAEENCFIQKSDDKVT |
| | | LEERLDKACEPGVDYVYKTRLVKVQLSNDF |
| | | DEYIMAIEQTIKSGSDEVQVGQQRTFISPI |
| | | KCREALKLEEKKHYLMWGLSSDFWGEKPNL |
| | | SYIIGKDTWVEHWPEEDECQDEENQKQCQD |
| | | LGAFTESMVVFGCPN |
| 15 | Human C3a | SVQLTEKRMDKVGKYPKELRKCCEDGMREN |
| | UniProt: P01024; | PMRFSCQRRTRFISLGEACKKVFLDCCNYI |
| | Entry version 242 | TELRRQHARASHLGLAR |
| | (11 Dec. 2019) | |
| | Sequence version 2 | |
| | (12 Dec. 2006); | |
| | residues 672-748 | |
| 16 | Human C3b α′ chain | SNLDEDIIAEENIVSRSEFPESWLWNVEDL |
| | fragment 1 | KEPPKNGISTKLMNIFLKDSITTWEILAVS |
| | UniProt: P01024; | MSDKKGICVADPFEVTVMQDFFIDLRLPYS |
| | Entry version 242 | VVRNEQVEIRAVLYNYRQNQELKVRVELLH |
| | (11 Dec. 2019) | NPAFCSLATTKRRHQQTVTIPPKSSLSVPY |
| | Sequence version 2 | VIVPLKTGLQEVEVKAAVYHHFISDGVRKS |
| | (12 Dec. 2006); | LKVVPEGIRMNKTVAVRTLDPERLGREGVQ |
| | residues 749-1663 | KEDIPPADLSDQVPDTESETRILLQGTPVA |
| | | QMTEDAVDAERLKHLIVTPSGCGEQNMIGM |
| | | TPTVIAVHYLDETEQWEKFGLEKRQGALEL |
| | | IKKGYTQQLAFRQPSSAFAAFVKRAPSTWL |
| | | TAYVVKVFSLAVNLIAIDSQVLCGAVKWLI |
| | | LEKQKPDGVFQEDAPVIHQEMIGGLRNNNE |
| | | KDMALTAFVLISLQEAKDICEEQVNSLPGS |
| | | ITKAGDFLEANYMNLQRSYTVAIAGYALAQ |
| | | MGRLKGPLLNKFLTTAKDKNRWEDPGKQLY |
| | | NVEATSYALLALLQLKDFDFVPPVVRWLNE |
| | | QRYYGGGYGSTQATFMVFQALAQYQKDAPD |
| | | HQELNLDVSLQLPSRSSKITHRIHWESASL |
| | | LRSEETKENEGFTVTAEGKGQGTLSVVTMY |
| | | HAKAKDQLTCNKFDLKVTIKPAPETEKRPQ |
| | | DAKNTMILEICTRYRGDQDATMSILDISMM |
| | | TGFAPDTDDLKQLANGVDRYISKYELDKAF |
| | | SDRNTLIIYLDKVSHSEDDCLAFKVHQ |
| | | YFNVELIQPGAVKVYAYYNLEESCTRFYHP |
| | | EKEDGKLNKLCRDELCRCAEENCFIQKSDD |
| | | KVTLEERLDKACEPGVDYVYKTRLVKVQLS |
| | | NDFDEYIMAIEQTIKSGSDEVQVGQQRTFI |
| | | SPIKCREALKLEEKKHYLMWGLSSDFWGEK |
| | | PNLSYIIGKDTWVEHWPEEDECQDEENQKQ |
| | | CQDLGAFTESMVVFGCPN |
| 17 | Human C3 α′ chain | SEETKENEGFTVTAEGKGQGTLSVVTMYHA |
| | fragment 2 | KAKDQLTCNKFDLKVTIKPAPETEKRPQDA |
| | UniProt: P01024; | KNTMILEICTRYRGDQDATMSILDISMMTG |
| | Entry version 242 | FAPDTDDLKQLANGVDRYISKYELDKAFSD |
| | (11 Dec. 2019) | RNTLIIYLDKVSHSEDDCLAFKVHQYFNVE |
| | Sequence version 2 | LIQPGAVKVYAYYNLEESCTRFYHPEKEDG |
| | (12 Dec. 2006); | KLNKLCRDELCRCAEENCFIQKSDDKVTLE |
| | residues 1321-1663 | ERLDKACEPGVDYVYKTRLVKVQLSNDFDE |
| | | YIMAIEQTIKSGSDEVQVGQQRTFISPIKC |
| | | REALKLEEKKHYLMWGLSSDFWGEKPNLSY |
| | | IIGKDTWVEHWPEEDECQDEENQKQCQDLG |
| | | AFTESMVVFGCPN |
| 18 | C3f | SSKITHRIHWESASLLR |
| | UniProt: P01024; | |
| | Entry version 242 | |
| | (11 Dec. 2019) | |
| | Sequence version 2 | |
| | (12 Dec. 2006) | |
| | residues 1304-1320 | |
| 19 | Human C3c α′ chain | SNLDEDIIAEENIVSRSEFPESWLWNVEDL |
| | fragment 1 | KEPPKNGISTKLMNIFLKDSITTWEILAVS |
| | UniProt: P01024; | MSDKKGICVADPFEVTVMQDFFIDLRLPYS |
| | Entry version 242 | VVRNEQVEIRAVLYNYRQNQELKVRVELLH |
| | (11 Dec. 2019) | NPAFCSLATTKRRHQQTVTIPPKSSLSVPY |
| | Sequence version 2 | VIVPLKTGLQEVEVKAAVYHHFISDGVRKS |
| | (12 Dec. 2006); | LKVVPEGIRMNKTVAVRTLDPERLGR |
| | residues 749-954 | |
| 20 | FH MS peptide | VTYKCFE |
| 21 | FHL-1 MS peptide | NGWSPTPRCIRVSFTL |
| 22 | FHR1 MS peptide | ATFCDFPKINHGILYDEE |
| 23 | FHR2 MS peptide 1 | RGWSTPPKCRSTISAE |
| 24 | FHR2 MS peptide 2 | AMFCDFPKINHGILYDEE |
| 25 | FHR3 MS peptide | VACHPGYGLPKAQTTVTCTE |
| 26 | FHR4 MS peptide | YQCQSYYE |
| 27 | FHR5 MS peptide | RGWSTPPICSFTKGE |
| 28 | C3.1 peptide | GTAFVIFGIQDGE |
| 29 | C3.2 peptide | LRRQHARASHLGLARSNLDE |
| 30 | C3.3 peptide | LRRQHARASHLGLAR |
| 31 | C3.4 peptide | LNLDVSLQLPSRSSKITHRIHWE |
| 32 | C3.5 peptide | LNLDVSLQLPSR |
| 33 | C3.6 peptide | RLGRE |
| 34 | C3.7 peptide | SSKITHRIHWE |
| 35 | C3.8 peptide | SASLLR |
| 36 | C3.9 peptide | RLGR |
| 37 | C3.10 peptide | HLIVTPSGCGE |
| 38 | FI peptide 1 | CAGTYDGSIDACKGDSGGPLVCMDANNVTY |
| | | VWGVVSWGE |
| 39 | FI peptide 2 | GTCVCKLPYQCPKNGTAVCATNRRSFPTYC |
| | | QQKSLE |
| 40 | FI peptide 3 | FPGVYTKVANYFDWISYHVGRPFISQYNV |
| 41 | FI peptide 4 | ANVACLDLGFQQGADTQRRFKLSDLSINST |
| | | E |
| 42 | FI peptide 5 | LPRSIPACVPWSPYLFQPNDTCIVSGWGRE |
| 43 | FI peptide 6 | KKCLAKKYTHLSCDKVFCQPWQRCIE |
| 44 | FI peptide 7 | LCCKACQGKGFHCKSGVCIPSQYQCNGE |
| 45 | FI peptide 8 | VKLVDQDKTMFICKSSWSMRE |
| 46 | FI peptide 9 | VKLISNCSKFYGNRFYE |
| 47 | FI peptide 10 | CLHPGTKFLNNGTCTAE |
| 48 | FI peptide 11 | NYNAGTYQNDIALIE |
| 49 | FI peptide 12 | GKFSVSLKHGNTDSE |
| 50 | FI peptide 13 | VGCAGFASVTQEE |
| 51 | FI peptide 14 | MKKDGNKKDCE |
| 52 | FI peptide 15 | YVDRIIFHE |
| 53 | FI peptide 16 | CLHVHCRGLE |
| 54 | FI peptide 17 | RVFSLQWGE |
| 55 | FI peptide 18 | ILTADMDAE |
| 56 | FI peptide 19 | KVTYTSQE |
| 57 | FI peptide 20 | VDCITGE |
| 58 | FI peptide 21 | NCGKPE |
| 59 | FI peptide 22 | TSLAE |
| 60 | FI peptide 23 | KDNE |
| 61 | C3.1.1 | AKIRYYTYLIMNKGRLLKAGRQVREPGQDL |
| | | VVLPLSITTDFIPSFRLVAYYTLIGASGQR |
| | | E |
| 62 | C3.1.2 | RSGIPIVTSPYQIHFTKTPKYFKPGMPFDL |
| | | MVFVTNPDGS PAYRVPVAVQGE |
| 63 | C3.1.3 | KVVLVSLQSGYLFIQTDKTIYTPGSTVLYR |
| | | IFTVNHKLLPVGRTVMVNIE |
| 64 | C3.1.4 | DTVQSLTQGDGVAKLSINTHPSQKPLSITV |
| | | RTKKQE |
| 65 | C3.1.5 | GDHGARVVLVAVDKGVFVLNKKNKLTQSKI |
| | | WDVVE |
| 66 | C3.1.6 | KADIGCTPGSGKDYAGVFSDAGLTFTSSSG |
| | | QQTAQRAE |
| 67 | C3.1.7 | QATRTMQALPYSTVGNSNNYLHLSVLRTE |
| 68 | C3.1.8 | AHDAQGDVPVTVTVHDFPGKKLVLSSE |
| 69 | C3.1.9 | GIPVKQDSLSSQNQLGVLPLSWDIPE |
| 70 | C3.1.10 | VVADSVWVDVKDSCVGSLVVKSGQSE |
| 71 | C3.1.11 | DLVGKSLYVSATVILHSGSDMVQAE |
| 72 | C3.1.12 | KTVLTPATNHMGNVTFTIPANRE |
| 73 | C3.1.13 | KGRNKFVTVQATFGTQVVE |
| 74 | C3.1.14 | VVLSRKVLLDGVQNPRAE |
| 75 | C3.1.15 | TLNVNFLLRMDRAHE |
| 76 | C3.1.16 | LVNMGQWKIRAYYE |
| 77 | C3.1.17 | SPMYSIITPNILRLE |
| 78 | C3.1.18 | DRQPVPGQQMTLKIE |
| 79 | C3.1.19 | VTITARFLYGKKVE |
| 80 | C3.1.20 | GTAFVIFGIQDGE |
| 81 | C3.1.21 | KFYYIYNE |
| 82 | C3.1.22 | NSPQQVFSTE |
| 83 | C3.1.23 | SLKRIPIE |
| 84 | C3.1.24 | YVLPSFE |
| 85 | C3.1.25 | QRISLPE |
| 86 | C3.1.26 | LQCPQPAA |
| 87 | C3.1.27 | VIVEPTE |
| 88 | C3.1.28 | TMVLE |
| 89 | C3.1.29 | LRPGE |
| 90 | C3.1.30 | FKSE |
| 91 | FHR1 peptide 2 | NYNIALRWTAKQKLYLRTGE |
| 92 | FHR2 peptide 3 | YNFVSPSKSFWTRITCAEE |
| 93 | FHR3 peptide 2 | KGWSPTPRCIRVRTCSKSDIE |
| 94 | FHR3 peptide 3 | NGYNQNYGRKFVQGNSTE |
| 95 | FHR3 peptide 4 | QVKPCDFPDIKHGGLFHE |
| 96 | FHR3 peptide 5 | FMCKLGYNANTSILSFQAVCRE |
| 97 | FHR3 peptide 6 | YQCQPYYE |
| 98 | FHR4 peptide 2 | NSRAKSNGMRFKLHDTLDYE |
| 99 | FHR4 peptide 3 | DGWSHFPTCYNSSE |
| 100 | FHR4 peptide 4 | ISYGNTTGSIVCGE |
| 101 | FHR4 peptide 5 | FMCKLGYNANTSVLSFQAVCRE |
| 102 | FHR5 peptide 2 | GTLCDFPKIHHGFLYDEE |
| 103 | FHR5 peptide 3 | YAMIGNNMITCINGIWTE |
| 104 | FHR5 peptide 4 | YGYVQPSVPPYQHGVSVE |
| 105 | FHR5 peptide 5 | GDTVQIICNTGYSLQNNE |
| 106 | FHR5 peptide 6 | IVCKDGRWQSLPRCVE |
| 107 | FHR5 peptide 7 | DYNPFSQVPTGE |
| 108 | FHR5 peptide 8 | QVKTCGYIPE |
| 109 | FHR5 peptide 9 | ANVDAQPKKE |
| 110 | FHR5 peptide 10 | WTTLPTCVE |
| 111 | FHR5 peptide 11 | KVAVLCKE |
| 112 | FH peptide 2 | SNTGSTTGSIVCGYNGWSDLPICYE |
| 113 | FH peptide 3 | NGWSPTPRCIRVKTCSKSSIDIE |
| 114 | FH peptide 4 | LPKIDVHLVPDRKKDQYKVGE |
| 115 | FH peptide 5 | YYCNPRFLMKGPNKIQCVDGE |
| 116 | FH peptide 6 | NYNIALRWTAKQKLYSRTGE |
| 117 | FH peptide 7 | KWSHPPSCIKTDCLSLPSFE |
| 118 | FH peptide 8 | HGWAQLSSPPYYYGDSVE |
| 119 | FH peptide 9 | ISHGVVAHMSDSYQYGEE |
| 120 | FH peptide 10 | FDHNSNIRYRCRGKE |
| 121 | FH peptide 11 | ITCKDGRWQSIPLCVE |
| 122 | FH peptide 12 | GWIHTVCINGRWDPE |
| 123 | FH peptide 13 | KAKYQCKLGYVTADGE |
| 124 | FH peptide 14 | TTCYMGKWSSPPQCE |
| 125 | FH peptide 15 | SYAHGTKLSYTCE |
| 126 | FH peptide 16 | RVRYQCRSPYE |
| 127 | FH peptide 17 | GFGIDGPAIAKCLGE |
| 128 | FH peptide 18 | HGTINSSRSSQE |
| 129 | FH peptide 19 | YQCQNLYQLE |
| 130 | FH peptide 20 | WTTLPVCIVEE |
| 131 | FH peptide 21 | KIPCSQPPQIE |
| 132 | FH peptide 22 | SQYTYALKE |
| 133 | FH peptide 23 | QVQSCGPPPE |
| 134 | FH peptide 24 | KKDVYKAGE |
| 135 | FH peptide 25 | GLPCKSPPE |
| 136 | FH peptide 26 | KVSVLCQE |
| 137 | FH peptide 27 | HLKNKKE |
| 138 | FH peptide 28 | GGFRISEE |
| 139 | FH peptide 29 | LLNGNVKE |
| 140 | FH peptide 30 | YPTCAKR |
| 141 | FH peptide 31 | STCGDIPE |
| 142 | C3dg | EGVQKEDIPPADLSDQVPDTESETRILLQG |
| | UniProt: P01024; | TPVAQMTEDAVDAERLKHLIVTPSGCGEQN |
| | Entry version 242 | MIGMTPTVIAVHYLDETEQWEKFGLEKRQG |
| | (11 Dec. 2019) | ALELIKKGYTQQLAFRQPSSAFAAFVKRAP |
| | Sequence version 2 | STWLTAYVVKVFSLAVNLIAIDSQVLCGAV |
| | (12 Dec. 2006) | KWLILEKQKPDGVFQEDAPVIHQEMIGGLR |
| | residues 955-1303 | NNNEKDMALTAFVLISLQEAKDICEEQVNS |
| | | LPGSITKAGDFLEANYMNLQRSYTVAIAGY |
| | | ALAQMGRLKGPLLNKFLTTAKDKNRWEDPG |
| | | KQLYNVEATSYALLALLQLKDFDFVPPVVR |
| | | WLNEQRYYGGGYGSTQATFMVFQALAQYQK |
| | | DAPDHQELNLDVSLQLPSR |
| 143 | C3g | EGVQKEDIPPADLSDQVPDTESETRILLQG |
| | UniProt: P01024; | TPVAQMTEDAVDAERLK |
| | Entry version 242 | |
| | (11 Dec. 2019) | |
| | Sequence version 2 | |
| | (12 Dec. 2006) | |
| | residues 955-1001 | |
| 144 | C3d | HLIVTPSGCGEQNMIGMTPTVIAVHYLDET |
| | UniProt: P01024; | EQWEKFGLEKRQGALELIKKGYTQQLAFRQ |
| | Entry version 242 | PSSAFAAFVKRAPSTWLTAYVVKVFSLAVN |
| | (11 Dec. 2019) | LIAIDSQVLCGAVKWLILEKQKPDGVFQED |
| | Sequence version 2 | APVIHQEMIGGLRNNNEKDMALTAFVLISL |
| | (12 Dec. 2006) | QEAKDICEEQVNSLPGSITKAGDFLEANYM |
| | residues 1002-1303 | NLQRSYTVAIAGYALAQMGRLKGPLLNKFL |
| | | TTAKDKNRWEDPGKQLYNVEATSYALLALL |
| | | QLKDFDFVPPVVRWLNEQRYYGGGYGSTQA |
| | | TFMVFQALAQYQKDAPDHQELNLDVSLQLP |
| | | SR |
| 145 | Human Complement | GHCQAPDHFLFAKLKTQTNASDFPIGTSLK |
| | Receptor 1; | YECRPEYYGRPFSITCLDNLVWSSPKDVCK |
| | consensus | RKSCKTPPDPVNGMVHVITDIQVGSRINYS |
| | sequence for CCPs | CTTGHRLIGHSSAECILSGNX₁AHWSTKPP |
| | 8-10, 15-17 | ICQRIPCGLPPTIANGDFISTNRENFHYGS |
| | (UniProt: P17927 | VVTYRCNX₂GSX₃GRKVFELVGEPSIYCTS |
| | residues | NDDQVGIWSGPAPQCII |
| | 491 to 684; | |
| | residues 941 to | |
| | 1134) | |
| | Without leader | |
| | sequence | |
| 146 | Human Complement | GHCQAPDHFLFAKLKTQTNASDFPIGTSLK |
| | Receptor 1 CCPs 8- | YECRPEYYGRPFSITCLDNLVWSSPKDVCK |
| | 10 (UniProt: P17927 | RKSCKTPPDPVNGMVHVITDIQVGSRINYS |
| | residues 491 to 684) | CTTGHRLIGHSSAECILSGNAAHWSTKPPI |
| | Without leader | CQRIPCGLPPTIANGDFISTNRENFHYGSV |
| | sequence | VTYRCNPGSGGRKVFELVGEPSIYCTSNDD |
| | | QVGIWSGPAPQCII |
| 147 | Human Complement | GHCQAPDHFLFAKLKTQTNASDFPIGTSLK |
| | Receptor 1 CCPs | YECRPEYYGRPFSITCLDNLVWSSPKDVCK |
| | 15-17 (UniProt: | RKSCKTPPDPVNGMVHVITDIQVGSRINYS |
| | P17927 residues | CTTGHRLIGHSSAECILSGNTAHWSTKPPI |
| | 941 to 1134) | CQRIPCGLPPTIANGDFISTNRENFHYGSV |
| | Without leader | VTYRCNLGSRGRKVFELVGEPSIYCTSNDD |
| | sequence | QVGIWSGPAPQCII |
| 148 | Human Complement | GHCQAPDHFLFAKLKTQTNASDFPIGTSLK |
| | Receptor 1 | YECRPEYYGRPFSITCLDNLVWSSPKDVCK |
| | CCPs 8-10 and | RKSCKTPPDPVNGMVHVITDIQVGSRINYS |
| | 15-17 | CTTGHRLIGHSSAECILSGN [symbol] AHWSTKPPI |
| | (contiguous; | CQRIPCGLPPTIANGDFISTNRENFHYGSV |
| | without | VTYRCN [symbol] GS [symbol] GRKVFELVGEPSIYCTSNDD |
| | leader sequence) | QVGIWSGPAPQCIIGHCQAPDHFLFAKLKT |
| | [amino acid | QTNASDFPIGTSLKYECRPEYYGRPFSITC |
| | differences between | LDNLVWSSPKDVCKRKSCKTPPDPVNGMVH |
| | CCPs 8-10 and 15- | VITDIQVGSRINYSCTTGHRLIGHSSAECI |
| | 17 indicated with | LSGNIAHWSTKPPICQRIPCGLPPTIANGD |
| | wavy underline] | FISTNRENFHYGSVVTYRCN [symbol] GS [symbol] GRKVFE |
| | | LVGEPSIYCTSNDDQVGIWSGPAPQCII |
| 149 | Human | IVGGKRAQLGDLPWQVAIKDASGITCGGIY |
| | Complement Factor | IGGCWILTAAHCLRASKTHRYQIWTTVVDW |
| | I proteolytic | IHPDLKRIVIEYVDRIIFHENYNAGTYQND |
| | domain | IALIEMKKDGNKKDCELPRSIPACVPWSPY |
| | (UniProt: P05156 | LFQPNDTCIVSGWGREKDNERVFSLQWGEV |
| | residues 340-574) | KLISNCSKFYGNRFYEKEMECAGTYDGSID |
| | | ACKGDSGGPLVCMDANNVTYVWGVVSWGEN |
| | | CGKPEFPGVYTKVANYFDWISYHVG |
| 150 | Human | EDCNELPPRRNTEILTGSWSDQTYPEGTQA |
| | Complement Factor | IYKCRPGYRSLGNVIMVCRKGEWVALNPLR |
| | H co-factor region | KCQKRPCGHPGDTPFGTFTLTGGNVFEYGV |
| | (UniProt: P08603 | KAVYTCNEGYQLLGEINYRECDTDGWTNDI |
| | residues 19-264) | PICEVVKCLPVTAPENGKIVSSAMEPDREY |
| | | HFGQAVRFVCNSGYKIEGDEEMHCSDDGFW |
| | | SKEKPKCVEISCKSPDVINGSPISQKIIYK |
| | | ENERFQYKCNMGYEYSERGDAVCTESGWRP |
| | | LPSCEE |
| 151 | Human | GHCQAPDHFLFAKLKTQTNASDFPIGTSLK |
| | Complement | YECRPEYYGRPFSITCLDNLVWSSPKDVCK |
| | Receptor 1 CCPs | RKSCKTPPDPVNGMVHVITDIQVGSRINYS |
| | 8-10 (UniProt: | CTTGHRLIGHSSAECILSGNAAHWSTKPPI |
| | P17927 residues | CQRIPCGLPPTIANGDFISTNRENFHYGSV |
| | 491 to 684) | VTYRCNPGSGGRKVFELVGEPSIYCTSNDD |
| | | QVGIWSGPAPQCII |
| 152 | Human | GHCQAPDHFLFAKLKTQTNASDFPIGTSLK |
| | Complement | YECRPEYYGRPFSITCLDNLVWSSPKDVCK |
| | Receptor 1 CCPs | RKSCKTPPDPVNGMVHVITDIQVGSRINYS |
| | 15-17 (UniProt: | CTTGHRLIGHSSAECILSGNTAHWSTKPPI |
| | P17927 residues | CQRIPCGLPPTIANGDFISTNRENFHYGSV |
| | 941 to 1134) | VTYRCNLGSRGRKVFELVGEPSIYCTSNDD |
| | | QVGIWSGPAPQCII |
| 153 | GluC | MKGKFLKVSSLFVATLTTATLVSSPAANAL |
| | (Uniprot Q7A6A6 | SSKAMDNHPQQTQSSKQQTPKIKKGGNLKP |
| | including | LEQREHANVILPNNDRHQITDTTNGHYAPV |
| | N-terminal | TYIQVEAPTGTFIASGVVVGKDTLLTNKHV |
| | signal peptide | VDATHGDPHALKAFPSAINQDNYPNGGFTA |
| | residues 1-29 and | EQITKYSGEGDLAIVKFSPNEQNKHIGEVV |
| | propeptide | KPATMSNNAETQVNQNITVTGYPGDKPVAT |
| | residues | MWESKGKITYLKGEAMQYDLSTTGGNSGSP |
| | 30-68) | VFNEKNEVIGIHWGGVPNEFNGAVFINENV |
| | | RNFLKQNIEDIHFANDDQPNNPDNPDNPNN |
| | | PDNPNNPDNPNNPDEPNNPDNPNNPDNPDN |
| | | GDNNNSDNPDAA |
| 154 | GluC (Uniprot | VILPNNDRHQITDTTNGHYAPVTYIQVEAP |
| | Q7A6A6; without | TGTFIASGVVVGKDTLLTNKHVVDATHGDP |
| | signal peptide or | HALKAFPSAINQDNYPNGGFTAEQITKYSG |
| | propeptide) | EGDLAIVKFSPNEQNKHIGEVVKPATMSNN |
| | | AETQVNQNITVTGYPGDKPVATMWESKGKI |
| | | TYLKGEAMQYDLSTTGGNSGSPVFNEKNEV |
| | | IGIHWGGVPNEFNGAVFINENVRNFLKQNI |
| | | EDIHFANDDQPNNPDNPDNPNNPDNPNNPD |
| | | NPNNPDEPNNPDNPNNPDNPDNGDNNNSDN |
| | | PDAA |
| 155 | FI peptide 24 | VGCAGFASVTQE |
| 156 | C3a-desArg | LRRQHARASHLGLA |
| 157 | C3f-desArg | SASLL |
| 158 | Forward | TGGTCTCTGTGAGAAAGTTCGCCCTCGCAA |
| | mutagenesis | TAGTAGATACT |
| | primer | |
| 159 | Reverse | AGTATCTACTATTGCGAGGGCGAACTTTCT |
| | mutagenesis | CACAGAGACCA |
| | primer | |
| 160 | Human GAPDH F | AGGTATGACAACGAATTTGGC |
| 161 | Human GAPDH R | GTGTGGCAGGGACTCCC |
| 162 | Human IDO1 F | AGCAGACTGCTGGTGGAGGACATG |
| 163 | Human IDO1 R | TTCACACAGGCGTCATAAGCTTCC |
| 164 | Human CFH N- | GAATCTGGATGGCGTCCGTT |
| | term F | |
| 165 | Human CFH N- | TGGTACGTGATTTCATCTCCAGT |
| | term R | |
| 166 | Human CFH C- | GGAAGCACCACTGGTTCCAT |
| | term F | |
| 167 | Human CFH C- | TGGTCTTTCTTGCGATCAGGA |
| | term R | |
| 168 | Human FHL-1 F | CCTGGCTACGCTCTTCCAAA |
| 169 | Human FHL-1 R | AGGGTAAAGCTGACACGGAT |
| 170 | Mouse GAPDH F | TGGGAGTTGCTGTTGAAGTC |
| 171 | Mouse GAPDH R | TGACATCAAGAAGGTGGTGA |
| 172 | Mouse IDO1 F | AGCAGACTGCTGGTGGAGGACATG |
| 173 | Mouse IDO1 R | TTCACACAGGCGTCATAAGCTTCC |
| 174 | Human IDO1 | GCUUAUGACGCCUGUGUGAUU |
| | SiRNA sense | |
| 175 | Human IDO1 | UCACACAGGCGUCAUAAGCUU |
| | SiRNA antisense | |

Numbered Paragraphs
1. A method for detecting at least one complement protein in a sample, the method comprising:
   digesting the protein(s) with endoproteinase GluC to obtain one or more peptides; and
   detecting the one or more peptides by mass spectrometry.
2. A method for determining the level of at least one complement protein in a sample, the method comprising:
   digesting the protein(s) with endoproteinase GluC to obtain one or more peptides; and
   determining the level of the one or more peptides by mass spectrometry.
3. A method according to para 1 or para 2, wherein the method comprises determining the concentration of the complement protein(s) in the sample.
4. A method for preparing at least one complement protein for analysis, the method comprising digesting the protein(s) with endoproteinase GluC to obtain one or more peptides.
5. A method according to any one of paras 1 to 4, wherein the complement protein(s) is one or more of FH, FHL-1, FHR1, FHR2, FHR3, FHR4 and/or FHR5.
6. A method according to any one of paras 1 to 5, wherein the complement protein is FH and/or FHL-1.
7. A method according to any one of paras 1 to 6, wherein the complement protein(s) is involved in the complement amplification loop and/or C3 convertase activity.
8. A method according to any one of paras 1 to 7, wherein the complement protein(s) is a breakdown product of C3b.
9. A method according to any one of paras 1 to 8, wherein the complement protein(s) is one or more of C3, C3b, C3a, iC3b, C3f, C3c, C3dg, and/or C3d.
10. A method according to any one of paras 1 to 9, wherein the complement protein(s) is C3b and/or iC3b.
11. A method according to any one of paras 1 to 10, wherein the complement protein is FI.
12. A method according to any one of paras 1 to 11, wherein the sample has been obtained from a subject.
13. A method according to any one of paras 1 to 11, wherein the method comprises a step of obtaining the sample from a subject.
14. A method according to any one of paras 1 to 13, wherein the sample comprises, or is derived from, blood, lymph, plasma, serum, tissue or cells.
15. A method according to any one of paras 1 to 14, wherein the one or more peptides are selected from the group consisting of:

(a)
(SEQ ID NO: 20)
VTYKCFE;
(b)
(SEQ ID NO: 21)
NGWSPTPRCIRVSFTL;
(c)
(SEQ ID NO: 22)
ATFCDFPKINHGILYDEE;
(d)
(SEQ ID NO: 23)
RGWSTPPKCRSTISAE
or
(SEQ ID NO: 24)
AMFCDFPKINHGILYDEE;
(e)
(SEQ ID NO: 25)
VACHPGYGLPKAQTTVTCTE;
(f)
(SEQ ID NO: 26)
YQCQSYYE;
(g)
(SEQ ID NO: 27)
RGWSTPPICSFTKGE;
(h)
(SEQ ID NO: 28)
GTAFVIFGIQDGE;
(i)
(SEQ ID NO: 29)
LRRQHARASHLGLARSNLDE;
(j)
(SEQ ID NO: 30)
LRRQHARASHLGLAR;
(k)
(SEQ ID NO: 31)
LNLDVSLQLPSRSSKITHRIHWE;
(l)
(SEQ ID NO: 32)
LNLDVSLQLPSR;
(m)
(SEQ ID NO: 33)
RLGRE;
(n)
(SEQ ID NO: 34)
SSKITHRIHWE;
(o)
(SEQ ID NO: 35)
SASLLR;
(p)
(SEQ ID NO: 36)
RLGR;
(q)
(SEQ ID NO: 37)
HLIVTPSGCGE;
and/or
(r)
any one or more of SEQ ID NO: 38
to 60, or 155.

16. Use of endoproteinase GluC for preparing at least one complement protein for detection by mass spectrometry, optionally for preparing at least two complement proteins for detection by mass spectrometry.
17. A method of determining the presence and/or level of a complement protein in a subject, the method comprising performing a method for detecting at least one complement protein in a sample according to any one of paras 1 to 15.
18. A method of identifying a subject having a complement-related disorder or at risk of developing a complement-related disorder, the method comprising:
   d) digesting at least one complement protein in a sample obtained from the subject with endoproteinase GluC to obtain one or more peptides;
   e) determining the presence and/or level of the one or more peptides by mass spectrometry; and
   f) using the results of (b) to determine whether the subject has or is likely to develop a complement-related disorder.
19. A method according to para 18, comprising (d) treating a subject who has been determined to be at risk of developing, or to have, a complement-related disorder, optionally wherein treating a subject comprises administering a therapeutically effective amount of a complement-targeted therapeutic to the subject.
20. A method of selecting a subject for treatment of a complement-related disorder with a complement-targeted therapeutic, the method comprising:
   a) digesting at least one complement protein in a sample obtained from the subject with endoproteinase GluC to obtain one or more peptides;
   b) determining the presence and/or level of the one or more peptides by mass spectrometry; and
   c) using the results of (b) to determine whether the subject is in need of a complement-targeted therapeutic.
21. A complement-targeted therapeutic for use in a method of treating a complement-related disorder in a subject, the method comprising:
   a) digesting at least one complement protein in a sample obtained from the subject with endoproteinase GluC to obtain one or more peptides;
   b) determining the presence and/or level of the one or more peptides by mass spectrometry; and
   c) based on the results of (b), administering an effective amount of the complement-targeted therapeutic.
22. A method according to any one of paras 17 to 21, wherein the method comprises obtaining a sample from a subject comprising at least one complement protein, optionally wherein the sample comprises or is derived from blood, lymph, plasma, serum, tissue or cells.
23. A method according to any one of paras 18 to 22, wherein the complement-related disorder is selected from macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, stroke, Parkinson's disease, and/or Alzheimer's disease.
24. A method according to any one of paras 17 to 23, wherein an increase in the level of one or more of C3, C3b, C3a, iC3b, FHR1, FHR2, FHR3, FHR4 and/or FHR5; and/or a decrease in the level of one or more of iC3b, C3f, C3c, C3dg, C3d, C3g, FI, FH, FHL-1, FHR1, FHR2, FHR3, FHR4 and/or FHR5 as compared to reference value(s), indicates that the subject is at risk of developing or has a complement-related disorder.
25. A kit for use in a method of detecting and/or determining the level of one or more complement protein(s) in a sample, the kit comprising endoproteinase GluC.
A. A method of identifying a subject having a complement-related disorder or at risk of developing a complement-related disorder, the method comprising:
   (a) determining the level of a complement protein selected from one or more of FHR1, FHR2, FHR3, FHR4 and/or FHR5 in a blood sample obtained from the subject;
   (b) determining that the subject has or is likely to develop a complement-related disorder if the level of the complement protein determined in (a) is elevated as compared to the level of that complement protein in blood in a control subject that does not have a complement-related disorder.
B. The method according to para A, wherein step (a) comprises determining the level of two or three of the complement proteins selected from FHR1, FHR2 and/or FHR3.
C. The method according to para B, wherein step (a) further comprises determining the level of FHR4 and/or FHR5.
D. The method according to any one of paras A-C, wherein the method further comprises determining the level of FHL-1 and/or FH, optionally determining that the subject has or is likely to develop a complement-related disorder if the level of FHL-1 is elevated as compared to the level of FHL-1 in blood in a control subject that does not have a complement-related disorder.
E. The method according to any one of the preceding paras, wherein the method comprises determining the level of:
   (a) FHR1 and FHR2;
   (b) FHR2 and FHR3;
   (c) FHR1 and FHR3;
   (d) FHR1, FHR2 and FHR3;
   (e) FHR1, FHR2, FHR3 and FHR4;
   (f) FHR1, FHR2, FHR3, FHR4 and FHR5; or
   (g) any of (a) to (f) above in combination with FHL-1 and/or FH.
F. The method according to any one of the preceding paras, wherein the level of the one or more complement protein(s) is determined by mass spectrometry.
G. The method according to any one of the preceding paras, wherein the complement-related disorder is Macular Degeneration.
H. The method according to para G wherein the macular degeneration is one or more of Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, and/or autoimmune uveitis.
I. The method according to any one of paras A to F, wherein the complement-related disorder is selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, Alzheimer's disease, dementia, Lewy body disease, Amyotrophic lateral sclerosis (ALS), Huntington's disease, prion diseases, cancer, lung cancer, and glioblastoma multiforme (GBM).
J. The method according to any one of the preceding paras wherein the step of determining involves:
   (i) digesting at least one complement protein in a sample obtained from the subject with endoproteinase GluC to obtain one or more peptides;
   (ii) determining the presence and/or level of the one or more peptides by mass spectrometry; and
   (iii) using the results of (ii) to determine whether or not the level of the complement protein is elevated.
K. A method for selecting a subject for treatment with a complement-targeted therapy, the method comprising:
   (a) determining the level of a complement protein selected from one or more of FHR1, FHR2, FHR3, FHR4 and/or FHR5, and optionally FHL-1, in a blood sample obtained from the subject;
   (b) selecting the subject for treatment with the complement-targeted therapy if the level of the complement protein determined in (a) is elevated as compared to the level of that complement protein in blood in a control subject that does not have a complement-related disorder.
L. A method for selecting a therapeutic agent for a subject, the method comprising:
   (a) determining the level of a complement protein selected one or more of FHR1, FHR2, FHR3, FHR4 and/or FHR5, and optionally FHL-1, in a blood sample obtained from the subject;
   (b) determining whether the level of the complement protein(s) is elevated as compared to the level of that complement protein(s) in blood in a control subject that does not have a complement-related disorder; and
   (c) selecting a therapeutic agent that targets one or more complement protein(s) that are elevated in the sample from the subject.
M. A method of treatment comprising:
   (a) determining the level of a complement protein selected from one or more of FHR1, FHR2, FHR3, FHR4 and/or FHR5, and optionally FHL-1, in a blood sample obtained from the subject;
   (b) determining whether the level of the complement protein is elevated as compared to the level of that complement protein in blood in a control subject that does not have a complement-related disorder; and
   (c) treating the subject with a complement-targeted therapy.
N. A complement-targeted therapeutic agent for use in a method of treatment, wherein the method comprises:
   (a) determining the level of a complement protein selected one or more of FHR1, FHR2, FHR3, FHR4 and/or FHR5, and optionally FHL-1, in a blood sample obtained from the subject;
   (b) determining whether the level of the complement protein(s) in (a) is elevated as compared to the level of that complement protein(s) in blood in a control subject that does not have a complement-related disorder; and
   (c) treating the subject with the complement-targeted therapeutic agent.
O. Use of a complement-targeted therapeutic agent in the manufacture of a medicament for the treatment of Macular Degeneration in a subject, wherein the method of treatment comprises:
   (a) determining the level of a complement protein selected from one or more of FHR1, FHR2, FHR3, FHR4 and/or FHR5, and optionally FHL-1, in a blood sample obtained from the subject;
   (b) determining whether the level of the complement protein(s) in (a) is elevated as compared to the level of that complement protein(s) in blood in a control subject that does not have a complement-related disorder; and
   (c) treating the subject with the complement-targeted therapeutic agent.
P. The method according to any one of paras K to O wherein the therapy or therapeutic agent is an siRNA, miRNA, shRNA, antisense oligonucleotide, gapmer, antibody, aptamer, SOMAmer, or small molecule.
Q. The method according to para P wherein the therapy or therapeutic agent reduces the level of one or more of FHR1, FHR2, FHR3, FHR4 and/or FHR5.
R. A method of determining whether a subject has, or is at risk of developing, a complement-related disorder, the method comprising:
   (a) digesting at least one complement protein selected from one or more of FHR1, FHR2, FHR3, FHR4 and/or FHR5, and optionally FHL-1, in a sample obtained from the subject with endoproteinase GluC to obtain one or more peptides;
   (b) determining the level of the one or more peptides by mass spectrometry; and
   (c) 4 using the results of (b) to determine the level of the one or more complement proteins; and
   (d) determining that the subject has, or is at risk of developing, a complement-related disorder if the level of one or more complement proteins determined in (c) is elevated as compared to the level of that complement protein in blood in a control subject that does not have a complement-related disorder.
S. A method according to any one of paras K to R, wherein the method further comprises determining the level of FH.
T. A method according to any one of paras K to S, wherein the level of a complement protein is determined by mass spectrometry.

Aspects and embodiments of the present invention will now be discussed with reference to the accompanying figures. Further aspects and embodiments will be apparent to those skilled in the art. All documents mentioned in this text are incorporated herein by reference.

### FIGURES

Embodiments and experiments illustrating the principles of the invention will now be discussed with reference to the accompanying figures in which:

FIG. 1. Schematic showing the C3 proteolytic cascade and the proteolytic events leading to the generation, breakdown and inactivation of C3b (modified from Maillard et al, J Am Soc Nephrol. 2015 July; 26(7):1503-12). Proteoform-specific peptides for mass spectrometry are underlined.

FIG. 2. LC-SRM Trace showing detection of the heavy-labelled synthetic standard peptides of each individual RCA locus protein from a plasma sample.

FIG. 3. Linearity data for peptides derived from FH, FHL-1, and FHR1-5.

FIGS. 4A to 4D. Data confirming that C3 and C3 breakdown products in human plasma can be detected by MS with sufficient specificity and sensitivity. 4A: Total ion chromatograph from SRM-MS analysis showing specific and simultaneous detection of C3b fragment-specific peptides. 4B: Linearity data for seven of the ten peptides spiked into a plasma background. 4C: Coomassie-stained electrophoresis gel of C3 breakdown products obtained in vitro. 4D: MS quantification of key C3 fragments from the in vitro assay products shown in 4C.

FIG. 5. Correlation matrix showing the Pearson correlation coefficients between the different variables (absolute concentration levels of various studied proteins).

FIG. 6. Scatterplot showing differences in protein levels between subjects with AMD and control individuals (mean and p values shown (p<0.05 considered statistically significant)).

FIG. 7. Area under the Receiver Operating Characteristic curve for various models.

FIG. 8. Receiver Operating Characteristic curve for a model that uses FHR1, FHR2, FHR3, FHR4, FHR5, FHL-1 & CFH levels (with 2- & 3-way interactions) to predict whether an individual is an AMD case or a control subject.

FIG. 9. GWASs of circulating FHR-1 to FHR-5 protein levels reveal a strong genome-wide significant signal spanning the CFH locus. Regional plots show the genome-wide significant (P-value 5×10⁻⁸) association signals from the GWASs of FHR-1 to FHR-5 protein levels (panels A-E) at the CFH locus on chromosome 1q31.3. Panel F shows the equivalent CFH region for the GWAS of FHL-1 protein levels (no genome-wide significant association regions observed). The most associated variant is denoted by a purple diamond and is labelled by its rs-number. The other surrounding variants are shown by circles coloured to reflect the extent of linkage disequilibrium with the most associated variant (based on the European (EUR) population genotype data originated from the 1000 Genomes Project, November 2014). A diagram of the genes within the relevant regions is depicted below each plot. Physical positions are based on NCBI RefSeq hg19 human genome reference assembly.

FIG. 10. Established AMD risk variants at the CFH locus are associated at genome-wide significance level with circulating FHR-1, FHR-2, FHR-3 and FHR-4 protein levels in 252 Cambridge controls. Box plots of FHR protein levels by variant genotype for those established AMD risk variants at the CFH locus from the IAMDGC study that showed genome-wide significant (P-value≤5×10⁻⁸) associations in 252 controls from the Cambridge AMD cohort (Table 2). P-values and Beta values from Wald tests using linear regression models adjusted for sex, age and the first two genetic principal components (as estimated within the IAMDGC study) are indicated in the note at the bottom of each plot.

FIG. 11. Mendelian randomization analysis shows highly significant elevation of circulating FHR-1, FHR-2, FHR-4 and FHR-5 protein levels in advanced AMD. Mendelian randomization estimates of the association of FHR-1 (panel A), FHR-2 (panel B), FHR-3 (panel C), FHR-4 (panel D) and FHR-5 (panel E) are presented together with the corresponding traditional epidemiologic odds ratio (OR) estimates obtained from logistic regression models (352 advanced AMD cases and 252 controls from the Cambridge AMD study). The Mendelian randomization estimates were obtained using the Wald ratio (if a single instrument was available; FHR1, FHR2, FHR4, FHR5) or the inverse-variance weighted (IVW) method under a fixed-effect model (if multiple instruments were available; FHR3). Raw data used to calculate the Mendelian randomization estimates are provided in Table S7. The variance of each protein explained by its genetic instrument(s) is indicated in the note at the bottom of each plot.

FIGS. 12A to 12E. Tumor cell IDO mediates immune suppression in-part through an IDO-dependent tryptophan metabolism-independent mechanism. (12A) Schematic representation of the protocol used to generate IDO^{-/-}tGBM cells that express vectors with or without IDO cDNA. (12B) Left panel: Western blotting and quantitative RT-PCR for the detection of IDO in IDO^{-/-}tGBM cells expressing an empty vector (Vector^{EMPTY}), wild-type IDO cDNA (IDO^{WT}), or enzyme-null IDO cDNA (IDO^{H350A}); Center panel: HPLC quantification of kynurenine (Kyn) and tryptophan (Trp) levels in cell culture supernatants of genetically modified IDO^{-/-}tGBM cell lines (n=3 per cell line reflecting one representative experiment from more than 15 experimental repeats); Right panel: Cell proliferation assay to compare the in vitro growth of genetically modified IDO^{-/-}tGBM cells (n=4 per cell line reflecting one representative experiment from 8 experimental repeats). (12C) IDO^{-/-}tGBM mice were intracranially-engrafted with genetically modified IDO^{-/-}tGBM cells, with or without treatment of anti-CD4 and anti-CD8 mAbs beginning at day −3 prior to intracranial engraftment and twice/week for up to 30 days post-intracranial engraftment followed by monitoring for overall survival (n=7-22/group). The long-term survival rate (LTS) and median overall survival (mOS) is labeled on the graph. Survival monitoring of mice depleted for leukocytes ended at 58 days post-engraftment. (12D) Tumor tissue samples were isolated at 4-weeks post-tumor cell engraftment followed by the analysis of tumor infiltrating leukocyte phenotypes. The percentage and absolute cell numbers of CD8⁺ T cells, CD4⁺ T cells, and CD4⁺CD25⁺FoxP3⁺ were quantified (n=8 per group). (12E) Left panel: Flow cytometric analysis of genetically modified IDO^{-/-}tGBM cells co-cultured with splenic CD11 b⁺ monocytes isolated from IDO^{-/-}tGBM mice (n=3 per group that reflect one representative experiment from 5 experimental repeats). Mature macrophage surface markers: CD11 b⁺Ly6G^{-/low}Ly6C⁺. Right panel: HPLC measurement of Trp and Kyn from the co-culture cells experiment. **, P<0.01; ***, P<0.001. ND: not detectable, ns: not significant. All bar graphs represent mean±SEM and dot plots show the median value as a horizontal line.

FIGS. 13A to 13E. Indoleamine 2,3 dioxygenase 1 (IDO) non-metabolically increases complement factor H (CFH) levels in human glioblastoma (GBM). (13A) Flow chart for the microarray experimental design, data analysis, as well as validation of results. Real-time RT-PCR confirmation of IDO regulatory effects on CFH expression in cultured (13B) U87 or (13C) PDX43 GBM. Cells were exposed to a variety of conditions including transfection with IDO specific siRNA (20 nM) or scrambled control siRNA for 16 hours, treatment with or without 100 ng/mL human interferon-gamma (IFN-γ), treatment with or without the IDO enzyme inhibitor, BGB-5777 (1 uM), or IFNγ plus BGB-5777 for 24 hours. Quantitative RT-PCR was performed (n=3 reflecting one representative experiment from 4 experimental repeats). (13D) Quantitative RT-PCR analysis of IDO and CFH mRNA expression on IFNγ-stimulated U87 cells treated with CFH siRNA or scrambled control siRNA. Same experimental conditions as in [13B] (n=3/group reflecting one representative experiment from 3 experimental repeats). (13E) mRNA expression levels for human IDO, CFH, and CD3 were quantified and compared among intracranial PDX43 and patient-resected newly-diagnosed or recurrent intracranial human GBM (n=4-9/group). Intracranially-engrafted (ic.) PDX43 was isolated from humanized mice with or without treatment of CD4′ and CD8⁺ T cell depleting antibodies between 14-21 days post-intracranial injection. ***, P<0.001; ND: not detectable, ns: not significant. All bar graphs represent mean±SEM.

FIGS. 14A to 14I. Indoleamine 2,3 dioxygenase 1 (IDO) and complement factor H (CFH) mRNA levels positively correlate with T cell infiltration in patient-resected glioblastoma (GBM). (14A) mRNA expression levels for IDO and CFH in grade II (green; n=226), grade III (blue; n=249), and grade IV (GBM; red; n=172) glioma of the RNA Hi-Seq. Illumina dataset as analyzed in the cancer genome atlas (TCGA). Horizontal lines in the scatter plots represents mean±SEM. (14B) Pearson's correlation analysis for IDO and CFH mRNA levels within GBM and all-grade glioma. (14C) Kaplan-Meier (KM) survival analysis of grade II (left), grade III (center), and grade IV (GBM, right) glioma patients stratified by low CFH (blue) and high CFH (red) expression levels. (14D) Kaplan-Meier analysis of GBM recurrence. Recurrent GBM samples were identified from the ‘days to tumor recurrence’ section listed in the TCGA GBM clinical dataset. CFH mRNA expression levels were extracted from the Affymetrix U133a microarray dataset. (14E) CFH mRNA levels were compared among grade II (IDHwt; blue circle and IDHmut; red square), grade III (IDHwt; green triangle and IDHmut; purple circle), as well as grade IV (IDHwt; orange circle and IDHmut; black triangle) glioma. (14F) CFH DNA methylation analysis at two distinct genomic loci, cg06377993 and cg23557926 in grade II (IDHwt; blue circle and IDHmut; red square), grade III (IDHwt; green triangle and IDHmut; purple circle), and grade IV (IDHwt; orange circle and IDHmut; black triangle) glioma. (14G) Expression of CFH and IFNγ mRNA levels in patient-resected GBM tissue samples as categorized by CD3E and CD8A expression levels while accessing the TGGA GBM RNA-Seq. dataset. (14H) Detection of CFH mRNA in the human GBM cell-T cell co-culture system in vitro. CD3⁺ human T cells were isolated under positive selection from GBM patient peripheral blood mononuclear cells (PBMCs). CFH mRNA levels were analyzed in U87 GBM cells co-cultured with either naïve or activated T cells or conditioned medium from activated T cells in the presence or absence of IFNγ-neutralizing antibodies. Data were compiled from three independent experiments. (14I) In vitro expression analysis of human CFH mRNA in different GBM cells with or without the addition of human IFNγ. Data represent pooled data from four independent experiments. **, P<0.01; ***, P<0.001; ND: not detectable. All bar graphs represent mean±SEM.

FIGS. 15A to 15H. Indoleamine 2,3 dioxygenase 1 (IDO) enhances the expression of both complement factor H (CFH) isoforms in human glioblastoma (GBM). (15A) Schematic representation of CFH transcript variants reflecting the full-length (CFH) and truncated (FHL-1) sequences. Pearson's correlation analysis of (15B) IDO and (15C) CD3E with CFH and FHL-1 using the TCGA GBM RNA-Seq dataset. (15D) Left panel: Western blot analysis of surgically-resected tumor tissue samples from both newly diagnosed GBM patients and recurrent GBM patients. Right panel: Protein expression of full-length and truncated CFH variants and IDO in IFNγ-stimulated U87 cells. Cells were treated with IFNγ and cultured in serum-free medium for 24 to 48 hours. Both cell lysates and cell culture supernatants were collected. The supernatants were concentrated with ultrafiltration and analyzed by Western blot along with cell lysate samples. One representative result is shown that reflects 4 experimental repeats. (15E) Time course analysis of IDO, CFH, FHL-1 mRNA expression after treatment with IFNγ in U87 (top row) and PDX43 (bottom row) GBM cells. The U87 cells were stimulated with 100 ng/ml human IFNγ and RNA lysates were extracted followed by quantitative RT-PCR analysis (n=4). (15F) Western blot of cell lysates and supernatants collected from the same experimental design as in top panel. Data from one representative experiment from 2 experiments are shown. (15G) Top panel: Western blot showing IDO protein levels in unmodified (Unmod.) as well as in CRISPR-Cas9 IDO-deleted (IDOKO) U87 cells. Unmod. U87 cells and IDOKO U87 cells were stimulated with 100 ng/mL human IFNγ for 24 hours. Protein lysates were collected from both untreated and IFNγ treated cells followed by Western blotting analysis; Lower panel: HPLC measurement of kynurenine (Kyn) and tryptophan (Trp) in Unmod. U87 and IDOKO U87 cells. (15H) Comparison of IDO, CFH, and FHL-1 mRNA expression induction in Unmod. and IDOKO U87 cells using quantified RT-PCR (n=3 per group that reflects 1 representative experiment after 2 repeats). ***, P<0.001; ND: not detectable. All bar graphs represent mean±SEM.

FIGS. 16A to 16F. Complement factor H (CFH) increases immunosuppressive factor expression and decreases overall survival in a syngeneic mouse brain tumor model. (16A) Splenic CD11 b monocytes were isolated from IDO^{-/-}tGBM mice and co-cultured with either IDO^{-/-}tGBM cells expressing Vector^{EMPTY} or FHL-1 cDNA. RT-PCR quantification for ARG1, CCL2, and IL-6 mRNA levels were determined in cultured macrophages (blue bar), macrophages co-cultured with IDO^{-/-}tGBMs expressing Vector^{EMPTY} (red bar), or macrophages co-cultured with FHL-1 cDNA (green bar) (n=3 per group reflecting data compiled from 2 independent experiments). (16B) RT-PCR quantification for ARG1 and CCL2 in cultured IDO^{-/-}tGBMs alone (blue bar), IDO^{-/-}tGBM cells expressing FHL-1 cDNA (red), IDO^{-/-}tGBM cells expressing Vector^{EMPTY} co-cultured with macrophages (green bar) and IDO^{-/-}tGBM cells expressing FHL-1 cDNA co-cultured with macrophages (purple bar) (n=3 per group reflecting data compiled from 2 independent experiments). (16C) Kaplan-Meier (KM) survival analysis of IDO^{-/-}tGBM mice intracranially engrafted with either IDO^{-/-}tGBM cells expressing Vector^{EMPTY} (blue) or IDO^{-/-}tGBM cells expressing FHL-1 cDNA (red) (n=17/group). Colorful numbers represent median survival (MS). Plots without labeled numbers indicate undefined MS. (16D) Kaplan-Meier (KM) survival analysis of IDO^{-/-}tGBM mice intracranially engrafted with either IDO^{-/-}tGBM cells expressing Vector^{EMPTY} or IDO^{-/-}tGBM cells expressing FHL-1 cDNA in the presence or absence of anti-mouse-CD4 mAb, -CD8 mAb, and -NK1.1 mAb (n=8-10/group). Colorful numbers represent median survival (MS). (16E) Flow cytometry analysis of tumor infiltrating lymphocytes and MDSCs from GBM tissue samples collected at 3-week post intracranial injection; (16F) Gene expression analysis on mouse GBM tumor tissues and contralateral non-tumor brain samples. Mice intracranially injected with modified IDO^{-/-}tGBM cells were euthanized when displaying endpoint symptoms. Brain tumor tissues and contralateral brain tissues were collected at stored at Trizol reagent. At the end of survival analysis [65-days post injection, (16C)], all samples were subjected to RNA extraction and real-time RT-PCR. Plots without labeled numbers indicate undefined MS. *, P<0.05; **, P<0.01; ***, P<0.001; ****, P<0.0001. All bar graphs represent mean±SEM.

FIGS. 17A to 17E. Systemic and local complement factor H levels and the relationship with other immunosuppressive factors in patient-resected glioblastoma (GBM). (17A, 17B) Quantification of protein levels of full-length and truncated CFH in patient plasma samples by mass spectrometry. (17C) Pearson's correlation analysis for CFH mRNA with PD-L1, PD-L2, PD-1, CTLA-4, STAT3, CD39, BTLA, LAG3, FOXP3 and FGL2 in GBM. Each small circle in the plot represents the expression in a single patient. (17D) Canonical-correlation analysis of CFH with major tumor immune cell types. The signature genes of each type of immune cells are defined as: CD8⁺ T cell (CD3, CD8), Treg (CD3, CD4, CD25, FoxP3), MDSCs (CD14, CD11b, CD33, and Arg1), TAM (CD14, HLA-DR, CD312, CD115, CD163, CD204, CD301, CD206), and neutrophil (CD11b, CD16, CD66b, ELANE). (17E) Schematic presentation of a hypothesis based on the findings reported here and elsewhere. Standard of care treatment radiation (RT) and temozolomide (TMZ) enhances inflammatory mechanisms that alter the immune tolerant (cold) GBM microenvironment into a more inflamed (hot) conditions which is partially caused by tumor-infiltrating IFNγ⁺CD8⁺ T cells. The IFNγ acts on human GBM cells to induce IDO expression, which in-turn, enhances CFH expression levels through a non-enzymic mechanism. CFH acts on complement receptors in an autocrine and paracrine manner; the latter of which elicits CCL2 expression in TAMs. The TAMs then facilitate Treg and additional monocyte recruitment into the GBM which reinforces the immunosuppressive microenvironment.

FIG. 18. Overall survival of syngeneic mice with intracranial IDO^{-/-}tGBM tumors and depleted for CD4⁺ T, CD8⁺ T, and NK1.1⁺ immune cells. IDO^{-/-}tGBM mice were intracranially-engrafted with genetically modified IDO^{-/-}tGBM cells and depleted with anti-CD4, anti-CD8 mAbs, and anti-NK1.1 mAbs, and monitored for overall survival (n=13-14/group). mOS=median overall survival. Undef.=Undefined.

FIG. 19. Histological evaluation of tumor progression and confirmation of IDO-mGFP expression in brain tumors. IDO^{-/-}tGBM mice intracranially-engrafted with different engineered cells lines followed by tumor isolation at the time of endpoint symptoms. Dashed lines highlight the border line of GBM and adjacent parenchymal brain tissue. One representative field is shown from 5 different observation fields.

FIG. 20. Table showing demographics of patient-isolated tissue samples.

FIG. 21. Table showing nucleic acid-based reagents utilized.

FIG. 22. Table showing differentially expressed genes that possess the strongest correlation with IDO in human GBM cells.

FIG. 23. Table showing mass spectrometry parameters for detecting CFH and FHL-1 in plasma.

FIG. 24. Graphs showing the levels of the indicated complement proteins in the blood samples obtained from test 1 of COVID-19 patients having eventual asymptomatic disease (A), mild disease (B), disease requiring hospitalization but not supplemental oxygen (C), disease requiring hospitalization and low flow supplemental oxygen (D), disease requiring assisted ventilation (E), or blood samples obtained from COVID-19-negative subjects (control).

FIG. 25. ROC curves showing ability of the indicated complement proteins to predict severity of COVID disease, using first-test blood samples obtained from COVID-19 patients that eventually required assisted ventilation (Group E above) compared with healthy controls.

### EXAMPLES

### Example 1: Generation of Peptides from Complement Proteins for Mass Spectrometry

GluC digestion was performed on FH, FHL-1, FHR1-5, FI, C3, C3b and C3b breakdown products to achieve distinct peptides for mass spectrometry. GluC digestion is described in Example 2.2.

Peptides that can be used to detect each protein or protein fragment are set out in Tables 1-4 below.

TABLE 1
Distinct FH family peptides after
GluC digestion.

| | | | SEQ |
|---|---|---|---|
| | | | ID |
| Protein | Peptide Sequence | Mass | No: |
| Factor H | VTYKCFE | 888.4051 | 20 |
| FHL1 | NGWSPTPRCIRVSFTL | 1832.9355 | 21 |
| FHR1 | ATFCDFPKINHGILYDEE | 2110.9669 | 22 |
| FHR2 | RGWSTPPKCRSTISAE | 1774.8784 | 23 |
| | and | | |
| | AMFCDFPKINHGILYDEE | 2140.9598 | 24 |
| FHR3 | VACHPGYGLPKAQTTVTCTE | 2074.9816 | 25 |
| FHR4 | YQCQSYYE | 1082.4015 | 26 |
| FHR5 | RGWSTPPICSFTKGE | 1664.7980 | 27 |

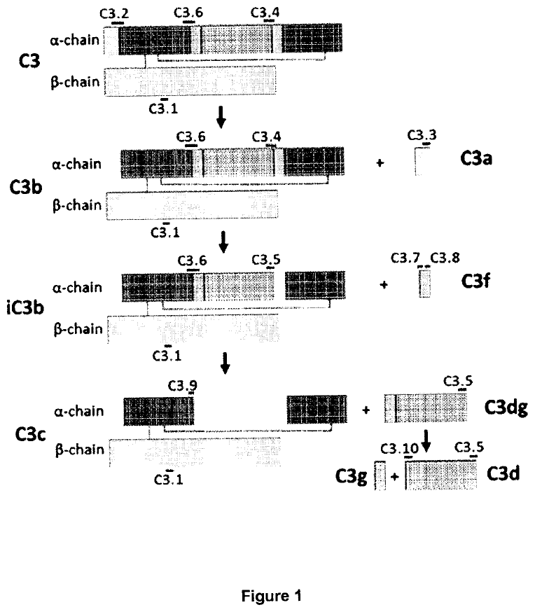

The series of proteolytic events leading to the generation, breakdown and inactivation of C3 are shown in FIG. 1. Proteoform-specific peptides produced by GluC digestion are underlined in FIG. 1 and are shown in Table 2. Table 3 shows how each protein can be detected individually using the peptides in Table 2.

TABLE 2
Peptide sequences for MS resulting from GluC
digestion of C3, C3b and breakdown products.

| | | Contained | SEQ |
|---|---|---|---|
| | Peptide | in C3b | ID |
| Peptide | sequence | products | No: |
| C3.1 | GTAFVIFGIQDGE | C3 + C3b + | 28 |
| | | iC3b + C3c | |
| C3.2 | LRRQHARASHLG | C3 only | 29 |
| | LARSNLDE | | |
| C3.3 | LRRQHARASHLGLAR | C3a only | 30 |
| C3.4 | LNLDVSLQLPSR | C3 + C3b | 31 |
| | SSKITHRIHWE | | |
| C3.5 | LNLDVSLQLPSR | iC3b + C3dg + C3d | 32 |
| C3.6 | RLGRE | C3 + C3b + iC3b | 33 |
| C3.7 | SSKITHRIHWE | C3f | 34 |
| C3.8 | SASLLR | C3f | 35 |
| C3.9 | RLGR | C3c | 36 |
| C3.10 | HLIVTPSGCGE | C3d | 37 |

TABLE 3
Methodology for determining concentration of all C3/C3b breakdown
products using GluC digestion peptides of Table 2.

| Protein or fragment | Peptide(s) |
|---|---|
| C3 (total) | C3.1 |
| C3 only | C3.2 |
| C3a | C3.3 |
| C3b | C3.4-C3.2 |
| iC3b | C3.6-C3.4 |
| C3f | C3.7 or C3.8 |
| C3c | C3.9 |
| C3dg | C3.5-C3.10-iC3b [C3.6-C3.4] |
| C3d | C3.10 |

TABLE 4
Alternative peptides for C3.1 resulting from GluC
digestion, to measure total C3 content.

| | | | | SED |
|---|---|---|---|---|
| Peptide | Mass | Position | Peptide sequence | ID No. |
| C3.1.1 | 6957.8210 | 463-523 | AKIRYYTYLIMNKGRLLKAGRQVREPGQDLV | 61 |
| | | | VLPLSITTDFIPSFRLVAYYTLIGASGQRE | |
| C3.1.2 | 5778.9734 | 321-372 | RSGIPIVTSPYQIHFTKTPKYFKPGMPFDLMV | 62 |
| | | | FVTNPDGSPAYRVPVAVQGE | |
| C3.1.3 | 5650.1364 | 97-146 | KVVLVSLQSGYLFIQTDKTIYTPGSTVLYRIFT | 63 |
| | | | VNHKLLPVGRTVMVNIE | |
| C3.1.4 | 3904.1017 | 373-408 | DTVQSLTQGDGVAKLSINTHPSQKPLSITVRT | 64 |
| | | | KKQE | |
| C3.1.5 | 3861.1628 | 565-599 | GDHGARVVLVAVDKGVFVLNKKNKLTQSKI | 65 |
| | | | WDVVE | |
| C3.1.6 | 3807.7645 | 600-637 | KADIGCTPGSGKDYAGVFSDAGLTFTSSSG | 66 |
| | | | QQTAQRAE | |
| C3.1.7 | 3263.6357 | 414-442 | QATRTMQALPYSTVGNSNNYLHLSVLRTE | 67 |
| C3.1.8 | 2845.4609 | 24-50 | AHDAQGDVPVTVTVHDFPGKKLVLSSE | 68 |
| C3.1.9 | 2819.4705 | 150-175 | GIPVKQDSLSSQNQLGVLPLSWDIPE | 69 |
| C3.1.10 | 2691.3425 | 524-549 | VVADSVWVDVKDSCVGSLVVKSGQSE | 70 |
| C3.1.11 | 2618.3261 | 296-320 | DLVGKSLYVSATVILHSGSDMVQAE | 71 |
| C3.1.12 | 2511.2904 | 51-73 | KTVLTPATNHMGNVTFTIPANRE | 72 |
| C3.1.13 | 2108.1378 | 78-96 | KGRNKFVTVQATFGTQVVE | 73 |
| C3.1.14 | 1992.1480 | 278-295 | VVLSRKVLLDGVQNPRAE | 74 |
| C3.1.15 | 1827.9414 | 448-462 | TLNVNFLLRMDRAHE | 75 |
| C3.1.16 | 1769.8923 | 176-189 | LVNMGQWKIRAYYE | 76 |
| C3.1.17 | 1745.9386 | 1-15 | SPMYSIITPNILRLE | 77 |
| C3.1.18 | 1738.9036 | 550-564 | DRQPVPGQQMTLKIE | 78 |
| C3.1.19 | 1623.9348 | 231-244 | VTITARFLYGKKVE | 79 |
| C3.1.20 | 1352.6612 | 245-257 | GTAFVIFGIQDGE | 80 |
| C3.1.21 | 1138.5335 | 219-226 | KFYYIYNE | 81 |
| C3.1.22 | 1135.5146 | 190-199 | NSPQQVFSTE | 82 |
| C3.1.23 | 954.5862 | 265-272 | SLKRIPIE | 83 |
| C3.1.24 | 853.4221 | 205-211 | YVLPSFE | 84 |
| C3.1.25 | 841.4657 | 258-264 | QRISLPE | 85 |
| C3.1.26 | 826.4007 | 638-645 | LQCPQPAA | 86 |
| C3.1.27 | 785.4171 | 212-218 | VIVEPTE | 87 |
| C3.1.28 | 591.2938 | 19-23 | TMVLE | 88 |
| C3.1.29 | 570.3125 | 443-447 | LRPGE | 89 |
| C3.1.30 | 509.2485 | 74-77 | FKSE | 90 |

GluC digestion of Factor I (FI) produced the candidate peptides in Table 5 for MS analysis. SEQ ID NO:45 to 56 and 155 contain 8-21 amino acids and are a good length for MS analysis.

TABLE 5
Peptide sequences resulting from GluC digestion of Fl.

| | | | | SEQ |
|---|---|---|---|---|
| Peptide | Mass | Position | Peptide sequence | ID NO: |
| 1 | 3996.7183 | 510-548 | CAGTYDGSIDACKGDSGGPLVCMDANN | 38 |
| | | | VTYVWGVVSWGE | |
| 2 | 3994.8853 | 57-92 | GTCVCKLPYQCPKNGTAVCATNRRSFPTYCQQKSLE | 39 |
| 3 | 3467.7211 | 555-583 | FPGVYTKVANYFDWISYHVGRPFISQYNV | 40 |
| 4 | 3397.6804 | 150-180 | ANVACLDLGFQQGADTQRRFKLSDLSINSTE | 41 |
| 5 | 3388.6605 | 446-475 | LPRSIPACVPWSPYLFQPNDTCIVSGWGRE | 42 |
| 6 | 3155.5773 | 31-56 | KKCLAKKYTHLSCDKVFCQPWQRCIE | 43 |
| 7 | 2991.2861 | 254-281 | LCCKACQGKGFHCKSGVCIPSQYQCNGE | 44 |
| 8 | 2531.2455 | 129-149 | VKLVDQDKTMFICKSSWSMRE | 45 |
| 9 | 2068.0320 | 489-505 | VKLISNCSKFYGNRFYE | 46 |
| 10 | 1805.8309 | 93-109 | CLHPGTKFLNNGTCTAE | 47 |
| 11 | 1698.7969 | 420-434 | NYNAGTYQNDIALIE | 48 |
| 12 | 1605.7867 | 110-124 | GKFSVSLKHGNTDSE | 49 |
| 13 | 1297.5729 | 291-303 | VGCAGFASVTQEE | 50 |
| 14 | 1168.272 | 291-302 | VGCAGFASVTQE | 155 |
| 15 | 1295.6082 | 435-445 | MKKDGNKKDCE | 51 |
| 16 | 1191.6156 | 411-419 | YVDRIIFHE | 52 |
| 17 | 1166.5557 | 181-190 | CLHVHCRGLE | 53 |
| 18 | 1121.5738 | 480-488 | RVFSLQWGE | 54 |
| 19 | 978.4448 | 306-314 | ILTADMDAE | 55 |
| 20 | 955.4731 | 19-26 | KVTYTSQE | 56 |
| 21 | 736.3182 | 282-288 | VDCITGE | 57 |
| 22 | 647.2817 | 549-554 | NCGKPE | 58 |
| 23 | 520.2613 | 191-195 | TSLAE | 59 |
| 24 | 505.2252 | 476-479 | KDNE | 60 |

### Example 2: Mass Spectrometry

### 2.1 Preparation of Stable Isotopic Standards (SIS) Spiking Solution

High purity heavy-labelled synthetic standards, with S-carboxymethylated (CAM) cysteine residues, were obtained (Cambridge Research Biochemicals, Cambridge, UK) and diluted to 1 μg/μL with 50:50 acetonitrile:water+0.1% formic acid (Table 6).

A mixed SIS solution was prepared by firstly diluting stock solution of FHL-1, FHR1, FHR2, FHR3, FHR4 and FHR5 by tenfold (no dilution of CFH stock was required), then adding the appropriate amounts of each individual diluted solution to a final volume of 200 μL in 0.1% TFA. This was then stored at −80° C. in 5 μL aliquots for further dilution immediately prior to spiking.

Spiking solution was prepared immediately prior to sample addition by adding 195 μL 50:50 acetonitrile:water to a 5 μL aliquot of the mixed SIS solution. 2 μL of this was carefully added to each digested sample prior to drying down.

TABLE 6
Stock solutions of stable isotopic standards
(SIS) at 1 ug/MI. The residues in bold were
chosen to carry a stable-heavy isotype to
enable quantitation.
Lower case ‘c’ denotes a S-carboxymethylated
(CAM) cysteine residue. Residue in bold type
contained an isotopically heavy amino acid,
where K(+8), R(+10), F(+10) and Y(+10).

| | | | | Net | Solvent | |
|---|---|---|---|---|---|---|
| Pro- | Peptide | MW, | Purity, | Content, | Volume, | Conc, |
| tein | Sequence | g | % | % | μL | pmol/μL |
| CFH | VTYKcFE | 953.4 | 98.6 | 72.6 | 716 | 1050 |
| FHL- | NGWSPTPR | 1900 | 97.4 | 78.9 | 768 | 526.3 |
| 1 | cIRVSFTL | | | | | |
| FHR1 | ATFcDFPKI | 2178 | 100.0 | 80.7 | 807 | 459.1 |
| | NHGILYDEE | | | | | |
| FHR2 | RGWSTPPK | 1845.9 | 100.0 | 55.5 | 555 | 541.7 |
| | RESTISAE | | | | | |
| FHR3 | VACHPGYG | 2207.1 | 98.4 | 75.0 | 738 | 453.1 |
| | LPKAQTTV | | | | | |
| | TcTE | | | | | |
| FHR4 | YQCQSYYE | 1149.7 | 97.6 | 84.1 | 821 | 869.8 |
| FHR5 | RGWSTPPI | 1739.8 | 95.6 | 74.0 | 707 | 574.8 |
| | CSFTKGE | | | | | |

### 2.2 Preparation of Samples for Analysis by LC-MS/MS

Frozen plasma samples were allowed to thaw to room temperature before being vortexed hard for 5 minutes to dissolve any soluble material, then centrifuged at 13,300g for 30 min to settle any insoluble material.

To a 5 μL plasma aliquot (equivalent to approximately 350 μL protein), 90 μL of 50 mM ammonium bicarbonate (pH 7.8), 2 μL of ProteaseMAX™ (Promega, Southampton, UK) solution (1% w/v in 50 mM ammonium bicarbonate) and 1 μL of 500 mM dithiothreitol prepared in 50 mM ammonium bicarbonate was added. This was vortexed briefly to mix, then given a pulse spin before incubating at 56° C. for 25 min.

After cooling to room temperature, 3 μL 500 mM iodoacetamide (prepared in 50 mM ammonium bicarbonate) was added. This was vortexed briefly to mix, then given a pulse spin before incubating at room temperature and in the dark for 15 min.

A further 1 μL of ProteaseMAX solution (1% w/v in 50 mM ammonium bicarbonate) and 5 μL of 1 μg/uL endoproteinase GluC (Roche, Mannheim, Germany) were added. The mixture was vortexed briefly, then given a pulse spin before incubating for 16 hours at 25° C. with slight shaking (400 rpm).

To the digested peptide mix obtained, 6 μL 10% v/v trifluoroacetic acid (TFA) and 2 μL of SIS spiking solution were added, vortexed briefly to mix, then pulse spin. The solution was placed into an evaporator and dried. Finally the peptides were reconstituted in 50 μL 0.1% TFA and vortexed to dissolve any residue before centrifuging at 13,300g for 30 min to settle any insoluble/particulate material. Approximately 48 μL (taking care to leave behind any precipitated material) was transferred to a LC autosampler vial for subsequent analysis by LC-MS/MS.

### 2.3 LC-SRM/MS Analysis of Plasma Digests

SRM analyses of plasma digests were performed on a 6495 triple quadrupole mass spectrometer with iFunnel-equipped electrospray ion source (Agilent, Santa Clara, CA, USA) coupled to an Infinity 1200 Series liquid chromatography system consisting of 1290 autosampler, 1260 Quat Pump VL pump and TCC column oven modules (Agilent, Santa Clara, CA, USA). Samples were injected directly (4 μL) onto a C18 column (250 mm×2.1 mm i.d., Thermo Scientific Acclaim 120, 3 μm particle size) that was maintained at a column temperature of 50° C. Compounds were developed using a gradient elution of increasing acetonitrile concentration with Buffer A consisting of Water+0.1% formic acid and Buffer B being Acetonitrile+0.1% formic acid. The flow rate was maintained at 250 μL/min with an initial composition of 5% Buffer B.

The following gradient elution profile was used to separate the peptides (time: % B): 0 min: 5% B; 2 min: 5% B; 3 min: 12% B; 12 min: 15% B; 15 min: 20% B; 30 min: 25% B; 31 min: 90% B; 39 min: 90% B; 40 min: 5% B; 49 min: 5% B.

Optimized SRM settings were determined using SIS solutions and are provided in Table 7.

TABLE 7
SRM transitions and optimal collision energies
for FH family peptides (Quantitation ions in bold).

| | | Precursor | Product | Collision |
|---|---|---|---|---|
| | | ion | ions | energy, |
| Protein | Peptide Sequence | m/z | m/z | eV |
| CFH | VTYKcFE (Light) | 473.7 | 583.3, | 16, 16, 16 |
| | | | 847.4, | |
| | | | 746.3 | |
| CFH | VTYKcFE (Heavy) | 477.7 | 591.3, | 16, 16, 16 |
| | | | 855.4, | |
| | | | 754.3 | |
| FHL-1 | NGWSPTPRcIRVSFTL | 631.2 | 723.9, | 19, 19, 19 |
| | (Light) | | 860.5, | |
| | | | 767.4 | |
| FHL-1 | NGWSPTPRcIRVSFTL | 634.3 | 728.9, | 19, 19, 19 |
| | (Heavy) | | 865.5, | |
| | | | 772.4 | |
| FHR1 | ATFcDFPKINHGILYDEE | 724.2 | 925.6, | 20, 16, 20 |
| | (Light) | | 1011.9, | |
| | | | 947.1 | |
| FHR1 | ATFcDFPKINHGILYDEE | 727.2 | 930.6, | 20, 16, 20 |
| | (Heavy) | | 1016.9, | |
| | | | 952.1 | |
| FHR2 | RGWSTPPKcRSTISAE | 459.1, | 539.2, | 8, 26 |
| | (Light) | 611.8 | 798.9 | |
| FHR2 | RGWSTPPKcRSTISAE | 462.6, | 543.9, | 8, 26 |
| | (Heavy) | 616.3 | 806.0 | |
| FHR3 | VACHPGYGLPKAQTTVTCTE | 730.7 | 1022.4, | 16, 18 |
| | (Light) | | 971.7 | |
| FHR3 | VACHPGYGLPKAQTTVTCTE | 736.7 | 1031.4, | 16, 18 |
| | (Heavy) | | 980.7 | |
| FHR4 | YQCQSYYE | 570.7 | 830.3, | 11, 10, 14 |
| | (Light) | | 993.1, | |
| | | | 311.1 | |
| FHR4 | YQcQSYYE | 575.7 | 840.3, | 11, 10, 14 |
| | (Heavy) | | 1003.1, | |
| | | | 311.1 | |
| FHR5 | RGWSTPPICSFTKGE | 575.2 | 828.4, | 16, 15, 20 |
| | (Light) | | 895.5, | |
| | | | 588.3 | |
| FHR5 | RGWSTPPICSFTKGE | 581.2 | 836.4, | 16, 15, 20 |
| | (Heavy) | | 905.5, | |
| | | | 598.3 | |

TABLE 8
Peptides and transitions for quantitation
of C3/C3b breakdown products.

| Pep- | | Contained | |
|---|---|---|---|
| tide | Sequence | in . . . | Transitions |
| C3.1 | GTAFVIFGIQDGE | C3 + C3b + | 569.0/483.2 |
| | | iC3b + C3c | 569.0/654.3 |
| | | | 569.0/801.4 |
| C3.2 | LRRQHARASHLG | C3 only | 384.2/431.6 |
| | LARSNLDE | | 384.2/510.4 |
| | | | 461.0/503.3 |
| C3.3 | LRRQHARASHLGLAR | C3a only | 291.2/304.3 |
| | | | 291.2/395.8 |
| | | | 436.3/598.7 |
| C3.4 | LNLDVSLQLPSRS | C3 + C3b | 546.8/578.7 |
| | SKITHRIHWE | | 683.2/797.1 |
| | | | 683.2/834.9 |
| C3.5 | LNLDVSLQLPSR | iC3b + | 677.9/1014.4 |
| | | C3dg + | 677.9/359.1 |
| | | C3d | 677.9/800.2 |
| C3.6 | RLGRE | C3 + | 251.3/175.1 |
| | | C3b + | 251.3/232.1 |
| | | iC3b | 251.3/345.2 |
| C3.7 | SSKITHRIHWE | C3f | 349.3/354.2 |
| | | | 349.3/436.4 |
| | | | 349.3/489.8 |
| C3.8 | SASLLR | C3f | 323.7/159.1 |
| | | | 323.7/288.2 |
| | | | 323.7/488.3 |
| C3.9 | RLGR | C3c | 251.3/175.1 |
| | | | 251.3/232.1 |
| | | | 251.3/345.2 |
| C3.10 | HLIVTPSGCGE | C3d | 585.3/251.1 |
| | | | 585.3/394.5 |
| | | | 585.3/404 |

In order to protect the source region from unwanted contaminants, a switching valve located between the column and source was diverted to the waste position at points in the chromatogram when the analyte peptides were not eluting. This allowed for six windows (two of the peptides, FHR-2 and FHL-1, eluted within the same window) of acquisition, of approximately one minute each, to be acquired with the column on-line to the mass spectrometer. SRM data was processed using a dedicated project in Skyline (v19.1.0.193; www.skyline.ms).

### 2.4 Results

FH Family Proteins

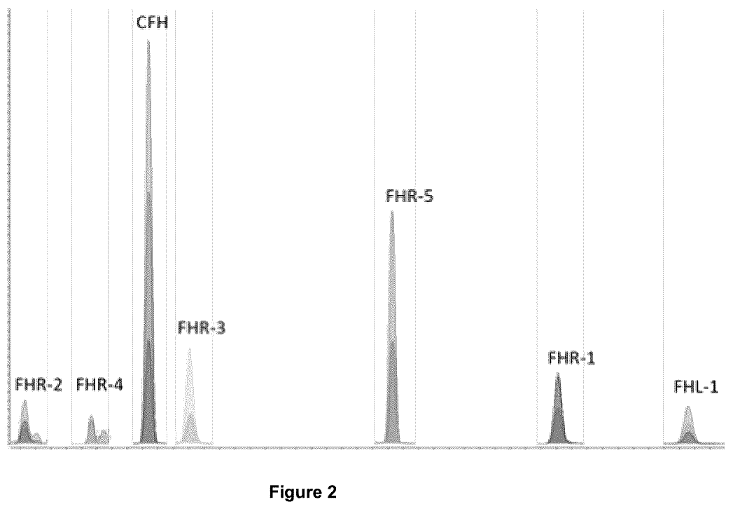

FIG. 2 shows a LC-SRM Trace showing detection of the heavy-labelled synthetic standards of each individual RCA locus protein from a plasma sample. This demonstrates that the method is feasible, specific and has the required sensitivity to distinguish between peptides from these seven proteins, in particular between splice variants FH and FHL-1.

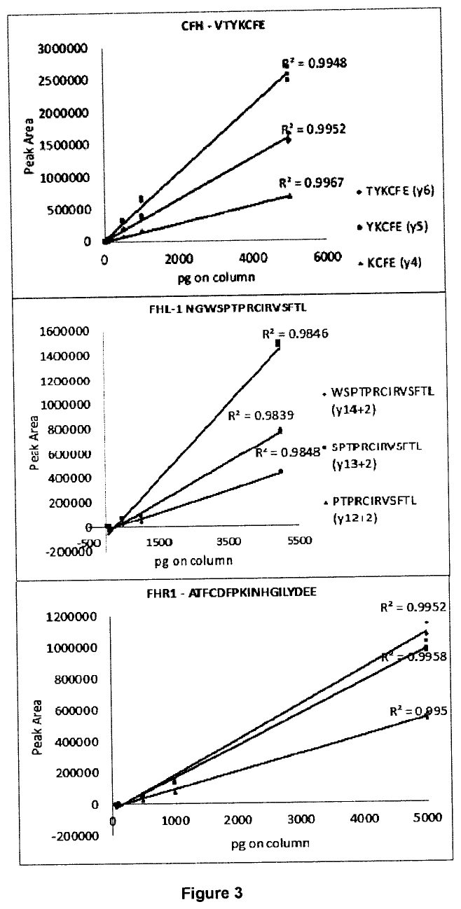

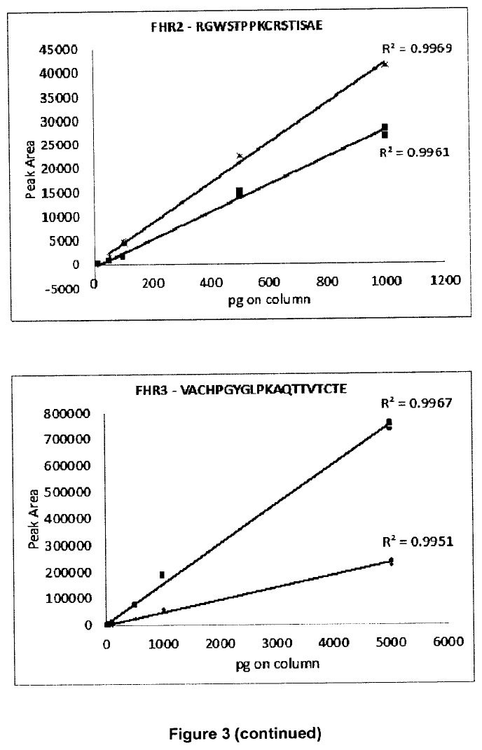

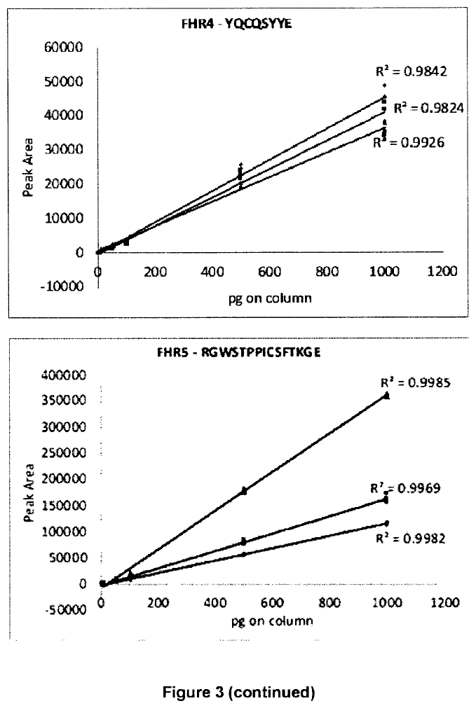

FIG. 3 shows linearity data for FH, FHL-1, and FHR1-5. This demonstrates that the GluC digestion produces peptides that can be detected individually and specifically in native serum at endogenous levels. It also shows that the assay is capable of quantifying the level of each protein in the sample. Increasing amounts of protein increase the signal in a predictable manner, allowing determination of the levels, as well as the presence, of each of the proteins. Also demonstrated is that the assay is free from interference.

Lower limits of quantitation were defined as plasma concentrations of FH=25 nM, FHL-1=0.25 nM, FHR-1=2 nM, FHR-2=1 nM, FHR-3=1 nM, FHR-4=4 nM and FHR-5=3 nM.

C3 and C3 Breakdown Products

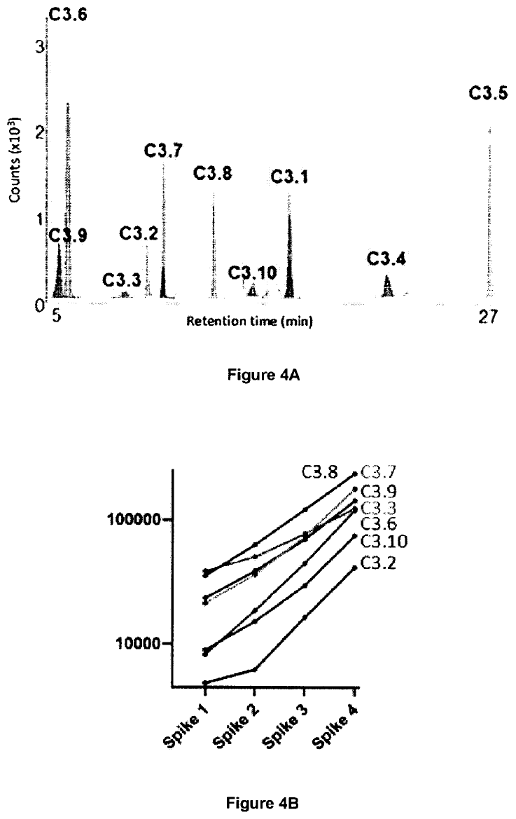

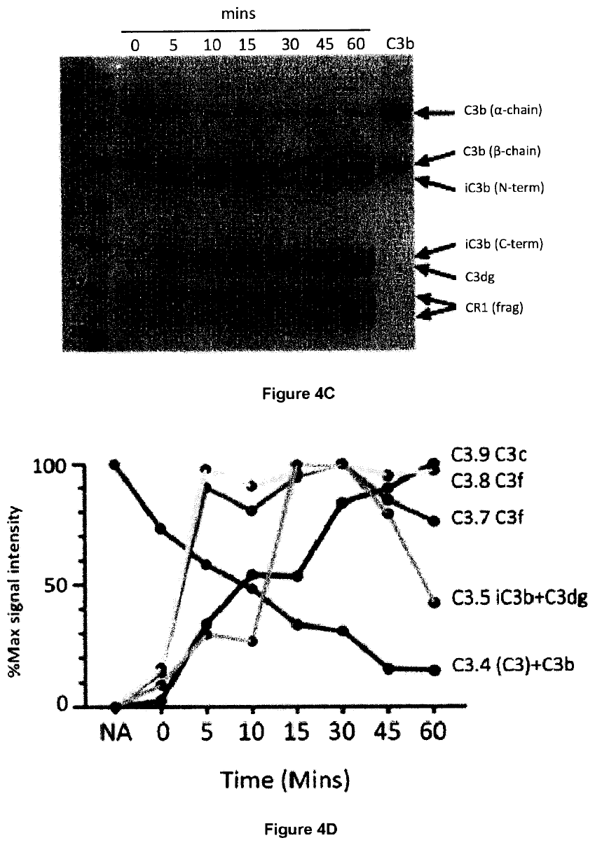

Synthetic versions of the peptides in Table 2 were synthesised to confirm and optimise their detection by MS to confirm that they could be quantified in a linear manner, and to demonstrate that they could be detected at endogenous levels in a serum or plasma sample. This is shown by FIGS. 4A to 4D.

FIG. 4A shows that all peptides in Table 2 can be detected individually in a plasma sample by SRM-MS using at least three transitions. The specificity of the assay for the peptides of interest is confirmed by the relative intensities of the transitions matching the relative intensity of the relevant product ions in an MS/MS scan. FIG. 4B confirms the specificity of the peptides, showing experiments in which the plasma sample was spiked with crude synthetic peptide which demonstrated the appropriate increase in signal.

C3b breakdown was further analysed in an in vitro assay. C3b was incubated along with FI and a fragment of cofactor CR1, selected over FH as CR1 drives the reaction to cleavage of iC3b to C3c+C3dg, whereas FH will only support cleavage of C3b to iC3b. Sequential samples were taken from the reaction and stopped by boiling.

FIG. 4C shows the time course of the C3b breakdown via gel electrophoresis. Analysis using MS and the peptides of Table 2 demonstrates that the formation of C3b fragments iC3b, C3f and C3c, and loss of intact C3b can be clearly detected over time (FIG. 4D). Not all peptides are shown since some (e.g. C3a) will not be present in the in vitro set-up, and others represent multiple products.

These data demonstrate that C3/C3b breakdown can be measured in a quantitative manner using GluC-derived peptides and MS. This enables the presence and levels of complement proteins to be detected in complement-related diseases such as AMD, as well as providing information as to successful treatment outcomes.

A single assay which can measure all FH family, C3 fragments and FI proteins allows for the simultaneous analysis of all key proteins in the complement amplification loop from just one sample and with efficient throughput.

### Example 3: Detection of the Complementome in AMD Patients

### 3.1 Sample Collection and Processing

Plasma samples were collected during the Cambridge AMD study; a case-control study with subjects recruited from the southeast and northwest of England between 2002 and 2006. The original study and its results are described in Yates et al., (2007). N. Engl. J. Med. 357, 553-561. All 246 affected subjects had advanced, end-stage AMD (choroidal neovascularization and/or geographic atrophy). 166 control subjects were spouses, partners or friends of index patients. Blood samples were obtained at the time of interview; EDTA and lithium-heparin plasma samples were used for the measurement of CFH, FHL-1 and the FHR proteins.

Analysis of plasma samples by Mass Spectrometry was performed as per Example 2 to determine levels of CFH, FHL-1 and FHR proteins. Samples were prepared for LC-MS/MS analysis by digestion and addition of SIS spiking solution as described in (2.1 and 2.2). Samples were then analysed by LC-SRM/MS as described in (2.3).

All statistical analyses were performed using GraphPad Prism (v8.4.3).

### 3.2 Results

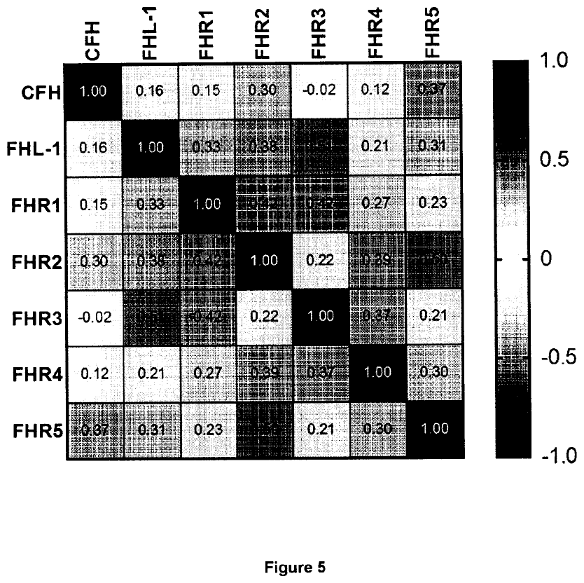

The data was analysed for evidence of correlation between the levels of the studied proteins. All Pearson correlation coefficients were found to be weaker than +/−0.55. It was therefore concluded that there is no strong linear relationship between different protein levels and little pair-wise correlation. Notably, inspection of the scatterplots did not reveal evidence of nonlinear relationship between variables. The relevant correlation matrix is shown in FIG. 5.

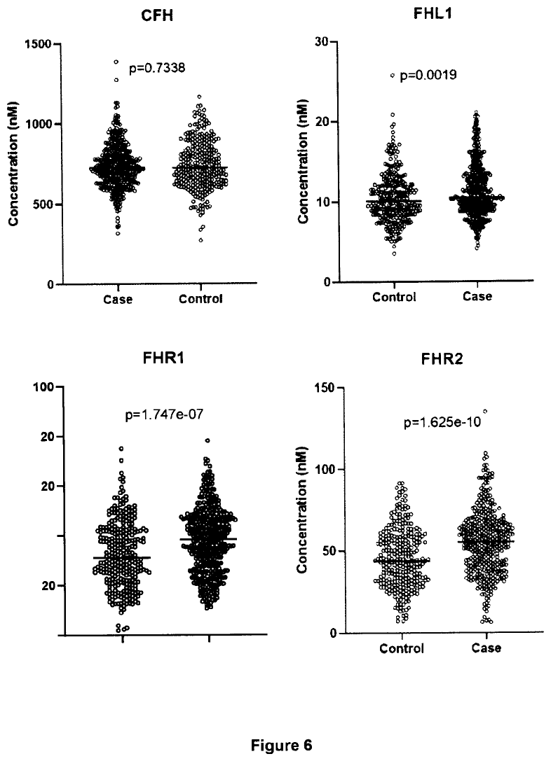

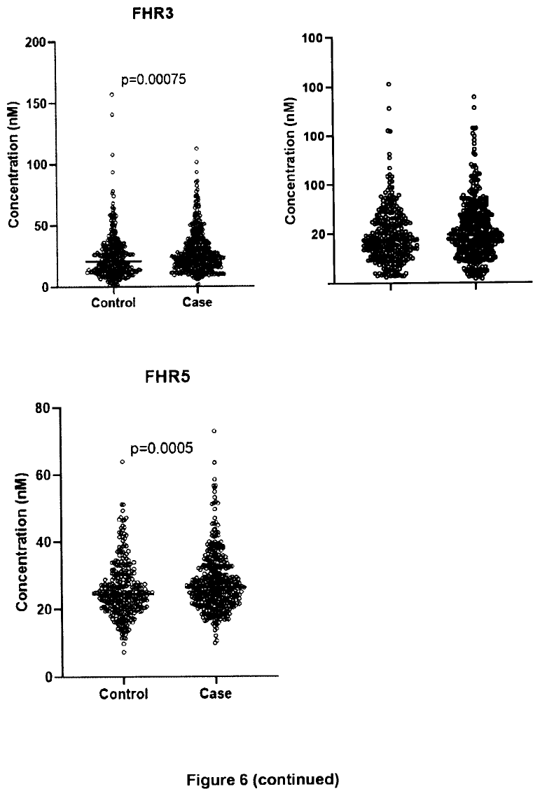

Protein levels were then compared between AMD cases (n=399; subjects having CNV, geographic atrophy or a mixed phenotype) and controls (n298). Non-parametric tests (Mann-Whitney test) for the absolute protein levels were performed and statistically significant differences were detected between cases and controls for FHR1, FHR2, FHR3, FHR4, FHR5 and FHL-1 (FIG. 6); p<0.05. Thus, circulating FHL-1 and FHR-1 to FHR-5 levels are higher in people with advanced AMD.

The association of advanced AMD with each of the FH, FHL-1 and five FHR levels was assessed via Wald tests using linear regression models adjusted for sex, age and the first two genetic principal components (as estimated within the IAMDGC study). The association of levels with advanced AMD was also reported via odds ratio (OR) expressed as per one standard deviation (SD) change of log-levels using logistic regression models adjusted for sex, age and the first two genetic principal components. The results are displayed in Table 1.

TABLE 1
Demographics of study samples and association analyses between
AMD and circulating FH, FHL-1, FHR-1 to FHR-5 protein levels.

| | Controls | Cases | |
|---|---|---|---|
| N | 252 | 352 | |
| Age, ys (SD) | 75.2 (7.9) | 73.9 (8.3) | |
| Male (%) | 39.3 | 45.7 | |
| AMD phenotype | | | |
| CNV only | | 218 | |
| GA only | | 73 | |
| Mixed | | 61 | |
| | | | OR (95% Cl)^{b} |
| Mean FH levels, | 737.3 | 736.5 | |
| nM (95% Cl) | (718.2-756.5) | (721.3-751.6) | |
| Association with | 0.005, 0.23, 0.982 | | 1.01 |
| AMD, Beta, SE, P^{a} | (0.02, 0.23, 0.936) | | (0.86-1.20) |
| Mean FHL-1 levels, | 10.4 | 11.3 | |
| nM (95% Cl) | (10.1-10.8) | (11.0-11.7) | |
| Association with | 0.08, 0.02, 1.4 × 10⁻³ | | 1.35 |
| AMD, Beta, SE, P^{a} | (0.08, 0.02, 4.9 × 10⁻⁴) | | (1.14-1.60) |
| Mean FHR-1 levels, | 31.2 | 38.4 | |
| nM (95% Cl) | (29.4-32.9) | (37.0-39.8) | |
| Association with | 7.22, 1.12, 2.1 × 10⁻¹⁰ | | 1.81 |
| AMD, Beta, SE, P^{a} | (7.21, 1.12, 2.4 × 10⁻¹⁰) | | (1.47-2.24) |
| Mean FHR-2 levels, | 45.3 | 55.3 | |
| nM (95% Cl) | (43.1-47.6) | (53.2-57.4) | |
| Association with | 0.71, 0.12. 1.9 × 10⁻⁹ | | 1.66 |
| AMD, Beta, SE, P^{a} | (0.74, 0.12, 6.0 × 10⁻¹⁰) | | (1.38-1.98) |
| Mean FHR-3 levels, | 24.1 | 28.9 | |
| nM (95% Cl) | (21.7-26.5) | (27.1-30.8) | |
| Association with | 0.55, 0.13, 4.4 × 10⁻⁵ | | 1.54 |
| AMD, Beta, SE, P^{a} | (0.59, 0.13, 1.4 × 10⁻⁵) | | (1.29-1.84) |
| Mean FHR-4 levels, | 46.1 | 53.8 | |
| nM (95% Cl) | (42.7-49.6) | (50.5-57.1) | |
| Association with | 0.53, 0.17, 2.1 × 10⁻³ | | 1.27 |
| AMD, Beta, SE, P^{a} | (0.56, 0.17, 1.3 × 10⁻³) | | (1.08-1.50) |
| Mean FHR-5 levels, | 25.5 | 27.9 | |
| nM (95% Cl) | (24.5-26.5) | (27.0-28.9) | |

| | | |
|---|---|---|
| Association with | 0.09, 0.03, 1.9 × 10⁻⁴ | 1.38 |
| AMD, Beta, SE, P^{a} | (0.10, 0.03, 1.9 × 10⁻⁴) | (1.16-1.63) |

^{a}Wald tests using linear regression models; adjusted P-values for sex, age and first two genetic principal components as estimated in Fritsche et al.² are displayed in parentheses; ^{b}Odds ratio (OR) of advanced disease expressed as per standard deviation change of log-levels using logistic regression models adjusted for sex, age and the first two genetic principal components. AMD = age-related macular degeneration; CNV = choroidal neovascularization; GA = geographic atrophy; SE = standard error; CI = confidence interval.

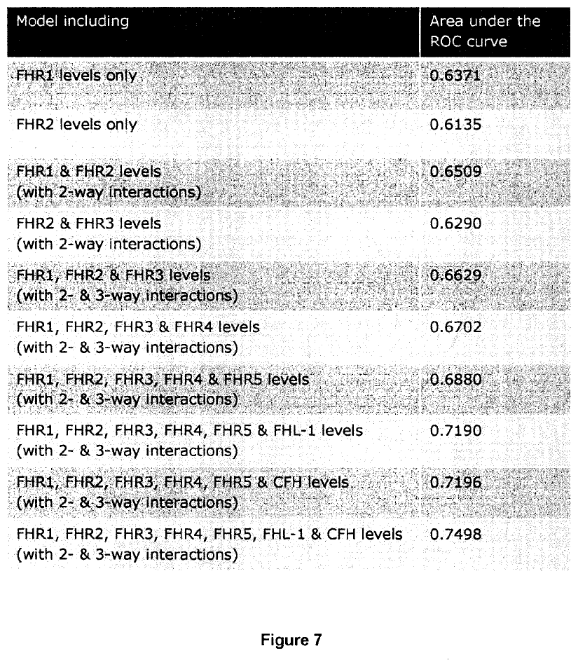

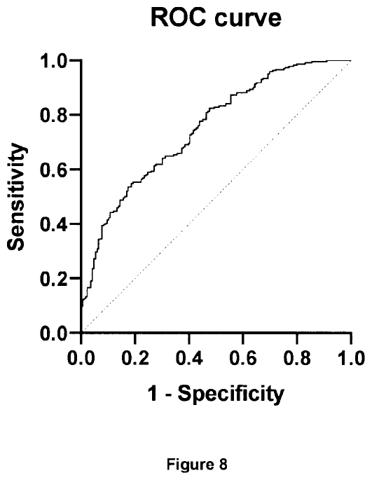

The data were examined to determine to what extent the clinical outcome (AMD or no AMD) could be predicted based on the different protein level values. Multiple logistic regression analysis was used for this purpose and the findings for different models are shown in FIG. 7. It was found that a model including all studied proteins (FHR1, FHR2, FHR3, FHR4, FHR5, FHL-1 & CFH protein levels) had the highest discrimination ability (AUROC (area under ROC curve) of 0.7498; FIG. 8), although all of the models tested were capable of discriminating between subjects with AMD and control subjects.

Genotype Data and Genome-Wide Association Analysis

Genome-wide association analyses were performed of the protein levels that were found to be elevated in advanced AMD cases (i.e., FHL-1 and FHR-1 to FHR-5).

All individuals included in the study had been previously genotyped with a custom-modified Illunina HumanCoreExome array at the Centre for Inherited Disease Research (CIDR, Baltimore, Maryland, USA) and analysed within the International AMD Genomics Consortium (IAMDGC) GWAS (43,566 subjects; 16,144 advanced AMD cases and 17,832 controls of European ancestry in the primary analysis dataset). Quality control and genotype imputation using the 1000 Genomes Project (Abecasis et al., Nature 2012, 491, 56-65) reference panel were performed by the IAMDGC as described in Fritsche et al., Nature Genetics, 2016, 48, 134-143.

GWASs were performed for FH, FHL-1 and the five FHR levels (transformed to ensure normality) in controls only, using linear regression models adjusted for sex, age and the first two genetic principal components, and variants with Minor Allele Frequency, MAF≥1%. The GWASs were carried out using the EPACTS software (http://genome.sph.umich.edu/wiki/EPACTS, version 3.3.2) and Wald tests were performed on the variant genotypes coded as 0, 1 and 2 according to the number of minor alleles for the directly typed variants or allele dosages for the imputed variants. Manhattan and Q-Q plots were generated using the qqman R package (version 0.1.4). Regional plots of association were generated using LocusZoom.org. Finally, linkage disequilibrium (LD) measures (R² and D′) were calculated using LDlink (https://ldlink.nci.nih.gov/), based on the European (EUR) population genotype data originated from the Phase 3 (Version 5) of the 1000 Genomes Project.

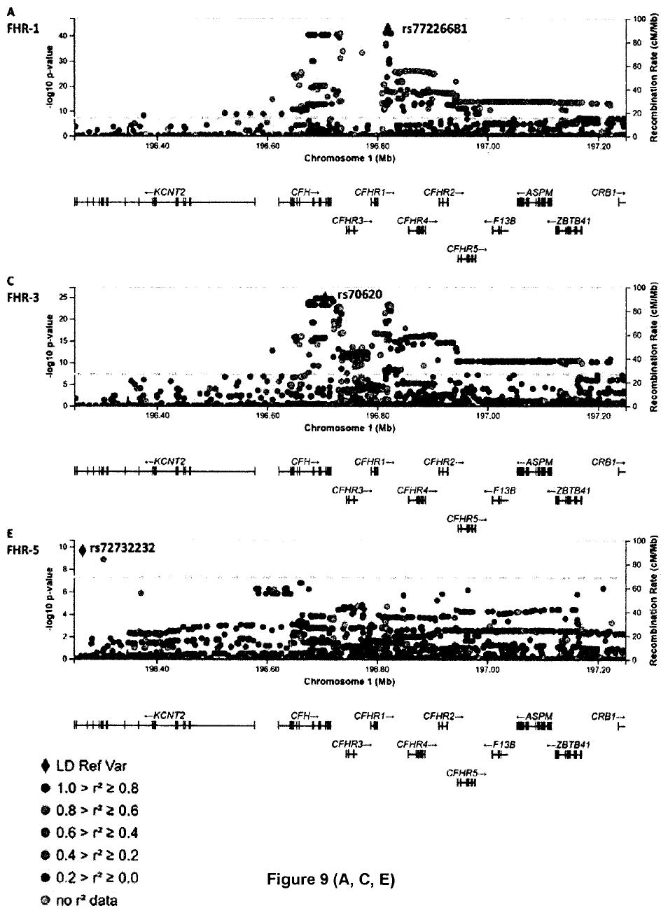

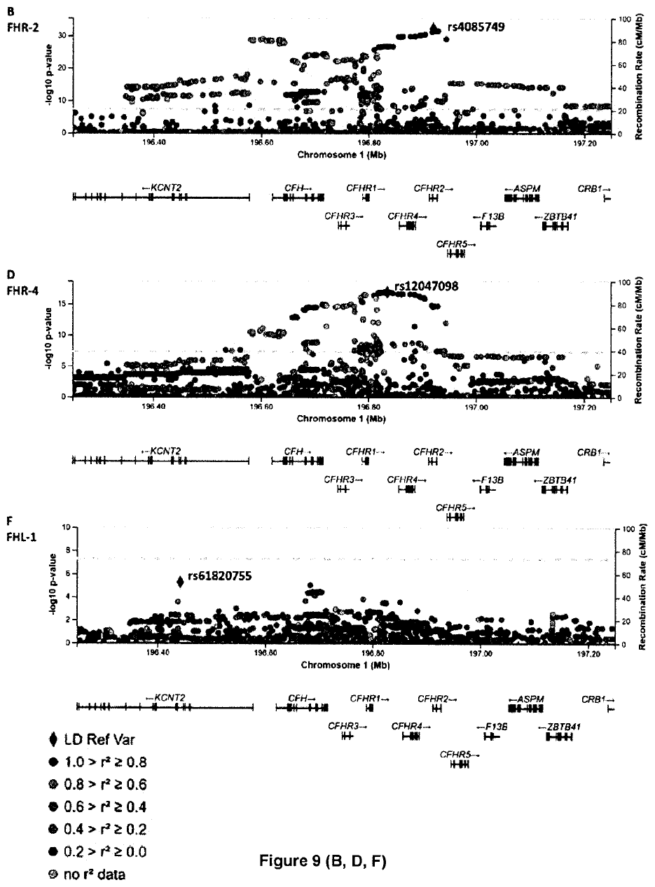

Remarkably, all GWASs of the five FHR protein levels in 252 controls showed a genome-wide significant (P≤5×10⁻⁸) peak at the CFH locus, see FIG. 9. For FHR-1, FHR-2, FHR4 and FHR-5, the CFH locus was the only genome-wide significant peak observed.

FHR-3 showed a more polygenic profile, with genome-wide significant signals at rs113721756 on chromosome 10 (P-value=1.7×10⁻⁸), rs111260777 on chromosome 11 (P-value=1.5×10⁻⁹), rs117468955 on chromosome 12 (P-value=3.0×10⁻⁸), rs4790395 on chromosome 17 (P-value=3.6×10⁻⁸) and rs117115124 on chromosome 19 (P-value=2.5×10⁻⁸) in addition to the CFH locus. The strongest signal from the GWAS of FHL-1 levels was observed at rs200404865 on chromosome 13 (P-value=9.6×10⁻⁷), with the strongest signal at the CFH locus observed at intronic KCNT2 rs61820755 (P-value=5.3×10⁻⁶).

The CFH locus genome-wide significant regions from the analyses of FHR-1 to FHR-5 levels overlapped among the different levels, but showed nominally different top signals (i.e., intergenic CFHR1/CFHR4 rs149369377 for FHR-1 with P-value=2.6×10⁻⁴³ and β=−18.2, synonymous CFHR2 rs4085749 for FHR-2 with P-value=6.3×10⁻³³ and β=−1.5, intronic CFH rs70620 for FHR-3 with P-value=1.5×10⁻²⁵ and β=2.0, intergenic CFHR1-CFHR4 rs12047098 for FHR-4 with P-value=1.1×10⁻¹⁷ and β=−1.7, and intronic KCNT2 rs72732232 for FHR-5 with P-value=2.2×10⁻¹⁰ and β=−0.5). These top signals are not in high LD with each other, except for rs4085749 of FHR-2 and rs12047098 of FHR-4 (R²=0.83, D′=0.95).

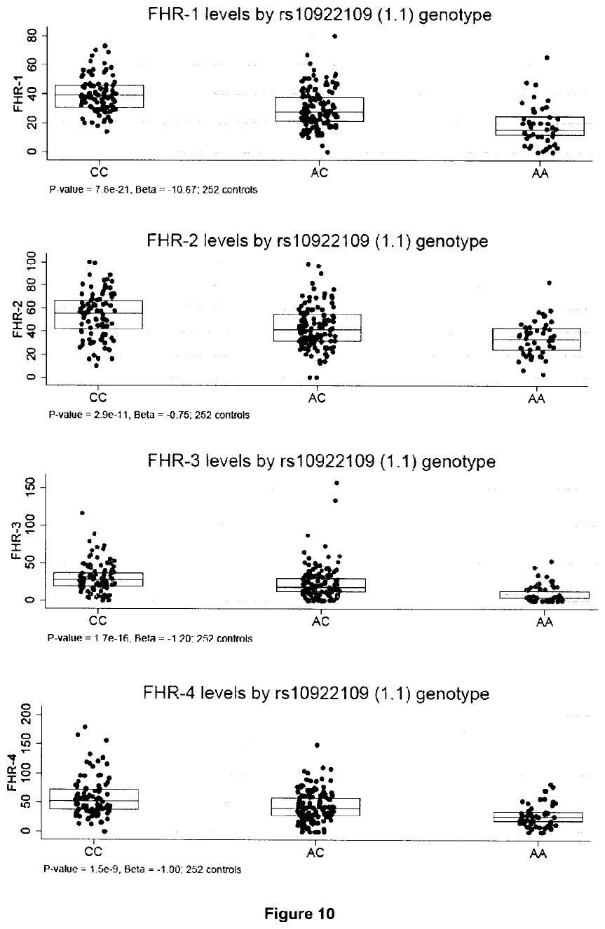

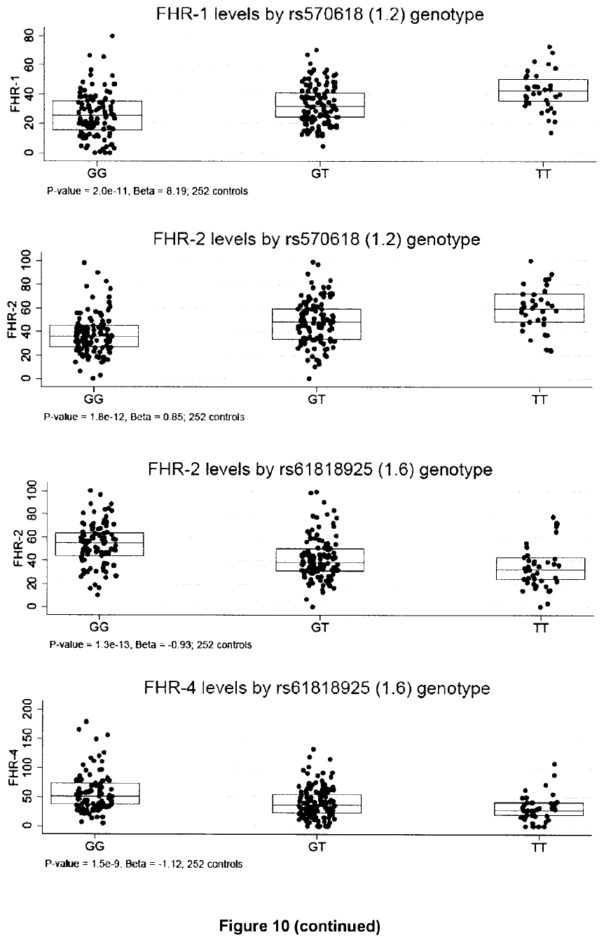

Next, it was assessed whether the GWAS top signals of FHR-1 to FHR-5 protein levels were in LD with any of the independently AMD-associated variants at the CFH locus reported by the IAMDGC GWAS, which also included the Cambridge samples analysed in this study (i.e., intronic CFH rs10922109 [1.1]; intronic CFH rs570618 [1.2], proxy for Y402H; CFH R1210C, rs121913059 [1.3]; intergenic rs148553336 [1.4], 8 kb upstream CFH/35 kb downstream KCNT2; intronic KCNT2 rs187328863 [1.5]; intergenic rs61818925 [1.6], 14 kb downstream CFHR1/156 kb upstream CFHR4; intronic CFH rs35292876 [1.7]; intronic CFHR5 rs191281603 [1.8]; see Table 2 and FIG. 10). The rare CFH variant R1210C, rs121913059 [1.3] was present heterozygously in a single Cambridge case and excluded from this analysis.

The top signal for FHR-1 was in modest LD with the top AMD-associated variant 1.1 (R²=0.30) and low LD with the proxy for Y402H 1.2 (R²=0.12); the top signal for FHR-2 was in modest LD with 1.1 (R²=0.35) and 1.6 (R²=0.36), and low LD with 1.2 (R²=0.16); similarly for the top signal of FHR-4 (R² equal to 0.38, 0.42 and 0.16 with 1.1, 1.6 and 1.2, respectively); low LD was seen with 1.1, 1.2 and 1.6 (R² equal to 0.16, 0.12 and 0.11, respectively) for the top signal of FHR-3, while the top signal of FHR-5 was in low/modest LD with 1.4 (R²=0.26).

Furthermore, genome-wide significant associations were observed at the top IAMDGC variant rs10922109 (1.1) with P-values 8.6×10⁻²¹, 2.9×10⁻¹⁰, 2.2×10⁻¹⁶ and 1.7×10⁻⁹ for FHR-1, FHR-2, FHR-3 and FHR-4, respectively, at the proxy for Y402H 1.2 with P-values 2.0×10⁻¹¹ and 1.8×10⁻¹² for FHR-1 and FHR-2, respectively, and at the variant 1.6 with P-values 1.8×10⁻¹¹ and 2.4×10⁻⁹ for FHR-2 and FHR-4, respectively. All these genetic associations showed direction of allelic effect on levels concordant with that on disease as estimated in the IAMDGC GWAS study (Table 2, FIG. 10). Altogether, these GWAS findings support that the CFH locus AMD-risk variants increase disease risk through increase of FHR protein levels.

Mendelian Randomization Estimates of the Effects of Circulating Levels of Complement Regulatory Proteins on Susceptibility to AMD

A Mendelian randomization approach was used to test if genetically proxied FHR protein levels are associated with risk of AMD.

Independent (LD, R2<0.001) genetic variants associated with the exposure were selected as instrumental variables (a protein at a time) at genome-wide significance level in controls only. If a single instrument was available, the ratio of coefficients method was used, also known as the Wald method, to estimate the effect of genetically proxied protein levels on the disease risk. The Wald ratio for a single genetic variant as instrumental variable is defined as its genetic association with the outcome (i.e. risk of AMD) over the genetic association with the exposure (i.e. protein level). Using a one-sample approach, the genetic association was derived with the exposure from the GWASs based on linear regression models for the FHR protein levels conducted on the Cambridge controls only (n=252). The genetic associations with the outcome were obtained from the GWAS based on a logistic regression model with AMD status as outcome conducted on the Cambridge samples (252 controls and 353 cases). If multiple instruments were available for a protein, the inverse-variance weighted (IVW) method was used under a fixed-effect model. Instrument strength was evaluated using R2 as the proportion of the variance of the protein explained by the genetic variant(s). The Mendelian randomization analysis was performed using the MendelianRandomization (version 0.4.2) and TwoSampleMR (version 0.5.5) R packages.

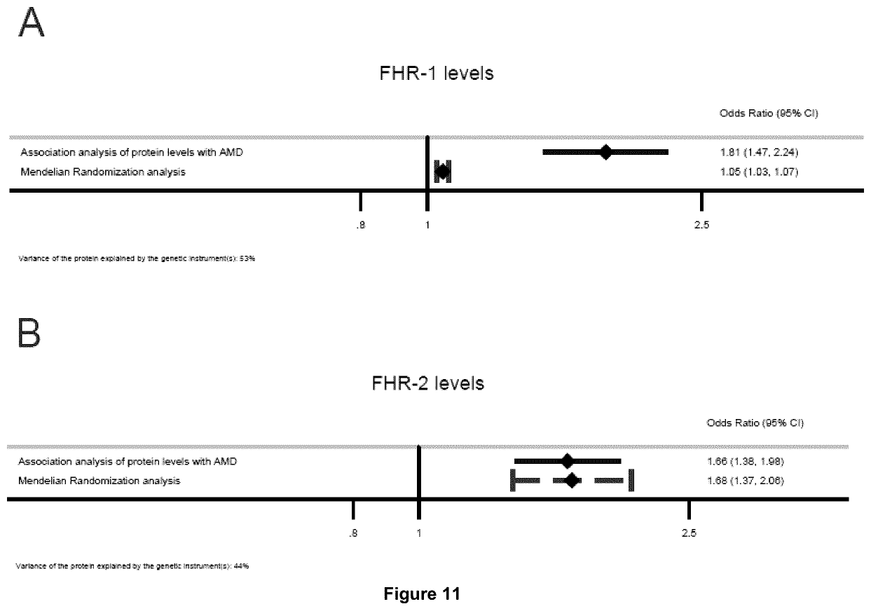

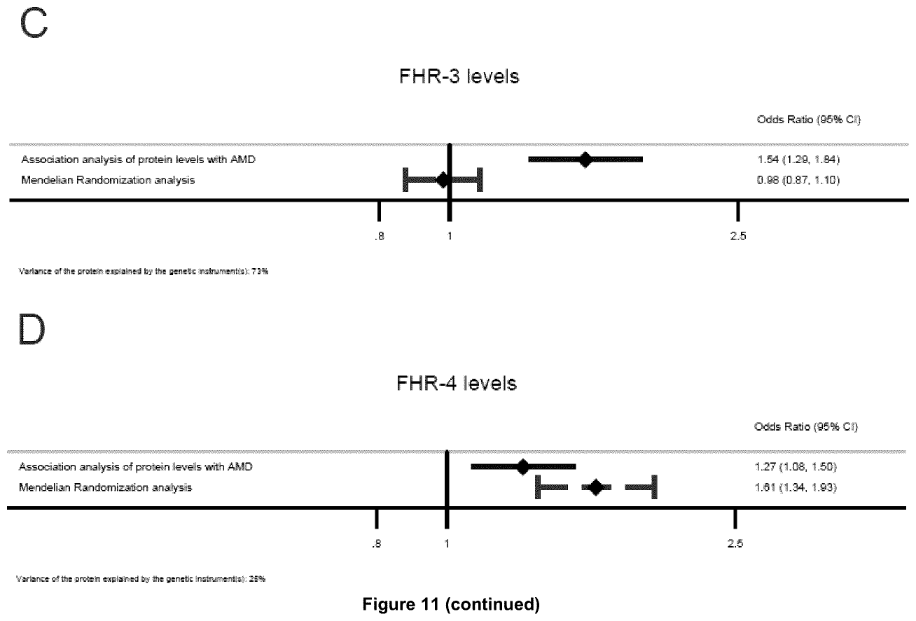

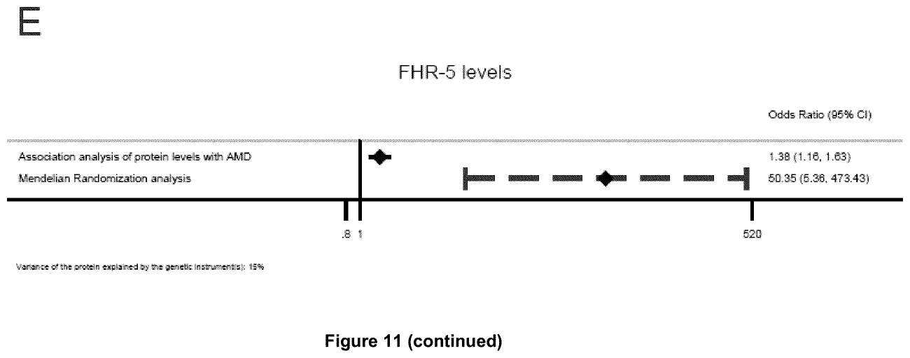

FIG. 11 shows the Mendelian randomization estimates of the FHR protein levels obtained using the (one-sample) Wald ratio (if a single instrument was available; FHR1, FHR2, FHR4, FHR5) or the IVW method (if multiple instruments were available; FHR3) together with the traditional epidemiologic estimates of the association of the levels with AMD obtained from logistic regression models and ORs (Table 1).

The variance of the FHR protein levels explained by the corresponding genetic instrument(s) varied from 15% for FHR5 to 73% for FHR3. The Mendelian randomization estimates were statistically significant and of concordant direction with the observational OR estimates for FHR-1, FHR-2, FHR-4 and FHR-5, providing evidence in support of a causal effect (FIG. 11). For FHR3, the Mendelian randomization estimate did not support an association of the protein levels with the disease (0.98, 95% CI 0.87-1.10). The GWAS of FHL-1 levels did not show any genome-wide significant signals to use as genetic instruments in the Mendelian randomization analysis. It is worth noticing that the strongest FHL-1 GWAS signal at the CFH locus was observed at rs61820755 (P-value=5.3×10⁻⁶, β=0.22) and that this variant did not show association with AMD in the Cambridge samples (P-value=0.32; β=0.22).

As such, while these data strongly support a causal role of increased FHR-1, FHR-2, FHR4 and FHR-5, the consequences of the observed increase in FHL-1 and FHR-3 circulating levels in individuals with AMD remain less clear.

Applying a cut off for ‘high’ expression of FHR proteins according to the 90^{th} percentile of the healthy controls group, it was calculated that ˜30% of AMD cases in this cohort displayed elevated levels of at least one FHR protein. This is consistent with the observation that around 34% of AMD patients carry an AMD risk variant at this locus. This is likely an underestimate, since our controls in this study contain individuals with AMD risk variants but do not (yet) have AMD.

Conclusions

Using 252 non-AMD controls to get insights into the genetic determinants of the circulating protein levels measured in this study, it was discovered that variants at the CFH locus regulate all five FHR protein levels (with genome-wide significant associations in our analyses of the FHR protein levels overlapping with the AMD-associated CFH region). Notably, established genetic associations with AMD risk at the non-coding variants 1.1, proxy for Y402H 1.2 and 1.6 translated into genetic associations at genome-wide significant level with FHR-1, FHR-2, FHR-3 and FHR-4 from the GWASs in our control group

The identification of the CFH locus as a cis protein quantitative trait locus (cis-pQTL) for the five FHR levels prompted the use of the available genetic data in a Mendelian randomization fashion to triangulate this evidence. For FHR-1, FHR-2, FHR-4 and FHR-5, the support provided by Mendelian randomization analyses for a potential casual role in susceptibility to AMD is striking, with Mendelian randomization estimates corroborating the preliminary evidence shown by the observational OR estimates (see Table 1).

This reframes our understanding of the aetiology of AMD, and the role of the non-coding risk variants on chromosome 1q31.3, demonstrating a prominent role for the FHR proteins. It also demonstrates that targeting (and lowering) of FHR proteins in the circulation as a viable therapeutic avenue for AMD, including systemic therapeutic strategies.

Identifying patients with risk factors for AMD will allow patients to avoid surgical procedures, especially in the early stages of disease before the loss of visual acuity, where therapeutic intervention may yield the most benefit. Patient stratification will be important as only a proportion of AMD patients are likely to suffer from FHR-mediated disease. However, as demonstrated above a patient's genetic-risk profile, coupled with measurements of their circulating FHR protein levels, is able to identify and stratify those patients most likely to benefit from such treatments, and to monitor their response to FHR-lowering agents.

TABLE 2
Single-variant association analyses for the 8 AMD independently associated variants at the
CFH locus from the IAMDGC study with FH, FHL-1, FHR-1 to FHR-5 levels in 252 controls.

| IAMDGC | dbSNP ID | | | Association with levels in Cambridge controls^{a} | | | | | | |
|---|---|---|---|---|---|---|---|---|---|---|
| association | (Chr:Position)^{c} | | | FH | FHL-1 | FHR-1 | FHR-2 | FHR-3 | FHR-4 | FHR-5 |
| signal | Major/Minor | IAMDGC OR | MAF | Beta | Beta | Beta | Beta | Beta | Beta | Beta |
| number | allele | (MAF in | Cambridge | (SE) | (SE) | (SE) | (SE) | (SE) | (SE) | (SE) |
| (direction^{b}) | (Imputation R²)^{d} | controls) | controls | P | P | P | P | P | P | P |
| 1.1 | rs10922109 | 0.38 | 0.422 | 0.49 | −0.10 | −10.67 | −0.75 | −1.20 | −1.0 | −0.04 |
| (−) | (1:196704632) | (0.426) | | (0.25) | (0.02) | (1.04) | (0.11) | (0.14) | (0.17) | (0.03) |
| | C/A | | | 0.056 | 3.7 × | 7.8 × | 2.9 × | 1.7 × | 1.5 × | 0.184 |
| | (1.00) | | | | 10⁻⁵ | 10⁻²¹ | 10⁻¹¹ | 10⁻¹⁶ | 10⁻⁹ | |
| 1.2 | rs570618 | 2.38 | 0.357 | 0.27 | 0.05 | 8.19 | 0.85 | 0.16 | 0.62 | 0.10 |
| (+) | (1:196657064) | (0.364) | | (0.26) | (0.03) | (1.17) | (0.11) | (0.16) | (0.18) | (0.03) |
| | G/T | | | 0.296 | 0.046 | 2.0 × | 1.8 × | 0.304 | 6.8 × | 7.8 × |
| | (1.00) | | | | | 10⁻¹¹ | 10⁻¹² | | 10⁻⁴ | 10⁻⁴ |
| 1.3 | rs121913059 | 20.28 | 0 | | No control carrier observed; not analyzed | | | | | |
| (+) | (1:196716375) | (0.00014) | | | | | | | | |
| | C/T (Genotyped) | | | | | | | | | |
| 1.4 | rs148553336 | 0.29 | 0.017 | −2.29 | −0.09 | 0.06 | −1.99 | 0.33 | 0.74 | −0.55 |
| (−) | (1:196613173) | (0.009) | | (1.02) | (0.10) | (5.00) | (0.48) | (0.62) | (0.72) | (0.11) |
| | T/C (Genotyped) | | | 0.025 | 0.353 | 0.990 | 4.6 × | 0.603 | 0.302 | 6.3 × |
| | | | | | | | 10⁻⁵ | | | 10⁻⁷ |
| 1.5 | rs187328863 | 2.27 | 0.013 | 1.12 | 0.06 | 0.75 | 1.13 | −0.51 | 0.61 | 0.01 |
| (+) | (1:196380158) | (0.028) | | (1.34) | (0.13) | (6.52) | (0.64) | (0.81) | (0.94) | (0.15) |
| | C/T | | | 0.404 | 0.660 | 0.908 | 0.080 | 0.536 | 0.515 | 0.956 |
| | (0.83) | | | | | | | | | |
| 1.6 | rs61818925 | 0.60 | 0.405 | −0.42 | 0.001 | 0.99 | −0.93 | 0.85 | −1.12 | −0.07 |
| (−) | (1:196815450) | (0.385) | | (0.27) | (0.03) | (1.33) | (0.12) | (0.16) | (0.18) | (0.03) |
| | G/T | | | 0.124 | 0.962 | 0.459 | 1.3 × | 1.7 × | 1.5 × | 0.014 |
| | (0.87) | | | | | | 10⁻¹³ | 10⁻⁷ | 10⁻⁹ | |

| | | | | |
|---|---|---|---|---|
| 1.7 | rs35292876 | 2.42 | 0.004 | MAF <= 1%; not analyzed |
| (+) | (1:196706642) | (0.009) | | |
| | C/T (Genotyped) | | | |
| 1.8 | rs191281603 | 1.07 | 0.008 | MAF <= 1%; not analyzed |
| (+) | (1:196958651) | (0.006) | | |
| | C/G | | | |
| | (0.42) | | | |

### Example 4: Tumor Cell IDO Enhances Immune Suppression and Decreases Survival Independent of Tryptophan Metabolism in Glioblastoma

### 4.1 Abstract

Purpose: Glioblastoma (GBM) is an incurable primary brain tumor that has not benefited from immunotherapy to-date. Greater than 90% of GBM expresses the tryptophan (Trp) metabolic enzyme, indoleamine 2,3-dioxygenase 1 (IDO). This observation supported the historical hypothesis that IDO suppresses the antitumor immune response solely through a mechanism that requires intratumoral Trp depletion. However, recent findings led us to investigate the alternative hypothesis that IDO suppresses the anti-GBM immune response independent of its association with Trp metabolism.

Experimental Design: IDO-deficient GBM cell lines reconstituted with IDO wild-type or IDO enzyme-null cDNA were created and validated in vitro and in vivo. Microarray analysis was conducted to search for genes that IDO regulates, followed by the analysis of human GBM cell lines, patient GBM and plasma, and the TCGA database. Ex vivo cell co-culture assays, syngeneic and humanized mouse GBM models were used to test the alternative hypothesis.

Results: Non-enzymic tumor cell IDO activity decreased the survival of experimental animals and increased the expression of complement factor H (CFH) and its isoform, factor H like protein 1 (FHL-1) in human GBM. Tumor cell IDO increased CFH and FHL-1 expression independent of tryptophan metabolism. Increased intratumoral CFH and FHL-1 levels were associated with poorer survival among glioma patients. Similar to IDO effects, GBM cell FHL-1 expression increased intratumoral Tregs and MDSCs while it decreased overall survival in mice with GBM.

Conclusions: Our study reveals a newly non-metabolic IDO-mediated enhancement of CFH expression and provides a new therapeutic target in patients with GBM.

### 4.2 Translational Relevance

Since the ECHO-301 phase III clinical trial results were reported, questions have been raised as to why IDO enzyme inhibition fails to improve the survival of patients with cancer. In the current study, this question was addressed and it was confirmed that tumor cell IDO possesses activities that extend beyond tryptophan metabolism and suppress the anti-cancer immune response. This study discovered that non-metabolic IDO increases the expression of immunosuppressive complement factor H (CFH) expression and in-turn, suppressed the anti-tumor immune response and decreased the survival of experimental animals with brain tumors. High intratumoral CFH levels were associated with a substantial decrease in glioma patient survival. These findings help elucidate our understanding of clinical trial results that have targeted IDO enzyme activity to-date and provide a new target for improving immunotherapeutic efficacy in patients with malignant glioma.

### 4.3 Introduction

Glioblastoma (GBM) is the most common malignant primary central nervous system (CNS) cancer in adults (1). Despite an aggressive standard of care treatment that includes maximal surgical resection when possible, followed by tumor-targeted radiation and chemotherapy with temozolomide (TMZ), the prognosis remains dismal. Median survival for GBM is 14.6 months (2) with a five-year overall survival (OS) of ˜4.7% in the United States (3). These grim statistics provide compelling rationale to develop more effective treatments for patients with GBM.

Immune checkpoint blockade and chimeric antigen receptor (CAR) T cell treatment have improved the lifespan of patients diagnosed with select advanced cancers (4). Patients with GBM are among the malignancies that are uniquely unresponsive to cancer immunotherapy and have yet to benefit from this approach in accordance with all phase III clinical trials to-date (5-7). A contributing factor to the immune resistance of GBM cells is indoleamine 2, 3-dioxygenase 1 (IDO) that is frequently expressed in wild-type isocitrate dehydrogenase (IDH) GBM (8). IDO is canonically characterized as a rate-limiting immunosuppressive enzyme that converts the essential amino acid, tryptophan (Trp), into downstream metabolites that are collectively referred to as kynurenines (Kyn) (9). Tumor cell expression of IDO increases the intratumoral accumulation of immunosuppressive regulatory T cells (Tregs; CD4⁺CD25⁺FoxP3⁺) and decreases overall survival in experimental mice with brain tumors (10). Although GBM cells do not normally express IDO, its expression is induced by tumor-infiltrating T cells (11). Higher levels of GBM-infiltrating T cells are therefore associated with higher intratumoral IDO expression levels and an associated decreased overall survival of GBM patients. (11, 12). Since IDO is expressed among a wide variety of adult cancers (13), pharmacologic enzyme inhibitor treatment approaches have been evaluated for their potential to improve cancer patient survival outcomes (14, 15). There have been no objective survival benefits noted among randomized clinical trials evaluating this approach in patients with aggressive cancer to-date (16). This may be due to a combination of factors including (i) a requirement to inhibit IDO and other immunosuppressive pathways simultaneously (17, 18), (ii) age-dependent increases of IDO that are unresponsive to pharmacologic enzyme inhibition (19), and/or (iii) immunosuppressive IDO effects that are independent of its association with acting as a tryptophan metabolic enzyme (20).

Previous work in mice demonstrated that Tregs accumulate in IDO-expressing brain tumors despite the treatment with a potent blood brain barrier-penetrating pharmacologic IDO enzyme inhibitor (18). Also unexpectedly, IDO-mediated Trp metabolism was predominantly mediated by non-GBM cells rather than by the tumor cells in the brain (21). These findings collectively challenge the historical hypothesis that tumor cell IDO increases Tregs and decreases survival through a mechanism that solely depends on Trp metabolism and motivated us to explore the alternative hypothesis that GBM cell IDO suppresses antitumor immunity independent of its enzyme function(s).

### 4.4 Materials and Methods

### 4.4.1 Patient Samples

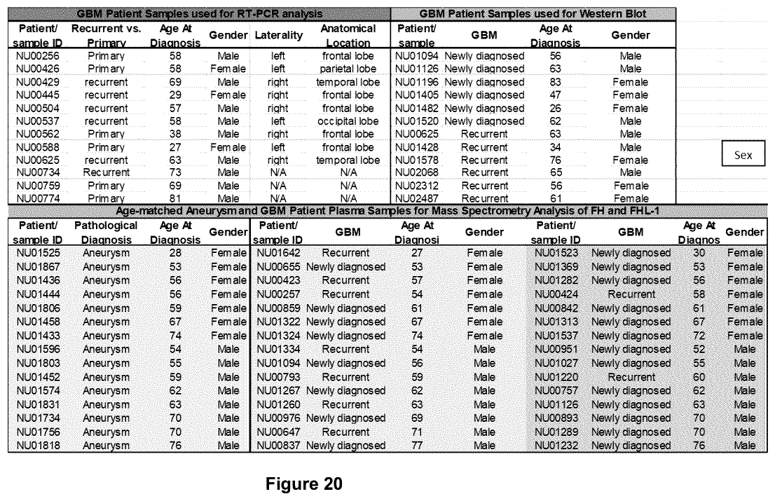

Peripheral blood from GBM and aneurysm patients were collected from the Northwestern Central Nervous System Tissue Bank (NSTB). Plasma samples were stored at −80° C. until batch analysis. Peripheral blood mononuclear cells (PBMCs) were isolated using Ficoll-Paque (GE Healthcare) density gradient separation and stored in liquid nitrogen prior to initiating co-culture experiments. Snap-frozen tissue from surgically-resected GBM were collected from the NSTB. All tumors were diagnosed according to WHO diagnostic criteria by Dr. Craig Horbinski, M.D./Ph.D. Detailed information for patient tissue samples used in this study is shown in FIG. 20.

### 4.4.2 The Cancer Genome Atlas (TCGA) Sample Description

The TCGA data for all cancer types analyzed in the current study were accessed from the UCSC Xena browser (http://xena.ucsc.edu/). RNA expression data assayed by RNASeq (Illumina Hi-seq platform) includes RSEM normalized level 3 data that is present in the TCGA as of Apr. 13, 2017. DNA methylation data were extracted from the same TCGA dataset. TCGA GBM gene expression data by AffyU133a array analysis were acquired from the UCSC Xena browser.

### 4.4.3 Glioblastoma Cell Lines and Patient-Derived GBM Xenografts (PDXs)

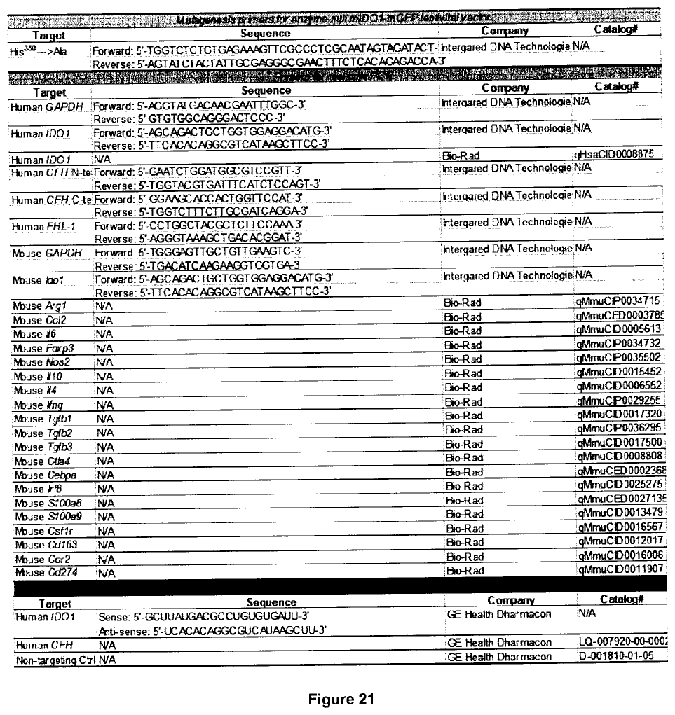

The human malignant glioma cell line U87 stably expressing luciferase (U87), the human IDO-overexpressing U87 cells (IDO-OE U87), and the mouse IDO^{-/-}tGBM cell line were created and maintained as previously described (11, 22, 23). To generate the mIDO-overexpressing tGBM cell lines, a lentiviral vector that expresses mIDO-mGFP fusion protein was purchased from Origene (catalog #CW303099). To obtain lentiviral vector encoding enzyme-null mIDO, site mutagenesis of His³⁵⁰ into Ala was performed on the wild-type mIDO-mGFP lentiviral vector using QuickChange II Site-directed Mutagenesis kit (Agilent Technologies, catalog #200523) following the product protocol. Mutagenic primers were designed by the online QuickChange Primer Design Program (www.agilent.com/genomics/qcpd) and sequence information is shown in FIG. 21 (SEQ ID NOs 158, 159). A GFP lentiviral vector, pCDH-CMV-MCS-EF1-copGFP-T2A-Puro (System Biosciences, Cat #CD513B-1) was provided by the Northwestern University SBDRC Gene Editing, Transduction and Nanotechnology (GET iN) Core as a control vector. The lentiviral vector that expresses FHL-1-mGFP fusion protein was purchased from Origene (catalog #CW304772). All vectors were sequenced before applied for viral packaging and cell transduction. Lentiviral particles were generated by transfecting 293FT cells with the lentiviral expression vector and packaging vectors following routine protocol at the SBDRC Gene Editing, Transduction and Nanotechnology (GET iN) Core which is supported by NIH award P30AR075049. IDO-tGBM cells were transduced with the lentiviral particles at a ratio of 5 infectious units of virus per cell in the presence of 8 μg/mL polybrene for 6 hours. The transduced cells were further selected by fluorescence-activated cell sorting (FACS) based on the GFP intensity. Cells within the top 1% GFP intensity were enriched for subsequent experiments.

IDO-deficient U87 cells (IDO-KO U87) were generated by the Applied StemCell Inc. using CRISPR-CAS9 technique. Briefly, human IDO guide RNAs targeting the exon 8 of human IDO gene were designed at CRISPR design web tool (Deskgene and CRISPOR) with at least three mismatches for NGG PAM sites. The crRNA-tracrRNA duplex were prepared by mixing equimolar concentration of Alt-R crRNA, Alt-R tracrRNA and ATTO 550 (catalog #1075298; Integrated DNA Technologies) followed by heating at 95° C. for 5 min and slowly cooled to room temperature. To prepare the Cas9/RNP complex, the crRNA-tracrRNA duplex and Alt-R S.p Cas9 nuclease V3 (catalog #1081059; Integrated DNA Technologies) were gently mixed and incubated at room temperature for 20 min. U87 cells were resuspended in SE nucleofection buffer (SE cell Line 4D-Nucleofector X kit L; V4XC-1024, Lonza) and incubated with Cas9/RNP complex at room temperature for 2 min and electroporated using a 4D nucleofector (4D-Nucleofector Core Unit: AAF-1002B; Lonza, 4D-Nucleofector X Unit: AAF-1002X, Lonza). 48 hours after transfection, cells were trypsinized and resuspended in phosphate-buffered saline (PBS) having 1% fetal bovine serum (FBS) and sorted by FACS based on ATTO signal intensity. After 7-14 days culture or formation of visible cell clones, genomic DNA were extract and subject to PCR. The PCR products were then Sanger-sequenced to identify clones that would result in frameshift mutation. IDO knockout at both mRNA and protein levels were confirmed by analyzing parental and IDO-KO U87 cells using RT-PCR and Western blotting, respectively.

The glioma cells from patient-derived GBM xenografts (PDX) were provided by the laboratory of Dr. C. David James at the Northwestern University and prepared as previously reported (24, 25). Except for the PDXs-derived human GBM cells, all the other cell lines used in this study were tested for mycoplasma prior to analysis and cultured in the DMEM/F12 medium (ThermoFisher Scientific, catalog #11320) supplemented with 10% FBS and 100 units/ml penicillin as well as 100 ug/ml streptomycin under 5% CO2 incubation condition unless described for specific experiments.

### 4.4.4 Animal and Tissue Preparation

Humanized mice reconstituted with human immune cells (NSG-SGM3-BLT), NOD.CB17-Prkdc^{scid}/J (NOD-scid) mice, CrTac:NCr-Foxn1^{nu} mice were used as previously described (11). Cre^{-/-}IDO^{-/-}tGBM mice were previously generated (21) by crossing transgenic mice that spontaneously develop glioblastoma after intraperitoneal injections of tamoxifen (26) with B6.129-Ido1^{tm1Alm}/J (Jackson Laboratories). Mice were maintained under specific pathogen-free conditions in the Northwestern University Center for Comparative Medicine. For T cell depletion experiments, 200 μg anti-mouse CD4 (clone YTS191; BioXCell), 200 μg anti-mouse CD8 (clone YTS169.4; BioXCell) and 200 μg anti-mouse NK1.1 (clone PK136; BioXCell) were administered by intraperitoneal (i.p.) injection 3 days prior to and every 3 days after tumor cell engraftment up to 30 days after intracranial injection or at the declared experimental endpoints. Rat IgG2b (clone LTF-2, BioXCell) and mouse IgG2a (clone C1.18.4, BioXCell) were administrated at the same concentration and dosing schedule as for the leukocyte-depleting antibodies. For orthotopic brain tumor mouse modeling, 3×10⁵ tGBM or patient-derived xenograft (PDX) cells were intracranially-engrafted similar to previous studies (27). PDX tumor tissue was kindly provided by Dr. C. David James at the Northwestern University, from continuously propagated patient-resected GBM that was subcutaneously engrafted into nude mice. Mice were euthanized at the indicated time point(s). Brain tumor and non-tumor contralateral brain hemisphere tissue was collected, dissected, and washed in ice-cold phosphate-buffered saline (PBS), frozen in liquid nitrogen, and stored at −80° C. until analysis or processed for other techniques. Procedures for all mouse experiments were reviewed and approved by the Institutional Animal Care and Use Committee at Northwestern University and were in compliance with national and institutional guidelines.

### 4.4.5 Co-Culture Assays

For the tGBM cell-splenic monocyte co-culture, monocytes were isolated and enriched from mouse spleens using EasySep™ Mouse CD11b Positive Selection Kit (Catalog #18970, STEMCELL) according to the product protocol. Viability of the isolated cells was typically >90% as seen by trypan blue staining. CD11 b⁺ cells were seeded onto 12-well plate at a density of ˜1.5×10⁶ per well and cultured in RPMI-1640 medium supplemented with 10% heat-inactivated FBS, 100 mg/ml streptomycin, 100 U/ml penicillin, 10 ng/ml mouse recombinant IL-2 (R&D System, catalog #402-ML-020) over night. The next day non-attached cells were removed and adherent cells were washed once with PBS and incubated in fresh RPMI-1640 medium as described above for another 5 days. After counting the macrophages, tGBM cells were seeded on a 0.4 μm Transwell insert at 1:1 ratio and placed into the 12-well plate for co-culture of 48 hours. At the end of co-culture, tGBM cells and some macrophage cells were lysed using RNA Lysis Buffer from the PureLink RNA Mini Kit (Thermo Fisher Scientific, catalog #12183020) and stored at −80° C. for RT-PCR. The remaining macrophages were washed with PBS containing 5 mM EDTA and gently de-attached by cell scrappers followed by twice washing with PBS containing 2% FBS, then subject to flow cytometry analysis. Conditioned media from the co-culture were collected and filtered through a 40-um cell strainer and stored at −80° C. for HPLC analysis. The co-culture of U87 cells with patient PBMCs-derived T cells was performed as described in our previous study (11).

### 4.4.6 Hematoxylin and Eosin (H&E) Staining and Immunohistochemistry (IHC)

Brain tumors were dissected and fixed in 10% (w/v) neutral buffered formalin for 24˜72 hours. Formalin-fixed tissues were processed into paraffin blocks and sectioned at a thickness of 4 um. After deparafinization, antigen retrieval was performed using sodium citrate pH6 buffer. The slides were incubated in decloaking chamber (Biocare Medical) at 110° C. for 5 minutes; rinsed in distilled water 2 times and in 1×phosphate buffered saline (PBS) for 5 minutes, then incubated with anti-mGFP antibody (Origene, catalog #TA150122) (1:5000 dilution) in antibody diluent, overnight at 4° C. After rinsing with Tris-Buffered NaCl Solution with 0.1% Tween 20 (TBST), sections were further incubated with HRP-labelled anti-rabbit secondary antibody (BioCare Mach2 #RHRP520MM) for 1 hour. Slides were then washed for 3 minutes. Immunohistochemical reactions were visualized using a DAB substrate (DAKO). Tissue sections were counterstained with hematoxylin Gill II (Surgipath), mounted in the xylene based mounting medium, and visualized under a light microscope. Both H&E and IHC images were taken using a CRI Nuance camera on Zeiss Axioskop microscope at the Northwestern University Center for Advanced Microscopy Core. Histology services were provided by the Northwestern University Research Histology and Phenotyping Laboratory supported by NCI P30-CA060553.

### 4.4.7 Microarray Analysis

The microarray analysis was carried out at the Northwestern University NUSeq Core Facility using the human transcriptome analysis system, Clariom™ D Assay (Thermo Fisher Scientific). Briefly, 1×10⁵ cells per well of U87 cells or IDO-OE U87 cells were seeded on 12-well plate. After attachment, cells were transfected with human IDO-specific siRNA at a final concentration of 20 nM (GE Health Dharmacon) using either Lipofectamine RNAiMAX Transfection Reagent (Thermo Fisher Scientific, catalog #13778030) or jetPRIME siRNA transfection reagent (Polyplus-transfection). The sequences of hIDO siRNA duplex are shown in FIG. 21. 16-18 hours after siRNA transfection, U87 cells were further incubated for another 48 hours with or without human recombinant IFNΓ (Shenandoah Biotechnology, SKU #100-77-100 ug) at a concentration of 100 ng/ml. After the incubation, cells were lysed using RNA Lysis Buffer from the PureLink RNA Mini Kit (Thermo Fisher Scientific, catalog #12183020) and stored at −80° C. The IDO-OE U87 cells were transfected with human IDO-specific siRNA for 24 hours then subject to cell lysis as described above. The above experiment was repeated twice at different time points. Total 36 samples (6 groups×2 duplicate×3 repeats) were subject to the microarray analysis. Total RNA was quantified with a NanoDrop 3000 then further evaluated by a Bioanalzyer. Only samples with a RIN value≥9.0 were used for downstream analysis. The RNA selected was amplified and hybridized using the GeneChip® WT PLUS Reagent Kit (Thermo Fisher Scientific, USA) and further analyzed by the GeneChip® Scanner 3000 platform (Thermo Fisher Scientific, USA).

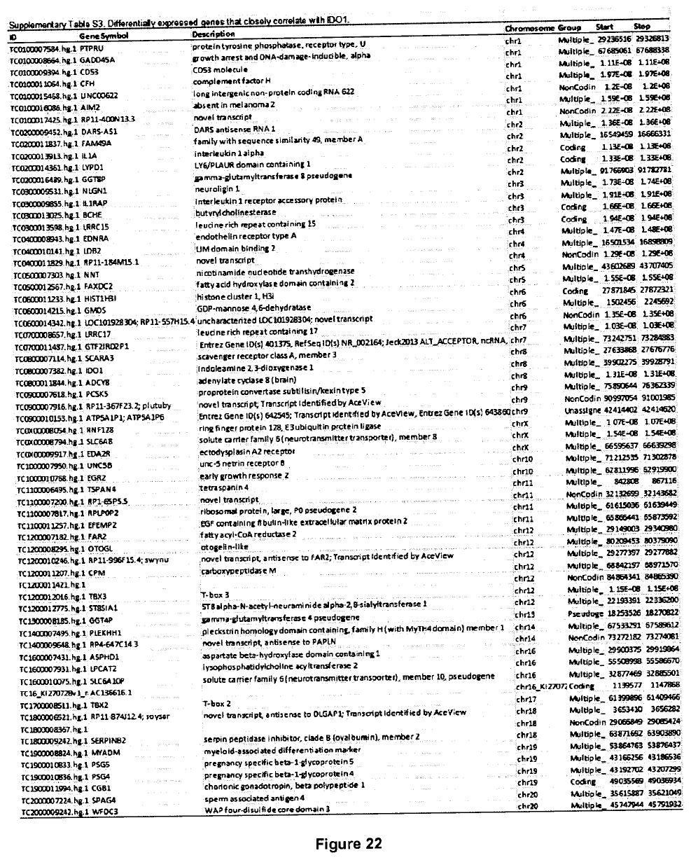

The Affymetrix Transcriptome Analysis Console (TAC Version 4.0.2, Thermo Fisher Scientific) was used for normalization, summarization, and quality control of the resulting microarray data using the signal space transformation-robust multi-array average (SST-RMA) algorithm. Analysis of variance (ANOVA) empirical Bayes (eBayes) method using adjusted statistical p-values (p<0.05; fold change±2), was used for determination of the differentially expressed genes within the TAC console. The eBayes method which is suitable for small sample sizes, uses moderated t-statistics, where instead of the global or single gene estimated variances, a weighted average of the global and single-gene variances is used (28). 65 genes were identified as the most differentially expressed genes by IDO siRNA treatment between U87 cells and IDO-OE U87 cells (FIG. 22). Two genes without a curated gene symbol (FIG. 22) associated with the Affymetrix probe set were excluded from downstream analyses. Gene expression pattern and KM analysis of these 63 genes were further compared to those of GBM IDO using the GlioVis online data portal (https://gliovis.shinyapps.io/GlioVis/). Pearson's correlation was also performed between mRNA level of each of these 65 genes with that of IDO using the TCGA GBM RNASeq data (as described in Methods). After the above screening, 4 genes showing highest correlation with IDO were identified, MYADM, GADD45A, TSPAN4, and CFH, of which only CFH shows the correlative expression with IDO as confirmed by the real-time RT-PCR analysis. The microarray data have been deposited in SRA and the accession number is GSE175700 (https://uridefense.com/v3/_https://www.ncbi.nlm.nih.gov/geo/query/acc.cgi?acc=GSE175700_; !!Dq0X 2DkFhyF93HkjWTBQKhk!Bep13fF052qW8RRWIdy7Muamf9MI8NMyGQJ8RTGR18jcBVqEAn7xY5YD8f m4gu4C6QplvwE$).

### 4.4.8 Western Blotting

For cultured cell samples, media were removed, and cells were lysed in ice-cold RIPA buffer supplemented with 1× Halt protease/phosphatase inhibitor cocktail (Thermo Fisher Scientific). For GBM tissue samples, ˜50 mg tissue sample was homogenized in the above protein lysis buffer using the gentleMACS Dissociator (Miltenyi Biotec) following product protocol. The protein lysate was centrifuged at 12,600×g for 15 min, the supernatant was stored at −80° C. for further analysis. Protein concentration was measured by the bicinchoninic acid assay (Thermo Fisher Scientific). Equal amounts of protein were loaded in pre-cast Mini-PROTEAN TGX Stain-Free gels (Bio-Rad). After electrophoresis, protein was transferred the PVDF membrane followed by blocking in 5% (w/v) non-fat milk in 1×TBST for one hour, then probed with primary antibodies: anti-mGFP antibody (Origene, catalog #TA150122) (1:1000 dilution), anti-hIDO (Cell Signaling Technology, clone: D5J4E) (1:1000 dilution), anti-FH/FHL-1 antibody (Origene, clone: OTI5H5, catalog #TA804532) (1:1000 dilution), anti-GAPDH (Cell Signaling Technology, clone: 14C10) (1:1000 dilution) overnight at 4° C. After 5 times washing with 1×TBST, membrane was incubated with donkey anti-rabbit/goat IgG antibodies conjugated with horseradish peroxidase (Jackson ImmunoResearch Inc.). The blotting membrane was then incubated with SuperSignal West Pico/Femto ECL substrate (Thermo Fisher Scientific) and visualized on ChemiDoc (BioRad).

### 4.4.9 Flow Cytometry

Flow cytometry was performed as previously described (27). All the conjugated antibodies were purchased from eBioscience and detailed information is shown in FIG. 21. Fc Blocking Ab (catalog #14-9161-73), anti-mouse CD16/CD32 (catalog #14-0161-82), fixation/Permeabilization concentrate (catalog #00-5123-56), fixation/Permeabilization diluent (catalog #00-5223-56), and permeabilization buffer (catalog #00-8333-56) were also purchased from eBioscience. Cytometry data were acquired on a BD LSRFortessa 6-Laser flow cytometer and analyzed on Flowjo 6 software. This work was supported by the Northwestern University—Flow Cytometry Core Facility supported by Cancer Center Support Grant (NCI CA060553).

### 4.4.10 RNA Isolation and Real-Time RT-PCR

Total RNA was extracted from freshly dissected tissue samples and cultured cells using the Trizol Reagent and PureLink RNA Mini Kit (Thermo Fisher Scientific), respectively. 1 μg of total RNA was reverse transcribed into mRNA using iScript cDNA Synthesis Kit (Bio-Rad). Quantitative real-time PCR was performed on a CFX96 Touch Real-Time PCR Detection System using the default program setting (Bio-Rad). The sequences for all PCR primers are listed in FIG. 21 (SEQ ID NOs 160-173). The relative quantitation of gene expression was calculated using the 2-AACT method (29) with normalization of the target threshold cycle (CT) values to the internal housekeeping gene (GAPDH).

### 4.4.11 Trp and Kyn Analysis by High Performance Liquid Chromatography (HPLC)

Procedures for HPLC sample processing and analysis have been previously described (30).

### 4.4.12 Mass Spectrometry Quantification of CFH and FHL-1

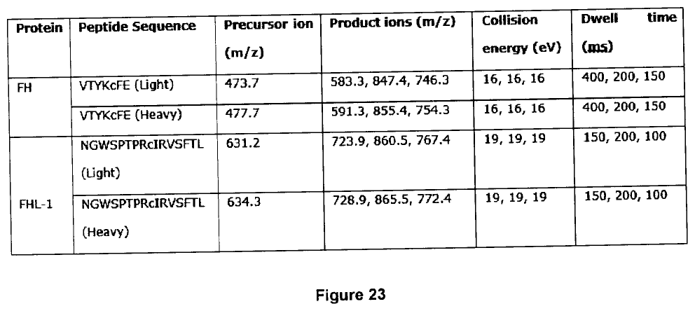

Evaluation of plasma levels for CFH and FHL-1 has been described previously (31). Briefly, plasma samples were thawed and vortexed, and a 5 μl aliquot taken and diluted with 90 μl 50 mM ammonium bicarbonate, 2 μl of 1% (w/v) ProteaseMax (Promega, Southampton, UK) and 1 μl 500 mM dithiothreitol. This was incubated at 56° C. for 25 min to reduce cysteine residues. 3 μl 500 mM iodoacetamide was then added and sample incubated in the dark for a further 15 min. To digest the protein, a further 43 μl of 50 mM ammonium bicarbonate and 1 μl ProteaseMAX solution was added alongside 5 μl of 1 μg/μl endoproteinase Glu-C(Roche, Mannheim, Germany). Sample was mixed and digested for 16 hours at 25° C. Digested samples were spiked with heavy-labelled synthetic peptide standards (FH: VTYKCFE; FHL-1: NGWSPTPRCIRVSFTL, each containing S-carboxymethylated cysteine and heavy labelled amino acids at the underlined residues) (Cambridge Research Biochemicals, Cambridge, UK) to a final concentration of 47.6 μg/μl for the FH and 0.95 μg/μl for FHL-1. Peptides were dried in a centrifugal evaporator and resuspended in 50 μl 0.1% (v/v) trifluroacetic acid. 4 μL of this peptide solution was analysed—providing final on-column standard peptides loads of 200 fmol FH and 2 fmol FHL-1 peptides, respectively. Peptides were separated using an Agilent 1200 series liquid chromatography system with a C18 column (250 mm×2.1 mm I.D., Thermo Scientific Acclaim 120, 3 μm particle size) at 50° C. Peptides were eluted using a gradient elution increasing from 5% acetonitrile to 25%. The flow rate was maintained at 250 μL/min with an initial composition of 5% Buffer B (acetonitrile with 0.1% (v/v) formic acid). The following gradient elution profile was used to separate the peptides (time: % acetonitrile): 0 min: 5%; 2 min: 5%; 3 min: 12%; 12 min: 15%; 15 min: 20%; 30 min: 25%; 31 min: 90%; 39 min: 90%; 40 min: 5%; 49 min: 5%. Eluted peptides were detected using an Agilent 6595 triple quadrupole mass spectrometer in SRM mode monitoring three transitions per peptide as shown in FIG. 23. Data were extracted using Skyline software (https://skyline.ms/) and protein concentration was calculated by comparison of peak areas between the heavy labelled standard peptides and its endogenous counterpart.

### 4.4.13 Cell Proliferation Assay

2000-3000 tGBM cells per well were seeded on 96-well plate. Cell growth at different time points were measured using the Cell Counting Kit 8 (abcam, catalog #ab228554) following the product protocol. Absorbance at 460 nm was measured using a Synergy™ 2 multi-mode microplate reader (BioTek).

### 4.4.14 Statistical Analysis

The cutoff value for gene expression levels were determined with Cutoff Finder software (http://molpath.charite.de/cutoff/) using significance as the cutoff optimization method (32). Kaplan-Meier (KM) survival analysis was performed to estimate the survival distribution, while the Bonferroni-corrected, Mantel-Cox, or Gehan-Breslow-Wilcoxon log-rank tests were used to assess the statistical significance of differences between the stratified survival groups using GraphPad Prism (version 9, GraphPad Software, Inc., La Jolla, CA). Renyi family of test statistics was computed via SAS software (version9.4, SAS Institute Inc., Cary, NC) to determine the survival difference between two groups given the presence of crossing hazard rates. Pearson's correlation was used to analyze the relationship between each two genes' mRNA expression level.

Canonical-correlation analysis (CCA) was performed using concoro function in R package CCR. F-approximations of Wilks' Lambda was used to test the statistical significance of canonical correlation coefficients, using p.asym( ) function in R package CCP. Comparisons between multiple groups were analyzed by One-way ANOVA using GraphPad Prism software. Differences were considered to be statistically significant when P<0.05. Standard error of the mean (SEM) is presented as the error bar in all bar graphs and mean±SEM was utilized to describe the data throughout the text unless specifically noted.

### 4.5 Results

### 4.5.1 Tumor Cell IDO Increases Immune Suppression Through Non-Enzyme Activity

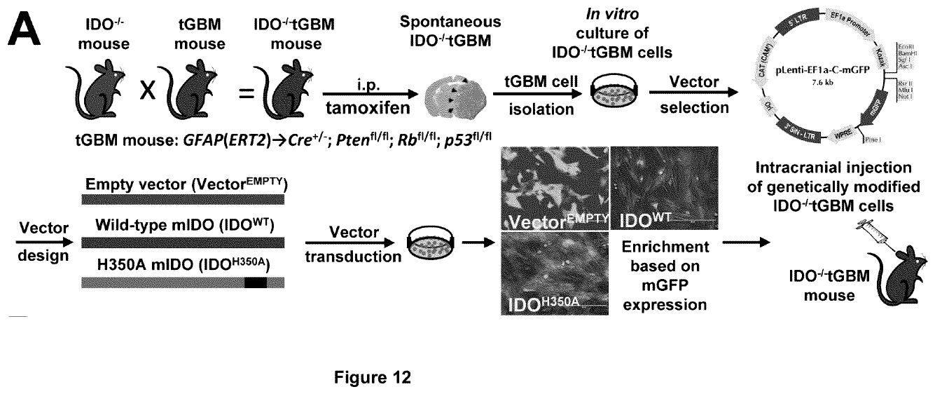

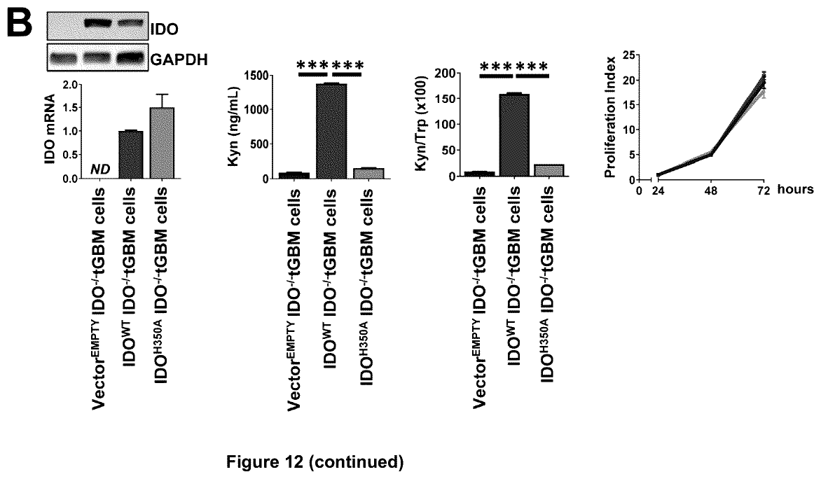

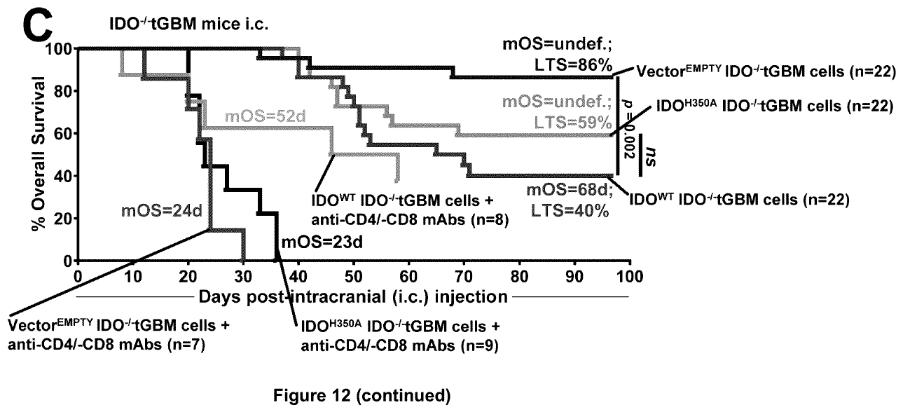

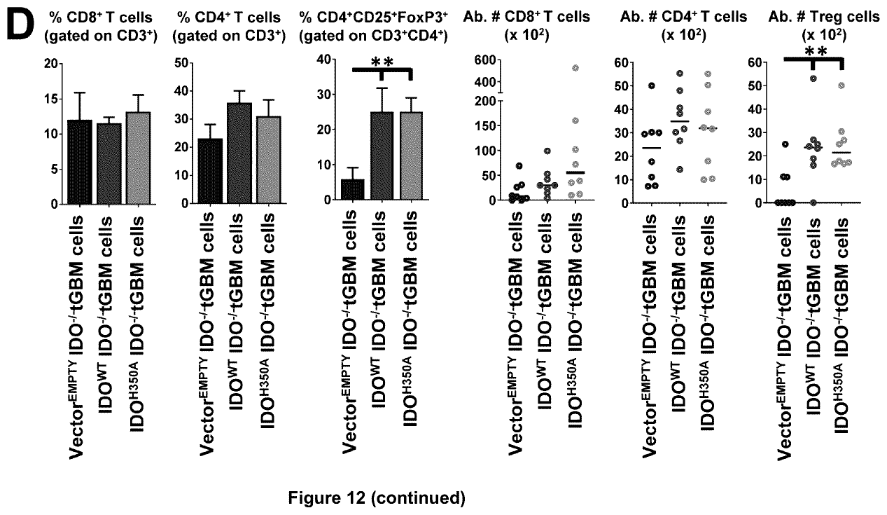

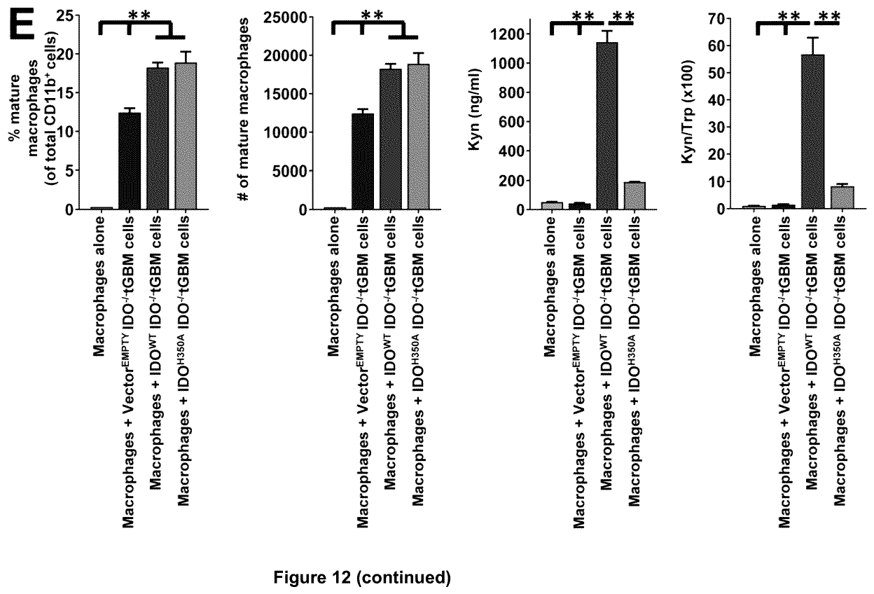

To determine if GBM cell IDO possesses non-enzyme activity, we bred B6.129-Ido1^{tm1Alm}/J (Ido^{-/-}) mice with GFAP(ERT²)→Cre; p53^{fl/fl}; Rb^{fl/fl}; pTEN^{fl/fl} mice (26) to generate an IDO-deficient tamoxifen-inducible transgenic mouse model of GBM (IDO^{-/-}tGBM; FIG. 12A; upper left). Tumor tissue was isolated from the mouse brain and disseminated into single cells (IDO^{-/-}tGBM cells) until growing at a rate of exponential proliferation, in vitro (FIG. 12A; upper right). Since the amino acid sequence for the mouse and human IDO enzyme active site is conserved (33), site directed mutagenesis was performed on a wild-type mouse IDO-mGFP cDNA fusion construct such that the derivative protein would change from a histidine to an alanine at the 350^{th} amino acid (H350A). IDO^{-/-}tGBM cells were then transduced with either an empty plasmid vector (Vector^{EMPTY}), a vector expressing wild-type murine IDO cDNA (IDO^{WT}), or a vector expressing the IDO enzyme null cDNA (IDO^{H350A}) (FIG. 12A; lower left). Fluorescence microscopy (FIG. 12A; lower right), real-time RT-PCR, Western blotting, and high-performance liquid chromatography (HPLC) for tryptophan (Trp) and kynurenine (Kyn) confirmed that both IDO^{WT}- and IDO^{H350A}-modified IDO^{-/-}tGBM cells express IDO mRNA and protein as compared to the Vector^{EMPTY}-expressing IDO^{-/-}tGBM cells that are absent for IDO expression (FIG. 12B). The modified IDO^{WT}-expressing IDO^{-/-}tGBM cells show a significant increase of Kyn accumulation as compared to both the Vector^{EMPTY}- and IDO^{H350A}-expressing IDO^{-/-}tGBM cells. The expression of IDO has no effect on the proliferation of IDO^{-/-}tGBM cells, in vitro (FIG. 12B, right most panel). These data validate the successful reconstitution of IDO-deficient tumor cells with constructs expressing enzymatically active or enzymatically null IDO protein.

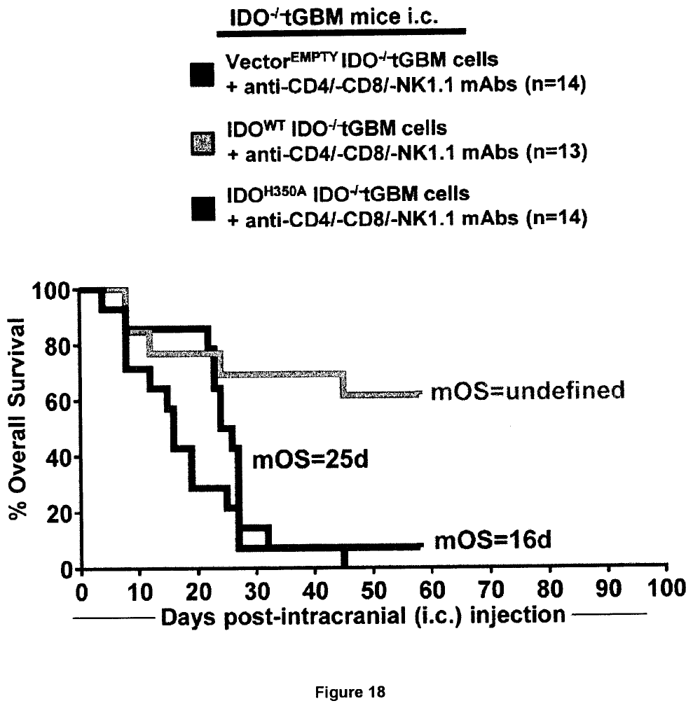

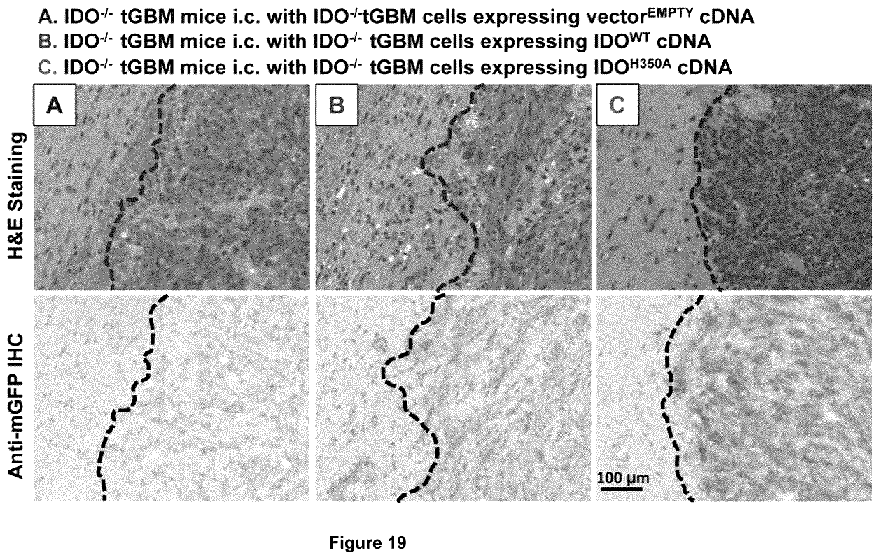

The in vivo role of Vector^{EMPTY}-, IDO^{WT}-, and IDO^{H350A}-expressing IDO^{-/-}tGBM cells was next characterised, after their intracranial injection into syngeneic IDO^{-/-}tGBM mice (FIG. 12A). Mice with intracranial IDO^{WT}- and IDO^{H350A}-expressing tumors have decreased overall survival with a median overall survival (mOS) of 67.5 days and undefined as compared to mice with Vector^{EMPTY}-expressing tumors, respectively (FIG. 12C). The percentage of mortalities due to IDO^{WT}- and IDO^{H350A} are not different from one another (P=0.294). To further address whether the survival difference is caused by IDO-mediated immunosuppression, this experiment was repeated in IDO^{-/-}tGBM mice treated with CD4⁺ T- and CD8⁺ T-cell depleting antibodies. T cell depletion decreases survival as compared to mice that are not depleted for those leukocytes with the fastest mortality rates in mice with intracranial Vector^{EMPTY} and IDO^{H350A}-expressing tGBM cells (FIG. 12C). Similar survival effects are also found in IDO^{-/-}tGBM mice with modified tumor cells and depleted for T cells and NK cells (FIG. 18). The phenotype of tumor infiltrating lymphocytes at 4 weeks post-injection show a similar increase of Treg levels in IDO^{WT}-expressing (25.06±6.67%) and IDO^{H350A}-expressing (25.05±4.03%) brain tumors as compared to mice engrafted with Vector^{EMPTY}-expressing tumors (5.89±3.25%, P<0.05) (FIG. 12D). The expression of IDO is stable in both the IDO^{WT}- and IDO^{H350A}-expressing brain tumors as compared to the Vector^{EMPTY}-expressing tumors that are absent for IDO expression in vivo (FIG. 19). The in vitro co-culture of both the IDO^{WT}- and IDO^{H350A}-expressing tumor cells with splenic CD11 b monocytes induce a greater number of CD11 b⁺Ly6c⁺Ly6g^{-/low} mature macrophages at 18.25±0.65% and 18.9±1.4%, respectively (FIG. 12E) as compared to only 12.45%±0.55 among macrophages co-cultured with Vector^{EMPTY}-expressing tumor cells (P<0.01) (FIG. 12E, left panel). The effects on macrophage differentiation are tumor cell IDO-dependent and tryptophan metabolism-independent (FIG. 12E, right panel).

### 4.5.2 IDO Enhances Complement Factor H Expression in Human GBM Cells

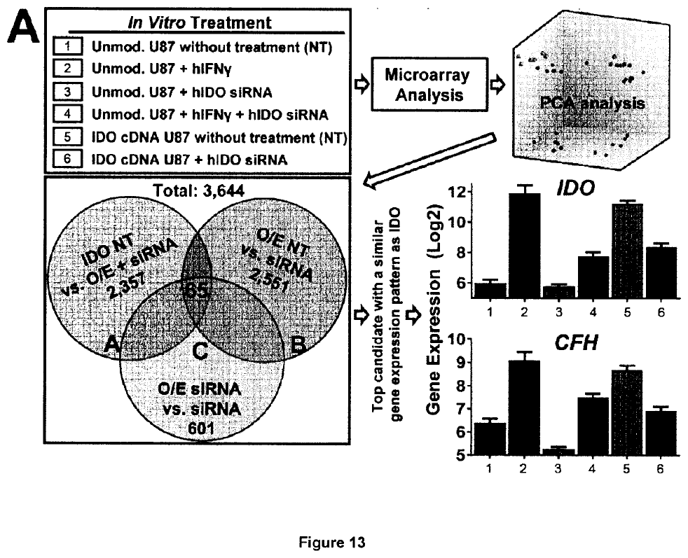

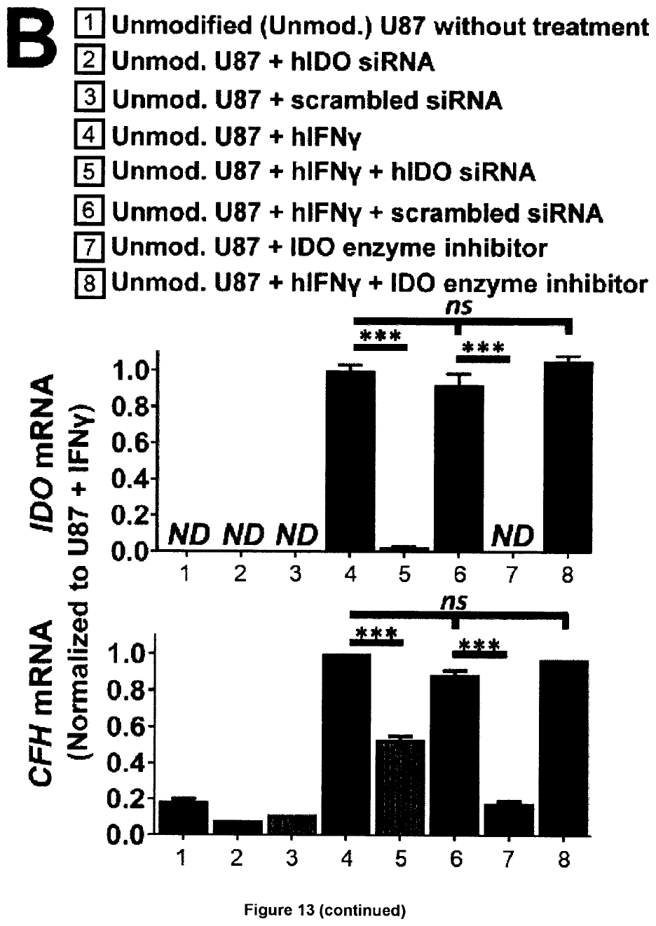

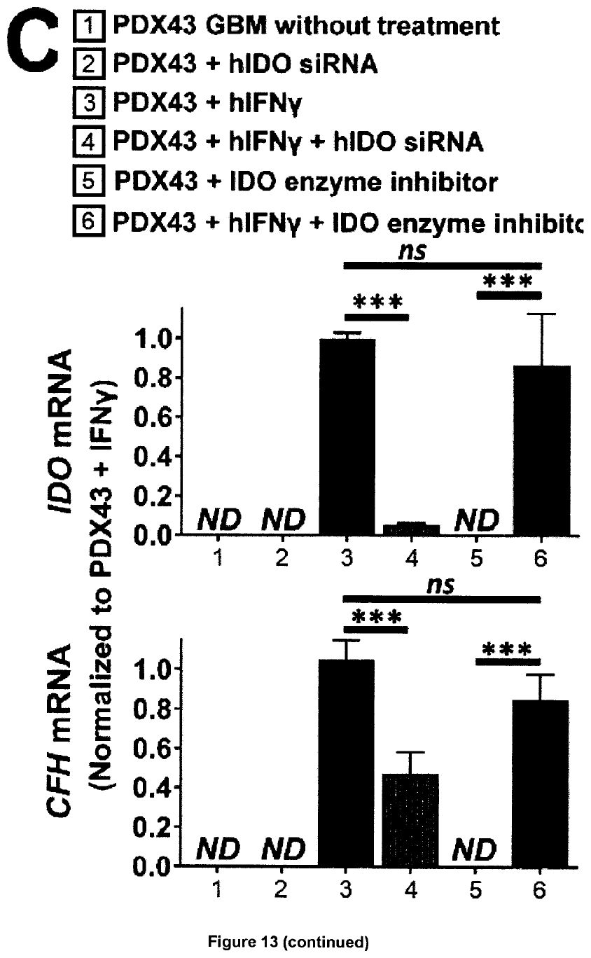

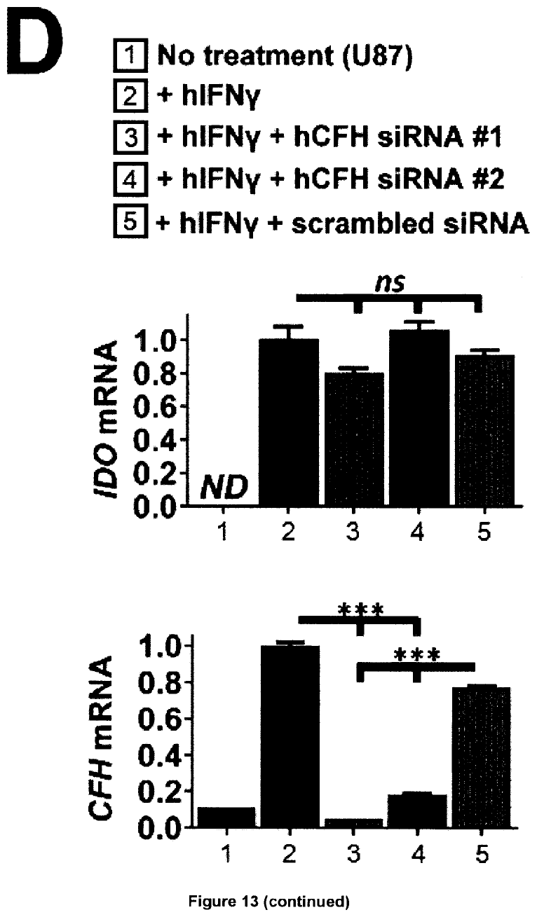

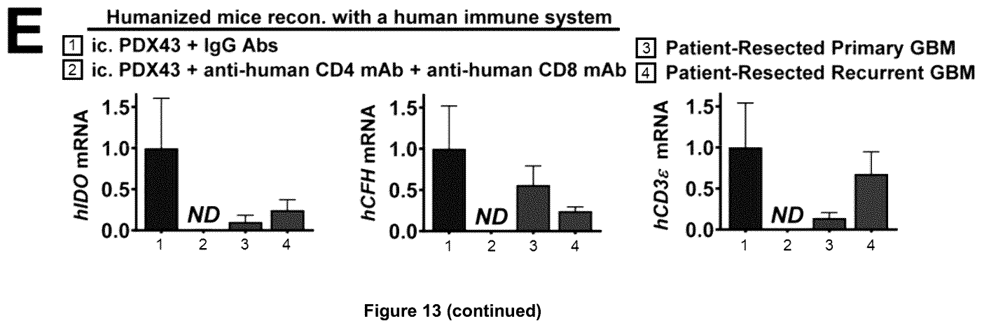

Since FIG. 12 collectively demonstrated that IDO enzyme activity does not fully account for its associated immunosuppressive and maladaptive effects in subjects with IDO-expressing glioma cells, the inventors next investigated the mechanism by which IDO facilitates non-enzyme-mediated effects. Utilizing the Clariom D microarray platform, unmodified human U87 GBM cells were either left untreated or treated with human interferon-gamma (IFNγ) and/or human IDO siRNA. A human U87 GBM cell line expressing wild-type human IDO cDNA described previously (11) was also treated with or without human IDO siRNA and all samples were analyzed collectively (FIG. 13A, top left). PCA analysis confirms the experimental reproducibility among the treatment conditions that were performed at different times and confirm the intra-group molecular similarity and inter-group molecular differences. Sixty-five gene candidates were identified based on their similar pattern of gene expression with IDO (FIG. 13A Venn diagram). Complement factor H (CFH) has the closest correlation with IDO gene expression changes in human GBM cells (FIG. 13A, bottom right). Quantitative RT-PCR confirms the IFNΓ-dependent increase of CFH expression, and in contrast, the decreased expression of CFH when IDO expression is either absent or inhibited with IDO-specific siRNA (FIG. 13B). The IDO-mediated enhancement of CFH expression is independent of Trp metabolism since the treatment of U87 cells with the previously characterised IDO enzyme inhibitor BGB-5777 (18), has no effect on CFH expression levels in U87 (FIG. 13B) and PDX43 (FIG. 13C) GBM cells. In contrast, the inhibition of CFH expression has no effect on IDO expression levels (FIG. 13D). Since previous work showed that human GBM-infiltrating T cells induce IDO expression in vitro and in vivo (11, 34), CFH expression levels were analyzed in human immune system-reconstituted humanized mice with intracranial human PDX43 and in patient-resected human GBM. Similar to intratumoral IDO and CD3 levels, human CFH expression is absent in PDX43 when engrafted into humanized mice that are co-depleted for human CD4′ and CD8⁺ T cells (FIG. 13E). Notably, the presence of IDO, CD3, and CFH are detectable in both newly-diagnosed and recurrent patient-resected GBM. These data collectively suggest that while tumor cell IDO and CFH are increased through a mechanism that depends on human tumor-infiltrating T cells, maximal CFH expression potential requires IDO-dependent tryptophan metabolism-independent effects.

### 4.5.3 IDO and CFH Demonstrate Similar Patterns of Expression in Patient-Resected GBM and Correlate with Glioma Patient Survival

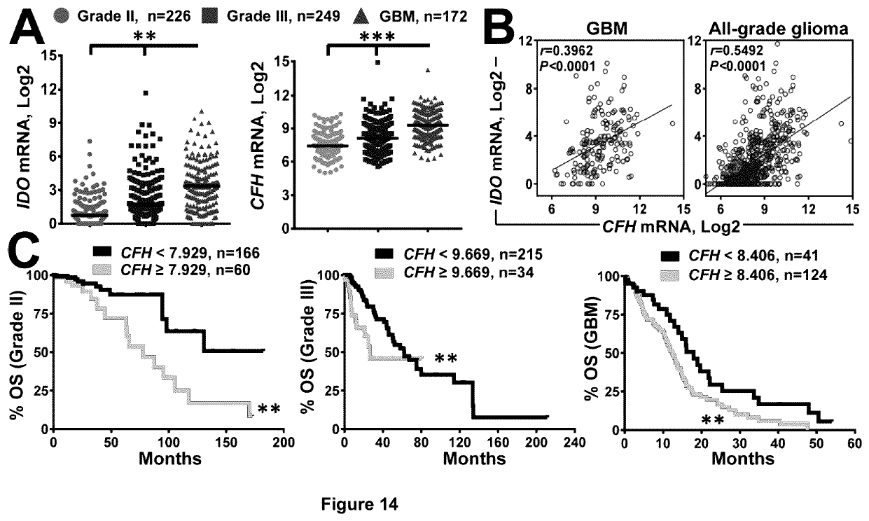

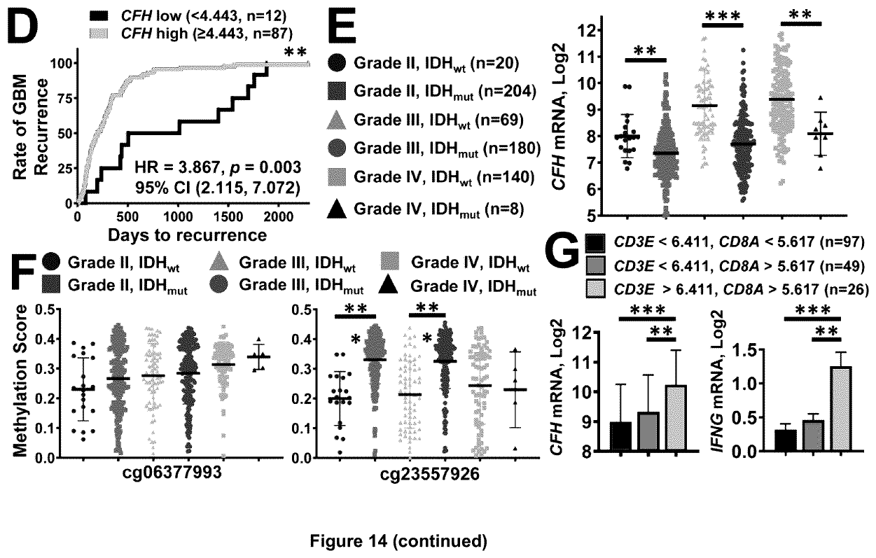

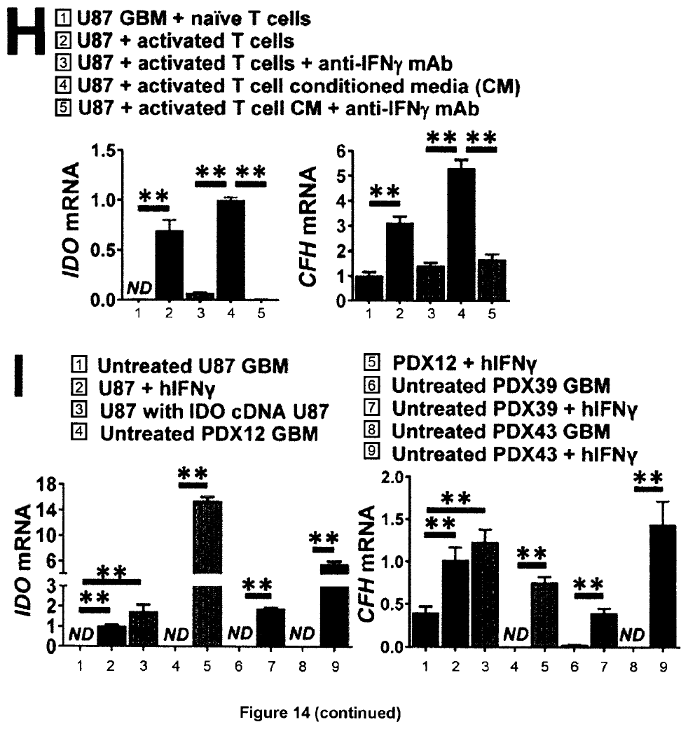

Based on the similar patterns of IDO and CFH expression in the established human U87 GBM cell line (FIG. 13B) and in human PDX43 GBM (FIG. 13C), the inventors next explored whether such a relationship exists in patient-resected GBM. FIG. 14A shows TCGA analysis of IDO and CFH mRNA levels, both of which progressively increase with glioma grade and are maximally expressed in GBM. Pearson's correlation analysis indicates that CFH and IDO mRNA levels positively correlate in patient-resected GBM (r=0.3962, P<0.0001) as well as when all grades of glioma are analyzed simultaneously (r=0.549, P<0.0001) (FIG. 14B). Kaplan-Meier analysis further demonstrates that higher CFH mRNA levels are inversely associated with glioma patient survival (FIG. 14C) and predictive of a faster rate to GBM recurrence (FIG. 14D). Intra-glioma CFH expression is also affected by isocitrate dehydrogenase (IDH) status such that a higher level of CFH expression is observed among wild-type IDH (wtIDH)-expressing tumors (within grade II gliomas, P=0.0037; within grade III gliomas, P<0.0001; within GBM, P=0.0056 (FIG. 14E). CFH methylation for the cg23557926 locus is significantly different among grade II and grade III wtIDH and mutant IDH (mIDH)-expressing glioma (P<0.0001) but is not significantly different within GBM (FIG. 14F). Consistent with the analysis of humanized mice with intracranial PDX43 (FIG. 13D), GBM samples with a higher mRNA profile indicative of CD8⁺ T cells (CD3E, CD8A) possess significantly higher levels of CFH and IFNγ expression (P<0.01) (FIG. 13D, FIG. 14G). To further understand the relationship of human T cells with IDO and CFH in human GBM, the co-culture of either naïve or activated T cells, conditioned media from activated T cells, and/or the treatment of anti-IFNγ was assessed in cultures of U87 GBM cells. FIG. 14H demonstrates that activated human T cells and the associated conditioned media containing human IFNγ directly induce both IDO and CFH gene expression. This observation was further extended with the in vitro culturing of U87, PDX12, PDX39, and PDX43 treated with or without human IFNγ (FIG. 14I). Under all conditions, IFNγ increased CFH expression in human GBM. Collectively, the data indicate that IDO and CFH coordinately increase in patient-resected glioma, that higher CFH expression is inversely associated with glioma patient survival regardless of tumor grade, and that IFNγ-expressing GBM-infiltrating T cells enhance the expression of intratumoral CFH levels.

### 4.5.4 IDO Enhances CFH Isoform Expression

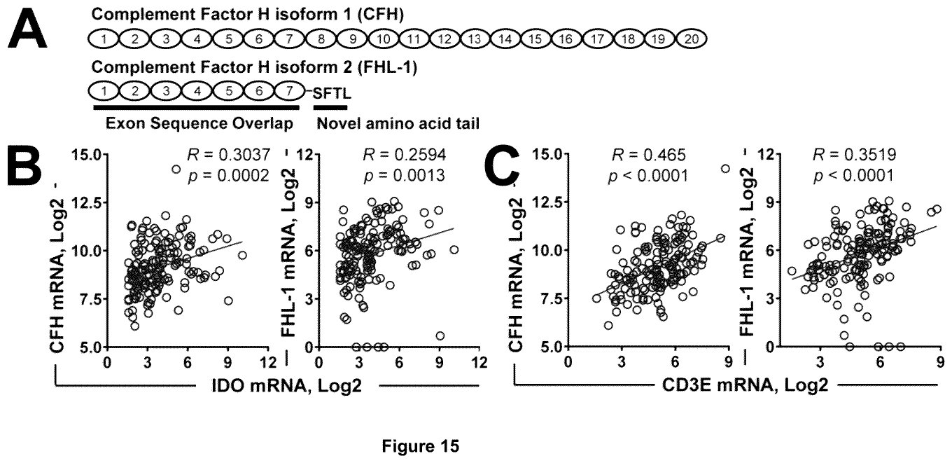

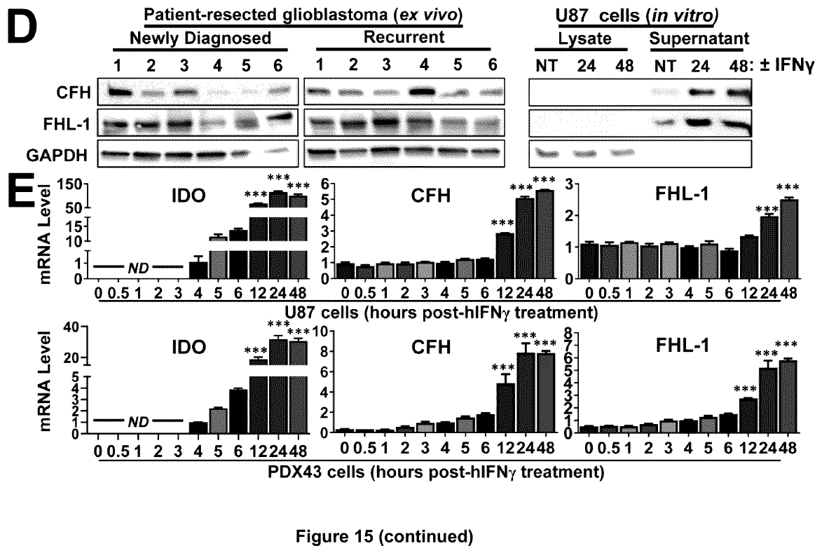

The human CFH gene locus is on chromosome 1q32 in the regulators of complement activation (RCA) gene cluster. CFH encodes for an ˜155 kDa secreted glycoprotein comprised of 20 contiguous complement control protein (CCP) modules (FIG. 15A, top panel) and a truncated splice variant referred to as factor H like protein 1 (FHL-1) that encodes a second isoform composed of CCPs 1-7 followed by a unique 4-amino acid sequence (FIG. 15A, lower panel) (35). Analysis of the TCGA indicates that both the full-length CFH and truncated CFH variant, FHL-1, positively correlate with IDO (FIG. 15B) and the T cell surface marker, CD3 (FIG. 15C), in patient-resected GBM. Both CFH isoforms are present in tumor lysate isolated from fresh patient-resected newly diagnosed GBM or recurrent GBM (FIG. 15D, left pane).

Protein expression for CFH and FHL-1 is also expressed in cell culture supernatants but not in intracellular U87 GBM cell lysate and is higher after treatment with IFNΓ (FIG. 15D, right panel). To further elucidate the dynamic mRNA expression of both CFH transcript variants as they relate to IDO levels over time, primers were designed for targeting the different CFH variants including the full-length variant by targeting CCPs 10-11 and the truncated variant by targeting the unique 4-amino acid sequence (FIG. 15A). IDO mRNA expression is detectable as early as 4 hours after treatment with IFNγ. In contrast, the earliest that the full-length and truncated CFH variants increased is at 12-hours post-IFNγ treatment in U87 (FIG. 15E, top row) and PDX43 (FIG. 15E, bottom row) GBM cells. The protein expression kinetic profile (FIG. 15F) is similar to the mRNA profile (FIG. 15E). To confirm that IDO possesses a direct regulatory effect on CFH expression, a homologously IDO-deleted U87 (IDOKO U87) cell line was created using the CRISPR-Cas9 gene editing approach (FIG. 15G). Whereas unmodified U87 treated with IFNγ expresses IDO protein and metabolizes Trp into Kyn, IDOKO U87 fails to express IDO protein and does not metabolize tryptophan. Strikingly, while IDO and both CFH splice variants are induced and upregulated after treatment with IFNγ in unmodified U87, respectively, no such increase takes place in IDOKO U87 cells (FIG. 15H). These data collectively confirm that upon stimulation with the T cell effector cytokine, IFNγ, the increase of CFH splice variant expression levels is dependent on the co-expression of IDO in human GBM cells.

### 4.5.5 Tumor Cell CFH Isoform Expression Enhances Intratumoral Immune Suppression and Decreases Survival in a Syngeneic Brain Tumor Model

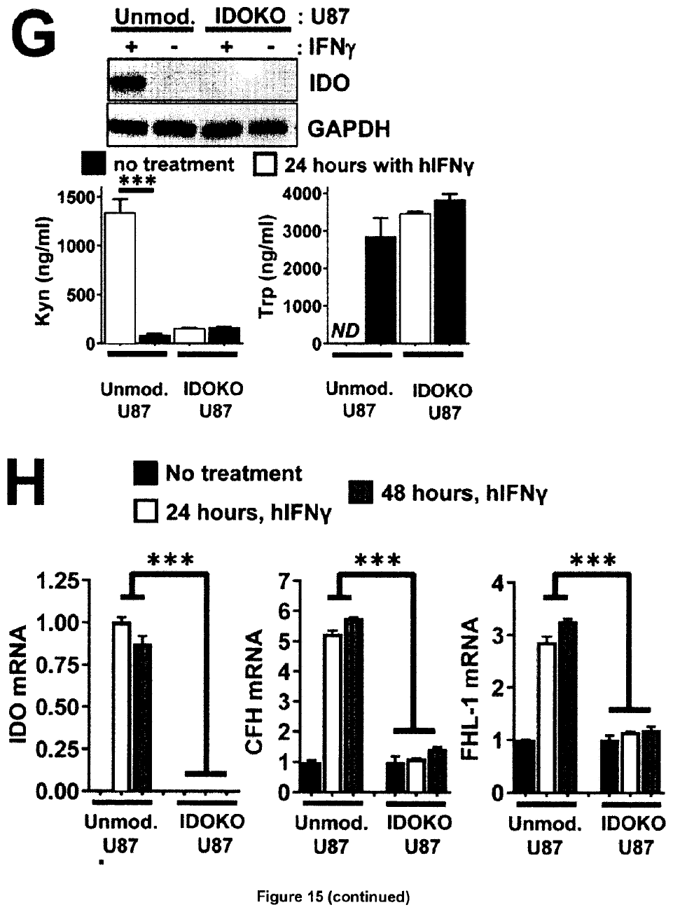

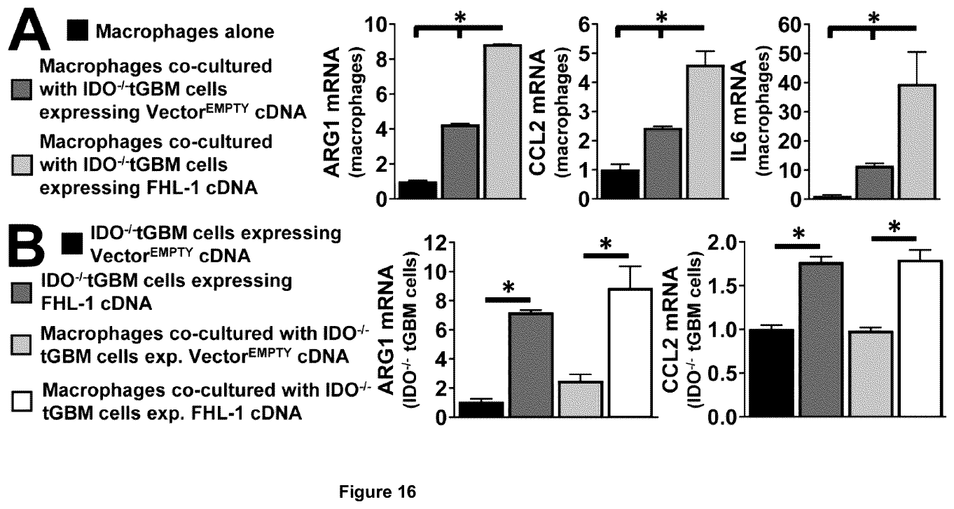

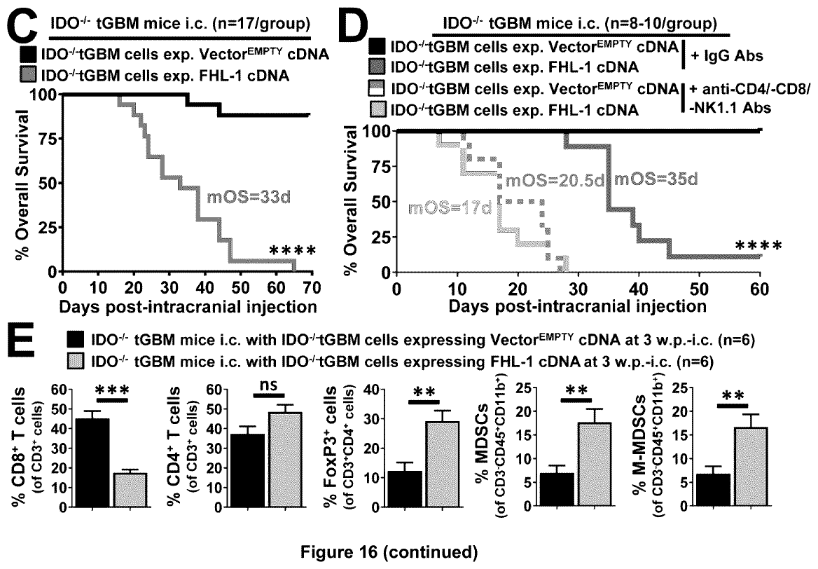

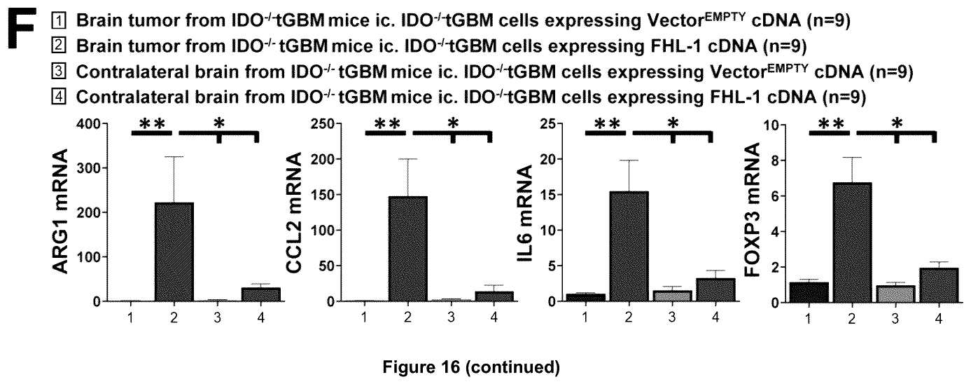

To better understand the downstream effects of IDO-enhanced tumor cell CFH expression, IDO^{-/-}tGBM cells were engineered to express the truncated CFH splice variant, FHL-1 cDNA (FIG. 12A). The co-culture of FHL-1-expressing tumor cells with splenic CD11 b macrophages leads to a maximal expression of ARG1, CCL2, and IL-6 in macrophages as compared to Vector^{EMPTY}-expressing cells that are co-cultured with macrophages or in macrophages cultured alone (P<0.05, FIG. 16A). FHL-1 expression also directly increases ARG1 and CCL2 levels in tumor cells (FIG. 16B) suggesting that FHL-1 binds to at least one receptor on macrophages and on tumor cells for carrying out its immunosuppressive gene reprogramming effects. The intracranial injection of FHL-1 cDNA-expressing IDO^{-/-}tGBM cells into syngeneic IDO^{-/-}tGBM mice leads to 100% mortality and a mOS of 33 days that is significantly lower than mice with brain tumor cells expressing the Vector^{EMPTY} (P<0.0001) (FIG. 16C). The survival benefit of mice with tumor cells expressing Vector^{EMPTY} and treated with a non-specific IgG antibody is reduced to a mOS of 20.5 days when T cells and NK cells are co-depleted (P<0.0001) (FIG. 16D). The decreased mOS of 35 days in mice with tumors expressing FHL-1 cDNA treated with non-specific IgG antibodies is also reduced to a mOS of 17 days in mice co-depleted for T and NK cells. Flow cytometric analysis of brain tumors isolated at 3 weeks post-intracranial injection show a marked decrease of tumor infiltrating CD8⁺ T cells (45.17%±3.78% versus 17.52%±−1.72%, P<0.001), an increase of Tregs (12.23%±2.79% versus 29.23%±3.535, P<0.01), and an increase of total MDSCs (CD3⁻CD45⁺CD11 b⁺Ly6C⁺) (6.98%±1.56% versus 17.71%±2.80%, P<0.01) that primarily reflected monocytic-type MDSCs (M-MDSCs: CD11 b⁺Ly6G⁻Ly6C^{hi}) in FHL-1-expressing tumors as compared to tumors expressing Vector^{EMPTY} (FIG. 16E). Gene expression analysis of FHL-1-expressing brain tumors isolated at 3 weeks post-intracranial injection showed an immunosuppressive signature with increases of ARG1, CCL2, IL-6, and Foxp3 expression as compared to Vector^{EMPTY}-brain tumors (P<0.01), as well as to contralateral non-tumor brain (P<0.05) (FIG. 16F). Collectively, the data show that when IDO-deficient brain tumor cells are genetically engineered to express a CFH variant, the resulting cells potently increase intratumoral immune suppression and decrease overall survival.

### 4.5.6 Circulating and Intratumoral CFH Correlations in Patients with GBM

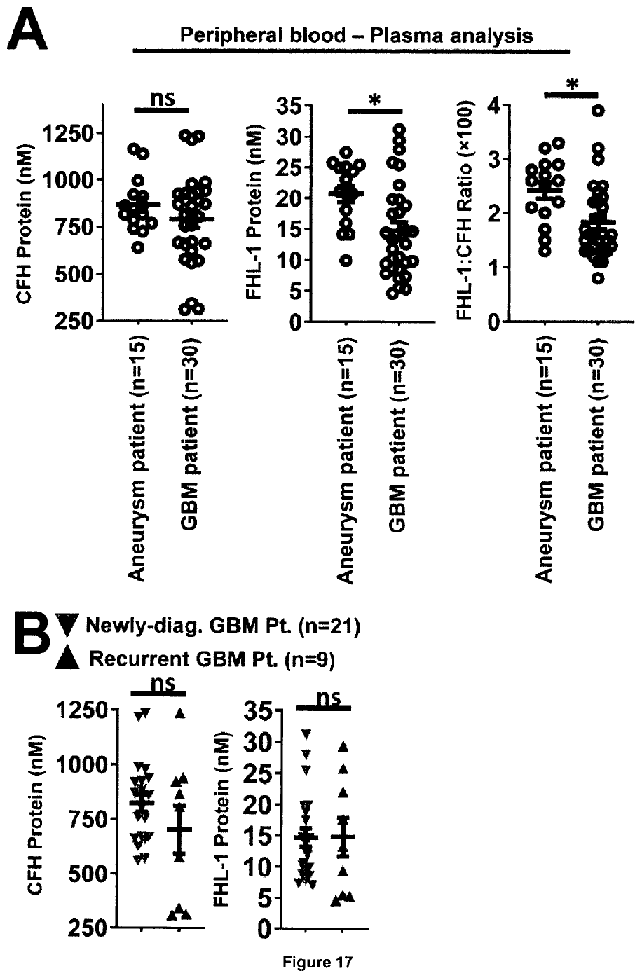

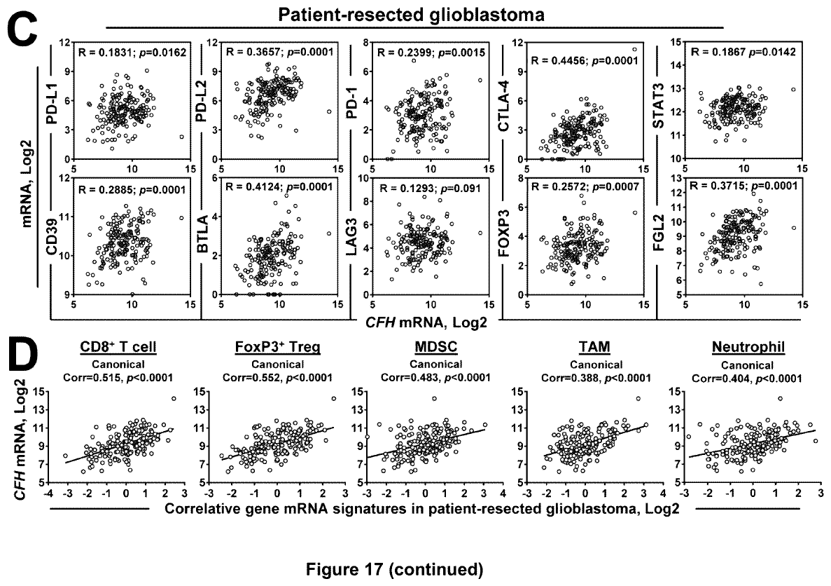

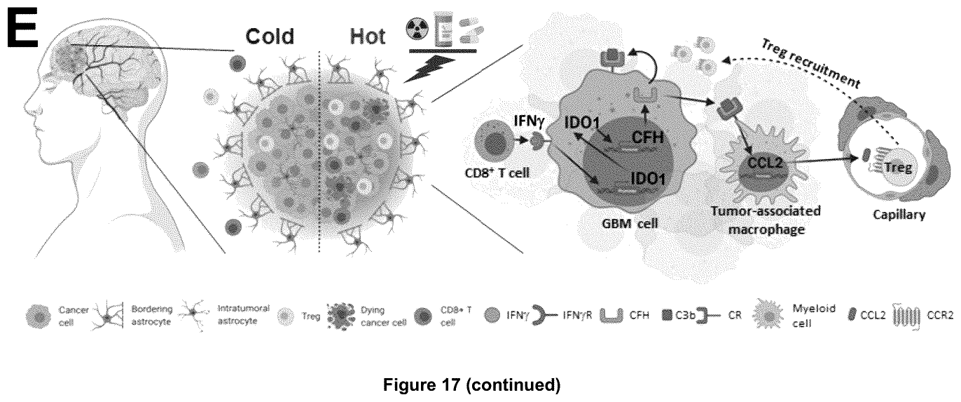

Since the two CFH isoforms are normally found in human plasma (35), the inventors next compared the protein levels for the full length and truncated CFH variants in non-tumor aneurysm- and age-matched GBM-patient plasma. FIG. 17A shows the systemic levels of CFH at 865 nM±37.42 nM and 788 nM±43.85 nM in plasma from aneurysm and GBM patients, respectively. It further shows the systemic levels for FHL-1 at 20.67 nM±1.34 nM in aneurysm patients that is decreased to 14.69±1.39 nM in GBM patients (P<0.05). The ratio of CFH:FHL-1 is also decreased in GBM patients as compared to the aneurysm control group (P<0.05, FIG. 17A). No difference was observed regarding systemic CFH and FHL-1 levels when comparing the plasma of newly diagnosed and recurrent GBM patients (FIG. 17B). Intratumorally, CFH expression positively correlates with mRNA levels for many other immunosuppressive modulators including PD-L1, PD-L2, PD-1, CTLA-4, LAG3, BTLA, and FGL2 in GBM (FIG. 17C). Notably, CFH also broadly correlates with mRNA signatures for infiltrating leukocytes. The data collectively suggest that there is a unique profile of CFH variant expression in the circulation as compared to the intratumoral CFH expression. Additionally, increases of intratumoral CFH expression positively correlate with increases for other molecular and cellular mediators of inflammation and immune suppression (FIG. 17D). A hypothetical model based on the data here and previously published shows how the CFH contributes to immunosuppressive Treg accumulation in GBM (FIG. 17E).

### 4.6 Discussion

The relationship between IDO and its regulatory effects on the complement cascade was initially described in the anatomical setting of placenta (36). At the time, there was no description of how treatment with the IDO pathway inhibitor, 1-methyl tryptophan (1-MT), affected intra-placental CFH expression levels or tryptophan and kynurenine levels. In a subsequent study, Li et al. demonstrated that pharmacologic IDO pathway modulation with either 1-MT or NLG919 triggered chemo-radiation-dependent complement C3 deposition at sites of tumor growth in the GL261-based mouse orthotopic brain tumor model (37). However, there was no description of how 1-MT or NLG919 affected intratumoral CFH expression levels or tryptophan and kynurenine levels. This is of significant notability since D1-MT, which has the most potent anti-brain tumor effects (17) and is used as the exclusive stereoisomer of 1-MT in clinical trials (15), does not effectively inhibit tryptophan metabolism (38, 39).

Complement C3 functions as a pivotal inducer by activating the complement-mediated inflammatory pathways, while CFH/FHL-1 plays a critical inhibitory role that suppresses complement-mediated inflammatory responses. With respect to the observations of our study, it's possible that the mechanistic effects of IDO enzyme activity on C3 activation are independent from those underlying the IDO non-enzyme effects on CFH/FHL-1 regulation. Since IDO protein is expressed in a majority of human cancers including GBM (13), further investigation that focuses on the molecular mechanism(s) underlying IDO regulation of C3 activation and CFH/FHL-1 is warranted. The present study, shows for the first time that human tumor cells utilize IDO non-enzyme activity to enhance the expression level of immunosuppressive CFH and its truncated isoform, FHL-1. It is further demonstrated that tumor cell FHL-1: (i) enhanced macrophage maturity, (ii) enhanced macrophage expression for ARG1, CCL2, and IL-6, and (iii) decreased the survival of mice with brain tumors in-part by suppressing the anti-GBM T and NK cell immune response. Translationally-relevant, it is also shown that IDO and CFH expression are positively correlated in patient-resected GBM and that increased intratumoral CFH/FHL-1 levels are associated with decreased GBM patient survival. This study therefore contributes a mechanistic understanding for why pharmacologic IDO enzyme inhibitor treatment fails to reverse the immunosuppressive effects of IDO when administered as a monotherapy (15).

Questions regarding the immunosuppressive role of CFH/FHL-1 remain to be addressed in the setting of GBM. First, does full-length CFH play an identically immune tolerant role as the isoform, FHL-1? Previous work showed that CFH-treated monocyte-derived dendritic cells (MoDCs) had a tolerogenic state, such as the production of immunomodulatory mediators including IL-10 and TGF-β, a reduced expression for CCR7 and chemotactic migration, impaired CD4⁺ T cell alloproliferation, and an induction of CD4⁺CD127^{low/-}CD25^{high}Foxp3⁺ regulatory T cells (40). One future direction to investigate how CFH/FHL-1 regulates complement pathway activation in GBM. However, as demonstrated in FIG. 17E and by Olivar et al., CFH/FHL-1 may possess immunomodulatory activities that are independent of complement regulation, raising important considerations for further mechanistic study (40).

Microarray analysis discovered a similar pattern of regulation between IDO and CFH in human GBM, in vitro, and this effect was confirmed in patient-resected tumors, in vivo. It is further shown that both the full length CFH and truncated variant, FHL-1, suppress the immune response in GBM. Although both CFH and FHL-1 have been previously associated with mechanisms of immune evasion (41, 42), no previous investigation focused on the role of FHL-1 in tumor-induced immunosuppression. Interestingly, FHL-1 is not expressed by mice (43) which allowed us to ignore the potential for multi-species gene expression competition of similar protein product during our investigation of IDO^{-/-}tGBM cells expressing human FHL-1 cDNA. In summary, the inventors have revealed a non-enzymic function of IDO in human tumor cells that non-metabolically increases immunosuppression and contributes to poorer survival outcomes.

### 4.7 References for Example 4

1. Adamson C, Kanu O O, Mehta Al, Di C, Lin N, Mattox A K, et al. Glioblastoma multiforme: a review of where we have been and where we are going. Expert Opin Investig Drugs. 2009; 18:1061-83.
2. Stupp R, Mason W P, van den Bent M J, Weller M, Fisher B, Taphoorn M J, et al. Radiotherapy plus concomitant and adjuvant temozolomide for glioblastoma. N Engl J Med. 2005; 352:987-96.
3. Ries L A G S M, Gurney J G, Linet M, Tamra T, Young J L, Bunin G R. Cancer Incidence and Survival among Children and Adolescents: United States SEER Program 1975-1995. Bethesda, MD: National Cancer Institute, SEER Program; 1999.
4. Couzin-Frankel J. Breakthrough of the year 2013. Cancer immunotherapy. Science. 2013; 342:1432-3.
5. Kong D-S, Nam D-H, Kang S-H, Lee J W, Chang J-H, Kim J-H, et al. Phase III randomized trial of autologous cytokine-induced killer cell immunotherapy for newly diagnosed glioblastoma in Korea. Oncotarget. 2017; 8:7003-13.
6. Liau L M, Ashkan K, Tran D D, Campian J L, Trusheim J E, Cobbs C S, et al. First results on survival from a large Phase 3 clinical trial of an autologous dendritic cell vaccine in newly diagnosed glioblastoma. J Trans Med. 2018; 16:142.
7. Reardon D A, Brandes A A, Omuro A, Mulholland P, Lim M, Wick A, et al. Effect of Nivolumab vs Bevacizumab in Patients With Recurrent Glioblastoma: The CheckMate 143 Phase 3 Randomized Clinical Trial. JAMA Oncol. 2020; 6:1003-10.
8. Uyttenhove C, Pilotte L, Theate I, Stroobant V, Colau D, Parmentier N, et al. Evidence for a tumoral immune resistance mechanism based on tryptophan degradation by indoleamine 2,3-dioxygenase. Nat Med. 2003; 9:1269-74.
9. Shimizu T, Nomiyama S, Hirata F, Hayaishi O. Indoleamine 2,3-dioxygenase. Purification and some properties. J Bio Chem. 1978; 253:4700-6.
10. Wainwright D A, Balyasnikova I V, Chang A L, Ahmed A U, Moon K S, Auffinger B, et al. IDO expression in brain tumors increases the recruitment of regulatory T cells and negatively impacts survival. Clin Cancer Res. 2012; 18:6110-21.
11. Zhai L, Ladomersky E, Lauing K L, Wu M, Genet M, Gritsina G, et al. Infiltrating T cells increase IDO1 expression in glioblastoma and contribute to decreased patient survival. Clin Cancer Res. 2017; 23:11.
12. Zhai L, Ladomersky E, Lenzen A, Nguyen B, Patel R, Lauing K L, et al. IDO1 in cancer: a Gemini of immune checkpoints. Cell Mol Immunol. 2018; 15:447-57.
13. Uyttenhove C, Pilotte L, Theate I, Stroobant V, Colau D, Parmentier N, et al. Evidence for a tumoral immune resistance mechanism based on tryptophan degradation by indoleamine 2,3-dioxygenase. Nature Med. 2003; 9:1269-74.
14. Zhai L, Lauing K L, Chang A L, Dey M, Qian J, Cheng Y, et al. The role of IDO in brain tumor immunotherapy. J Neurooncol. 2015; 123:395-403.
15. Zhai L, Spranger S, Binder D C, Gritsina G, Lauing K L, Giles F J, et al. Molecular Pathways: Targeting IDO1 and Other Tryptophan Dioxygenases for Cancer Immunotherapy. Clin Cancer Res. 2015; 21:5427-33.
16. Muller A J, Manfredi M G, Zakharia Y, Prendergast G C. Inhibiting IDO pathways to treat cancer: lessons from the ECHO-301 trial and beyond. Semin Immunopath. 2019; 41:41-8.
17. Wainwright D A, Chang A L, Dey M, Balyasnikova I V, Kim C K, Tobias A, et al. Durable therapeutic efficacy utilizing combinatorial blockade against IDO, CTLA-4, and PD-L1 in mice with brain tumors. Clin Cancer Res. 2014; 20:5290-301.
18. Ladomersky E, Zhai L, Lenzen A, Lauing K L, Qian J, Scholtens D M, et al. IDO1 Inhibition synergizes with radiation and PD-1 blockade to durably increase survival against advanced glioblastoma. Clin Cancer Res. 2018; 24:2559-73.
19. Ladomersky E, Zhai L, Lauing K L, Bell A, Xu J, Kocherginsky M, et al. Advanced age increases immunosuppression in the brain and decreases immunotherapeutic efficacy in subjects with glioblastoma. Clin Cancer Res. 2020; 26:5232-45.
20. Zhai L, Bell A, Ladomersky E, Lauing K L, Bollu L, Sosman J A, et al. Immunosuppressive IDO in Cancer: Mechanisms of Action, Animal Models, and Targeting Strategies. Frontiers in Immunology. 2020; 11:1185.
21. Zhai L, Ladomersky E, Dostal C R, Lauing K L, Swoap K, Billingham L K, et al. Non-tumor cell IDO1 predominantly contributes to enzyme activity and response to CTLA-4/PD-L1 inhibition in mouse glioblastoma. Brain, Behav Immun. 2017; 62:24-9.
22. Hashizume R, Ozawa T, Dinca E B, Banerjee A, Prados M D, James C D, et al. A human brainstem glioma xenograft model enabled for bioluminescence imaging. J Neurooncol. 2010; 96:151-9.
23. Zhai L, Ladomersky E, Dostal C R, Lauing K L, Swoap K, Billingham L K, et al. Non-tumor cell IDO1 predominantly contributes to enzyme activity and response to CTLA-4/PD-L1 inhibition in mouse glioblastoma. Brain, Behav Immun. 2017; 62:6.
24. Giannini C, Sarkaria J N, Saito A, Uhm J H, Galanis E, Carlson B L, et al. Patient tumor EGFR and PDGFRA gene amplifications retained in an invasive intracranial xenograft model of glioblastoma multiforme. Neuro-Oncology. 2005; 7:164-76.
25. Sarkaria J N, Carlson B L, Schroeder M A, Grogan P, Brown P D, Giannini C, et al. Use of an orthotopic xenograft model for assessing the effect of epidermal growth factor receptor amplification on glioblastoma radiation response. Clin Cancer Res. 2006; 12:2264-71.
26. Chow L M, Endersby R, Zhu X, Rankin S, Qu C, Zhang J, et al. Cooperativity within and among Pten, p53, and Rb pathways induces high-grade astrocytoma in adult brain. Cancer Cell. 2011; 19:305-16.
27. Wainwright D A, Balyasnikova I V, Chang A L, Ahmed A U, Moon K-S, Auffinger B, et al. IDO Expression in Brain Tumors Increases the Recruitment of Regulatory T Cells and Negatively Impacts Survival. Clin Cancer Res. 2012; 18:6110-21.
28. Efron B, Tibshirani R. Empirical bayes methods and false discovery rates for microarrays. Genetic Epidemiology. 2002; 23:70-86.
29. Livak K J, Schmittgen T D. Analysis of relative gene expression data using real-time quantitative PCR and the 2(-Delta Delta C(T)) Method. Methods. 2001; 25:402-8.
30. Zhai L, Dey M, Lauing K L, Gritsina G, Kaur R, Lukas R V, et al. The kynurenine to tryptophan ratio as a prognostic tool for glioblastoma patients enrolling in immunotherapy. J Clin Neurosci. 2015.
31. Cipriani V, Tierney A, Griffiths J R, Zuber V, Sergouniotis P I, Yates J R W, et al. Beyond factor H: The impact of genetic-risk variants for age-related macular degeneration on circulating factor-H-like 1 and factor-H-related protein concentrations. Am J Hum Genet. 2021; 108:1385-400.
32. Budczies J, Klauschen F, Sinn B V, Gyorffy B, Schmitt W D, Darb-Esfahani S, et al. Cutoff Finder: a comprehensive and straightforward Web application enabling rapid biomarker cutoff optimization. PloS one. 2012; 7:e51862.
33. Littlejohn T K, Takikawa O, Truscott R J W, Walker M J. Asp274 and His346 Are Essential for Heme Binding and Catalytic Function of Human Indoleamine 2,3-Dioxygenase. Journal of Biological Chemistry. 2003; 278:29525-31.
34. O'Rourke D M, Nasrallah M P, Desai A, Melenhorst J J, Mansfield K, Morrissette J J D, et al. A single dose of peripherally infused EGFRvIII-directed CAR T cells mediates antigen loss and induces adaptive resistance in patients with recurrent glioblastoma. Sci Transl Med. 2017; 9.
35. Parente R, Clark S J, Inforzato A, Day A J. Complement factor H in host defense and immune evasion. Cellular and Molecular Life Sciences. 2017; 74:1605-24.
36. Munn D H, Zhou M, Attwood J T, Bondarev I, Conway S J, Marshall B, et al. Prevention of allogeneic fetal rejection by tryptophan catabolism. Science (New York, NY). 1998; 281:1191-3.
37. Li M, Bolduc A R, Hoda M N, Gamble D N, Dolisca S B, Bolduc A K, et al. The indoleamine 2,3-dioxygenase pathway controls complement-dependent enhancement of chemo-radiation therapy against murine glioblastoma. Journal for immunotherapy of cancer. 2014; 2:21.
38. Lob S, Konigsrainer A, Zieker D, Brucher B L, Rammensee H G, Opelz G, et al. IDO1 and IDO2 are expressed in human tumors: levo- but not dextro-1-methyl tryptophan inhibits tryptophan catabolism. Cancer immunology, immunotherapy: CII. 2009; 58:153-7.
39. Qian F, Liao J, Villella J, Edwards R, Kalinski P, Lele S, et al. Effects of 1-methyltryptophan stereoisomers on IDO2 enzyme activity and IDO2-mediated arrest of human T cell proliferation. Cancer immunology, immunotherapy: CII. 2012; 61:2013-20.
40. Olivar R, Luque A, Cárdenas-Brito S, Naranjo-Gómez M, Blom A M, Borràs F E, et al. The Complement Inhibitor Factor H Generates an Anti-Inflammatory and Tolerogenic State in Monocyte-Derived Dendritic Cells. The Journal of Immunology. 2016; 196:4274.
41. Junnikkala S, Hakulinen J, Jarva H, Manuelian T, Bjorge L, Butzow R, et al. Secretion of soluble complement inhibitors factor H and factor H-like protein (FHL-1) by ovarian tumour cells. British journal of cancer. 2002; 87:1119-27.
42. Junnikkala S, Jokiranta T S, Friese M A, Jarva H, Zipfel P F, Meri S. Exceptional Resistance of Human H2 Glioblastoma Cells to Complement-Mediated Killing by Expression and Utilization of Factor H and Factor H-Like Protein 1. The Journal of Immunology. 2000; 164:6075-81.
43. Pouw R B, Vredevoogd D W, Kuijpers T W, Wouters D. Of mice and men: The factor H protein family and complement regulation. Molecular Immunology. 2015; 67:12-20.

### Example 5: Analysis of Complement Proteins in the Blood of COVID-19 Patients

In further experiments, the inventors investigated the levels of complement proteins CFH, FHL1, FHR1, FHR2, FHR3, FHR4 and FHR5 in samples of blood obtained from 200 COVID-19 patients having varying severity of disease, and in samples of blood obtained from healthy control subjects (not having COVID-19). Blood samples were obtained from subjects in April-July 2020 at the time of the first COVID test. Progression of infection and clinical severity were monitored, with patients subsequently falling into one of five groups: asymptomatic disease (A), mild disease (B), disease requiring hospitalization but not supplemental oxygen (C), disease requiring hospitalization and low flow supplemental oxygen (D), disease requiring assisted ventilation (E). Detection of FH, FHL1, FHR1, FHR2, FHR3, FHR4 and FHR5 in samples from each group was performed using mass spectrometry as described in Examples 1 and 2.

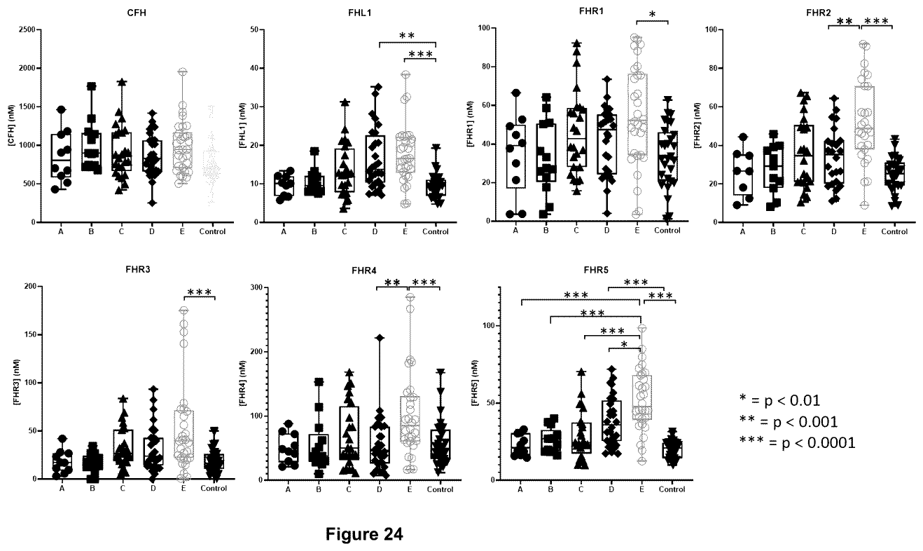

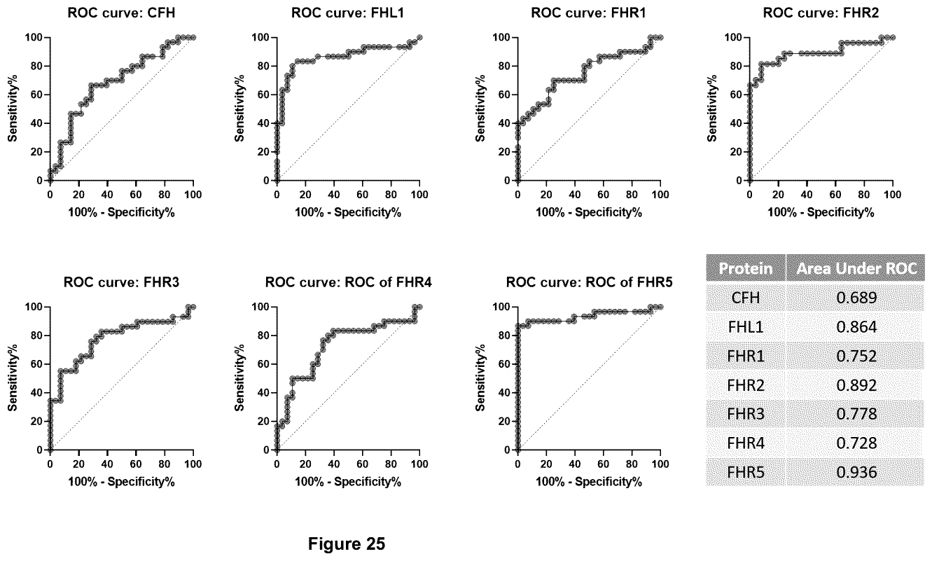

The results are shown in FIGS. 24 and 25. Statistically-significant elevations (one-way ANOVA) in the levels of FHL1, FHR1, FHR2, FHR3, FHR4 and FHR5 were detected in the blood of COVID-19 patients having severe disease requiring assisted ventilation (group E), relative to levels in the blood of healthy control subjects. There was a general trend towards elevated levels of FHL1, FHR1, FHR2, FHR3, FHR4 and FHR5 with increasing COVID-19 severity. FIG. 25 shows the comparison of group E patients with uninfected controls. ROC curves were produced using GraphPad Prism v8. Predictive ability was most pronounced for FHR5, where statistically-significant differences were observed between COVID-19 patients having differing disease severity, although all proteins tested were capable of discriminating between subjects with severe COVID-19 infection and control subjects.

1.-26. (canceled)

27. A method of treating a macular degeneration in a subject, the method comprising:
(a) determining in a blood-derived or liver sample obtained from the subject the level of one or more of FHR1, FHR2, FHR3, and/or FHR5, optionally in combination with FHR4, FHL-1, and/or FH;
(b) determining that the subject has or is likely to develop a macular degeneration if the level of the protein(s) in (a) is elevated as compared to the level of that protein(s) in blood or liver tissue in a control subject that does not have a macular degeneration; and
(c) treating the subject with a therapeutic agent that comprises or encodes a polypeptide comprising an amino acid sequence with at least 85% sequence identity to SEQ ID NO:146, wherein the polypeptide has a total length of 450 amino acids or fewer; and/or treating the subject with a nucleic acid agent that reduces expression of one or more of FHR1, FHR2, FHR3, FHR4, and/or FHR5.

28. The method according to claim 27, wherein the macular degeneration is selected from Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, and autoimmune uveitis.

29. The method according to claim 27, wherein the nucleic acid agent is an siRNA, miRNA, shRNA, antisense oligonucleotide, or gapmer.

30. The method according to claim 27, wherein the level of the one or more protein(s) is determined by mass spectrometry.

31. The method according to claim 27, wherein step (a) comprises:
(i) digesting at least one of the protein(s) in the sample obtained from the subject with endoproteinase GluC to obtain one or more peptides;
(ii) performing mass spectrometry to determine the presence and/or level of the one or more peptides; and
(iii) using the results of (ii) to determine if the level of the protein(s) is elevated.

32. The method according to claim 27, wherein step (a) comprises determining the level of two or more of FHR1, FHR2, FHR5 and/or FHR3.

33. The method according to claim 32, wherein step (a) further comprises determining the level of FHR4.

34. The method according to claim 27, wherein the method further comprises determining the level of FHL-1 and/or FH, and optionally determining that the subject has or is likely to develop a macular degeneration if the level of FHL-1 is elevated as compared to the level of FHL-1 in blood from a control subject that does not have a macular degeneration.

35. The method according to claim 27, wherein the method comprises determining the level of:
(i) FHR1 and FHR2;
(ii) FHR2 and FHR3;
(iii) FHR1 and FHR3;
(iv) FHR1, FHR2, and FHR3;
(v) FHR1, FHR2, and FHR5;
(vi) FHR1, FHR2, FHR3, and FHR4;
(vii) FHR1, FHR2, FHR3, and FHR5;
(viii) FHR1, FHR2, FHR3, FHR4, and FHR5; or
(ix) any of (i) to (viii) above in combination with FHL-1 and/or FH.

36. A method of treating a macular degeneration in a subject, the method comprising administering to the subject a therapeutic agent that comprises or encodes a polypeptide comprising an amino acid sequence with at least 85% sequence identity to SEQ ID NO:146, wherein the polypeptide has a total length of 450 amino acids or fewer; and/or administering to the subject a nucleic acid agent that reduces expression of one or more of FHR1, FHR2, FHR3, FHR4, and/or FHR5;
wherein the subject has been determined to have or be likely to have a macular degeneration by:
(a) determining in a blood-derived or liver sample obtained from the subject the level of one or more of FHR1, FHR2, FHR3, and/or FHR5, optionally in combination with FHR4, FHL-1 and/or FH;
(b) determining that the subject has or is a likely to have a macular degeneration if the level of the protein(s) in (a) is elevated as compared to the level of that protein(s) in blood or liver tissue in a control subject that does not have a macular degeneration.

37. The method according to claim 36, wherein the macular degeneration is selected from Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, and autoimmune uveitis.

38. The method according to claim 36, wherein the nucleic acid agent is an siRNA, miRNA, shRNA, antisense oligonucleotide, or gapmer.

39. The method according to claim 36, wherein step (a) comprises determining the level of two or more of FHR1, FHR2, FHR5, and/or FHR3.

40. The method according to claim 39, wherein step (a) further comprises determining the level of FHR4.

41. The method according to claim 36, wherein the method further comprises determining the level of FHL-1 and/or FH, optionally determining that the subject has or is likely to develop a macular degeneration if the level of FHL-1 is elevated as compared to the level of FHL-1 in blood in a control subject that does not have a macular degeneration.

42. The method according to claim 36, wherein the method comprises determining the level of:
(i) FHR1 and FHR2;
(ii) FHR2 and FHR3;
(iii) FHR1 and FHR3;
(iv) FHR1, FHR2, and FHR3;
(v) FHR1, FHR2, and FHR5;
(vi) FHR1, FHR2, FHR3, and FHR4;
(vii) FHR1, FHR2, FHR3, and FHR5;
(viii) FHR1, FHR2, FHR3, FHR4, and FHR5; or
(ix) any of (i) to (viii) above in combination with FHL-1 and/or FH.

43. The method according to claim 36, wherein the level of the one or more protein(s) is determined by mass spectrometry.

44. The method according to claim 36, wherein step (a) comprises:
(i) digesting at least one of the protein(s) in the sample obtained from the subject with endoproteinase GluC to obtain one or more peptides;
(ii) performing mass spectrometry to determine the presence and/or level of the one or more peptides; and
(iii) using the results of (ii) to determine if the level of the protein(s) is elevated.


SEQUENCE LISTING (175 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 3 - protein, 330 aa, synthetic construct
  REGION 1..330: note = synthetic construct
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 4 - protein, 270 aa, synthetic construct
  REGION 1..270: note = synthetic construct
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 5 - protein, 243 aa, synthetic construct
  REGION 1..243: note = synthetic construct
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTSSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 6 - protein, 330 aa, synthetic construct
  REGION 1..330: note = synthetic construct
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 7 - protein, 269 aa, synthetic construct
  REGION 1..269: note = synthetic construct
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVNSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 8 - protein, 540 aa, synthetic construct
  REGION 1..540: note = synthetic construct
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYA

SEQ ID NO: 9 - protein, 331 aa, synthetic construct
  REGION 1..331: note = synthetic construct
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 10 - protein, 569 aa, synthetic construct
  REGION 1..569: note = synthetic construct
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 11 - protein, 583 aa, synthetic construct
  REGION 1..583: note = synthetic construct
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 12 - protein, 1663 aa, synthetic construct
  REGION 1..1663: note = synthetic construct
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 13 - protein, 645 aa, Homo sapiens
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 14 - protein, 915 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 15 - protein, 77 aa, Homo sapiens
SVQLTEKRMDKVGKYPKELRKCCEDGMRENPMRFSCQRRTRFISLGEACKKVFLDCCNYITELRRQHARASHLGLAR

SEQ ID NO: 16 - protein, 915 aa, Homo sapiens; identical to SEQ ID NO: 14

SEQ ID NO: 17 - protein, 343 aa, Homo sapiens
SEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 18 - protein, 17 aa, synthetic construct
  REGION 1..17: note = synthetic construct
SSKITHRIHWESASLLR

SEQ ID NO: 19 - protein, 206 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGR

SEQ ID NO: 20 - protein, 7 aa, synthetic construct
  REGION 1..7: note = synthetic construct
VTYKCFE

SEQ ID NO: 21 - protein, 16 aa, synthetic construct
  REGION 1..16: note = synthetic construct
NGWSPTPRCIRVSFTL

SEQ ID NO: 22 - protein, 18 aa, synthetic construct
  REGION 1..18: note = synthetic construct
ATFCDFPKINHGILYDEE

SEQ ID NO: 23 - protein, 16 aa, synthetic construct
  REGION 1..16: note = synthetic construct
RGWSTPPKCRSTISAE

SEQ ID NO: 24 - protein, 18 aa, synthetic construct
  REGION 1..18: note = synthetic construct
AMFCDFPKINHGILYDEE

SEQ ID NO: 25 - protein, 20 aa, synthetic construct
  REGION 1..20: note = synthetic construct
VACHPGYGLPKAQTTVTCTE

SEQ ID NO: 26 - protein, 8 aa, synthetic construct
  REGION 1..8: note = synthetic construct
YQCQSYYE

SEQ ID NO: 27 - protein, 15 aa, synthetic construct
  REGION 1..15: note = synthetic construct
RGWSTPPICSFTKGE

SEQ ID NO: 28 - protein, 13 aa, synthetic construct
  REGION 1..13: note = synthetic construct
GTAFVIFGIQDGE

SEQ ID NO: 29 - protein, 20 aa, synthetic construct
  REGION 1..20: note = synthetic construct
LRRQHARASHLGLARSNLDE

SEQ ID NO: 30 - protein, 15 aa, synthetic construct
  REGION 1..15: note = synthetic construct
LRRQHARASHLGLAR

SEQ ID NO: 31 - protein, 23 aa, synthetic construct
  REGION 1..23: note = synthetic construct
LNLDVSLQLPSRSSKITHRIHWE

SEQ ID NO: 32 - protein, 12 aa, synthetic construct
  REGION 1..12: note = synthetic construct
LNLDVSLQLPSR

SEQ ID NO: 33 - protein, 5 aa, synthetic construct
  REGION 1..5: note = synthetic construct
RLGRE

SEQ ID NO: 34 - protein, 11 aa, synthetic construct
  REGION 1..11: note = synthetic construct
SSKITHRIHWE

SEQ ID NO: 35 - protein, 6 aa, synthetic construct
  REGION 1..6: note = synthetic construct
SASLLR

SEQ ID NO: 36 - protein, 4 aa, synthetic construct
  REGION 1..4: note = synthetic construct
RLGR

SEQ ID NO: 37 - protein, 11 aa, synthetic construct
  REGION 1..11: note = synthetic construct
HLIVTPSGCGE

SEQ ID NO: 38 - protein, 39 aa, synthetic construct
  REGION 1..39: note = synthetic construct
CAGTYDGSIDACKGDSGGPLVCMDANNVTYVWGVVSWGE

SEQ ID NO: 39 - protein, 36 aa, synthetic construct
  REGION 1..36: note = synthetic construct
GTCVCKLPYQCPKNGTAVCATNRRSFPTYCQQKSLE

SEQ ID NO: 40 - protein, 29 aa, synthetic construct
  REGION 1..29: note = synthetic construct
FPGVYTKVANYFDWISYHVGRPFISQYNV

SEQ ID NO: 41 - protein, 31 aa, synthetic construct
  REGION 1..31: note = synthetic construct
ANVACLDLGFQQGADTQRRFKLSDLSINSTE

SEQ ID NO: 42 - protein, 30 aa, synthetic construct
  REGION 1..30: note = synthetic construct
LPRSIPACVPWSPYLFQPNDTCIVSGWGRE

SEQ ID NO: 43 - protein, 26 aa, synthetic construct
  REGION 1..26: note = synthetic construct
KKCLAKKYTHLSCDKVFCQPWQRCIE

SEQ ID NO: 44 - protein, 28 aa, synthetic construct
  REGION 1..28: note = synthetic construct
LCCKACQGKGFHCKSGVCIPSQYQCNGE

SEQ ID NO: 45 - protein, 21 aa, synthetic construct
  REGION 1..21: note = synthetic construct
VKLVDQDKTMFICKSSWSMRE

SEQ ID NO: 46 - protein, 17 aa, synthetic construct
  REGION 1..17: note = synthetic construct
VKLISNCSKFYGNRFYE

SEQ ID NO: 47 - protein, 17 aa, synthetic construct
  REGION 1..17: note = synthetic construct
CLHPGTKFLNNGTCTAE

SEQ ID NO: 48 - protein, 15 aa, synthetic construct
  REGION 1..15: note = synthetic construct
NYNAGTYQNDIALIE

SEQ ID NO: 49 - protein, 15 aa, synthetic construct
  REGION 1..15: note = synthetic construct
GKFSVSLKHGNTDSE

SEQ ID NO: 50 - protein, 13 aa, synthetic construct
  REGION 1..13: note = synthetic construct
VGCAGFASVTQEE

SEQ ID NO: 51 - protein, 11 aa, synthetic construct
  REGION 1..11: note = synthetic construct
MKKDGNKKDCE

SEQ ID NO: 52 - protein, 9 aa, synthetic construct
  REGION 1..9: note = synthetic construct
YVDRIIFHE

SEQ ID NO: 53 - protein, 10 aa, synthetic construct
  REGION 1..10: note = synthetic construct
CLHVHCRGLE

SEQ ID NO: 54 - protein, 9 aa, synthetic construct
  REGION 1..9: note = synthetic construct
RVFSLQWGE

SEQ ID NO: 55 - protein, 9 aa, synthetic construct
  REGION 1..9: note = synthetic construct
ILTADMDAE

SEQ ID NO: 56 - protein, 8 aa, synthetic construct
  REGION 1..8: note = synthetic construct
KVTYTSQE

SEQ ID NO: 57 - protein, 7 aa, synthetic construct
  REGION 1..7: note = synthetic construct
VDCITGE

SEQ ID NO: 58 - protein, 6 aa, synthetic construct
  REGION 1..6: note = synthetic construct
NCGKPE

SEQ ID NO: 59 - protein, 5 aa, synthetic construct
  REGION 1..5: note = synthetic construct
TSLAE

SEQ ID NO: 60 - protein, 4 aa, synthetic construct
  REGION 1..4: note = synthetic construct
KDNE

SEQ ID NO: 61 - protein, 61 aa, synthetic construct
  REGION 1..61: note = synthetic construct
AKIRYYTYLIMNKGRLLKAGRQVREPGQDLVVLPLSITTDFIPSFRLVAYYTLIGASGQRE

SEQ ID NO: 62 - protein, 52 aa, synthetic construct
  REGION 1..52: note = synthetic construct
RSGIPIVTSPYQIHFTKTPKYFKPGMPFDLMVFVTNPDGSPAYRVPVAVQGE

SEQ ID NO: 63 - protein, 50 aa, synthetic construct
  REGION 1..50: note = synthetic construct
KVVLVSLQSGYLFIQTDKTIYTPGSTVLYRIFTVNHKLLPVGRTVMVNIE

SEQ ID NO: 64 - protein, 36 aa, synthetic construct
  REGION 1..36: note = synthetic construct
DTVQSLTQGDGVAKLSINTHPSQKPLSITVRTKKQE

SEQ ID NO: 65 - protein, 35 aa, synthetic construct
  REGION 1..35: note = synthetic construct
GDHGARVVLVAVDKGVFVLNKKNKLTQSKIWDVVE

SEQ ID NO: 66 - protein, 38 aa, synthetic construct
  REGION 1..38: note = synthetic construct
KADIGCTPGSGKDYAGVFSDAGLTFTSSSGQQTAQRAE

SEQ ID NO: 67 - protein, 29 aa, synthetic construct
  REGION 1..29: note = synthetic construct
QATRTMQALPYSTVGNSNNYLHLSVLRTE

SEQ ID NO: 68 - protein, 27 aa, synthetic construct
  REGION 1..27: note = synthetic construct
AHDAQGDVPVTVTVHDFPGKKLVLSSE

SEQ ID NO: 69 - protein, 26 aa, synthetic construct
  REGION 1..26: note = synthetic construct
GIPVKQDSLSSQNQLGVLPLSWDIPE

SEQ ID NO: 70 - protein, 26 aa, synthetic construct
  REGION 1..26: note = synthetic construct
VVADSVWVDVKDSCVGSLVVKSGQSE

SEQ ID NO: 71 - protein, 25 aa, synthetic construct
  REGION 1..25: note = synthetic construct
DLVGKSLYVSATVILHSGSDMVQAE

SEQ ID NO: 72 - protein, 23 aa, synthetic construct
  REGION 1..23: note = synthetic construct
KTVLTPATNHMGNVTFTIPANRE

SEQ ID NO: 73 - protein, 19 aa, synthetic construct
  REGION 1..19: note = synthetic construct
KGRNKFVTVQATFGTQVVE

SEQ ID NO: 74 - protein, 18 aa, synthetic construct
  REGION 1..18: note = synthetic construct
VVLSRKVLLDGVQNPRAE

SEQ ID NO: 75 - protein, 15 aa, synthetic construct
  REGION 1..15: note = synthetic construct
TLNVNFLLRMDRAHE

SEQ ID NO: 76 - protein, 14 aa, synthetic construct
  REGION 1..14: note = synthetic construct
LVNMGQWKIRAYYE

SEQ ID NO: 77 - protein, 15 aa, synthetic construct
  REGION 1..15: note = synthetic construct
SPMYSIITPNILRLE

SEQ ID NO: 78 - protein, 15 aa, synthetic construct
  REGION 1..15: note = synthetic construct
DRQPVPGQQMTLKIE

SEQ ID NO: 79 - protein, 14 aa, synthetic construct
  REGION 1..14: note = synthetic construct
VTITARFLYGKKVE

SEQ ID NO: 80 - protein, 13 aa, synthetic construct
  REGION 1..13: note = synthetic construct
GTAFVIFGIQDGE

SEQ ID NO: 81 - protein, 8 aa, synthetic construct
  REGION 1..8: note = synthetic construct
KFYYIYNE

SEQ ID NO: 82 - protein, 10 aa, synthetic construct
  REGION 1..10: note = synthetic construct
NSPQQVFSTE

SEQ ID NO: 83 - protein, 8 aa, synthetic construct
  REGION 1..8: note = synthetic construct
SLKRIPIE

SEQ ID NO: 84 - protein, 7 aa, synthetic construct
  REGION 1..7: note = synthetic construct
YVLPSFE

SEQ ID NO: 85 - protein, 7 aa, synthetic construct
  REGION 1..7: note = synthetic construct
QRISLPE

SEQ ID NO: 86 - protein, 8 aa, synthetic construct
  REGION 1..8: note = synthetic construct
LQCPQPAA

SEQ ID NO: 87 - protein, 7 aa, synthetic construct
  REGION 1..7: note = synthetic construct
VIVEPTE

SEQ ID NO: 88 - protein, 5 aa, synthetic construct
  REGION 1..5: note = synthetic construct
TMVLE

SEQ ID NO: 89 - protein, 5 aa, synthetic construct
  REGION 1..5: note = synthetic construct
LRPGE

SEQ ID NO: 90 - protein, 4 aa, synthetic construct
  REGION 1..4: note = synthetic construct
FKSE

SEQ ID NO: 91 - protein, 20 aa, synthetic construct
  REGION 1..20: note = synthetic construct
NYNIALRWTAKQKLYLRTGE

SEQ ID NO: 92 - protein, 19 aa, synthetic construct
  REGION 1..19: note = synthetic construct
YNFVSPSKSFWTRITCAEE

SEQ ID NO: 93 - protein, 21 aa, synthetic construct
  REGION 1..21: note = synthetic construct
KGWSPTPRCIRVRTCSKSDIE

SEQ ID NO: 94 - protein, 18 aa, synthetic construct
  REGION 1..18: note = synthetic construct
NGYNQNYGRKFVQGNSTE

SEQ ID NO: 95 - protein, 18 aa, synthetic construct
  REGION 1..18: note = synthetic construct
QVKPCDFPDIKHGGLFHE

SEQ ID NO: 96 - protein, 22 aa, synthetic construct
  REGION 1..22: note = synthetic construct
FMCKLGYNANTSILSFQAVCRE

SEQ ID NO: 97 - protein, 8 aa, synthetic construct
  REGION 1..8: note = synthetic construct
YQCQPYYE

SEQ ID NO: 98 - protein, 20 aa, synthetic construct
  REGION 1..20: note = synthetic construct
NSRAKSNGMRFKLHDTLDYE

SEQ ID NO: 99 - protein, 14 aa, synthetic construct
  REGION 1..14: note = synthetic construct
DGWSHFPTCYNSSE

SEQ ID NO: 100 - protein, 14 aa, synthetic construct
  REGION 1..14: note = synthetic construct
ISYGNTTGSIVCGE

SEQ ID NO: 101 - protein, 22 aa, synthetic construct
  REGION 1..22: note = synthetic construct
FMCKLGYNANTSVLSFQAVCRE

SEQ ID NO: 102 - protein, 18 aa, synthetic construct
  REGION 1..18: note = synthetic construct
GTLCDFPKIHHGFLYDEE

SEQ ID NO: 103 - protein, 18 aa, synthetic construct
  REGION 1..18: note = synthetic construct
YAMIGNNMITCINGIWTE

SEQ ID NO: 104 - protein, 18 aa, synthetic construct
  REGION 1..18: note = synthetic construct
YGYVQPSVPPYQHGVSVE

SEQ ID NO: 105 - protein, 18 aa, synthetic construct
  REGION 1..18: note = synthetic construct
GDTVQIICNTGYSLQNNE

SEQ ID NO: 106 - protein, 16 aa, synthetic construct
  REGION 1..16: note = synthetic construct
IVCKDGRWQSLPRCVE

SEQ ID NO: 107 - protein, 12 aa, synthetic construct
  REGION 1..12: note = synthetic construct
DYNPFSQVPTGE

SEQ ID NO: 108 - protein, 10 aa, synthetic construct
  REGION 1..10: note = synthetic construct
QVKTCGYIPE

SEQ ID NO: 109 - protein, 10 aa, synthetic construct
  REGION 1..10: note = synthetic construct
ANVDAQPKKE

SEQ ID NO: 110 - protein, 9 aa, synthetic construct
  REGION 1..9: note = synthetic construct
WTTLPTCVE

SEQ ID NO: 111 - protein, 8 aa, synthetic construct
  REGION 1..8: note = synthetic construct
KVAVLCKE

SEQ ID NO: 112 - protein, 25 aa, synthetic construct
  REGION 1..25: note = synthetic construct
SNTGSTTGSIVCGYNGWSDLPICYE

SEQ ID NO: 113 - protein, 23 aa, synthetic construct
  REGION 1..23: note = synthetic construct
NGWSPTPRCIRVKTCSKSSIDIE

SEQ ID NO: 114 - protein, 21 aa, synthetic construct
  REGION 1..21: note = synthetic construct
LPKIDVHLVPDRKKDQYKVGE

SEQ ID NO: 115 - protein, 21 aa, synthetic construct
  REGION 1..21: note = synthetic construct
YYCNPRFLMKGPNKIQCVDGE

SEQ ID NO: 116 - protein, 20 aa, synthetic construct
  REGION 1..20: note = synthetic construct
NYNIALRWTAKQKLYSRTGE

SEQ ID NO: 117 - protein, 20 aa, synthetic construct
  REGION 1..20: note = synthetic construct
KWSHPPSCIKTDCLSLPSFE

SEQ ID NO: 118 - protein, 18 aa, synthetic construct
  REGION 1..18: note = synthetic construct
HGWAQLSSPPYYYGDSVE

SEQ ID NO: 119 - protein, 18 aa, synthetic construct
  REGION 1..18: note = synthetic construct
ISHGVVAHMSDSYQYGEE

SEQ ID NO: 120 - protein, 15 aa, synthetic construct
  REGION 1..15: note = synthetic construct
FDHNSNIRYRCRGKE

SEQ ID NO: 121 - protein, 16 aa, synthetic construct
  REGION 1..16: note = synthetic construct
ITCKDGRWQSIPLCVE

SEQ ID NO: 122 - protein, 15 aa, synthetic construct
  REGION 1..15: note = synthetic construct
GWIHTVCINGRWDPE

SEQ ID NO: 123 - protein, 16 aa, synthetic construct
  REGION 1..16: note = synthetic construct
KAKYQCKLGYVTADGE

SEQ ID NO: 124 - protein, 15 aa, synthetic construct
  REGION 1..15: note = synthetic construct
TTCYMGKWSSPPQCE

SEQ ID NO: 125 - protein, 13 aa, synthetic construct
  REGION 1..13: note = synthetic construct
SYAHGTKLSYTCE

SEQ ID NO: 126 - protein, 11 aa, synthetic construct
  REGION 1..11: note = synthetic construct
RVRYQCRSPYE

SEQ ID NO: 127 - protein, 15 aa, synthetic construct
  REGION 1..15: note = synthetic construct
GFGIDGPAIAKCLGE

SEQ ID NO: 128 - protein, 12 aa, synthetic construct
  REGION 1..12: note = synthetic construct
HGTINSSRSSQE

SEQ ID NO: 129 - protein, 10 aa, synthetic construct
  REGION 1..10: note = synthetic construct
YQCQNLYQLE

SEQ ID NO: 130 - protein, 11 aa, synthetic construct
  REGION 1..11: note = synthetic construct
WTTLPVCIVEE

SEQ ID NO: 131 - protein, 11 aa, synthetic construct
  REGION 1..11: note = synthetic construct
KIPCSQPPQIE

SEQ ID NO: 132 - protein, 9 aa, synthetic construct
  REGION 1..9: note = synthetic construct
SQYTYALKE

SEQ ID NO: 133 - protein, 10 aa, synthetic construct
  REGION 1..10: note = synthetic construct
QVQSCGPPPE

SEQ ID NO: 134 - protein, 9 aa, synthetic construct
  REGION 1..9: note = synthetic construct
KKDVYKAGE

SEQ ID NO: 135 - protein, 9 aa, synthetic construct
  REGION 1..9: note = synthetic construct
GLPCKSPPE

SEQ ID NO: 136 - protein, 8 aa, synthetic construct
  REGION 1..8: note = synthetic construct
KVSVLCQE

SEQ ID NO: 137 - protein, 7 aa, synthetic construct
  REGION 1..7: note = synthetic construct
HLKNKKE

SEQ ID NO: 138 - protein, 8 aa, synthetic construct
  REGION 1..8: note = synthetic construct
GGFRISEE

SEQ ID NO: 139 - protein, 8 aa, synthetic construct
  REGION 1..8: note = synthetic construct
LLNGNVKE

SEQ ID NO: 140 - protein, 7 aa, synthetic construct
  REGION 1..7: note = synthetic construct
YPTCAKR

SEQ ID NO: 141 - protein, 8 aa, synthetic construct
  REGION 1..8: note = synthetic construct
STCGDIPE

SEQ ID NO: 142 - protein, 349 aa, synthetic construct
  REGION 1..349: note = synthetic construct
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 143 - protein, 47 aa, synthetic construct
  REGION 1..47: note = synthetic construct
EGVQKEDIPPADLSDQVPDTESETRILLQGTPVAQMTEDAVDAERLK

SEQ ID NO: 144 - protein, 302 aa, synthetic construct
  REGION 1..302: note = synthetic construct
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 145 - protein, 194 aa, Homo sapiens
  SITE 111: note = misc_feature - Xaa can be any naturally occurring amino acid
  SITE 157: note = misc_feature - Xaa can be any naturally occurring amino acid
  SITE 160: note = misc_feature - Xaa can be any naturally occurring amino acid
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNXAHWSTKPPICQRIPCGLPPTIANGDFISTNRENFHYGSVVTYRCNXGSXGRKVFELVGEPSIYCTSNDDQVGIWSGPAPQCII

SEQ ID NO: 146 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 147 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 148 - protein, 388 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 149 - protein, 235 aa, Homo sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVG

SEQ ID NO: 150 - protein, 246 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 151 - protein, 194 aa, Homo sapiens; identical to SEQ ID NO: 146

SEQ ID NO: 152 - protein, 194 aa, Homo sapiens; identical to SEQ ID NO: 147

SEQ ID NO: 153 - protein, 342 aa, synthetic construct
  REGION 1..342: note = synthetic construct
MKGKFLKVSSLFVATLTTATLVSSPAANALSSKAMDNHPQQTQSSKQQTPKIKKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 154 - protein, 274 aa, synthetic construct
  REGION 1..274: note = synthetic construct
VILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 155 - protein, 12 aa, synthetic construct
  REGION 1..12: note = synthetic construct
VGCAGFASVTQE

SEQ ID NO: 156 - protein, 14 aa, synthetic construct
  REGION 1..14: note = synthetic construct
LRRQHARASHLGLA

SEQ ID NO: 157 - protein, 5 aa, synthetic construct
  REGION 1..5: note = synthetic construct
SASLL

SEQ ID NO: 158 - DNA, 41 bp, Mus musculus
  source 1..41: mol_type = unassigned DNA
tggtctctgtgagaaagttcgccctcgcaatagtagatact

SEQ ID NO: 159 - DNA, 41 bp, Mus musculus
  source 1..41: mol_type = unassigned DNA
agtatctactattgcgagggcgaactttctcacagagacca

SEQ ID NO: 160 - DNA, 21 bp, Homo sapiens
  source 1..21: mol_type = unassigned DNA
aggtatgacaacgaatttggc

SEQ ID NO: 161 - DNA, 17 bp, Homo sapiens
  source 1..17: mol_type = unassigned DNA
gtgtggcagggactccc

SEQ ID NO: 162 - DNA, 24 bp, Homo sapiens
  source 1..24: mol_type = unassigned DNA
agcagactgctggtggaggacatg

SEQ ID NO: 163 - DNA, 24 bp, Homo sapiens
  source 1..24: mol_type = unassigned DNA
ttcacacaggcgtcataagcttcc

SEQ ID NO: 164 - DNA, 20 bp, Homo sapiens
  source 1..20: mol_type = unassigned DNA
gaatctggatggcgtccgtt

SEQ ID NO: 165 - DNA, 23 bp, Homo sapiens
  source 1..23: mol_type = unassigned DNA
tggtacgtgatttcatctccagt

SEQ ID NO: 166 - DNA, 20 bp, Homo sapiens
  source 1..20: mol_type = unassigned DNA
ggaagcaccactggttccat

SEQ ID NO: 167 - DNA, 21 bp, Homo sapiens
  source 1..21: mol_type = unassigned DNA
tggtctttcttgcgatcagga

SEQ ID NO: 168 - DNA, 20 bp, Homo sapiens
  source 1..20: mol_type = unassigned DNA
cctggctacgctcttccaaa

SEQ ID NO: 169 - DNA, 20 bp, Homo sapiens
  source 1..20: mol_type = unassigned DNA
agggtaaagctgacacggat

SEQ ID NO: 170 - DNA, 20 bp, Mus musculus
  source 1..20: mol_type = unassigned DNA
tgggagttgctgttgaagtc

SEQ ID NO: 171 - DNA, 20 bp, Mus musculus
  source 1..20: mol_type = unassigned DNA
tgacatcaagaaggtggtga

SEQ ID NO: 172 - DNA, 24 bp, Mus musculus
  source 1..24: mol_type = unassigned DNA
agcagactgctggtggaggacatg

SEQ ID NO: 173 - DNA, 24 bp, Mus musculus
  source 1..24: mol_type = unassigned DNA
ttcacacaggcgtcataagcttcc

SEQ ID NO: 174 - RNA, 21 nt, Homo sapiens
  modified_base 3..4: mod_base = OTHER; note = thymine
  modified_base 6: mod_base = OTHER; note = thymine
  modified_base 13: mod_base = OTHER; note = thymine
  modified_base 15: mod_base = OTHER; note = thymine
  modified_base 17: mod_base = OTHER; note = thymine
  modified_base 20..21: mod_base = OTHER; note = thymine
  source 1..21: mol_type = unassigned RNA
gcttatgacgcctgtgtgatt

SEQ ID NO: 175 - RNA, 21 nt, Homo sapiens
  source 1..21: mol_type = unassigned RNA
  modified_base 1: mod_base = OTHER; note = thymine
  modified_base 12: mod_base = OTHER; note = thymine
  modified_base 15: mod_base = OTHER; note = thymine
  modified_base 20..21: mod_base = OTHER; note = thymine
tcacacaggcgtcataagctt